(19) United States
(12) Patent Application Publication
(10) Pub. No.: US 2024/0318159 A1
(43) Pub. Date: Sep. 26, 2024

(54) CONTEXT-DEPENDENT, DOUBLE-STRANDED DNA-SPECIFIC DEAMINASES AND USES THEREOF

(71) Applicant: MITOLAB INC., Allston, MA (US)

(72) Inventors: Fahim Farzadfard, Boston, MA (US); Nava Gharaei, Boston, MA (US); Giyoung Jung, Cambridge, MA (US); Leanne Lin, Cambridge, MA (US); Jeong Seuk Kang, Cambridge, MA (US)

(21) Appl. No.: 18/271,625

(22) PCT Filed: Jan. 12, 2022
(86) PCT No.: PCT/US2022/012204

§ 371 Date: Jul. 10, 2023

Related U.S. Application Data
(60) Provisional application No. 63/136,524, filed on Jan. 12, 2021.

(51) Int. Cl. C12N 9/78; C12N 15/10

(52) U.S. Cl. CPC C12N 9/78; C12N 15/102; C07K 2319/80

(57) ABSTRACT

Deaminase domains that are capable of deaminating cytosine nucleotides in double-stranded DNA in a context-dependent manner are described. Also disclosed are non-naturally occurring or engineered targeted base editors containing the deaminase domains in combination with one or more targeting domains (e.g., Cas9, Cpf1, ZF, TALE) that recognize and/or bind a specific target sequence. The base editors facilitate specific and efficient editing of targeted sites within the genome of a cell or subject, e.g., within the human mitochondrial genome, with low off-target effects. Methods of using the deaminase domains and base editors are also provided.

### CROSS-REFERENCE TO RELATED APPLICATIONS

This application claims the benefit of and priority to U.S. Application No. 63/136,524 filed Jan. 12, 2021, the contents of which is incorporated by reference in its entirety.

### REFERENCE TO SEQUENCE LISTING

The Sequence Listing submitted Jan. 12, 2022, as a text file named “MILA100_ST25.txt,” created on Jan. 12, 2022, and having a size of 374,384 bytes is hereby incorporated by reference pursuant to 37 C.F.R. § 1.52(e)(5).

### FIELD OF THE INVENTION

The disclosed invention generally relates to compositions and methods for targeting and editing nucleic acids, in particular programmable deamination at a target sequence of interest.

### BACKGROUND OF THE INVENTION

Targeted editing of nucleic acid sequences, for example, the targeted cleavage or the targeted introduction of a specific modification into genomic DNA, is a highly promising approach for the study of gene function and also has the potential to provide new therapies for human genetic diseases. Current genome engineering tools, including engineered zinc finger nucleases (ZFNs), transcription activator like effector nucleases (TALENs), and the CRISRPR-Cas system, effect sequence-specific DNA cleavage in a genome. This programmable cleavage can result in mutation of the DNA at the cleavage site via non-homologous end joining (NHEJ) or replacement of the DNA surrounding the cleavage site via homology-directed repair (HDR). However, a drawback to these technologies is that that they typically result in modest gene editing efficiencies as well as unwanted gene alterations that can compete with the desired alteration.

Since many genetic diseases in principle can be treated by effecting a specific nucleotide change at a specific location in the genome (for example, a C to T change in a specific codon of a gene associated with a disease), base editors have been contemplated as a programmable way to achieve such precision gene editing without the need for introduction of double stranded DNA (dsDNA) breaks. Because previously described (cytidine or adenosine) deaminases act on single-stranded nucleic acids, their use in base editing requires the unwinding of double-stranded DNA (dsDNA)-for example by Cas9 system or similar RNA-guided enzymes. Thus, existing base-editors use a DNA-modifying domain (i.e. a ssDNA-specific deaminase domain) fused to Cas9 or other RNA-guided enzymes. Since the binding of Cas9 enzyme with its guide-RNA to a genomic target results in the generation of an R-loop that exposes a single-stranded DNA region, base-editors modify bases within a small window defined by the exposed ssDNA region. Base-editors that use cytidine deaminases have enabled C->T mutations (Komor, A., et al., Nature 533, 420-424 (2016)), and base-editors fused to adenosine deaminases have allowed for A->G mutations (Gaudelli, N., et al., Nature 551, 464-471 (2017)). However, due to strict requirement for ssDNA as their substrate, efforts to utilize the ssDNA-specific deaminases in combination with dsDNA-specific DNA binding domains such as Zinc Fingers and TALEs have not resulted in efficient base editors.

Recently, a cytidine deaminase with double-stranded DNA activity that enabled mitochondrial genome editing was reported (Mok B Y., et al., Nature, 583(7817):631-637 (2020); WO 2021/155065A1). This cytidine deaminase, named DddA, creates C->U conversions on double stranded DNA, which is then converted to C->T by the cellular repair and replication machinery. However, DddA has a strict context specificity and can only edit deoxycytidines that precede with a Thymine (thus converting TC to TT) which limits its applicability to very narrow sequence contexts. Thus, despite much progress, there is an ongoing need for compositions, systems, and methods to expand current base editing capabilities, especially in organelles such as mitochondria that are not amenable to editing by RNA-guided editors.

Therefore, it is an object of the invention to provide compositions and methods for nucleic acid editing.

It is an object of the invention to provide compositions and methods that enable base editing of dsDNA without the requirement for unwinding of DNA or reliance on any accessory nucleic acid moiety (e.g., guide RNA) for its function.

It is an object of the invention to provide compositions and methods that enable introduction of a desired modification (e.g., base edit) of cytidines in dsDNA with high efficiency in any given sequence context (e.g., NACN, NCCN, NGCN, NTCN).

It is an object of the invention to provide compositions and methods that enable nucleic acid base editing with minimal off-target activity.

It is another object of the invention to provide compositions and methods that enable nucleic acid base editing with improved precision.

It is another object of the invention to provide compositions and methods that enable tuning the window of activity of the base editor to maximize on-target editing and minimize by-stander off-targets.

It is another object of the invention to provide compositions and methods that enable nucleic acid base editing across a broad range of target nucleic acids.

It is another object of the invention to provide compositions and methods for nucleic acid base editing at any site in the human (nuclear or mitochondrial) genome.

It is another object of the invention to provide compositions and methods for nucleic acid editing of dsDNA in vitro for applications including diversity generation and epigenetic sequencing.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### BRIEF SUMMARY OF THE INVENTION

Deaminase domains that are capable of deaminating cytosine in double-stranded DNA have been discovered. Some of the disclosed deaminase domains are more sequence specific while others can edit a broader range of target sequences (i.e., possess broader context-specificity) than previously characterized deaminases. Based on these and other features, the deaminases are believed to exhibit reduced off-target editing and/or enable introducing edits in broader contexts as compared with previously characterized dsDNA-specific deaminase. Reagents, compositions, kits and methods for targeting and editing nucleic acids, including editing a single target site within the genome of a cell or subject, using the deaminase domains are provided.

In particular, disclosed is an isolated deaminase domain that can deaminate double-stranded DNA. The deaminase domain can have greater deaminase activity on double-stranded DNA containing a target nucleotide sequence as compared to the deaminase activity of the deaminase domain on double-stranded DNA that does not contain the target nucleotide sequence. Typically, the target nucleotide sequence contains two or more target nucleotides each of which are individually fully or partially defined, and are in a fixed sequential relationship to each other. In some forms, the target nucleotide sequence contains two or more target nucleotides, wherein the target nucleotides are each individually fully or partially defined and are in a fixed sequential relationship to each other.

In some forms, the deaminase context specificity can be represented as a probability sequence logo wherein heterogeneity in the context of the target nucleotides edited at a certain threshold (e.g., 25% or 50%) by the deaminase is represented with a group of aligned sequences. The alignment is depicted as a stack of letters present at a given position, and the observed frequency of each nucleic acid in the alignment is represented by the height of each letter in a stack.

In preferred forms, the deaminase domain is not the deaminase domain of DddA from Burkholderia cenocepacia. In some forms, the deaminase domain is not the deaminase domain of a homolog of DddA from Burkholderia cenocepacia. In some forms, the deaminase domain is not the deaminase domain of DddA from Burkholderia.

In some forms, the deaminase domain can be split into two portions whereby the deaminase domain is only capable of deaminating the target nucleotide sequence when the two portions are brought into proximity or combined together. This is useful for preventing deaminase activity except where the targeting domains bring the deaminase portions into proximity near the target sequence. In some forms each portion of a split deaminase domain includes more than 50% of the intact deaminase domain, such that the combined portions includes two copies of at least some parts of the deaminase domain. In some forms, each portion of a split deaminase domain includes at least 55%, 60%, 65%, 70%, 75%, 80%, 85%, 90%, 95% or more than 95% of the intact deaminase domain. In other forms, each portion of a split deaminase domain includes exactly 50% of the intact deaminase domain, such that combination of the two portions provides exactly 100% of the structural components of a deaminase domain. Typically, the two portions of a split deaminase domain are brought into proximity of each other by one or more accessory domains.

In some forms, the deaminase domain can deaminate cytosine nucleotides (hereby referred to as “cytosine deaminase”). Exemplary target nucleotide sequences in which a cytosine nucleotide can be deaminated include, without limitation, AC, CC, GC, TC in any given context. The target nucleotide sequences can been usefully shown as the dominate sequence by frequency sequence logo analysis. In some forms of the foregoing, the 3′ end C is deaminated. Exemplary cytosine deaminases include deaminase domains having the amino acid sequence of any one of SEQ ID NO:1, SEQ ID NO:2, SEQ ID NO:3, SEQ ID NO:4, SEQ ID NO:9, SEQ ID NO:11, SEQ ID NO:14, SEQ ID NO:15, and SEQ ID NO:16.

In some forms, the deaminase domain can deaminate adenine nucleotides (herein referred to as “adenosine deaminase”).

In some forms, the deaminase domain includes BE_R1_11, having an amino acid sequence of SEQ ID NO:1, or an amino acid having at least 70%, 75%, 80%, 85%, 90%, 95%, or 99% sequence identity to SEQ ID NO:1, or fragment thereof. In some forms, the deaminase domain includes BE_R1_12, having an amino acid sequence of SEQ ID NO:2, or an amino acid having at least 70%, 75%, 80%, 85%, 90%, 95%, or 99% sequence identity to SEQ ID NO:2, or fragment thereof. In some forms, the deaminase domain includes BE_R1_28, having an amino acid sequence of SEQ ID NO:3, or an amino acid having at least 70%, 75%, 80%, 85%, 90%, 95%, or 99% sequence identity to SEQ ID NO:3, or fragment thereof.

Targeted base editors including a deaminase domain and a targeting domain, That specifically binds to a base editor target sequence are also described. Exemplary targeting domains include a TALE, BAT, CRISPR-Cas9, Cfp1, and Zinc finger.

In some forms, the targeted base editor target sequence is selected to be present in a target nucleic acid within 20 nucleotides of an instance of the target nucleotide sequence of the deaminase domain, wherein the instance of the target nucleotide sequence is selected to be base edited by the targeted base editor. In some forms, the base editor target sequence within 30 nucleotides of the instance of the target nucleotide sequence selected to be base edited by the targeted base editor is the only base editor target sequence in the target nucleic acid that is within 20 nucleotides of any instance of target nucleotide sequence. In some forms, the instance of the target nucleotide sequence in the target nucleic acid is the only instance of the target nucleotide sequence of the deaminase domain within 20 nucleotides of the base editor target sequence in the target nucleic acid within 20 nucleotides of the instance of the target nucleotide sequence.

In any of the foregoing, the base editor target sequence can be present in mitochondrial DNA, or chloroplast DNA, or plastid DNA, or any other membranous organelle with a genome. The base editor can also be used in vitro to act on, for example, synthetic or natural DNA in a test tube.

In some forms, the base editor includes two portions whereby the first portion includes a first split deaminase domain, and the second portion includes a second split deaminase domain. In some forms, the first portion includes a split deaminase domain including an amino acid sequence of any one of SEQ ID NOs:122-181, and the second portion includes a split deaminase domain including an amino acid sequence of any one of SEQ ID Nos:127-181, where the first and second split deaminase domains are inactive alone but are capable of deamination when brought into proximity together. In some forms, the first split deaminase domain includes an amino acid sequence of any one of SEQ ID Nos:122-126. In other forms. both the first and second split deaminase domains include a wild-type deaminase domain active site.

In certain forms, the first and second split deaminase domains each include a fragment or variant of BE_R1_11. For example, in some forms, the first split deaminase domain includes any one of SEQ ID NOs:122, or 127-135, or 150, and the second split deaminase domain includes any one of SEQ ID NOs:127-135 or 150. In some forms, the first split deaminase domain includes SEQ ID NO:122, and the second split deaminase domain includes any one of SEQ ID NOs:127-134 or 150. In a particular form, the first split deaminase domain includes SEQ ID NO:129, and the second split deaminase domain includes SEQ ID NO:150.

In certain forms, the first and second split deaminase domains each include a fragment or variant of BE_R1_12. For example, in some forms, the first split deaminase domain includes any one of SEQ ID NOs:124, or 136-140, or 156-167, and
the second split deaminase domain includes any one of SEQ ID NOs:136-140, or 156-167. In some forms, the first split deaminase domain includes SEQ ID NO:124, and the second split deaminase domain includes any one of SEQ ID NOs:156-166. In a particular form, the first split deaminase domain includes SEQ ID NO:137, and
the second split deaminase domain includes SEQ ID NO:142. In another form, the first split deaminase domain includes SEQ ID NO:139, and the second split deaminase domain includes SEQ ID NO:144.

In certain forms, the first and second split deaminase domains each include a fragment or variant of BE_R1_41. For example, in some forms, the first split deaminase domain includes any one of SEQ ID NOs:168-171, and the second split deaminase domain includes any one of SEQ ID Nos: 172-175. In particular forms, the first split deaminase domain includes SEQ ID NO:168, and the second split deaminase domain includes SEQ ID NO:173. In another form, the first split deaminase domain includes SEQ ID NO:171, and the second split deaminase domain includes SEQ ID NO:175. In other forms, the first split deaminase domain includes SEQ ID NO:171, and the second split deaminase domain includes SEQ ID NO:173.

In certain forms, the first and second split deaminase domains each include a fragment or variant of BE_R1_28. For example, in some forms, the first split deaminase domain includes any one of SEQ ID NOs:123, or 146-149, or 151-155, and the second split deaminase domain includes any one of SEQ ID NOs:146-149, or 151-155. In particular forms, the first split deaminase domain includes SEQ ID NO:123, and the second split deaminase domain includes any one of SEQ ID NOs:149, or 151-153.

In certain forms, the first and second split deaminase domains each include a fragment or variant of BE_R4_21. For example, in some forms, the first split deaminase domain includes any one of SEQ ID NOs:125, or 176-177, and the second split deaminase domain includes any one of SEQ ID NOs:176-177. In particular forms, the first split deaminase domain includes SEQ ID NO:125, and the second split deaminase domain includes SEQ ID NO:177. In other forms, the first split deaminase domain includes SEQ ID NO:176, and the second split deaminase domain includes SEQ ID NO:177.

In certain forms, the first and second split deaminase domains each include a fragment or variant of BE_R2_11. For example, in some forms, the first split deaminase domain includes any one of SEQ ID NOs:126, or 180-181, and the second split deaminase domain includes any one of SEQ ID NOs:180-181. In particular forms, the first split deaminase domain includes SEQ ID NO:125, and the second split deaminase domain includes any one of SEQ ID NOs:180-181. In another form, the first split deaminase domain includes SEQ ID NO:180, and the second split deaminase domain includes SEQ ID NO:181.

Other deaminases can be split in analogous ways to produce analogous results. Further, other splits and edits can also be used to achieve the purpose of keeping the deaminases portions inactive until brought into proximity.

In some forms, the first, or the second portion, or both the first and second portions includes a programmable DNA binding domain selected from a TALE, BAT, CRISPR-Cas9, Cfp1, or Zinc finger.

For example, in some forms, one programmable DNA binding domain is a TALE selected from the group consisting of a Left hand side TALE and a Right hand side TALE. The use of the terms “Left” and “Right” are used only for convenience and do not connote on which side of the target sequence the DNA binding domain binds. Further, different classes of DNA binding domains (e.g., TALE and ZF, ZF and TALE, BAT and TALE, dCas9 and TALE) can be used together. In an exemplary form, one programmable DNA binding domain is a Left hand side TALE including an amino acid sequence of any one of SEQ ID NOs:90, 92, 95, 97-106. In another exemplary form, one programmable DNA binding domain is a Right hand side TALE including an amino acid sequence of any one of SEQ ID NOs:91, 93-94, 96, 108-113. In some forms, one or more programmable DNA binding domain is TALE that binds to mitochondrial mND1 DNA, having an amino acid sequence including any one of SEQ ID NOS:95-96. Therefore, in a particular form, one programmable DNA binding domain is a Right hand side TALE that binds to mitochondrial mND1 DNA, having an amino acid sequence including SEQ ID NO:96. In another particular form one programmable DNA binding domain is a Left hand side TALE that binds to mitochondrial hND1 DNA, having an amino acid sequence including SEQ ID NO:95. In some forms, one or more programmable DNA binding domain is a TALE that binds to mitochondrial mCOX1 DNA, having an amino acid sequence including any one of SEQ ID NOs:99-106, or 108-113. For example, in some forms, one programmable DNA binding domain is a Right hand side TALE that binds to mitochondrial mCOX1 DNA, having an amino acid sequence including any one of SEQ ID NOs:108-113. In some forms, one programmable DNA binding domain is a Left hand side TALE that binds to mitochondrial mCOX1 DNA, having an amino acid sequence including any one of SEQ ID NOs:90-106. In other forms, one or more programmable DNA binding domain is TALE that binds to h12 DNA, having an amino acid sequence including SEQ ID NO:98. In other forms, one programmable DNA binding domain is a TALE with NT(G)N-terminal domain, having an amino acid sequence including SEQ ID NO:114. In some forms, one programmable DNA binding domain is a TALE with NT(bn)N-terminal domain, having an amino acid sequence including SEQ ID NO:115. In other forms, one or more programmable DNA binding domain is TALE that binds to the mitochondrial ND6 DNA, having an amino acid sequence including any one of SEQ ID NOs:92-94. In some forms, one programmable DNA binding domain is a Right hand side TALE that binds to the mitochondrial ND6 DNA, having an amino acid sequence including any one of SEQ ID NOs:93-94. In some forms, one programmable DNA binding domain is a Left hand side TALE that binds to mitochondrial mND6 DNA, having an amino acid sequence including SEQ ID NO:92. In other forms, one or more programmable DNA binding domain is TALE that binds to mitochondrial hND DNA, having an amino acid sequence including any one of SEQ ID NOs:90-91. For example, in some forms, one programmable DNA binding domain is a Right hand side TALE that binds to mitochondrial hND DNA, having an amino acid sequence including SEQ ID NO:90. In some forms, one programmable DNA binding domain is a Left hand side TALE that binds to mitochondrial hND DNA, having an amino acid sequence including SEQ ID NO:91. In other forms, one programmable DNA binding domain is a TALE that binds to h11 DNA, having an amino acid sequence including SEQ ID NO:97. The programmable DNA binding domains can be designed to target any desired target sequence.

In some forms, one or both of the first and second portions independently comprise a zinc finger programmable DNA binding domain. For example, in some forms, one programmable DNA binding domain is a zinc finger selected from Left hand side zinc finger and a Right hand side zinc finger. In exemplary forms one programmable DNA binding domain is a zinc finger that binds to mitochondrial mCOX1 DNA, having an amino acid sequence including any one of SEQ ID NOs:82-89. In some forms, one programmable DNA binding domain is a Right hand side zinc finger that binds to mitochondrial mCOX1 DNA, having an amino acid sequence of any one of SEQ ID NOS:82-86, or 87-89. In some forms, one programmable DNA binding domain is a Left hand side zinc finger that binds to mitochondrial mCOX1 DNA, having an amino acid sequence including any one of SEQ ID NOs:82-86. In other forms, one programmable DNA binding domain is a zinc finger that binds to hND DNA, having an amino acid sequence including any one of SEQ ID NOs:74-81. For example, in some forms one programmable DNA binding domain is a Right hand side zinc finger that binds to hND DNA, having an amino acid sequence of any one of SEQ ID NOs:78-81. In some forms, one programmable DNA binding domain is a Left hand side zinc finger that binds to hND DNA, having an amino acid sequence including any one of SEQ ID NOs:74-77.

In some forms, one or both of the first and second portions independently comprise a BAT programmable DNA binding domain. For example, in some forms, one programmable DNA binding domain is a BAT selected from the group consisting of a Left hand side BAT and a Right hand side BAT. In some forms, one programmable DNA binding domain is a BAT that binds to mCOX1 DNA, having an amino acid sequence including any one of SEQ ID NOs:118-119. In some forms, one programmable DNA binding domain is a Right hand side BAT that binds to mCOX1 DNA, having an amino acid sequence of any one of SEQ ID NO:119. In some forms, one programmable DNA binding domain is a Left hand side BAT that binds to mCOX1 DNA, having an amino acid sequence including any one of SEQ ID NO:118. In some forms, one programmable DNA binding domain is a BAT that binds to ND6 DNA, having an amino acid sequence including any one of SEQ ID NOs:120-121. In some forms, one programmable DNA binding domain is a Right hand side BAT that binds to hND DNA, having an amino acid sequence of any one of SEQ ID NO:121. In some forms, one programmable DNA binding domain is a Left hand side BAT that binds to hND DNA, having an amino acid sequence including any one of SEQ ID NO:120.

In exemplary forms, the first portion of a targeted DNA editor includes a first split deaminase domain including an amino acid sequence of SEQ ID NO:120, and a Left hand TALE programmable DNA binding domain, whereby the second portion includes a second split deaminase domain including an amino acid sequence of any one of SEQ ID NOs:156, 158, 160 or 164, and a Right hand TALE programmable DNA binding domain.

In exemplary forms, the first portion of a targeted DNA editor includes a first split deaminase domain including an amino acid sequence of SEQ ID NO:169, and a Left hand TALE programmable DNA binding domain; whereby the second portion includes a second split deaminase domain including an amino acid sequence of any one of SEQ ID NOs:173, or 175, and a Right hand TALE programmable DNA binding domain.

In exemplary forms, the first portion of a targeted DNA editor includes a first split deaminase domain including an amino acid sequence of SEQ ID NO:171, and a Left hand TALE programmable DNA binding domain; whereby the second portion includes a second split deaminase domain including an amino acid sequence of any one of SEQ ID NO:175, and a Right hand TALE programmable DNA binding domain.

In exemplary forms, the first portion of a targeted DNA editor includes a first split deaminase domain including an amino acid sequence of a first split deaminase domain including an amino acid sequence of SEQ ID NO:169, and a Left hand BAT programmable DNA binding domain; whereby the second portion includes a second split deaminase domain including an amino acid sequence of any one of SEQ ID NOs:173, or 175, and a Right hand TALE programmable DNA binding domain.

In exemplary forms, the first portion of a targeted DNA editor includes a first split deaminase domain including a first split deaminase domain including an amino acid sequence of SEQ ID NO:169, and a first coiled coil domain, and optionally a Left hand TALE programmable DNA binding domain, whereby the second portion includes (d) a second split deaminase domain including an amino acid sequence of any one of SEQ ID NOs:173, or 175, and a second coiled coil domain, optionally a Right hand TALE programmable DNA binding domain, whereby the first and second coiled coil domains interact together upon combination of the first and second portions.

In some forms, the first and second portions each comprise a programmable DNA binding domain independently selected from the group consisting of a TALE, BAT, CRISPR-Cas9, Cfp1, and Zinc finger. In some forms, the first portion is a TALE and the second portion is a TALE, the first portion is a TALE and the second portion is a BAT, the first portion is a TALE and the second portion is a Zinc finger, the first portion is a TALE and the second portion is a CRISPR-Cas9, the first portion is a TALE and the second portion is a Cfp1, the first portion is a BAT and the second portion is a TALE, the first portion is a BAT and the second portion is a BAT, the first portion is a BAT and the second portion is a Zinc finger, the first portion is a BAT and the second portion is a CRISPR-Cas9, the first portion is a BAT and the second portion is a Cfp1, the first portion is a Zinc finger and the second portion is a TALE, the first portion is a Zinc finger and the second portion is a BAT, the first portion is a Zinc finger and the second portion is a Zinc finger, the first portion is a Zinc finger and the second portion is a CRISPR-Cas9, the first portion is a Zinc finger and the second portion is a Cfp1, the first portion is a CRISPR-Cas9 and the second portion is a TALE, the first portion is a CRISPR-Cas9 and the second portion is a BAT, the first portion is a CRISPR-Cas9 and the second portion is a Zinc finger, the first portion is a CRISPR-Cas9 and the second portion is a CRISPR-Cas9, the first portion is a CRISPR-Cas9 and the second portion is a Cfp1, the first portion is a Cfp1 and the second portion is a TALE, the first portion is a Cfp1 and the second portion is a BAT, the first portion is a Cfp1 and the second portion is a Zinc finger, the first portion is a Cfp1 and the second portion is a CRISPR-Cas9, or the first portion is a Cfp1 and the second portion is a Cfp1.

In some forms, one or both of the first and second portions of a targeted base editor includes at least one linker. In some forms, one or both of the first and second portions includes at least one linker, whereby the linker is positioned between the programmable DNA binding domain and the split deaminase domain. In some forms, both of the first and second portions comprise a linker between the programmable DNA binding domain and the split deaminase domain. Exemplary linkers are between 2 and 200 amino acids in length. For example, in some forms, the linker is between 2 and 16 amino acids in length.

In particular forms, the linker includes an amino acid sequence of any of GS, GSG, GSS, or SEQ ID NOs:23-27 or 30. The linkers also could be any form of rigid or flexible linkers known in state of the art (see for example: website ncbi.nlm.nih.gov/pmc/articles/PMC3726540/).

The base editor can be configured to place the target nucleic acid within a desired number of base pairs from a programmable binding domain binding site on a target DNA strand. In some forms, the base editor is configured such that the target nucleic acid is between 9 and 11 base pairs from a programmable binding domain binding site on a target DNA strand. In some forms, the distance between two binding sites of two programmable binding domains on a target DNA strand is between 12 and 22 base pairs. In other forms the distance between two binding sites of two programmable binding domains on a target DNA strand is between 14 and 19 base pairs.

Typically, at least one of the first and second portions of a base editor includes a cellular targeting moiety. Generally, both of the first and second portions includes a cellular targeting moiety, such as the same cellular targeting moiety. Exemplary cellular targeting moieties include a mitochondrial targeting sequence (MTS), and a nuclear localization sequence (NLS). An exemplary NLS includes an amino acid sequence of any one of SEQ ID NOs:34-39. An exemplary MTS includes an amino acid sequence of any one of SEQ ID NOs:22, 69, 71, 182 or 183.

In some forms, at least one of the first and second portions of a targeted base editor includes a base excision repair inhibitor. In some forms, the base excision repair inhibitor is a mammalian nuclear or mitochondrial DNA glycosylase inhibitor, such as a uracil glycosylase inhibitor. Exemplary base excision repair inhibitors have an amino acid sequence including any one of SEQ ID NOs:21 or 70.

Methods of using the disclosed deaminase domains and base editors are also provided. In some forms, the base editors can be used to perform base editing on a target nucleic acid. For example, disclosed is a method that includes bringing into contact a target nucleic acid and a targeted base editor, wherein the target nucleic acid is double-stranded DNA, whereby the instance of the target nucleotide sequence is deaminated by the targeted base editor. Typically, a deaminated nucleotide in the target nucleotide sequence is converted to a thymine or a guanine nucleotide. The conversion completes a base edit of the target nucleotide sequence.

In some forms of the method, the target nucleic acid is mitochondrial DNA. Exemplary target nucleotide sequences in which a nucleotide can be deaminated include, without limitation, AC, CC, GC, and TC. In some forms, the last C in the target nucleotide sequence is deaminated by the targeted base editor. In some forms, the instance of the target nucleotide sequence in the mitochondrial DNA is comprised in the mitochondrial DNA sequence. Base editing can be achieved when the instance of the target nucleotide sequence is between, for example, 1 and 25 bases, inclusive, of the base editor target DNA-binding sequence. In some forms, optimal base editing is achieved when the instance of the target nucleotide sequence is between 15 and 20 bases, inclusive, of the base editor target DNA-binding sequence. In some forms, the window of activity of base editing within a DNA target region is increased or reduced by changing the length, rigidity, or flexibility of a linker domain, or by changing the specificity or type of DNA binding domain, or by changing the split site within one or both of the split deaminase domains in one or both of two portions of a base editor, or by changing the type of the deaminase, or by changing the distance between DNA binding sites. For example, in some forms, the window of activity of base editing within a DNA target region is increased by increasing the length of a linker domain in one or both of two portions of a base editor. In other forms, the window of activity of base editing within a DNA target region is reduced by increasing the length of a linker domain in one or both of two portions of a base editor.

In some forms, the window of activity of base editing within a DNA target region is increased by reducing the length of a linker domain in one or both of two portions of a base editor. In other forms, the window of activity of base editing within a DNA target region is reduced by reducing the length of a linker domain in one or both of two portions of a base editor. In some forms, the window of activity of base editing within a DNA target region is increased by changing the specificity or type of DNA binding domain in one or both of two portions of a base editor. In other forms, the window of activity of base editing within a DNA target region is reduced by changing the specificity or type of DNA binding domain in one or both of two portions of a base editor.

In some forms, the window of activity of base editing within a DNA target region is increased by changing the split site in one or both of the split deaminase domains in each of two portions of a base editor. In other forms, the window of activity of base editing within a DNA target region is reduced by changing the split site in one or both of the split deaminase domains in each of two portions of a base editor.

The target nucleic acid can be in a cell. Thus is some forms of the method, bringing into contact the target nucleic acid and the targeted base editor is accomplished by facilitating entry of the targeted base editor into the cell. In some forms, the cell is in an animal. Thus, in some forms of the method, bringing into contact the target nucleic acid and the targeted base editor is accomplished by administering the targeted base editor to the animal.

Also described are methods for identifying modified (e.g., methylated) nucleotides in a target nucleic acid by enzymatic methods. In particular, disclosed is a method that includes bringing into contact one or more target nucleic acids and one or more deaminase domains that are differentially active on different modifications of cytidines, and subsequently sequencing the target nucleic acid. For example, in some forms, the one or more deaminase domains are collectively or individually active on one or more of unmodified cytosines (C), methylated cytosines (mC), or oxidized mC bases, including hmC, fC and caC, or combinations thereof. Therefore, in some forms, the methods include bringing into contact one or more target nucleic acids and one or more a deaminase domains that are differentially active on different modifications of cytidines, including one or more or unmodified (C), methylated (mC), or oxidized mC bases (e.g., hmC, fC, and caC) and subsequently sequencing the target nucleic acid.

Preferably, the target nucleic acid is double-stranded cytosine-methylated DNA and the deaminase domain can deaminate double-stranded DNA. Cytosine-methylated DNA refers to DNA where one, a few, many, or most cytosines are methylated. Natural DNA, such as genomic DNA has only some cytosines methylated. Exemplary double-stranded cytosine-methylated DNA includes genomic DNA, such as plant genomic DNA, animal genomic DNA and human genomic DNA. In some forms, the deaminase domain deaminates substantially only non-methylated cytosine nucleotides in the target nucleic acid. In some forms, substantially all of the non-methylated cytosine nucleotides in the target nucleic acid are deaminated by the deaminase domain, but the modified cytidines are not modified (or modified to much lesser extent than unmodified bases). Preferably, the deaminase domain deaminates 90% or more of the non-methylated cytosine nucleotides in the target nucleic acid. In some forms, the deaminase domains collective deaminate substantially only non-methylated cytosine nucleotides in the target nucleic acid. In some forms, substantially all of the non-methylated cytosine nucleotides in the target nucleic acid are deaminated by the deaminase domains collectively, but the modified cytidines are not modified (or modified to much lesser extent than unmodified bases). Preferably, the deaminase domains collectively deaminate 90% or more of the non-methylated cytosine nucleotides in the target nucleic acid. By sequencing the deaminated target nucleic acid, methylated cytosine nucleotides in the target nucleic acid are identified (i.e., these are the cytidines that are not edited by the deaminase(s)).

Methods for generating sequence diversity in a pool of target nucleic acids, either inside or outside of living cells, are also provided. For example, the deaminases disclosed herein can be used to introduce random, non-targeted mutations in a pool of DNA sequences by non-targeted base editing. An exemplary method includes bringing into contact a deaminase domain and a plurality of copies of a target nucleic acid for a time and under conditions that results in deamination of an average of 0.1 to 5.0 nucleotides per copy of the target nucleic acid. Preferably, the target nucleic acid is double-stranded DNA and the deaminase domain can deaminate double-stranded DNA.

In some forms, the copies of the target nucleic acid are in vitro. In some forms, the deaminated nucleotides in the copies of the target nucleic acid are converted to a thymine or a guanine nucleotide via an in vitro reaction. In some forms, the method further includes converting deaminated nucleotides to the canonical counterpart, such as dU to dT, and dI to dA, followed by a selection procedure, such as, but not limited to, mRNA display, ribosome display, or SELEX. In some forms, the conversion is carried out by PCR amplification. In other forms, the diversified DNA is transformed into cells for in vivo selection and directed evolution applications. Methods for DNA diversity generation provide an alternative to error-prone PCR for making randomized DNA, especially in cases where the fragments to be diversified are much larger than a size that can be readily PCR amplified.

In some forms, when the deaminated nucleotides in the copies of the target nucleic acid are converted to a thymine or a guanine nucleotide, the conversion completes one or more base edits of some or all of the copies of target nucleic acid. In some forms, the deaminated nucleotides in the copies of the target nucleic acid are converted to a thymine or a guanine nucleotide by incubating the copies of the target nucleic acid in cells. For example, the copies of the target nucleic acid can be in cells, and facilitating entry of the deaminase domain into the cells brings into contact the deaminase domain and the copies of a target nucleic acid.

Methods of treating or preventing a mitochondrial genetic disease in a subject by editing one or more nucleic acids in mitochondrial DNA in a cell of the subject, are also described. In some forms, the methods introduce to the cell a targeted cytosine deaminase base editor including a deaminase domain and a DNA interacting domain that interacts with the target nucleotide (or a sequence at the vicinity of the target nucleotide), wherein a target nucleic acid within mitochondrial DNA is deaminated by the targeted base editor. In some forms the DNA interacting domain is a DNA binding domain or a transcription factor that interacts with its target site, an RNA or DNA polymerase that interact with a promoter or origin of replication and carry the deaminase along a certain region on the dsDNA. In some forms, the deaminated nucleotide in the target nucleotide sequence is converted to a thymine or a guanine nucleotide. Typically, the methods edit the mitochondrial DNA to a non-pathogenic form. In some forms, the deaminated nucleotide is at a position selected from m.583G>A, m.616T>C, m.1606G>A, m.1644G>A, m.3258T>C, m.3271T>C, m.3460G>A, m.4298G>A, m.5728T>C, m.5650G>A, m.3243A>G, m.8344A>G, m.14459G>A, m.11778G>A, m.14484T>C, m.8993T>C, m.14484T>C, m.3460G>A, and m.1555A>G. I some forms, the cell is selected from the group consisting of a fibroblast, lymphocyte, pancreatic cell, muscle cell, neuronal cell, and a stem cell.

In some forms, the cells are in an animal, and bringing into contact the deaminase domain and the copies of a target nucleic acid is accomplished by administering the deaminase domain to the animal. In some forms, when the copies of the target nucleic acid are in cells, the deaminase domain can be encoded by a transgenic expression construct (e.g., an expression vector) in the cells. In such forms, bringing into contact the deaminase domain and the copies of a target nucleic acid is accomplished by transiently expressing the deaminase domain in the cells, either as a stand-alone enzyme or as a fusion to some other protein domains such as DNA binding domains, transcription factors, or DNA or RNA polymerase (e.g. T7 RNA polymerase).

Vectors including or expressing a targeted base editor are also provided. Exemplary vectors include altered adenovirus (AAV) vectors, or a Lentivirus vectors. In some forms, the targeted base editor is encapsulated within the vector. In some forms, the deaminase domain includes a targeted base editor within a vector.

Additional advantages of the disclosed methods will be set forth in part in the description which follows, and in part will be understood from the description, or can be learned by practice of the disclosed methods and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed methods and compositions and together with the description, serve to explain the principles of the disclosed method and compositions.

FIG. 1 is a schematic illustration of the step-wise system to produce and experimentally assess and characterize putative deaminase domains, and identify deaminases that are active on double stranded DNA (dsDNA), and determine their editing context-specificity; multiple domains from each deaminase protein family of the Cytidine deaminase-like (CDA) superfamily in the pfam database are synthesized and expressed by cell-free in vitro transcription/translation (from top to bottom, DNA sequences include ATCCGATCAGAGCT (SEQ ID NO:287), 5′-ATTTGATTAGAGTT-3′ (SEQ ID NO:289) and 3′-TAGGCTAGTTTTGA-5′ (SEQ ID NO:290)), then characterized by assays using ssDNA and dsDNA substrates to determine strand-bias and sequence specificity using next generation sequencing (NGS) techniques. These are just illustrative sequences. The sequences for the actual substrate used in the deamination assay shown in FIG. 2. The actual substrate used for the NGS assay is SEQ ID NO:73:

(SEQ ID NO: 73)
TAATAATTATATTATTATTTTAAATTAATTATTTAACCGTGGTGCGCGG
GGTCGCCCAGCAATAGTATAGGTTGTCGAGTATGAAGGGTCTAAAAGAT
TTTAAGACACCTTACGGACGAAGAGTTTCTCTCTTAGTCCCCTGATCTG
CAGAACCCAGGATATCAAGCACATTTCACTTCACGTGTTTTGATGAAAC
TATACATCACCCGCGCCACAGGCGCTGTGCGGTTTATAATATATTATAA
TTTATATTTATATTAAATT.

FIGS. 2A-2C are gel electrophoresis images showing activity of the deaminase domains on a double-stranded (FIGS. 2A, 2B) or single-stranded (FIG. 2C) FAM-labelled DNA substrate in a deamination assay. FIG. 2D is a gel electrophoresis image showing activity of the indicated deaminase domains on double-stranded DNA substrates, with each of lanes 1-6 containing the following sequences (1) A[15]TGCGCCA[15](SEQ ID NO:268), (2) A[15]ACA[15] (SEQ ID NO:269), (3) A[15]CCA[15] (SEQ ID NO:270), (4) A[15]GCA[15] (SEQ ID NO:271), (5) A[15]TCA[15] (SEQ ID NO:272), (6) A[15]ACGCCTCA[15] (SEQ ID NO:273) (ssDNA substrate sequences), respectively, in the absence (−) or presence (+) of each of the deaminase domains BE_R1_11, BE_R1_12, BE_R1_28, and BE_R1_41, respectively. For the double-stranded DNA substrate the complementary strands were annealed to the given substrates.

FIGS. 3A-3B are images showing NGS (FIG. 3A) and Sanger sequencing (FIG. 3B; from top to bottom, showing deaminase activity on sequence ATGAATCGGCCAACGCGCGGGGAGAGGCGGTTTGCGTATTGGGCGCCAGGGT GGTTT (SEQ ID NO:291) and ATGAATCGGTCAATGCGTGGGGAGAGGTGGTTTGTGTATTGGGTGCCAGGGTG GTTT (SEQ ID NO:292) results for the DNA deamination assay. These figures demonstrate an exemplary piece of data showing the outcome of the dsCDAs treatment on the dsDNA.

FIGS. 4A-4B are probability sequence logos of the region flanking mutated cytosines in dsDNA substrates incubated with the indicated deaminase based on editing efficiency at editing threshold levels of 50% (FIG. 4A), and 25% (FIG. 4B), respectively. FIG. 4A shows (top row) examples of context-independent deaminases (with mixed specificity) that can edit cytidines in any context (NCN) and (bottom two rows) examples of the identified context-dependent deaminases that are specific toward certain sequences that precede cytidines.

FIG. 5 showing deaminase assay for split deaminases either alone, or combined. Activity of various N- and C-terminal halves of BE11, BE12, and BE28 deaminase domains on a DNA substrate is shown by gel electrophoresis image, comparing each of control, and r N-terminal fragments (N1, N2, N3, N4, N5) and 5 C-terminal fragments (C1, C2, C3, C4, C5) alone, and combined, for each species of deaminase, respectively; diagrams of the N- and C-terminal portions of the base editors indicate the relative configurations of N- or C-terminal Deaminase (Deam_N/Deam_C) molecules within the base editors tested.

FIG. 6 shows sequence alignment logos for the members of MafB19-deam family that are active or inactive on dsDNA along with the signature motifs present in the dsDNA specific members of this deaminase family which can be used to as signatures to identify additional dsDNA-specific deaminases in this family.

FIG. 7 shows the distinct branch within MafB19-deam family where most of the identified dsDNA-specific deaminase of this family are located.

FIG. 8 shows sequence alignment logos for the members of SCP1201-deam family that are active or inactive on dsDNA along with the signature motifs present in the dsDNA-specific members of this deaminase family which can be used to as signatures to identify additional dsDNA-specific deaminases in this family.

FIG. 9 is a schematic representation of an in vitro system for rapid testing of Base editors. A base editor is made by cloning the deaminase domains downstream of designer TALE. The entire cassette is cloned downstream of a T7 promoter and used as template in the In Vitro Translation (IVT) reaction. The target (encoding binding sites for DNA binding domains of interest, e.g. designer TALEs) are cloned on plasmids which was then used as dsDNA substrate in the IVT reaction. Upon expression in the IVT system, the base editor protein (e.g., TALE-deaminase fusion protein) binds to its target on the substrate plasmid and introduce edits to the target plasmid. The substrate plasmid is then PCR amplified and the position/frequency of edits are determined by either sequencing or T7 endonuclease assay.

FIGS. 10A-10C are probability sequence logos results obtained from NGS sequencing of the region flanking targeted cytosines in different dsDNA substrates ACACACACACACACAC (SEQ ID NO:191) (FIG. 10A), ACGTGTACACGTACGT (SEQ ID NO:192), GCGCGCGCGCGCGCGCG (SEQ ID NO:193), and CCGGCCGGCCGGCCGG (SEQ ID NO:194) (FIG. 10B), or TCGAGATCTCGATCGA (SEQ ID NO:195), TCTCTCTCTCTCTCTC (SEQ ID NO:196) and CCCCCCCCCCCCCCCC (SEQ ID NO:197) (FIG. 10C), incubated with BER1_11, BE_R1_12, BE_R1_28 or BE_R1_41, respectively.

FIGS. 11A-11B are a diagrams showing (FIG. 11A) a schematic of an in vitro system for cloning deaminase split domains downstream of designer TALEs (called TALE_Left and TALE_Right) based on a modification of the scheme in FIG. 9; and (FIG. 11B) different split base editor design strategies, based on BE_R1_12, showing: BE_R1_12 (wt), the mutated active site sequence (HAE to HAA) in the inactive, “dead” protein, as well as three different truncated proteins, 20, 40 and 60. The domain organization including addition of TALE left (L) and right (R) domains is also shown, as well as the resulting combined, functional base editor that uses the TALE L and R binding domains to co-localize at the Target DNA.

FIG. 12 is a diagram showing results of base editor deaminase activity on a target (poly-cytosine) DNA substrate for each of the different base editor designs described in FIG. 11, including TALE_R only (control), as well as TALE_R_BE_R1_12 (truncated 20, 40 or 60), each in combination with TALE_L only (control), or TALE_L and the mutated active site sequence (HAE to HAA) in the inactive, “dead” BE_R1_12 protein. Edited bases (C to T) are indicated in the sequencing data shown for each construct pair, respectively. CCCCCCCCCCCCCCCC (SEQ ID NO:197), CCCCCCCTTTTTTCCC (SEQ ID NO:198), CCCCCCTTTTTTTCCC (SEQ ID NO:199) Partial editing is indicated as mixed peaks in the Sanger Chromatograms. In such cases, the base calling software calls the major peaks as the consensus base, while in fact that position contains a mixture of bases.

FIG. 13 is a diagram showing results of base editor deaminase activity on a variety of different target DNA substrates CCCCCCCCCCCCCCCC (SEQ ID NO:197), ACACACACACACACAC (SEQ ID NO:191), ACGTACGTACGTACGT (SEQ ID NO:200), CCGGCCGGCCGGCCGG (SEQ ID NO:201), and GCGCGCGCGCGCGCGC (SEQ ID NO:202), CTCTCTCTCTCTCTCT (SEQ ID NO:203), or TCGATCGATCGATCGA (SEQ ID NO:204), and sequence contexts for the base editor TALE_R_BE_R1_12 (truncated 30), in combination with TALE_L and the mutated active site sequence (HAE to HAA) in the inactive, “dead” BE_R1_12 protein. Edited bases (C to T) are indicated in the sequencing data shown for each substrate, respectively, including, CCCCCCCTTTTTTCCC (SEQ ID NO:205), ACACACACATACACAC (SEQ ID NO:191), ACGTGTATATGTACGT (SEQ ID NO:192), ACGTGTATATGTACGT (SEQ ID NO:206), GCGCGCGCGTGCGCGC (SEQ ID NO:207), TCTTTTTTTTTTTCTC (SEQ ID NO:208), TCGAGATCTCGATCGA (SEQ ID NO:195), or TCGAGATCTTGATCGA (SEQ ID NO:209). Partial editing is indicated as mixed peaks in the Sanger Chromatograms. In such cases, the base calling software calls the major peaks as the consensus base, while in fact that position contains a mixture of bases.

FIG. 14 is a diagram showing experiments to identify and optimize the editing window of activity of base editors. The diagram depicts design strategy, as well as the resulting combined, functional base editor that uses the TALE L and R binding domains to co-localize at the Target DNA, and results of base editor deaminase activity on a target (poly-cytosine) DNA substrate CCCCCCCCCCCCCCCC (SEQ ID NO:197), for each of 4 different base editors, based on BE_R1_41, including four different truncation mutants, resulting from splitting wt BE_R1_41 at positions G43, or G108 (located either side of the HVE binding site), and then re-combining the entire deaminase domains each of 4-ways, respectively. Edited bases (C to T) are indicated in the sequencing data shown for each substrate, respectively, including, CCCCCCTTTTTTCCCC (SEQ ID NO:210), CCCCCCTTTTTTTCCC (SEQ ID NO:199), CCCCCCCTTTTTTTTC (SEQ ID NO:211). The corresponding positional window of activity is depicted and quantified for each design.

FIG. 15 is a diagram showing results of base editor deaminase activity on a variety of different target DNA substrates CCCCCCCCCCCCCCCC (SEQ ID NO:197), ACACACACACACACAC (SEQ ID NO:191), ACGTACGTACGTACGT (SEQ ID NO:200), CCGGCCGGCCGGCCGG (SEQ ID NO:201), and GCGCGCGCGCGCGCGC (SEQ ID NO:202), TCTCTCTCTCTCTCTC (SEQ ID NO:196), GAGAGAGAGAGAGAGA (SEQ ID NO:212) or TCGATCGATCGATCGA (SEQ ID NO:204), for the base editor formed by recombining BE_R1_41 truncated at G108 (N) and G43 (C) having 2 active sites, using TALE L and R domains, as well as the base editor formed by recombining BE_R1_41 truncated at G108 (N) and G108 (C) having one active site, using TALE L and R domains, respectively. Edited bases (C to T) are indicated in the sequencing data shown for each substrate, CCCCCCCTTTTTCCCC (SEQ ID NO:213), CCCCCCCCCTTTTCC (SEQ ID NO:214), ACACACACATACACAC (SEQ ID NO:215), ACGTGTATATGTACGT (SEQ ID NO:206), CCGGCCGGTTGGCCGG (SEQ ID NO:216), TCTTTTTTTTTTTCTC (SEQ ID NO:217), TCTCTCTCTTTCTCTC (SEQ ID NO:218), GAGAAAAAAAAAGAGA (SEQ ID NO:219) or TCGAGATCTTGATCGA (SEQ ID NO:209), or TCGAGATTTTGATCGA (SEQ ID NO:220), respectively.

FIGS. 16A-16C are diagrams showing results of base editor deaminase activity on each of three CCCCCCCCCCCCCCCC (SEQ ID NO:197), ACGTACGTACGTACGT (SEQ ID NO:200), TCTCTCTCTCTCTCTC (SEQ ID NO:196) (FIG. 16A), and two GAGAGAGAGAGAGAGA (SEQ ID NO:212), TCGATCGATCGATCGA (SEQ ID NO:204) (FIG. 16B), and three CCGGCCGGCCGGCCGG (SEQ ID NO:201), ACACACACATACACAC (SEQ ID NO:191), or GCGCGCGCGCGCGCGC (SEQ ID NO:202) (FIG. 16C) different target DNA substrates, for each of negative control (no editor), as well as the base editor formed by recombining BE_R1_41 truncated at G108 (N) and G43 (C) having 2 active sites, using TALE L and R domains, as well as the base editor formed by recombining BE_R1_41 truncated at G108 (N) and G108 (C) having one active site, using TALE L and R domains, respectively. Edited bases (C to T) are indicated in the sequencing data shown for each substrate, respectively. The corresponding positional window of activity is depicted and quantified for each design.

FIGS. 17A-17B show the predicted model for the split deaminase base editor and position of window of activity on the forward and reverse strands on the target region (FIG. 17A) and data confirming that model (FIG. 17B). FIG. 17B is a diagram showing results of assays swapping the deaminase split halves of the base editor formed by recombining BE_R1_41 truncated at G108 (N) and G108 (C) (having one active site), with TALE L and R binding domains to assess editing efficiency and the position of window of activity on poly C or poly G DNA substrates CCCCCCCCCCCCCCCC (SEQ ID NO:197) and GGGGGGGGGGGGGGGG (SEQ ID NO:221). Edited bases (C to T or G to A) are indicated in the sequencing data shown for each substrate, including CCCCCCCCTTTTTTTC (SEQ ID NO:197), CCCCCCCCCCCCCTCC (SEQ ID NO:222) and GGAGGGGGGGGGGGGG (SEQ ID NO:223), respectively.

FIG. 18 is a diagram showing putative base editor window of activity on a target DNA substrate for the base editor formed by recombining BE_R1_41 truncated at G108 (N) and G43 (C) having 2 active sites, using TALE L and R domains, as well as the base editor formed by recombining BE_R1_41 truncated at G108 (N) and G108 (C) having one active site, using TALE L and R domains, respectively, which bind to the DNA sequence TCTAGCCTAGCCGTTTXXXXXXXXXXXXXXXXAGGGTGAGCATCAAACTCA (SEQ ID NO:224). The corresponding positional window of activity, shown as a function of interaction with the helical DNA changes based on the nature of deaminase, indicates a periodic and asymmetric activity window. The span and position of window of activity is dependent on multiple factors such as the position split design (i.e. position of the split/truncation sites for each of the two deaminase halves), type of linker and DNA binding domains etc. as described in the text.

FIG. 19 is a diagram showing results of base editor deaminase activity on poly C target DNA substrate CCCCCCCCCCCCCCCC (SEQ ID NO:197), for each of the base editor formed by recombining BE_R4_7, BE_R4_12, BE_R4_13, BE_R4_17, BE_R4_18, BE_R4_19, BE_R4_20 and BE_R4_21, each using TALE L and R domains. Edited bases (C to T) are indicated in the sequencing data shown for each substrate, respectively. The corresponding positional window of activity is depicted and quantified for each design.

FIG. 20 is a diagram showing putative base editor deaminase activity on a variety of target DNA substrates of different lengths (Poly C5-PolyC20, having sequences of CCCCC (SEQ ID NO:225), CCCCCC (SEQ ID NO:226), CCCCCCC (SEQ ID NO:227), CCCCCCCC (SEQ ID NO:228), CCCCCCCCC (SEQ ID NO:229), CCCCCCCCCC (SEQ ID NO:230), CCCCCCCCCCC (SEQ ID NO:231), CCCCCCCCCCCC (SEQ ID NO:232), CCCCCCCCCCCCC (SEQ ID NO:233), CCCCCCCCCCCCCC (SEQ ID NO:234), CCCCCCCCCCCCCCC (SEQ ID NO:235), CCCCCCCCCCCCCCCC (SEQ ID NO:236), CCCCCCCCCCCCCCCCC (SEQ ID NO:237), CCCCCCCCCCCCCCCCCC (SEQ ID NO:238), CCCCCCCCCCCCCCCCCCC (SEQ ID NO:239), CCCCCCCCCCCCCCCCCCCC (SEQ ID NO:240), respectively), for the base editor formed by recombining BE_R1_41 truncated at G108 (N) and G43 (C) having 2 active sites, using TALE L and R domains. Edited bases (C to T) are indicated in the sequencing data shown for each substrate, including CCCCCCTTTTTCCC (SEQ ID NO:241), CCCCCCCTTTTTCCCC (SEQ ID NO:242), CCCCCCCCTTTTTCCCC (SEQ ID NO:243), CCCCCCCCTTTTTTTCCCC (SEQ ID NO:244), CCCCCCCCCCCTTTCCCCCC (SEQ ID NO:245), respectively. The corresponding positional window of activity is depicted and quantified for each design.

FIGS. 21A-B show putative base editor deaminase activity on a variety of target DNA substrates, for the base editor formed by recombining BE_R1_41 truncated at G108 (N) and G43 (C) having 2 active sites, using either TALE L and R domains, or BAT_L and TALE_R domains, or TALE_L and BAT_R binding domains, respectively. FIG. 21A shows the effect of the abovementioned base editor combinations on a variety of target DNA substrates of different lengths (Poly C10-PolyC18, including CCCCCCCCCC (SEQ ID NO:230), CCCCCCCCCCCC (SEQ ID NO:232), CCCCCCCCCCCCCC (SEQ ID NO:234), CCCCCCCCCCCCCCC (SEQ ID NO:235), CCCCCCCCCCCCCCCC (SEQ ID NO:236), CCCCCCCCCCCCCCCCCC (SEQ ID NO:238), respectively, including CCCCCCTTTTTCCC (SEQ ID NO:241), CCCCCCCTTTTTCCCC (SEQ ID NO:242), CCCCCCTTTTTCCCC (SEQ ID NO:246), CCCCCCCCCTTTCCC (SEQ ID NO:247), CCCCCCCCCTTTCCCC (SEQ ID NO:248), CCCCCCCCCTTTTTCCCC (SEQ ID NO:249), CCCCCCCCCTTTTCCCCC (SEQ ID NO:250). FIG. 21B shows the effect of the abovementioned base editor deaminase on a polyC16 substrate and establishes that the nature of DNA binding domain affects the window of activity and editing efficiency of base editors. Edited bases (C to T) are indicated in the sequencing data shown for each substrate, including CCCCCCTTTTTCCCC (SEQ ID NO:246), CCCCCCCCCTTTCCC (SEQ ID NO:247), and CCCCCCCTTTCCCCCC (SEQ ID NO:251), respectively. The corresponding positional window of activity is depicted and quantified for each design.

FIG. 22 is a diagram showing different split base editor design strategies, based on BE_R1_41, showing the domain organization including BE_R1_41 (N or C) fragments, each with the addition of TALE left (L) and right (R) domains, as well as Coiled coil (“coil”) domains, to enhance flexibility and activity window size. Edited bases from a CCCCCCCCCCCCCCCC (SEQ ID NO:236) substrate, showing edits (C to T) are indicated in the sequencing data shown for each substrate, including CCCCCCTTTTTTTCCC (SEQ ID NO:252), CCCCCCCTTTTTTTTC (SEQ ID NO:253) and TTTTTTTTTTTTCCCC (SEQ ID NO:254), respectively.

FIGS. 23A-23B show data demonstrating the optimal position of the target base. FIG. 23A is a diagram showing results of base editor deaminase activity of the base editor TALE_L_“dead”dBE_R1_12, in combination with TALE_R_BE_R1_12 (truncated 60), on each of five different target DNA substrates, each corresponding to fixing a pathogenic mitochondrial mutation, mCoxl V421A in mouse mitochondria, corresponding to converting C6589 to T, and having a single base shift for C6589 relative to the TALE binding sites, respectively including GTAGGAGCAACATAA (SEQ ID NO: 255), CGTAGGAGCAACATA (SEQ ID NO: 256), TCGTAGGAGCAACAT (SEQ ID NO: 257), TTCGTAGGAGCAACA (SEQ ID NO: 258), ATTCGTAGGAGCAAC (SEQ ID NO: 259). Edited bases (C to T) are indicated in the sequencing data shown for each substrate, respectively, including TCGTAGGAGTAAACAT (SEQ ID NO: 260). The corresponding positional window of activity is depicted and quantified for each design. The edited base (C6589 C to T) is present when this C residue is 10 bps (corresponding to 1 turn of double helix) away from the Left-side TALE binding site. FIG. 23B is a graph of dC-dT editing efficiency over Distance of target dC from Left-side TALE binding site for each of the C nucleotides at C6589 (Distance=8-12) and C6593 (Distance=12-16), respectively. In this example, C6589 is the target base and C6593 is a bystander base. This approach (sliding the target window 1 bp at a time) could be used to maximize the editing efficiency on the target base and minimize the editing of bystander bases for any given target

FIG. 24 is a diagram summarizing the factors affecting the length and position of window of activity and different split base editor design rules determined according to the data in FIGS. 10-23. Each part of a two-part split base editor is shown on each opposing strand of double-stranded target DNA, with each nucleic acid shown as an X. Each part of the split base editor includes a DNA-binding domain and a Deaminase N or C domain connected via a linker (shown with the N-domain bound to the 5′ DNA strand and the C-domain bound to the 3′ DNA strand). In the depicted ample, the distance between the DNA binding domain recognition sites is shown as being 19 residues in total, with the window of deaminase activity including 7 nucleic acids on each strand with an overlap of 3 nucleic acids (indicated by arrows).

FIGS. 25A-25B show (FIG. 25A) a schematic of the domain organization of each of the two parts of split BE12 base editors, with each of the split deaminases (“dead” dBE_12-N—TALE_L; and BE_12-C—TALE_R) including the MTS targeting sequence, fused to UGI (to limit the activity of mitochondrial uracil DNA glycosylase) and GFP (in the case of Left-side TALE fusion) or mKate (in the case of right TALE fusion), the resulting combined, functional base editor that uses the TALE L and R binding domains to co-localize at the Target mitochondrial DNA (hND1 gene); and (FIG. 25B) a photomicrograph showing the results of base editing at the hND1 locus using BE_12-dead co-transfected with different BE_12-based deaminase truncation mutants in a HEK293T cell line, with the positions of the expected cleavage products by T7 endonuclease in edited samples indicated by arrows.

FIG. 26 is a schematic of the domain organization of split base editors based on BE12 or BE41, with each of the split deaminases including TALE_L and TALE_R DNA binding domains, the MTS targeting sequence, fused to UGI (to limit the activity of mitochondrial uracil DNA glycosylase) and GFP (in the case of Left-side TALE or BAT fusion) or mKate (in the case of right TALE or BAT fusion) for either dead dBE12 or BE41 cut at G108(N) and G43(C), respectively. Edited bases (C to T) in the target locus (hND1) (ACTCAATCCTCTGATC (SEQ ID NO:261)) are indicated in the sequencing data shown for each substrate, respectively.

FIGS. 27A-27B show (FIG. 27A) a schematic of the domain organization of each of four split BE41 base editors targeting mitochondrial hND1 gene, with each of the split deaminases including either TALE DNA binding domains (TALE_L-BE_41-N(1); and TALE_R-BE_41-C(2)), or BAT binding domains (BAT_L-BE_41-N(3); and BAT_R-BE_41-C(4)), each including the MTS targeting sequence, fused to UGI (to limit the activity of mitochondrial uracil DNA glycosylase) and GFP (in the case of Left-side TALE or BAT fusion) or mKate (in the case of right TALE or BAT fusion); and (FIG. 27B) a photomicrograph showing the results of different combinations of N-((1) or (2)) with C-((1) or (2)) constructs shown in FIG. 27A in a HEK293T cell line, with the positions of the expected cleavage products by T7 endonuclease in edited samples indicated by arrows.

FIGS. 28A-28B show (FIG. 28A) a schematic of the domain organization of two parts of a split BE41 base editor, with each of the split deaminases including either left hand side TALE DNA binding domains (TALE_L-BE_41-N) or Right Hand side Zinc Finger (ZF_R2), each including the MTS targeting sequence, fused to UGI (to limit the activity of mitochondrial uracil DNA glycosylase) and GFP (in the case of Left-side fusion) or mKate (in the case of right fusion); and (FIG. 28B) Edited bases (C to T) in the targeted DNA (ACTCAATCCTCTGATC (SEQ ID NO:261)) are indicated in the sequencing data and shown for treated and control DNA samples, and the corresponding positional window of activity is depicted and quantified for each design, respectively.

FIGS. 29A-29B show a schematic of the domain organization of two single AAV base editor designs for BE41-based base editors, including the MTS targeting sequence and Zinc Finger Left side (ZF_L) DNA binding domain, BE_41-C, fused to P2A and directly fused with MTS-BE_41-N fused to UGI (to limit the activity of mitochondrial uracil DNA glycosylase) Right-side ZF fused to GFP; or MTS targeting sequence and Zinc Finger Left side (ZF_L) DNA binding domain, BE_41-C, fused to TAA_IRES and directly fused with MTS-BE_41-N fused to UGI (to limit the activity of mitochondrial uracil DNA glycosylase) Right-side ZF fused to GFP (FIG. 29A). The result of T7 endonuclease assay at various MOI of the AAV particles harboring the constructs shown in A are shown (FIG. 29B).

FIG. 30 is a schematic of the domain organization of a split BE41-based base editor used to edit mND1 loci in the mouse NIH3T3 cell line, including the MTS targeting sequence and TALE Left side DNA binding domain fused to BE_41-N cut at G108, fused to UGI and GFP; and MTS targeting sequence and TALE Right side DNA binding domain fused to BE_41-C cut at G43 fused to UGI and mKate.

FIGS. 31A-31B show editing efficiency and off-targets determined based on NGS (FIG. 31A) and sanger chromatograms of the target locus in the base editor treated sample vs. the negative control sequence CATTAGTAGAACGCA (SEQ ID NO:262) (FIG. 31B). The edited (G to A) nucleic acid base in the sequence CATTAGTAAAACGCA (SEQ ID NO:263) at position G2820 is indicated.

FIGS. 32A-32D show that different dsDNA-specific deaminases (dsCDAs) have different activities on cytidine modifications. FIG. 32A is a schematic of the structures of cytosine (C), 5-methylcytosine (5mC), 5-hydroxymethylcytosine (5hmC), 5-formylcytosine (5fC) and 5-carboxylcytosine (5caC). FIGS. 32B-32D are micrographs of deaminase assays using each of deaminases BE_R1_I11, BE_R1_12, BE_R1_28, BE_R1_41, BE_R2_11, BE_R2_19, BE_R2_28, BE_R2_31, and DddA, on DNA substrates containing no methylation (FIG. 32B), 5-methylcytosine (5mC) (FIG. 32C), and 5-hydroxymethylcytosine (5hmC) (FIG. 32C), respectively.

FIGS. 33A-33B show the assay for protecting cytosine by methylation using BamH1 methylase, (coverts cytosine to methylated 5mC). FIG. 33A is a schematic of the assay for pre-treating dsDNA substrates with either No MTase (Control), BAMHI MTase, or CpG MTase, then adding ds-deaminase, then sequencing, whereby unmodified Cytosines are deaminated to uracil and are detected as a T, modified Cytosines are not deaminated. FIG. 33B shows the probability sequence logo of substrate DNA untreated (No MTase) or treated with (BamH1 MTase) then deaminated and sequenced.

FIGS. 34A-34C are sequencing chromatograms showing the activity of BE_R1_11 deaminase (FIG. 34A), BE_R1_28 deaminase (FIG. 34B), or BE_R1_41 deaminase (FIG. 34C), on DNA substrates GTACACCATCCGTCCC (SEQ ID NO:274) and GTGTTCTCTATTTCAC (SEQ ID NO:275) modified to include 5caC, 5fC, 5hmC or 5mC, respectively. GTGTTCTCTATTTCAC (SEQ ID NO:275)

FIG. 35 is a schematic showing the activity of Tet2 oxidation enzyme and BGT Glucosylation enzyme on a DNA substrate having a sequence CCGTCGGACCGC (SEQ ID NO:278) containing methyl Cytosine at position 5 and hydroxymethyl Cytosine at position 10, which is converted to CCGTCGGACCGC (SEQ ID NO:279) containing carboxyl Cytosine at position 5 and glucosyl-methyl Cytosine at position 10, respectively.

FIG. 36 shows sequencing chromatograms showing the differential activity of BE_R1_12 and BE_R1_41 deaminases on DNA substrate GTACACCATCCGTCCC (SEQ ID NO:274), including 5mC, 5hmC, 5fCand 5caC, respectively, alone (BE12/BE41), or following oxidation and glucosylation (BE12+TET2-BGT/BE41+TET2-BGT), at each of time points 1 hour (t1) and 2 hours (t2) incubation, respectively. In the absence of Oxidation and glucosylation by TET2 and BGT Deamination of 5mC to T by BE_R1_41 in GTACACCATCCGTCCC (SEQ ID NO:274), yielded GTACACCATTTGTCCC (SEQ ID NO:276); Deamination of 5hmC to T by BE_R1_41 yielded GTACACCATTTGTCCC (SEQ ID NO:276) and GTACACCATTTGTTCC (SEQ ID NO:277), respectively. This figure illustrates that for deaminases that are active on mC or hmC, like BE41, a TET2+BGT treatment can be used to protect the methylated DNA from deamination. Some deaminases like BE12, although able to edit in normal contexts, are inherently less active on modified DNA and can be used without the need for an initial TET2+BGT treatment.

FIG. 37 is a schematic showing the activity of one or more deaminases on a substrate DNA CTAACTTACCATGATTAATTTAAGAATTCTCATCGTCA (SEQ ID NO:280), leading to three different deamination products TTAATTTACTATGATTAATTTAAGAATTCTTATTGTTA (SEQ ID NO:281), CTAATTTACCATAATTAATTTAAGAATTCTTATCGTTA (SEQ ID NO:282), and CTAACTTATCATAATTAATTTAAAAATTCTTATCGTCA (SEQ ID NO:283), respectively.

FIGS. 38A-B8 show a frequency sequence logo (FIG. 38A) and aligned sequences of NGS (FIG. 38B) resulting from deaminase activity of BE_R1_12 deaminase on DNA substrate.

FIG. 39 is a schematics showing a base editor (BE) attached to the T7 RNA polymerase (T7 RNAP) as targeting domain to introduce diversity within a window defined by T7 promoter and terminator on a DNA substrate GATTGAATGGTACTGATCAGATCCTCAAGAGTAGCAGT (SEQ ID NO:284), deaminated to GATTGAATGGTACTGATTAGATTTTTAAGAGTAGCAGT (SEQ ID NO:285). This figure demonstrates the concept/workflow of epigenetic sequencing method.

FIG. 40 is a base editor (Split BE41) attached to the dCas9 binding site, where dCas9/gRNA serve as a road block for the polymerase on a double stranded DNA downstream of the T7 promoter region; One half of the split BE41 is shown fused to T7 polymerase and a second half is shown as a free-floating enzyme.

FIG. 41 is a diagram showing different forms of split deaminases.

### DETAILED DESCRIPTION OF THE INVENTION

The disclosed methods and compositions can be understood more readily by reference to the following detailed description of particular embodiments and the Examples included therein and to the Figures and their previous and following description.

Current genome-editing technologies introduce double-stranded (ds) DNA breaks at a target locus as the first step to gene correction. Although most genetic diseases arise from point mutations, approaches that rely on DNA cleavage followed by recombination to fix point mutations are inefficient and typically induce an abundance of random insertions and deletions (indels) at the target locus from the cellular response to dsDNA breaks. For most known genetic diseases, correction of a point mutation in the target locus, rather than stochastic disruption of the gene, is needed to address the underlying cause of the disease.

Base editing is a recent approach to genome editing that enables the direct, irreversible conversion of one target DNA base into another in a programmable manner, without requiring dsDNA backbone cleavage or a donor template. Current base editing approaches mainly leverage a ssDNA-specific DNA deaminase (e.g. APOBEC or AID) fused to an RNA-guided DNA binding domain (e.g. dCas9 or nCas9). The R-loop formation by the guide RNA/Cas9 at the target locus exposes a ssDNA region that serves as a substrate for the ssDNA deaminase enzyme. While powerful, base editing using RNA-guided proteins have inherent limitations. For example, it cannot be used to edit mitochondrial genome (or other membranous organelles that contain genomes like chloroplasts and plastids) since there are not currently efficient ways to deliver guide RNA or other nucleic acids to mitochondrial lumen.

Fusing ssDNA-specific deaminases to dsDNA binding domains such as Zinc Fingers and TALEs have not led to efficient base editors, mainly because the ssDNA-specific deaminases have little to no activity on the dsDNA. To address this limitation, the tree of life was mined and deaminases that are active on dsDNA and are able to edit dsDNA in various sequence contexts were discovered. As such, the deaminases enable editing dsDNA in much broader contexts than previously possible and exhibit reduced off-target editing than prior characterized deaminases. As shown in the Examples, these deaminases are active on double-stranded and single-stranded DNA substrates rather than just on single-stranded DNA as is the case for almost all the previously characterized deaminases (with the exception of DddA).

Cytosine deaminases are disclosed. Base editors containing such deaminases linked or associated with programmable targeting domains (e.g., DNA binding domains) are also provided. The deaminases and base editors thereof enable the precise editing of DNA both in vitro (e.g., in test tubes) and in vivo (e.g., in living cells). The base editors can efficiently correct a variety of point mutations relevant to human disease. Such custom-designed base editors afford a general and efficient way to introduce targeted (site-specific) base edits to the genome and makes targeted gene correction or genome editing a viable option in human cells. Due to their protein-only nature, and lack of requirement for any nucleic acid moiety (e.g. guide RNA), the described base editors can be effectively used in cases where delivery of nucleic acids to the location of target DNA is challenging, such as editing mitochondrial genome, chloroplast, and other plastids.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### I. Definitions

The term “deaminase” or “deaminase domain” refers to a polypeptide, protein or enzyme that catalyzes a deamination reaction. Deaminase is capable of deaminating an adenine (A) or cytosine (C) in DNA in a non-targeted manner, based on the sequence specificity of the deaminase. dsDNA-specific deaminase can perform deamination reaction on a double-stranded DNA, while the ssDNA-specific deaminase strictly acts on single-stranded DNA as the substrate.

The term “base editor (BE),” refers to a composition including a deaminase domain and one or more functional domains. The deaminase domain and functional domain(s) can be fused or conjugated via a linker. Thus, in some forms, a base editor is a fusion protein. A base editor is capable of making a modification to a base (e.g., A or C) within a target nucleotide sequence in a target nucleic acid (e.g., DNA or RNA). In some forms, the base editor is capable of deaminating a base within a nucleic acid, such as a double-stranded DNA molecule. Preferably, the base editor is capable of deaminating an adenine (A) or cytosine (C) in DNA in a targeted manner.

The term “linker” refers to a bond (e.g., covalent bond), chemical group, or a molecule linking two molecules or moieties, e.g., two domains of a fusion protein, such as, for example, an adenosine or cytosine deaminase domain and a targeting domain (e.g., DNA-binding protein or domain). Typically, the linker is positioned between, or flanked by, two groups, molecules, or other moieties and connected to each one via a covalent bond, thus connecting the two. In some forms, the linker is an amino acid or a plurality of amino acids (e.g., a peptide). In some forms, the linker is an organic molecule, group, polymer, or chemical moiety.

The term “mutation” refers to a change in a sequence resulting in an alteration from a given reference sequence. Mutations include a substitution of a residue within a sequence, e.g., a nucleic acid or amino acid sequence, with another residue, or a deletion or insertion of one or more residues within a sequence. In some form, mutations are described by identifying the original residue followed by the position of the residue within the sequence and by the identity of the newly substituted residue (e.g., D10A). In some forms, mutations are described by identifying the position of the residue within the sequence, the original residue followed by the identity of the newly substituted residue (e.g., 5650G>A). Mutations may or may not produce discernible changes in the observable characteristics (phenotype) of a subject.

The term “target nucleic acid” refers to a nucleic acid molecule which contains a target nucleotide sequence that can be recognized and/or deaminated by a deaminase domain or base editor. The target nucleic acid can be, without limitation, chromosomal DNA, mitochondrial DNA, RNA, plasmid, expression vector, and the like, either inside or outside of a living cell.

The term “target nucleotide sequence” refers to a nucleotide sequence containing a nucleotide that is preferentially deaminated by a deaminase domain over the nucleotide in different nucleotide sequences. Specific instances of a target nucleotide sequence can be targeted for deamination. The target nucleotide sequence can include two or more nucleotides (e.g., 2, 3, 4, 5, 6, 7, 8, 9, 10, or more). Two or more of the nucleotides in the target nucleotide sequence, referred to as target nucleotides, define the target specificity of the deaminase domain that deaminates that target sequence. In some forms, two or more target nucleotides in the target nucleotide sequence are each individually fully or partially defined and are in a fixed sequential relationship to each other. Generally, a specific nucleotide within the “target nucleotide sequence” is deaminated by the deaminase domain. For example, in the exemplary target nucleotide sequence CNAC, the last C in the target nucleotide sequence can be deaminated by the deaminase domain (e.g., a cytosine deaminase). This nucleotide selected for deamination can be referred to as the “target nucleotide.”

The term “base editor target sequence” refers to a sequence within a target nucleic acid molecule that is recognized and bound by a targeted base editor. Generally, the base editor target sequence is distinct from and/or non-overlapping with the target nucleotide sequence that is deaminated by the targeted base editor. Thus, the base editor target sequence encompasses a nucleic acid sequence that, once bound by the targeted base editor, positions the targeted base editor in the vicinity of an instance of the target nucleotide sequence in a nucleic acid molecule. This colocation of the base editor target sequence and instance of the target nucleotide sequence facilitates preferential and specific deamination of the instance of the target nucleotide sequence. Typically, the targeting domain, such as a DNA binding domain, associated with a the targeted base editor recognizes and binds the base editor target sequence.

“Deaminase activity on double-stranded DNA” refers to the deaminase activity of the deaminase on a set of one or more double-stranded DNA segments that all include the target nucleotide sequence. Deaminase activity on double-stranded DNA does not require activity of an accessory factor, such as a guide RNA, to unwind the double stranded DNA. Thus, this activity is distinct from deaminase activity of ssDNA-specific deaminases such as APOBEC and AID, which can only access and deaminate dsDNA at the presence of accessory factors such as RNA-guided DNA binding domains (i.e. dCas9 and guide RNA).

A nucleotide in a nucleotide sequence (such as a target nucleotide sequence) is “fully defined” if that nucleotide must be one particular nucleotide (e.g., C). A nucleotide in a nucleotide sequence (such as a target nucleotide sequence) is “partially defined” if that nucleotide can be two or more particular nucleotides (e.g., C or A) but cannot be any nucleotide (that is, cannot be N). A nucleotide in a nucleotide sequence (such as a target nucleotide sequence) is “undefined” if that nucleotide can be any nucleotide (that is, N).

A group of nucleotides in a nucleotide sequence “in a fixed sequential relationship to each other” refers to such nucleotides that, relative to each instance of the nucleotide sequence, are in the same order on the nucleotide sequence and are spaced from each other by the same number of nucleotides. In the case of spacing, this does not mean or require that the nucleotides in a given instance of the nucleotide sequence are all equally spaced from each other (e.g., all having one nucleotide between each other). Rather, this means that the nucleotides in each instance of the nucleotide sequence have the same spacing of the nucleotide as in all instances of the nucleotide sequence. For example, consider the target nucleotide sequence (C/T)NAC. In this nucleotide sequence the first nucleotide is partially defined, the second nucleotide is undefined, and the third and fourth nucleotides are fully defined. Thus, this represents a nucleotide sequence including three nucleotides that are fully or partially defined. Regarding spacing, the (C/T) nucleotide has one nucleotide between it and the A nucleotide and two nucleotides between it and the C nucleotide; the A nucleotide has no nucleotides between it and the C nucleotide. This same spacing would be present in each instance of this target nucleotide sequence. Regarding order of the nucleotide, the (C/T), A, and C would appear in the same order in each instance of this target nucleotide sequence.

By “isolated” or “purified” with respect to a polypeptide it is meant that the polypeptide is separated to some extent from the cellular components with which it is normally found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography.

“Introduce” refers to bringing in to contact. By “contact” or “contacting” is meant to allow or promote a state of immediate proximity or association between at least two elements. For example, to introduce a base editor, vector or other agent to a cell is to provide contact between the cell and the base editor, vector or agent. The term encompasses penetration of the contacted base editor, vector or agent to the interior of the cell by any suitable means, e.g., via transfection, electroporation, transduction, gene gun, nanoparticle delivery, etc., in any suitable formulation.

The term “expression” encompasses the transcription and/or translation of a particular nucleotide sequence driven by a promoter. “Expression vector” or “expression cassette” refers to a vector containing a recombinant polynucleotide having expression control sequences operably linked to a nucleotide sequence to be expressed. An expression vector contains sufficient cis-acting elements for expression; other elements for expression can be supplied by the host cell or in an in vitro expression system. Expression vectors include all those known in the art, such as cosmids, plasmids (e.g., naked or contained in liposomes), phagemids, BACs, YACs, and viral vectors (e.g., vectors derived from lentiviruses, retroviruses, adenoviruses, and adeno-associated viruses) that incorporate the recombinant polynucleotide.

The term “operably linked” or “operationally linked” refers to functional linkage between elements (e.g., a regulatory sequence and a heterologous nucleic acid sequence) permitting them to function in their intended manner (e.g., resulting in expression of the heterologous nucleic acid sequence). The term encompasses positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. An organelle localization sequence operably linked to protein will direct the linked protein to be localized at the specific organelle.

The term “nuclear localization sequence” or “NLS” refers to an amino acid sequence that promotes import of a peptide or protein into the cell nucleus, for example, by nuclear transport. Nuclear localization sequences are known in the art and would be apparent to the skilled artisan. For example, NLS sequences are described in International PCT Application No. PCT/EP2000/011690, the contents of which are incorporated herein by reference for their disclosure of exemplary nuclear localization sequences.

The term “effective amount,” as used herein, refers to an amount of a biologically active agent that is sufficient to elicit a desired biological response. For example, in some forms, an effective amount of a base editor may refer to the amount of the base editor that is sufficient to induce editing of a target nucleotide sequence. As will be appreciated by the skilled artisan, the effective amount of an agent, e.g., a deaminase domain or base editor, may vary depending on various factors, for example, the desired biological response, e.g., on the specific allele, genome, or target site to be edited, on the cell or tissue being targeted, and on the agent being used.

The terms “nucleic acid” and “nucleic acid molecule,” refer to a molecule including a nucleobase and an acidic moiety, e.g., a nucleoside, a nucleotide, or a polymer of nucleotides. Typically, polymeric nucleic acids, e.g., nucleic acid molecules including three or more nucleotides are linear molecules, in which adjacent nucleotides are linked to each other via a phosphodiester linkage. In some forms, “nucleic acid” refers to individual nucleic acid residues (e.g. nucleotides and/or nucleosides). In some forms, “nucleic acid” refers to an oligonucleotide chain including three or more individual nucleotide residues. As used herein, the terms “oligonucleotide” and “polynucleotide” can be used interchangeably to refer to a polymer of nucleotides (e.g., a sequence of at least three nucleotides). Nucleic acid encompasses RNA as well as single- and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some forms, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “peptide” refers to a class of compounds composed of amino acids chemically bound together. In general, the amino acids are chemically bound together via amide linkages (CONH); however, the amino acids can be bound together by other chemical bonds known in the art. For example, the amino acids can be bound by amine linkages. Peptide as used herein includes oligomers of amino acids and small and large peptides, including polypeptides. Thus, the terms “protein,” “peptide,” and “polypeptide” are used interchangeably herein. The protein, peptide, or polypeptide can be of any size, structure, or function. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The term “percent (%) sequence identity” describes the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software.

Appropriate parameters for measuring alignment, including any algorithms needed to achieve maximal alignment over the full-length of the sequences being compared can be determined by known methods.

“Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988). Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). In some forms, the default parameters can be used to determine the identity for the polynucleotides or polypeptides of the present disclosure.

In some forms, the % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “subject” means any individual, organism or entity. The subject can be a vertebrate, for example, a mammal. Thus, the subject can be a human or an animal, such as a mouse, rat, rabbit, goat, pig, nematode, chimpanzee, or horse. The term does not denote a particular age or sex. Thus, adult and newborn subjects, as well as fetuses, whether male or female, are intended to be covered. The subject may be healthy or suffering from or susceptible to a disease, disorder or condition. A patient refers to a subject afflicted with a disease or disorder. The term “patient” includes human and veterinary subjects.

Recitation of ranges of values herein are merely intended to serve as a shorthand method of referring individually to each separate value falling within the range, unless otherwise indicated herein, and each separate value is incorporated into the specification as if it were individually recited herein.

The term “bit,” as used in the context of a nucleic acid sequence logo is a measure of the height of the letters corresponding to a nucleic acid within a given nucleic acid sequence logo. A nucleic acid sequence logo includes a stack of letters corresponding to a nucleic acid at each position within the sequence. The relative sizes of the letters indicate the frequency of the corresponding nucleic acid(s) in a multitude of aligned nucleic acid sequences. The total height of the letters depicts the information content of the position, in bits.

Use of the term “about” is intended to describe values either above or below the stated value in a range of approximately +/−10%; in other forms the values may range in value either above or below the stated value in a range of approximately +/−5%; in other forms the values may range in value either above or below the stated value in a range of approximately +/−2%; in other forms the values may range in value either above or below the stated value in a range of approximately +/−1%. The preceding ranges are intended to be made clear by context, and no further limitation is implied.

### II. Compositions

Disclosed are reagents and compositions for targeting and editing nucleic acids. Such reagents and compositions include cytosine deaminase domains that are capable of deaminating target nucleotides in single-stranded and/or double-stranded DNA. Also disclosed are non-naturally occurring or engineered DNA base editors containing such deaminase domains in combination with one or more targeting domains such as Cas9, Cpf1, ZF, TALE, that recognize and/or bind a specific target sequence. The base editors facilitate specific and efficient editing of targeted sites within the genome of a cell or subject, e.g., within the human mitochondrial genome, with low off-target effects.

Compositions including one or more functional deaminase proteins that are a non-naturally occurring polypeptide having a double-stranded DNA deaminase activity are described. Generally, the compositions include one or more minimum domains conferring double-stranded DNA deaminase activity. Exemplary protein domains correspond to amino acid sequences of any of SEQ ID NOS: 1-16, 18-19, or 40-67, or a corresponding region of an amino acid sequence having at least 90% sequence identity to any of SEQ ID NOS: 1-16, 18-19, or 40-67.

In some forms the compositions include a non-naturally occurring polypeptide fragment of a functional double-stranded DNA deaminase protein that is obtained by cleaving the deaminase protein at a cleavage site within the functional deaminase domain. For example, in some forms, the fragment corresponds to an N-terminal fragment, wherein the fragment includes an N-terminal portion of a cleaved functional deaminase domain. In other forms, the fragment corresponds to a C-terminal fragment, wherein the fragment includes a C-terminal portion of a cleaved functional deaminase domain. The deaminase activity is restored upon co-localizing the N-terminal fragment with the C-terminal fragment, or upon co-localizing the C-terminal fragment with an N-terminal fragment.

Base editors including a heterodimer having first and second monomers, the first monomer including a first programmable DNA binding protein and an N-terminal or C-terminal fragment of a cleaved double-stranded DNA deaminase, and the second monomer including a second programmable DNA binding protein and an N-terminal or C-terminal fragment of a cleaved double-stranded DNA deaminase, are also described. Typically, dimerization of the first and second monomers reconstitutes the functional double-stranded DNA deaminase protein and the functional double-stranded DNA deaminase activity. In some forms, the first and/or second programmable DNA binding protein are the same. In other forms, the first and/or second programmable DNA binding protein are different. Exemplary first and/or second programmable DNA binding proteins include a Cas domain (e.g., Cas9), a nickase, a zinc-finger protein, a TALE protein, and a TALE-like protein. Therefore, in some forms the base editor includes a heterodimer having first and second monomers, the first monomer including: a Cas domain, a nickase, a zinc-finger protein or a TALE protein; and an N-terminal or C-terminal fragment of a cleaved double-stranded DNA deaminase, and a second monomer including: a Cas domain, a nickase, a zinc-finger protein or a TALE protein; and a second programmable DNA binding protein and an N-terminal or C-terminal fragment of a cleaved double-stranded DNA deaminase, whereby dimerization of the first and second monomers reconstitutes the double-stranded DNA deaminase activity. Exemplary Cas domains include Cas9, Casl2e, Casl2d, Casl2a, Casl2bl, Cas13a, Casl2c, and Argonaute.

In some forms, the base editors include linkers. Linkers could be rigid or flexible based on design parameters to accommodate higher efficiency or expanded or narrower window of activity. For example, in some forms, the first monomer includes a linker that joins the first programmable DNA binding protein with the N-terminal or C-terminal fragment of the cleaved double-stranded DNA deaminase. In some forms, the second monomer includes a linker that joins the first programmable DNA binding protein with the N-terminal or C-terminal fragment of the cleaved double-stranded DNA deaminase. Exemplary linkers include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. Preferred linkers include 2-10 amino acids.

In some forms, the base editors include one or more uracil glycosylase inhibitor (UGI) domains, and/or one or more targeting sequences. Exemplary targeting sequences include a nuclear localization sequence (NLS), a mitochondrial targeting sequence (MTS). Exemplary MTS sequences include an SOD2 sequence and a COX8 sequence.

Therefore, in certain forms, the base editor includes a first and/or second monomer having one of the following structures:
[A]-[programmable DNA binding protein]-[N-terminal or C-terminal fragment of a cleaved double-stranded DNA deaminase]-[B]; or
[A]-[N-terminal or C-terminal fragment of a cleaved double-stranded DNA deaminase] [programmable DNA binding protein]-[B],
where “[A]” and/or “[B]” represent optional one or more additional functional domains and wherein “]-[” is an optional linker.

In an exemplary form, the base editor has the following structure:
[SOD2]-[UGI] (1-2)-[mitoTALE]-[N-terminal or C-terminal fragment of a cleaved double-stranded DNA deaminase]-[UGI](1-2).

In some forms, the first and second monomers bind to first and second nucleotide sequences, respectively, on either side of a target site. An exemplary target site includes a target base which becomes deaminated by the base editor. In some forms, the target base is a C. For example, in some forms the C is within a 5′-TC-3′ sequence context. In other forms, the C is within a 5′-TCC-3′ sequence context. Typically, the nucleotide sequences are each on the same strand as the target base which becomes deaminated by the base editor. In a particular form, a first and second nucleotide sequences are each on the same strand as the strand including the target base which becomes deaminated by the base editor. In another form, a first and second nucleotide sequences are each on the opposite strand as the strand including the target base which becomes deaminated by the base editor. In some forms, the first and second nucleotide sequences are on opposing strands. Base editors including one or more guide RNAs are also described. For example, in some forms, the first and/or second programmable DNA binding protein is a nucleic acid programmable DNA binding protein, and the one or more guide RNAs directs the base editor to bind to the first or second nucleotide sequence at the target site. Isolated nucleic acids encoding the first or second monomers of the base editors are also described. Vectors including the isolated nucleic acids encoding the first or second monomers of the base editors are also described. Cells including the vectors including the isolated nucleic acids encoding the first or second monomers of the base editors are also described.

### A. Deaminase Domains

Disclosed are deaminases, deaminases domains and polypeptides including such deaminases domains. A “deaminase” or “deaminase domain” refers to a polypeptide protein, or enzyme that catalyzes a deamination reaction. Deamination reactions include, but are not limited to, the removal of an amino group from a molecule such as a nitrogenous base (e.g., cytosine, adenine). In some forms, the nitrogenous base is part of a nucleoside, nucleotide, or nucleic acid. Thus, the disclosed deaminases can catalyze deamination of free bases, free nucleosides, free nucleotides, and/or polynucleotides. In some forms, the deaminase domain is capable of deaminating a nitrogenous base in a ribonucleic acid (RNA) or deoxyribonucleic acid (DNA) substrate. In some forms, the deaminase domain catalyzes deamination of both RNA and DNA. The RNA or DNA substrate may be single stranded (ss) or double stranded (ds). In some forms, the deaminase domain catalyzes deamination of ssDNA or dsDNA. In some forms, the deaminase domain catalyzes deamination of both ssDNA and dsDNA.

The deaminase domains provided herein may be derived from any organism. Thus, the deaminase domains can be from a prokaryote or eukaryote. In some forms, the deaminase is a vertebrate deaminase or invertebrate deaminase. In some forms, the deaminase domain is a human, chimpanzee, gorilla, monkey, cow, dog, rat, mouse, fish, fly, worm, fungal, bacterial, viral, or bacteriophage deaminase domain.

In preferred forms, organisms from which the deaminase domain may be derived include, without limitation, Skermanella stibiiresistens, Erythranthe guttata, Citrus sinensis, Hydrocarboniphaga daqingensis, Tieghemostelium lacteum, Saprolegnia parasitica, Vitrella brassicaformis, Leishmania infantum, Simonsiella muelleri, Clostridiales bacterium, Kibdelosporangium aridum, Desmospora activa, Neisseria gonorrhoeae, Bacillus asahii, Saezia sanguinis, Bacillus anthracis, Hungateiclostridium clariflavum, Ruminococcus sp. CAG:563, Clostridium disporicum, Umezawaea tangerina, Conchiformibius steedae, Streptomyces coelicolor, Streptomycetaceae bacterium MP113-05, Verrucosispora sp. LHW63014, Vibrio aerogenes, Fusarium oxysporum, Verticillium longisporum, Chondromyces crocatus, Kitasatospora aureofaciens, Colletotrichum orchidophilum, Nonomuraea solani, Aquimarina spongiae, Dipodomys ordii, Patagioenas fasciata monilis, Streptomyces phaeoluteigriseus, Ictalurus punctatus, Corynespora cassiicola, Platysternon megacephalum, Streptomyces sp. AC1-42W, Gimesia maris, Burkholderia glumae, Nakamurella multipartita, Stackebrandtia nassauensis, Kitasatospora setae, Aspergillus kawachii, Streptomyces turgidiscabies, Anolis carolinensis, Serratia rubidaea, Ruminiclostridium cellulolyticum, Alloactinosynnema iranicum, Photorhabdus laumondii, Escherichia coli, Staphylococcus aureus, Salmonella typhi, Shewanella putrefaciens, Haemophilus influenzae, Caulobacter crescentus, Bacillus subtilis, and Caenorhabditis elegans

In some forms, organisms from which the deaminase domain may be derived include, without limitation, Skermanella sp., Erythranthe sp., Citrus sp., Hydrocarboniphaga sp., Tieghemostelium sp., Saprolegnia sp., Vitrella sp., Leishmania sp., Simonsiella sp., Clostridiales sp., Kibdelosporangium sp., Desmospora sp., Neisseria sp., Bacillus sp., Saezia sp., Bacillus sp., Hungateiclostridium sp., Ruminococcus sp., Clostridium sp., Umezawaea sp., Conchiformibius sp., Streptomyces sp., Streptomycetaceae sp., Verrucosispora sp., Vibrio sp., Fusarium sp., Verticillium sp., Chondromyces sp., Kitasatospora sp., Colletotrichum sp., Nonomuraea sp., Aquimarina sp., Dipodomys sp., Patagioenas sp., Ictalurus sp., Corynespora sp., Platysternon sp., Streptomyces sp., Gimesia sp., Burkholderia sp., Nakamurella sp., Stackebrandtia sp., Kitasatospora sp., Aspergillus sp., Anolis sp., Serratia sp., Ruminiclostridium sp., Alloactinosynnema sp., Photorhabdus sp., Escherichia sp., Staphylococcus sp., Salmonella sp., Shewanella sp., Haemophilus sp., Caulobacter sp., Bacillus sp., and Caenorhabditis sp.

The disclosed deaminase or deaminase domains may belong to any known deaminase clan or family. See, for example, Iyer L M, et al., Nucleic Acids Res., 39(22):9473-97 (2011), which is hereby incorporated by reference in its entirety. Exemplary deaminase clans include, but are not limited to, CDD/CDA cytidine deaminases, Blasticidin S-deaminase (BSD), Plant Des/Cda, LmjF36.5940-like, PITG_06599-like, DYW like, BURPS668_1122, Pput_2613, SCP1.201, YwgJ, MafB19, TadA-Tad2(ADAT2), Bd3614, Tad1, RibD-like (diamino-hydroxy-phosphoribosyl aminopyrimidinedeaminase), Guanine deaminase, dCMP deaminase and ComE, AID/APOBEC, ZK287.1, B3gp45, XOO_2897, and OTT_1508 (see Table 1 of Iyer L M, et al.). In preferred forms, the deaminase or deaminase domains are derived from Cytidine deaminase-like (CDA), MafB19-like deaminase, SCP1201-deam, SNAD1, SNAD2, SNAD4, CMP/dCMP, Pput2613-deam, LmjF365940-deam, LoxI_N, DAAD, DYW, YwgJ-deaminase, or SUKH-4 families.

The CDA clan contains both free nucleotide and nucleic acid deaminases that act on adenosine, cytosine, guanine and cytidine, and are collectively known as the deaminase superfamily. The conserved fold consists of a three-layered alpha/beta/alpha structure with 3 helices and 4 strands in the 2134 order (Liaw S H, et al., J Biol Chem., 279:35479-35485 (2004); Iyer L M, et al., Nucleic Acids Res., 39(22):9473-97 (2011)). This superfamily is further divided into two major divisions based on the presence of a helix (helix-4) that renders the terminal strands (strands 4 and 5) either parallel to each other in its presence, or anti-parallel in its absence. The active site of the deaminases is composed of three residues that coordinate a zinc ion between conserved helices 2 and 3. The residues are typically found as [HCD]xE and CxxC motifs at the beginning of helices 2 and 3. The zinc ion activates a water molecule, which forms a tetrahedral intermediate with the carbon atom that is linked to the amine group. This is followed by deamination of the base. The MafB19-like deaminase family is a member of the nucleic acid/nucleotide deaminase superfamily prototyped by Neisseria MafB19. Members of this family are present in a wide phyletic range of bacteria and are predicted to function as toxins in bacterial polymorphic toxin systems. SCP1.201-like deaminases are members of the nucleic acid/nucleotide deaminase superfamily prototyped by Streptomyces SCP1.201. Members of this family are predicted to function as toxins in bacterial polymorphic toxin systems.

The deaminase or deaminase domain can be a variant of a naturally-occurring deaminase from an organism, including any of the foregoing, such as a bacterium. In some forms, the deaminase or deaminase domain does not occur in nature. For example, in some forms, the deaminase or deaminase domain shows at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% sequence identity to a naturally-occurring deaminase domain.

The size of the deaminase or deaminase domain can vary. In some forms, the deaminase or deaminase domain is from about 50-250, 50-200, 50-150, 50-100, 100-250, 100-200, 100-150, 100-120, 120-160, 120-140, 140-160, 150-250, 150-200, 200-250, or 200-220 amino acids in length. In some forms, the deaminase or deaminase domain is about 40, 50, 60, 70, 80, 90, 100, 110, 120, 130, 140, 150, 160, 170, 180, 190, 200, 210, 220, 230, 240, or 250 amino acids in length.

In some forms, the disclosed deaminases or deaminase domains can be split into two or more distinct portions (e.g., 2, 3, 4, or 5). In such forms, a split deaminase domain is only capable of deaminating a substrate when the subcomponents are combined (e.g., co-expressed or co-introduced), and/or brought into proximity together (e.g. by DNA targeting domains). For example, Example 1 demonstrates that a single deaminase domain can be separated into N-terminal and C-terminal portions, which exhibit deaminase activity upon their combination. Those of ordinary skill in the art will understand that the deaminase domains can be split at different positions and will be able to determine where a single deaminase domain should be split in order to retain deaminase activity upon combination of its complementary components.

In some forms, the deaminase domain is a cytosine deaminase (also referred to herein as a cytidine deaminase), which catalyzes the hydrolytic deamination of cytidine or cytosine. In some forms, the cytosine deaminase catalyzes the hydrolytic deamination of cytidine or deoxycytidine to uridine or deoxyuridine, respectively. In some forms, the cytosine deaminase domain catalyzes the hydrolytic deamination of cytosine to uracil.

In some forms, the deaminase domain is an adenosine deaminase (also referred to herein as an adenine deaminase), which catalyzes the hydrolytic deamination of adenine or adenosine. In some forms, the adenosine deaminase catalyzes the hydrolytic deamination of adenosine or deoxyadenosine to inosine or deoxyinosine, respectively.

In a particular form, disclosed is an isolated deaminase domain, wherein the deaminase domain can deaminate double-stranded DNA. The deaminase domain can have greater deaminase activity on double-stranded DNA containing a target nucleotide sequence as compared to the deaminase activity of the deaminase domain on double-stranded DNA that does not contain the target nucleotide sequence. Preferably, the target nucleotide sequence contains two or more target nucleotides (e.g., 2, 3, 4, 5, 6, 7, 8, 9, 10, or more), wherein the target nucleotides are each individually fully or partially defined and are in a fixed sequential relationship to each other. In some forms, the target nucleotide sequence includes three or more target nucleotides. In some forms, the target nucleotide sequence includes four or more target nucleotides. In some forms, the target nucleotide sequence includes five or more target nucleotides. In such forms, the target nucleotides are each individually fully or partially defined and are in a fixed sequential relationship to each other. Preferably, the deaminase domain is not the deaminase domain of DddA from Burkholderia cenocepacia (see Mok B Y., et al., Nature, 583(7817):631-637 (2020)).

The deaminase domain can show a range of editing efficiencies in deaminating a nucleic acid substrate (e.g., ssDNA, dsDNA, RNA) containing a target nucleotide sequence. In some forms, the editing efficiency of a nucleic acid substrate containing a target nucleotide is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some forms, the editing efficiency of a nucleic acid substrate containing a target nucleotide is at least 1%. In some forms, the editing efficiency of a nucleic acid substrate containing a target nucleotide is at least 10%. In some forms, the editing efficiency of a nucleic acid substrate containing a target nucleotide is at least 25%. In some forms, the editing efficiency of a nucleic acid substrate containing a target nucleotide is at least 50%.

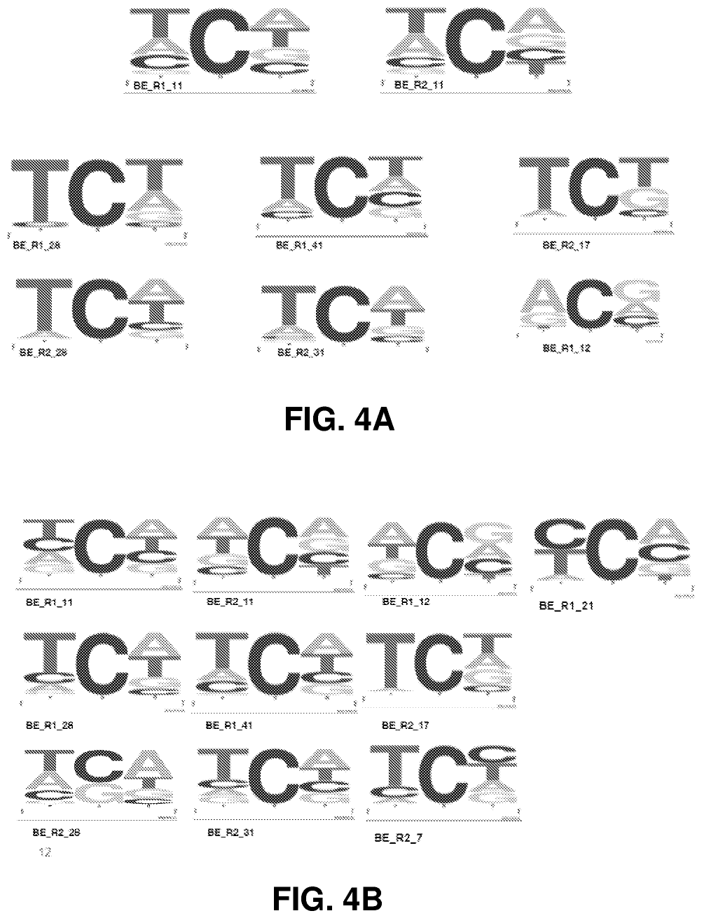

In some forms, the target nucleotide sequence that is recognized and/or deaminated by a deaminase domain can be represented as a sequence logo. A sequence logo is a graphical representation of an amino acid or nucleic acid multiple sequence alignment. See, for example, FIGS. 4A-4C. Each logo contains stacks of symbols, one stack for each position in the sequence. The overall height of the stack indicates the sequence conservation at that position, while the height of symbols within the stack indicates the relative frequency of each amino or nucleic acid at that position. Within each stack, the characters are ordered by their relative frequency, and the total height of the stack is determined by the information content of the position, in bits (see Dey, K K., et al., BMC Bioinformatics. 19, 473 (2018); Schneider T D., et al, Nucleic Acids Res., 18(20):6097-100 (1990)).

The target nucleotides can each exhibit a context specificity defined by the deaminase probability sequence logo at a defined editing threshold. The residue immediately before the target nucleotide is the most important specificity defining residue, so the meaningful specificities are ACN, CCN, GCN, TCN. Such specificities can be useful for reducing o-target editing. But broad specificity deaminases allow editing a wider variety of target, and off-target editing can be limited by other features and designs described herein.

As an example of deaminase specificity, BE_11_R1 can edit all the TC, AC and CC contexts with almost equal probability but it is less active on GC context. For the same deaminase, the position after the target nucleotide could be any nucleotide with almost equal probability. So, the preferred (most probable) site for BE_R1_11 based on the logo shown in FIG. 4 is TCA, but other sites are also very probable. For a narrow specificity deaminase like BE_R2_17, the most probable (observed) editing sites are TCT, TCG, and TCA (this means, out of all the 64 possible 3 nucleotide combinations in our substrate, these 3 combinations were the main combinations that got edited by this deaminase with at least 50% efficiency).

One of ordinary skill in the art could readily determine an appropriate method for deriving a sequence logo for any disclosed deaminase domain. A non-limiting example is described in Example 1. In brief, in some forms, the deaminase domain of interest can be incubated with different nucleic substrates (i.e. having different sequences) containing a target nucleotide (e.g., a C in case of a cytosine deaminase domain or an A in case of a adenosine deaminase domain) in various sequence contexts. The substrates are then sequenced. Sequence variants resulting from editing (deamination) of the target nucleotide are then identified, and a sequence logo can be generated from multiple sequence alignment of these sequence variants. A variety of tools are available in the art for generating sequence logos. Non-limiting examples include Seq2Logo (website cbs.dtu.dk/biotools/Seq2Logo/), WebLogo (internet site weblogo.berkeley.edu/logo.cgi), and Weblogo (Crooks G E, et al., Genome Research, 14:1188-1190 (2004)). In some forms, a sequence logo can be determined for different levels of editing (deaminating) efficiencies, such as 1%, 10%, 25%, or 50% (see e.g., FIGS. 4A-4C).

Thus, in some forms, a disclosed deaminase domain has deaminase activity on a nucleic acid substrate containing a target nucleotide sequence represented as a sequence logo. In some forms, the target nucleotides in a target nucleotide sequence (sequence logo) each exhibit from about 0.1 to 2.0 bit, inclusive. For example, in some forms, the target nucleotides in a target nucleotide sequence (sequence logo) each exhibit about 0.1, about 0.2, about 0.25, about 0.3, about 0.4, about 0.5, about 0.6, about 0.7, about 0.75, about 0.8, about 0.9, about 1.0, about 1.1, about 1.2, about 1.25, about 1.3, about 1.4, about 1.5, about 1.6, about 1.7, about 1.75, about 1.8, about 1.9, or about 2.0 bit.

In some forms, the target nucleotides in a target nucleotide sequence (sequence logo) each exhibit from about 0.1 to about 2.0 bit when from about 1% to about 90% of the target nucleotide sequence is edited. For example, in some forms, the target nucleotides each exhibit at least 0.1 bit when 1% or greater of the target nucleotide sequence is edited. In some forms, the target nucleotides each exhibit at least 0.1 bit when 10% or greater of the target nucleotide sequence is edited. In some forms, the target nucleotides each exhibit at least 0.1 bit when 25% or greater of the target nucleotide sequence is edited. In some forms, the target nucleotides each exhibit at least 0.1 bit when 50% or greater of the target nucleotide sequence is edited.

In some forms, the target nucleotides each exhibit at least 0.25 bit when 1% or greater of the target nucleotide sequence is edited. In some forms, the target nucleotides each exhibit at least 0.25 bit when 10% or greater of the target nucleotide sequence is edited. In some forms, the target nucleotides each exhibit at least 0.25 bit when 25% or greater of the target nucleotide sequence is edited. In some forms, the target nucleotides each exhibit at least 0.25 bit when 50% or greater of the target nucleotide sequence is edited. In some forms, the target nucleotides each exhibit at least 0.5 bit when 1% or greater of the target nucleotide sequence is edited. In some forms, the target nucleotides each exhibit at least 0.5 bit when 10% or greater of the target nucleotide sequence is edited. In some forms, the target nucleotides each exhibit at least 0.5 bit when 25% or greater of the target nucleotide sequence is edited. In some forms, the target nucleotides each exhibit at least 0.5 bit when 50% or greater of the target nucleotide sequence is edited.

In a particular form, the isolated deaminase domain can deaminate cytosine-containing nucleotides (referred to as a cytosine deaminase). Exemplary target nucleotide sequences that can be deaminated by the cytosine deaminase include, without limitation, AC, CC, GC, and TC. In some forms, target nucleotide sequences that can be deaminated by the cytosine deaminase include, without limitation, Ac, Cc, Gc, and Tc, where N represents, independently, any nucleotide, and the cytosine-containing nucleotide that is deaminated is in lowercase.

### 1. Exemplary Cytosine Deaminase Domains

In various forms, the dsDNA base editors or the polypeptides that comprise the dsDNA base editors (e.g., the DNAbps and CDA) may be engineered to include a cytosine deaminase (CDA), or an inactive or truncated fragment thereof. Amino acid sequences of exemplary cytosine deaminases that can be used in accordance with the disclosed compositions and methods are provided below.

In various forms, the CDA protein is BE11 (component of Uniprot ID NO.: A0A1Y5Y1M1_KIBAR), having the following amino acid sequence: TKSANSGGAAKDLAKYRERQGMPRAGSADDAHTAARLDVGGRSFYGHNAHGRNIDIKVNA QTKTHAEADVFQQAKNAKVSADRATLHVDRDLCDACGIKGGVGSLMRGVGISRLTVNSPS GRFEITASRPSVPRRING (SEQ ID NO:1), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:1, or a fragment thereof.

In various forms, the CDA protein is BE12 (component of Uniprot ID NO.: A0A2T4Z6L8_9BACL), having the following amino acid sequence: FSKAESGYIEIQRFRRILNMPRYSLTNGRTGTVARVEVNGRRIFGVNTSLIKNSKYAPRD MDLRRRWLREVNWVPPKKNKPNHLGHAQSLSHAESHALIRAYERMERLGGQLPKKLTMVV DRPTCNICRGEMPALLKRLGIEELTIYSGGRDAIIIKAIK (SEQ ID NO:2), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:2, or a fragment thereof.

In various forms, the CDA protein is BE28 (component of Uniprot ID NO.: AOAOK1EKV1_CHOCO), having the following amino acid sequence: GVGGAITATVGSTAGAAGRAAARAPSLPAYAGGKTSGVLRTTAGDTALLSGYKGPSASMP RGTPGMNGRIKSHVEAHAAAVMREQGMKEGTLYINRVPCSGATGCDAMLPRMLPPDAHLR VVGPNGYDQVFVGLPD (SEQ ID NO:3), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:3, or a fragment thereof.

In various forms, the CDA protein is BE_R1_41 (component of Uniprot ID NO.: C5ALM7_BURGB), having the following amino acid sequence: DPIGLMGGLNLYQYAPNSIAWTDWWGLAGSYTLGSYQISAPQLPAYNGQTVGTFYYVNGA GGLESRTFSSGGPTPYPNYANAGHVEGQSALFMRDNGISDGLVFHNNPEGTCGFCVNMTE TLLPENSKLTVVPPEGAIPVKRGATGETRTFTGNSKSPKSPVKGEC (SEQ ID NO:4) or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO: 4, or a fragment thereof.

In some forms, the CDA protein is BE_R2_7 (component of Uniprot ID NO.: A0A1U7ISE2_9CYAN) having the following amino acid sequence: MPPAGSETDKSTIAKLEISGQNFFGINSGSNPNPRQITFNVNPITKTHAEADAFQQAADV GIRGGKARLIVDRDLCAACGIRGGVNSMAWQLGIEELEIITPSVSKTIAVKPPNRRRQ (SEQ ID NO:8), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:8, or a fragment thereof.

In some forms, the CDA protein is BE_R2_11 (component of Uniprot ID NO.: AOA2T4Z7P2_9BACL) having the following amino acid sequence: SQFDNVRKDMGLPARIGDDDPYTTSVLRIDGHEYWGKNGKWVTKGKTSNYTDKAHYDKVR KELGTSAEVPGHAEGVAFNKAYQVRKNTGTKGGNAVLYVDKIPCVMCKPGIATLMRSAKV DHLDLHYLQDGKMHHVQYVRNPDTDAVYNPFSGKWTKPSKKK (SEQ ID NO:9), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:9, or a fragment thereof.

In some forms, the CDA protein is BE_R2_17 (component of Uniprot ID NO.: D2ZY33_NEIMU) having the following amino acid sequence: GRLKKDERVYRNAHQPFRLQNQYYDEETGLHYNLMRYYEPEAGRFVNQDPIGLLGGDNLY WFAPNAAMWLDPWGLAVVDAIFEMQGHTFTGTNPLDRNPRISSPIQGLSAVNNDKFKMHA EIDAMTQAHDKGLRGGKGVLKIKGKNACSYCKGDIKKMALKLDLDELEVHNHDGTVHKFS KGDLKPVKKGGKGWKKPKKSKKPGAC (SEQ ID NO:10), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:10, or a fragment thereof.

In some forms, the CDA protein is BE_R2_18 (component of Uniprot ID NO.: A0A0A8K6F0_9RHIZ) having the following amino acid sequence: RAPEAIQTLRDSYGTDLLGRPLLGDSDTVAHGIVDGETFMGVNSGAIVEYSQRDLNDAKR ALIPLVRKRPDIMSTHNIGQRPNDALFHAESTVLLRAARANDGTLSGKVIDITVDRPICS SCKKVLPLIGQELGNPIVRFTEPSGRVRTMHNGEWKDQD (SEQ ID NO:11), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:11, or a fragment thereof.

In some forms, the CDA protein is BE_R2_29 (component of Uniprot ID NO.: D2QYF9_PIRSD) having the following amino acid sequence: GALDNLAQTVTVADNATPSSADIFAEIAKSGDNASQSTVDTFTDLAKSLDEAPPLDQSNA PNRTPWDTIDHFRSHKQGMAELGDAIPVKGDKLGTVAFVEIEGSKVFGVNSTALVDDADK ALGRMWRDRLGFNSGQAQALFHGEAHSLMRAYEKFSGKLPKDLTLYVDRLTCGPCQGALP DLMKAMGIERLKIVTKSGRVGEISGGVFRWLE (SEQ ID NO:14), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:14, or a fragment thereof.

In some forms, the CDA protein is BE_R2_31 (component of Uniprot ID NO.: G8SI56_ACTS5) having the following amino acid sequence: GGGTVTVSSTASAQVYATAQTEVEVTKKTKELAAEQQQAQAYQCPVTGKACTGDPFNDLA AFRKRQGMPEAGTDADKDTAARLDVGGQIFYGRNGKGKVTDIPVNAYTRDHAEGDVFQQA KNAKITADRAVMYVDRPLCDGCGAYGGVGSLLRGTGIKEVVVVAPNGRFLITAARPSTPQ PLD (SEQ ID NO:15), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:15, or a fragment thereof.

In some forms, the CDA protein is BE_R2_48 (component of Uniprot ID NO.: A0A2T4Z6L8_9BACL) having the following amino acid sequence: GAASVGRGASHFSKAESGYIEIQRFRRILNMPRYSLTNGRTGTVARVEVNGRRIFGVNTS LIKNSKYAPRDMDLRRRWLREVNWVPPKKNKPNHLGHAQSLSHAESHALIRAYERMERLG GQLPKKLTMVVDRPTCNICRGEMPALLKRLGIEELTIYSGGRDAIIIKAIK (SEQ ID NO:16), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:16, or a fragment thereof.

In some forms, the CDA protein is BE_R1_10 (component of Uniprot ID NO.: A0A3P2ALZ1_9FIRM) having the following amino acid sequence: MEMGTRSLPQETEYMREALKEAEKAYALGETPIGCVIVWRGEIIGRGYNRRAIDKSVLAH AEITAIAEAERYLADWRLEEATLYVTLEPCPMCAGAIVQARVGRVVYATANLKAGSAGTV IDMMHVAGFNHQVEVVGGILEKECTDLLKRFFRELRAEKDKPYPPK (SEQ ID NO:40), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:40, or a fragment thereof.

In some forms, the CDA protein is BE_R_15 (component of Uniprot ID NO.: AOA433SEU4_9BURK) having the following amino acid sequence: EVQARLNGLAAEARQGLPPNKGNVAVAEINIPELADQPFITKAFSGYQTDKDGFVGKPSG NVDTWALQPQKSSPEFIGGPGAYFRDVDTEFKILENLAQKLGPNTNATGTVNLISEKVVC PSCTTVIMQFRERYPNIQLNIFTRD (SEQ ID NO:41), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:41, or a fragment thereof.

In some forms, the CDA protein is BE_R1_21 (component of Uniprot ID NO.: A0A3P2AOL6_9NEIS) having the following amino acid sequence: INYAKENGITGGRNVAVFEYIDLNGKIQTIIKASERGKGHAERLIAMELQNKGIPNSNVT RIYSELEPCSAPGGYCSNMIKYGSPNGLGPYSNAKVTYSFSYGGNPHNAEAARQGVDALR KAREQQKR (SEQ ID NO:42), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:42, or a fragment thereof.

In some forms, the CDA protein is BE_R2_1 (component of Uniprot ID NO.: A0A0F6W299_9DELT) having the following amino acid sequence: GGTPSCSTTLDGLVPTDALEEFATRAYTQEEGACSGYYVVGSANSARVEGVLTACDATTT SVGNEWREEAGTTRACQLFGWPGAIPESVEIDRARCRLAEQDWARLQQRREDCGLPPRTL VPNDGHTVAILTTPGEDEITGLNGRTGGAQPYRARAVEEGTCPPPLTRTYGEDATRYRGA GPTHCHAEGDALEQLSVLRMREPGTPGAGDPRQGATGGRTTGSAELIVDRDPCAMSCAPR GVDRMRSIAGLEELIVRSPQGTRRYADGLPETGVPLD (SEQ ID NO:43), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:43, or a fragment thereof.

In some forms, the CDA protein is BE_R2_3 (component of Uniprot ID NO.: A0A0N9HXW6_9PSEU) having the following amino acid sequence: GRLGSEVGEGVLAARPADGHTIKVTESGRIIRCSRCDDILDLLDEYRAVFADNPGYVERL GRIEDLADAARKARKAKNPNASQLADQAADDAAALLRDVRTSAQARGNLAREGQPLSGAG RLPAEVVQPISPARIQEGLNSLAAQRVQRGLPPAGSATDVSTVCRLDIGGESFYGVNAHH TTMDLHVNAQTATHAEGQAFQLGARSLPASRETRAVLYVDRELCRACGDFGGVESMAKQL GLLQLDVYTPNGLALTLDFAGR (SEQ ID NO:44), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:44, or a fragment thereof.

In some forms, the CDA protein is BE_R2_19 (component of Uniprot ID NO.: A0A1I4B7X1_9PSEU) having the following amino acid sequence: GSYASPDPLGLEAAPNNHAYVANPATAADPTGLIPCDVADDLAAYRQRQGMPVAGSAEDA HTAARLDVDGQSFYGRNGHGMDIDIRANAQTKTHAEAQAFQEAKNAGVSGKTGTLYVDRD FCRACGPNGGVGSLMRGLGLERLEVHTPSGRYTIDATKRPSIPVPWSEG (SEQ ID NO:45), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:45, or a fragment thereof.

In some forms, the CDA protein is BE_R2_20 (component of Uniprot ID NO.: A0A1M7DT37_9FIRM) having the following amino acid sequence: MPVAGSVDDKHTAAKLIFGDNEYYGHNGHGMQDEVKGAFSVNAQTATHAEGLAFYNAKTS GVEGTSATLITDRPACASCGYYGGIRSMAKDMGINDLTVVSPNNAPITFNPQVKPIPNPF PKPVPKTIR (SEQ ID NO:46), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:46, or a fragment thereof.

In some forms, the CDA protein is BE_R2_21 (component of Uniprot ID NO.: A0A1N6MQY7_9GAMM) having the following amino acid sequence: GLAGGEKPYAYVGNPAQAVDPLGLAGCEDPWKIVDRFRRSKNKMEPLGDRIPGAIDKDGL HTVAFFEMNGRRVFGVNSGTLYKKDKALGKQWNEKIDYLTKEEKGTSAFHAEGHALMRAH KKFGGVMPKEITMYVDRVTCNHCERFLPALMKEMGIEKLKLFSKNGTSSVLHAAR (SEQ ID NO:47), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:47, or a fragment thereof.

In some forms, the CDA protein is BE_R2_28 (component of Uniprot ID NO.: B9JGM2_AGRRK) having the following amino acid sequence: GSNGAIYSDVAAAQKAATTASRIGFNDLATFRVQLGLPPAGTAADKSTLAVIEINGQKIY GVNAHGQPVSGVNAISSTHAEIDALNQIKQQGIDVSGQNLTLYVDRTPCAACGTNGGIRS MVEQLGLKQLTVVGPDGPMIVTPR (SEQ ID NO:48), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:48, or a fragment thereof.

In some forms, the CDA protein is BE_R4_4 (component of Uniprot ID NO.: B9JGM2_AGRRK) having the following amino acid sequence: DKVADDVVEDAAKAIKGGSSSINLPEYDGKTTHGVLVLDDGTQVPFSSGNANPNYKNYIP ASHVEGKSAIYMRENGINNGTVFHNNTDGTCPYCDKMLPTLLEEGSTLTVVPPANANAPK PSWVDTVKTYIGNDKIPKKPK (SEQ ID NO:49), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:40, or a fragment thereof.

In some forms, the CDA protein is BE_R4_6 (component of Uniprot ID NO.: A0A7G9FZY2_9FIRM) having the following amino acid sequence: MSLPEYDGTTTHGVLVLDDGTQIGFTSGNGDPRYTNYRNNGHVEQKSALYMRENNISNAT VYHNNTNGTCGYCNTMTATFLPEGATLTVVPPENAVANNSRAIDYVKTYTGTSNDPKISP RYKGN (SEQ ID NO:50), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:50, or a fragment thereof.

In some forms, the CDA protein is BE_R4_7 (fragment of Uniprot ID NO.: AOA7X7XYI6_CLOSP) having the following amino acid sequence: MSITDRLAKQKEKQDNTNIIDNRPKLPDYDGKTTHGILVTPNSEHIPFSSGNPNPNYKNY IPASHVEGKSAIYMRENGITSGTIYYNNTDGTCPYCDKMLSTLLEEGSVLEVIPPINAKA PKPSWVDKPKTYIGNNKVPKPNK (SEQ ID NO:51), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:51, or a fragment thereof.

In some forms, the CDA protein is BE_R4_10 (component of Uniprot ID NO.: MBR1615955.1) having the following amino acid sequence: ELPPYDGKTTYGVLILDDGKQYSFNSGKPAPIYRNYIPASHVEGKAAIYMRENKIQSGTV YHNNTDGTCPYCDKMLPTLLEKDSTLKVVPPQNATSSKKGWITNEKIYIGNDKIPKT (SEQ ID NO:52), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:52, or a fragment thereof.

In some forms, the CDA protein is BE_R4_12 (component of Uniprot ID NO.: MGYP000605828529) having the following amino acid sequence: TDEFKLAYEQLKDIEQAYEYANIDKDKIDIPDFDGKITWGILVLEDGTCITFSSGNANPM FNHYIPASHAEGKAAIYMRQKGIKHGVIFHNNTDGTCPYCNTMLPTLLEENSTLIVVPPI NAVAKKRGWIDKIKIYTGNNKIPKTN (SEQ ID NO:53), or an amino acid sequence having at least 70%, at least 75%, at least 80%, at least 85%, at least 90%, at least 95%, at least 96%, at least 97%, at least 98%, at least 99% or 100% sequence identify with CDA of SEQ ID NO:53, or a fragment thereof.

In some forms, the CDA protein is BE_R4_13 (component of Uniprot ID NO.: WP_021798742) having the following amino acid sequence: GASGAAGHGLSTTGKNVLGHFEPTPTTPQGTSSDTIAEMLNSASQPGRTAGVLDIDGELT PLTSGRPSLPNYIASGHVEGQAAMIMRQQQVQSATVYHDNPNGTCGYCYSQLPTLLPEGA ALDVVPPAGTVPPSNRWHNGGPSFIGNSSEPKPWPR (SEQ ID NO:54), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:54, or a fragment thereof.

In some forms, the CDA protein is BE_R4_14 (component of Uniprot ID NO.: WP_059988487) having the following amino acid sequence: SHYAEEYKQLLKDIDTKREAEEAALLREAYPSMEGATLPPFDGKTTIGLMFYTDASGQYQ VKKLFSGEKVLSNYDATGHVEGKAALIMRNEKITEAVVMHNHPSGTCNYCDKQVETLLPK NATLRVIPPENAKAPTSYWNDQPTTYRGDGKDPKAPSKK (SEQ ID NO:55), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:55, or a fragment thereof.

In some forms, the CDA protein is BE_R4_15 (component of Uniprot ID NO.: WP_082507154) having the following amino acid sequence: ASASPSTNSAGSSGKNVRLPRDYASELPEYDGKTTYGVLVTNEGKVIQLRSGGKEVPYSG YKAVSASHVEGKAAIWIRENASSGGTVYHNNTTGTCGYCNSQVKALLPEGVELKIVPPAN AVARNSQAKAIPTINVGNATQPGRKP (SEQ ID NO:56), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:56, or a fragment thereof.

In some forms, the CDA protein is BE_R4_16 (component of Uniprot ID NO.: WP_112210906) having the following amino acid sequence: KPEALKDAREPKTKPPHNRVHQDPNTSWNPNNYPDTPSGQLPAYDGKNTLGRIEIDGEIY HVKNGKGQPGETLKTDPTVKAGAVSPSHAEGHAVAIMKETGTKEAVLDINHPTGPCGFCD KVLENMLPEGSKLTVNWPNGSQVFTGNSK (SEQ ID NO:57), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:57, or a fragment thereof.

In some forms, the CDA protein is BE_R4_17 (component of Uniprot ID NO.: WP 133186147) having the following amino acid sequence: SHYAKEYKQLLADIDALAEAREDALLREQFPSMDAVTLPPFDGKTTIGYMFYTDANGQYH VRKLYSGGKVLSNYDSSGHVEGMAALIMRKGRITEAVVMHNHPSGTCHYCNGQVETLLPK NAKLKVIPPANAKAPTKYWYDQPVDYLGNSNDPKPPS (SEQ ID NO:58), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:58, or a fragment thereof.

In some forms, the CDA protein is BE_R4_18 (component of Uniprot ID NO.: WP_157869269) having the following amino acid sequence: GGSAVVGGGIAATGAKALTTGKKLTESPGTLNAAQRLLASIGEEGKTAGVLEVDGALFPL VSGKSVLPNYAASGHVEGQAALLMQGMGATNGRLLIDNPNGICGYCTSQVPTLLPENAVL EVGTPLGTVTPSARWSASKPFIGNDREPKPWPR (SEQ ID NO:59), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:59, or a fragment thereof.

In some forms, the CDA protein is BE_R4_19 (component of Uniprot ID NO.: WP_165946289) having the following amino acid sequence: IGKVGKLRFAPKVESAESMLRSLSQEGKTAGVLDINGELIPLVSGTSSLKNYAASGHVEG QAALIMRERGVASARLIIDNPSGICGYCRSQVPTLLPAGATLEVTTPRGTVPPTARWSNG KTFVGNENDPKPWPR (SEQ ID NO:60), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:60, or a fragment thereof.

In some forms, the CDA protein is BE_R4_20 (component of Uniprot ID NO.: WP_174422267) having the following amino acid sequence: LEDKIDYDDLVRKREKAREDLLEAEKRLREEEIRAKYPTPEEAQLPPYDGDTTYALMYYT DEHGKSHVVELSSGGADDEHSNYAAAGHTEGQAAVIMRQRKITSAVVVHNNTDGTCPFCV AHLPTLLPSGAELRVVPPRSAKAKKPGWIDVSKTFEGNARKPLDNKNKKST (SEQ ID NO:61), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:61, or a fragment thereof.

In some forms, the CDA protein is BE_R4_21 (component of Uniprot ID NO.: WP_189594293) having the following amino acid sequence: GGSAVVGAGVVATGAKAVTTGKSLSESQATLSVAQRLLATIGEEGKTAGVLELDGELIPL VSGKSSLPNYAASGHVEGQAALIMRDRGATSGRLLIDNPSGICGYCKSQVATLLPENATL QVGTPLGTVTPSSRWSASRTFTGNDRDPKPWPR (SEQ ID NO:62), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:62, or a fragment thereof.

In some forms, the CDA protein is BE_R4_22 (component of Uniprot ID NO.: MGYP000498443267) having the following amino acid sequence: DSAVDRLEQELEKLDVRNFFEDESETESGSSSINLPEYDGKTTHGVLVLDDGTQVPFSSG NANPNYKNYIPASHVEGKSAIYMRENGINNGTVFHNNTDGTCPYCDKMLPTLLDEGSTLT VVPPTNASAPKPSWVDTVKTYIGNDKIPKKPK (SEQ ID NO:63), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:63, or a fragment thereof.

In some forms, the CDA protein is BE_R4_23 (component of Uniprot ID NO.: WP_195441564) having the following amino acid sequence: SGYDSQYPCKEEMSAGAGESGRKTISLPEYDGTTTHGVLVLDDGTQIGFTSGNGDPRYTN YRNNGHVEQKSALYMRENNISNATVYHNNTNGTCGYCNTMTATFLPEGATLTVVPPENAV ANNSRAIDYVKTYTGTSNDPKISPRYKGN (SEQ ID NO:64), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:64, or a fragment thereof.

In some forms, the CDA protein is BE_R4_24 (component of Uniprot ID NO.: WP_211232061) having the following amino acid sequence: ASPAVGTNAAGSSGKNVRMPRDYASELPEYDGKTTHGVLVTNEGKVIQLRSGGKEEPYTG YKAVSASHVEGKAAIWIRENGSSGGTVYHNNTTGTCGYCNSQVKALLPEGVELKIVPPTN AVAKNAQARAVPTINVGNGTQPGRKQK (SEQ ID NO:65), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:65, or a fragment thereof.

In some forms, the CDA protein is BE_R4_25 (component of Uniprot ID NO.: MGYP000402883179) having the following amino acid sequence: YVGENGVWVHNASSEYGEVPELPEFNGKKTEGVFRTADGKEIKFESGGSTEYKNPSASHA EGKAAIYMRENGIKEGTVFHNNPNGTCNYCDKGLATLLPEGARLTVVPPIGAVAPNKYWV DVPKTYTGNGNLPSMK (SEQ ID NO:66), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:66, or a fragment thereof.

In some forms, the CDA protein is BE_R4_26 (component of Uniprot ID NO.: MGYP000186340475) having the following amino acid sequence: HVGKCRLLVHNANCNQEKPVLPKYDGKTTEGVMVTPDGKQISFKSGNSSTPSYPQYKAQS ASHVEGKAALYMRENGINEATVFHNNPNGTCGFCDRQVPALLPKGAKLTVVPPSNSVANN VRAIPVPKTYIGNSTVPKIK (SEQ ID NO:67), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:67, or a fragment thereof.

In some forms, the CDA protein is one or more fragments of the following amino acid sequence: MALSRAVCGTSRQLAPVLGYLGSRQKHSLPDYPYDVPDYAGYPYDVPDYAGYPYDVPDYA MDIADLRTLGYSQQQQEKIKPKVRSTVAQHHEALVGHGFTHAHIVALSQHPAALGTVAVK YQDMIAALPEATHEAIVGVGKQWSGARALEALLTVAGELRGPPLQLDTGQLLKIAKRGGV TAVEAVHAWRNALTGAPLNLTPQQVVAIASNNGGKQALETVQRLLPVLCQAHGLTPEQVV AIASNIGGKQALETVQALLPVLCQAHGLTPEQVVAIASHDGGKQALETVQRLLPVLCQAH GLTPEQVVAIASHDGGKQALETVQRLLPVLCQAHGLTPEQVVAIASHDGGKQALETVQRL LPVLCQAHGLTPEQVVAIASHDGGKQALETVQRLLPVLCQAHGLTPEQVVAIASHDGGKQ ALETVQRLLPVLCQAHGLTPEQVVAIASNIGGKQALETVQALLPVLCQAHGLTPQQVVAI ASNGGGKQALETVQRLLPVLCQAHGLTPQQVVAIASNGGGRPALESIVAQLSRPDPALA ALTNDHLVALACLGGRPALDAVKKGLGGSGSYALGPYQISAPQLPAYNGQTVGTFYYVND AGGLESKVFSSGGPTPYPNYANAGHVEGQSALFMRDNGISEGLVFHNNPEGTCGFCVNMT ETLLPENAKMTVVPPEG (SEQ ID NO:68), or an amino acid sequence having at least 70%, at least 75%, at least 80%, at least 85%, at least 90%, at least 95%, at least 96%, at least 97%, at least 98%, at least 99%, or 100% sequence identify with CDA of SEQ ID NO:68, or a fragment thereof.

### MafB19 Deaminase Domains

In some forms the deaminase domain is a MafB19 deaminase domain. Sequence alignment of active and inactive members of the MafB19 deaminase family was used to identify signature motifs for dsDNA-specific deaminases in the MafB19 deaminase family. Particular signature motifs present in the dsDNA-specific CDAs in the MafB19 deaminase family include: (M/L)P motif; T(V/I/L/A)A(R/K/V) motif; (Y/F/W)G(V/H/I/R/K)N motif; HAE=>active site motif; VD(R/K) motif present in almost all members of MafB19-deam family that are active on dsDNA; and an CXXC motif (canonical CXXC zinc binding motif). Therefore, in some forms, a deaminase domain associated with the MafB19 deaminase family includes one or more structural features including an (M/L)P motif; T(V/I/L/A)A(R/K/V) motif; (Y/F/W)G(V/H/I/R/K)N motif; HAE active site motif; VD(R/K) motif and a canonical CXXC zinc binding motif.

### SCP1201 Deaminase Domains

In some forms the deaminase domain is a SCP1201 deaminase family deaminase domain. Sequence alignment of active and inactive members of the SCP1201 deaminase family was used to identify signature motifs for dsDNA-specific deaminases in the SCP1201 deaminase family. Particular signature motifs present in the dsDNA-specific CDAs in the SCP1201 deaminase family include: L(P/L) motif; (Y/F/E/Q)(D/E/N)G(K/R/D)(T/K/N)TXG(V/L/T)(L/M/F) motif; (P/S/T)(N/G/E/Q)Y motif; (G/S)HVE(G/A/Q)—G or S preceding conserved active site motif (HVE) which is followed by (G/A/Q); HNN motif (or (H/I)(N/D)(N/H) to lesser extent) G(T/I)C(G/P/N/H)(Y/F)C motif—G(T/I) preceding the canonical CXXC zinc binding motif; (T/A)LL(P/E) motif; L(E/D/R/K)V(V/I)PP motif and G(N/D)XXXPK motif. Cx(Y/F)C is prevalent motif in dsDNA-specific deaminases of the SCP1201 deaminase. With the exception of BE_R1_28, all active members of this family strictly have 2 amino acids between the two C residues in the zinc binding motif. Inactive members of the family all have more than two amino acid residues between the two C residues. A G(T/I) motif precede the zinc binding motif in the active members of this family. Therefore, in some forms, a deaminase domain associated with the SCP1201 deaminase family includes one or more structural features including L(P/L) motif; (Y/F/E/Q)(D/E/N)G(K/R/D)(T/K/N)TXG(V/L/T)(L/M/F) motif; (P/S/T)(N/G/E/Q)Y motif; (G/S)HVE(G/A/Q); HNN motif (or (H/I)(N/D)(N/H) to lesser extent) G(T/I)C(G/P/N/H)(Y/F)C motif; (T/A)LL(P/E) motif; L(E/D/R/K)V(V/I)PP motif and G(N/D)XXXPK motif.

In a particular form, the isolated deaminase domain can deaminate adenine-containing nucleotides (referred to as an adenosine deaminase). In some forms, an adenosine deaminase is a protein, a polypeptide, or one or more functional domain(s) of a protein or a polypeptide that is capable of catalyzing a hydrolytic deamination reaction that converts an adenine (or an adenine moiety of a molecule) to a hypoxanthine (or a hypoxanthine moiety of a molecule). The adenine-containing molecule can be an adenosine (A), and the hypoxanthine-containing molecule can be an inosine (I). The adenine-containing molecule can be DNA or RNA.

Additional suitable deaminase domains and sequences thereof will be apparent to those of skill in the art based on this disclosure. For example, the sequences of any one of SEQ ID NOs:1-16 or any of the accession numbers disclosed herein can be used as query sequences to identify homologues and other related proteins, polypeptides or domains thereof. It is contemplated that such homologues and other related proteins, polypeptides or domains thereof may exhibit deaminase activity towards RNA or DNA substrates and thus can be used in accordance with the disclosed compositions and methods.

In some forms, a suitable deaminase domain (e.g., adenosine deaminase or cytosine deaminase) has at least 50%, at least 60%, at least 70%, at least 75%, at least 80%, at least 85%, at least 90%, at least 95%, at least 96%, at least 97%, at least 98%, or at least 99% sequence identity with the sequences of any of the SEQ ID numbers or Uniprot accession numbers disclosed herein, such as SEQ ID NOs:1-16, and including nucleic acid sequences encoding amino acid sequences thereof. Preferably, the sequence identity is over at least 50%, 60%, 70%, 75%, 80%, 85%, 90%, 95%, 96%, 97%, 98%, 99%, or 100% of the length of the query sequence. Thus, in some forms, the isolated cytosine deaminase has at least 50%, 60%, 70%, 75%, 80%, 85%, 90%, 95%, 96%, 97%, 98%, 99%, or 100% sequence identity with the sequence of any of SEQ ID NOs:1-16, and including the nucleic acid sequence where the amino acid sequence is provided.

It should be appreciated that also disclosed are cytosine or adenosine deaminase variants including one or more mutations (e.g., conservative or non-conservative mutations) relative to any of the deaminases disclosed herein. It is also contemplated that other cytosine or adenosine deaminase variants can be evolved from those disclosed herein, for example, by targeted mutation of one or more amino acid residues in specific regions of the deaminase, either based on structural data, or by an array of direct evolution approaches (random mutagenesis and selection/screen). Thus, one or more mutations can be introduced into any of the disclosed deaminase domains. In some forms, such mutation(s) can alter substrate binding, alter conformation of bound substrate, alter substrate accessibility to the deaminase active site, alter tolerance to non-optimal presentation of a target nucleotide (e.g., C or A) to the deaminase active site, and/or alter target nucleotide sequence specificity (recognition) and/or editing efficiency. In some forms, a suitable cytosine or adenosine deaminase includes an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs:1-20, 40-68, or any of the deaminases otherwise described herein. In some forms, the cytosine or adenosine deaminase includes an amino acid sequence that has at least 5, at least 10, at least 15, at least 20, at least 25, at least 30, at least 35, at least 40, at least 45, at least 50, at least 60, at least 70, at least 80, at least 90, or at least 100 identical contiguous amino acid residues as compared to any one of the amino acid sequences set forth in SEQ ID NOs:1-16, or 40-68.

### B. Base Editors

Also disclosed are base editors including a deaminase domain and one or more functional domains. In some forms, the base editors include a “split” deaminase, for example, a deaminase that is cleaved into two or more distinct fragments. Each of the split fragments typically lacks deaminase activity, such that re-association of the two or more fragments, for example, by co-localization, restores or enhances the deaminase activity. Therefore, in some forms, the base editors are split base editors. Typically, the split base editors rely upon the specific interactions of one or more functional domains to co-localize the deaminase domains and reconstitute deaminase activity at a specific location within a nucleic acid. The functional domain can be a polypeptide or protein, or portion thereof, or any moiety that confers a desired property or function to the base editor. A desired property or function can be for example, localization to a cellular organelle, enzymatic activity, protein interaction, epitope tagging, or DNA and/or RNA binding. In some forms, a base editor includes (1) a programable DNA binding domain; and (2) a deaminase domain, and optionally one or more linkers between the DNA binding domain and the deaminase domain, and/or one or more additional functional domains, such as a targeting motif. In some forms, the deaminase domain is a split deaminase domain, i.e., an inactive deaminase domain or a fragment thereof. Typically, co-localization of two or more split deaminase domains (e.g., by association on a target DNA strand determined by the programmable DNA binding domain(s)) activates the deaminase activity in one or more of the two or more split deaminase domains.

### 1. Split Deaminase Domains

In some forms the compositions include a non-naturally occurring polypeptide fragment of a functional double-stranded DNA deaminase protein that is obtained by cleaving the deaminase protein at a cleavage site within the functional deaminase domain. For example, in some forms, the fragment corresponds to an N-terminal fragment, wherein the fragment includes an N-terminal portion of a cleaved functional deaminase domain. In other forms, the fragment corresponds to a C-terminal fragment, wherein the fragment includes a C-terminal portion of a cleaved functional deaminase domain. The deaminase activity is restored upon co-localizing the N-terminal fragment with the C-terminal fragment, or upon co-localizing the C-terminal fragment with an N-terminal fragment. Examples of different forms and configurations of split deaminases are shown in FIG. 41.

Base editors including a heterodimer having first and second monomers, the first monomer including a first programmable DNA binding protein and an N-terminal or C-terminal fragment of a cleaved double-stranded DNA deaminase, and the second monomer including a second programmable DNA binding protein and an N-terminal or C-terminal fragment of a cleaved double-stranded DNA deaminase, are also described. Typically, dimerization of the first and second monomers reconstitutes the functional double-stranded DNA deaminase protein and the functional double-stranded DNA deaminase activity.

i. Exemplary Split Deaminase Domains

Exemplary split deaminase domains that lack deaminase activity are described. Typically, split deaminase domains are inactivated by introduction of one or more mutations into the deaminase domain. The mutations include specific deletions, substitutions and additions of one or more amino acids at a given position within the deaminase domain. In some forms, split deaminase domains include one or more specific deletions, substitutions or additions of one or more amino acids at a given position(s) in any of the deaminase domains having an amino acid sequence of any one of SEQ ID NOs:1-17, 40-68.

a. Inactive Deaminase Domains

In some forms, the split deaminase is an inactive form of a deaminase protein. For example, in some forms, the split deaminase is a “dead” or completely inactive variant of a deaminase domain. In preferred forms, the dead deaminase domain is a deaminase protein having one or more mutants in the DNA binding region. Typically, co-localization of an inactive deaminase domain with one or more intact, truncated or cleaved deaminase domain fragments of the same type can reconstitute the activity of the truncated or cleaved deaminase domain fragment by providing the missing structural components of the truncated or cleaved fragments. This approach is especially useful for making split deaminases that require dimerization (or multimerization) for their activity, when cutting the deaminase at some split site may not be adequate.

In some forms, the dead deaminase domain is based on BE_R1_11 (BE_R1_11_dead) having an amino acid sequence: TKSANSGGAAKDLAKYRERQGMPRAGSADDAHTAARLDVGGRSFYGHNAHGRNIDIKVNA QTKTHAAADVFQQAKNAKVSADRATLHVDRDLCDACGIKGGVGSLMRGVGISRLTVNSPS GRFEITASRPSVPRRING (SEQ ID NO:122), or an amino acid having at least 70%, 75%, 80%, 85%, 90%, 95%, or 99% sequence identity to SEQ ID NO:122, or fragment thereof.

In some forms, the dead deaminase domain is based on BE_R1_28 (BE_R1_28_dead) having an amino acid sequence: GVGGAITATVGSTAGAAGRAAARAPSLPAYAGGKTSGVLRTTAGDTALLSGYKGPSASMP RGTPGMNGRIKSHVAAHAAAVMREQGMKEGTLYINRVPCSGATGCDAMLPRMLPPDAHLR VVGPNGYDQVFVGLPD (SEQ ID NO:123), or an amino acid having at least 70%, 75%, 80%, 85%, 90%, 95%, or 99% sequence identity to SEQ ID NO:123, or fragment thereof.

In some forms, the dead deaminase domain is based on BE_R1_12 (BE_R1_12_dead) having an amino acid sequence: IQRFRRILNMPRYSLTNGRTGTVARVEVNGRRIFGVNTSLIKNSKYAPRDMDLRRRWLRE VNWVPPKKNKPNHLGHAQSLSHAASHALIRAYERMERLGGQLPKKLTMVVDRPTCNICRG EMPALLKRLGIEELTIYSGGRDAIIIKAIK (SEQ ID NO:124), or an amino acid having at least 70%, 75%, 80%, 85%, 90%, 95%, or 99% sequence identity to SEQ ID NO:124, or fragment thereof.

In some forms, the dead deaminase domain is based on BE_R4_21 (BE_R4_21_dead) having an amino acid sequence: GGSAVVGAGVVATGAKAVTTGKSLSESQATLSVAQRLLATIGEEGKTAGVLELDGELIPL VSGKSSLPNYAASGHVAGQAALIMRDRGATSGRLLIDNPSGICGYCKSQVATLLPENATL QVGTPLGTVTPSSRWSASRTFTGNDRDPKPWPR (SEQ ID NO:125), or an amino acid having at least 70%, 75%, 80%, 85%, 90%, 95%, or 99% sequence identity to SEQ ID NO:125, or fragment thereof.

In some forms, the dead deaminase domain is based on BE_R2_11 (BE_R2_11_dead) having an amino acid sequence: SQFDNVRKDMGLPARIGDDDPYTTSVLRIDGHEYWGKNGKWVTKGKTSNYTDKAHYDKVR KELGTSAEVPGHAAGVAFNKAYQVRKNTGTKGGNAVLYVDKIPCVMCKPGIATLMRSAKV DHLDLHYLQDGKMHHVQYVRNPDTDAVYNPFSGKWTKPSKKK (SEQ ID NO:126), or an amino acid having at least 70%, 75%, 80%, 85%, 90%, 95%, or 99% sequence identity to SEQ ID NO:126, or fragment thereof. b. Truncated or Cleaved Split Deaminase Domains In some forms, the split deaminase is a truncated or cleaved form of a deaminase protein. The split proteins can be designed so that one or more (2×) active site are present on the target upon reconstitution. For example, in some forms, the split deaminase is a completely inactive truncated or cleaved fragment of a deaminase domain. In preferred forms, the truncated or cleaved deaminase domain is a deaminase protein having one or more amino acids removed from the amino (NH) or carboxyl (COOH) terminus regions of the deaminase protein, or both the amino (NH) and carboxyl (COOH) termini regions.

In some forms, the truncated or cleaved form of a deaminase protein is a truncated or cleaved deaminase protein lacking a specific number of contiguous amino acid residues counted from the amino (NH) terminus, or from the carboxyl (COOH) terminus, or from both the amino (NH) terminus, and from the carboxyl (COOH) terminus. For example, in some forms, the truncated or cleaved form of a deaminase protein is a truncated or cleaved deaminase protein lacking (A) 5 contiguous amino acid residues, or 10, 20, 25, 30, 35, 40, 45, 50, 55, 60, 65, 70, 75, 80, 85, 90, 95, or 100 contiguous amino acid residues counted from the amino (NH) terminus, or from the carboxyl (COOH) terminus, or from both the amino (NH) terminus and the carboxyl (COOH) terminus.

### (1) Split BE_R1_11 Deaminase Protein

In some forms, the truncated or cleaved form of a deaminase protein is a truncated or cleaved form of a BE_R1_11 deaminase protein.

Cleaved Amino (NH) Fragments of BE_R1_11 In some forms, the truncated or cleaved form of a deaminase protein is a truncated or cleaved BE_R1_1i deaminase protein cleaved at a specific amino acid residue to yield a fragment of the BE_R1_1i deaminase protein corresponding to the amino (NH) terminus. In some forms, the truncated or cleaved form of a deaminase protein is a cleaved BE_R1_11 deaminase protein fragment including amino acid residues at the (NH) terminus resulting from cleavage at a position including any of Gly30, or Gly41, or Ser70, or Gly90, or Gly100.

In some forms, the cleaved form of a deaminase protein is a cleaved form of a BE_R1_11 deaminase protein cleaved at amino acid Gly30 (BE_R1_11_N_G30), having an amino acid sequence: TKSANSGGAAKDLAKYRERQGMPRAGSADDAHTAARLDVG (SEQ ID NO:127), or an amino acid having at least 70%, 75%, 80%, 85%, 90%, 95%, or 99% sequence identity to SEQ ID NO:127, or fragment thereof.

In some forms, the cleaved form of a deaminase protein is a cleaved form of a BE_R1_11 deaminase protein cleaved at amino acid Gly41 (BE_R1_11_N_G41), having an amino acid sequence: TKSANSGGAAKDLAKYRERQGMPRAGSADDAHTAARLDVGGRSFYGHNAHG(SEQ ID NO:128), or an amino acid having at least 70%, 75%, 80%, 85%, 90%, 95%, or 99% sequence identity to SEQ ID NO:128, or fragment thereof.

In some forms, the cleaved form of a deaminase protein is a cleaved form of a BE_R1_11 deaminase protein cleaved at amino acid Ser70 (BE_R1_11_N_S70), having an amino acid sequence: TKSANSGGAAKDLAKYRERQGMPRAGSADDAHTAARLDVGGRSFYGHNAHGRNIDIKVNA QTKTHAEADVFQQAKNAKVS (SEQ ID NO:129), or an amino acid having at least 70%, 75%, 80%, 85%, 90%, 95%, or 99% sequence identity to SEQ ID NO:129, or fragment thereof.

In some forms, the cleaved form of a deaminase protein is a cleaved form of a BE_R1_11 deaminase protein cleaved at amino acid Gly90 (BE_R1_11_N_G90), having an amino acid sequence: TKSANSGGAAKDLAKYRERQGMPRAGSADDAHTAARLDVGGRSFYGHNAHGRNIDIKVNA QTKTHAEADVFQQAKNAKVSADRATLHVDRDLCDACGIK (SEQ ID NO:130), or an amino acid having at least 70%, 75%, 80%, 85%, 90%, 95%, or 99% sequence identity to SEQ ID NO:130, or fragment thereof.

In some forms, the cleaved form of a deaminase protein is a cleaved form of a BE_R1_11 deaminase protein cleaved at amino acid Gly100 (BE_R1_11_N_G100), having an amino acid sequence: TKSANSGGAAKDLAKYRERQGMPRAGSADDAHTAARLDVGGRSFYGHNAHGRNIDIKVNA QTKTHAEADVFQQAKNAKVSADRATLHVDRDLCDACGIKGGVGSLMRGVG (SEQ ID NO:131), or an amino acid having at least 70%, 75%, 80%, 85%, 90%, 95%, or 99% sequence identity to SEQ ID NO:131, or fragment thereof.

### Cleaved Carboxyl (COOH) Fragments of BE_R1_11

In some forms, the truncated or cleaved form of a deaminase protein is a truncated or cleaved BE_R1_11 deaminase protein cleaved at a specific amino acid residue to yield a fragment of the BE_R1_11 deaminase protein corresponding to the carboxyl (COOH) terminus. In some forms, the truncated or cleaved form of a deaminase protein is a cleaved BE_R1_11 deaminase protein fragment including amino acid residues at the carboxyl (COOH) terminus resulting from cleavage at a position including any of Gly30, or Gly41, or Ser70, or Gly90, or Gly100.

In some forms, the truncated or cleaved form of a deaminase protein is cleaved BE_R1_11 deaminase protein lacking amino acid residues at the amino (NH) terminus.

In some forms, the cleaved form of a deaminase protein is a cleaved form of a BE_R1_11 deaminase protein cleaved at amino acid Gly30 (BE_R1_11_C_G30), having an amino acid sequence: GRSFYGHNAHGRNIDIKVNAQTKTHAEADVFQQAKNAKVSADRATLHVDRDLCDACGIKG GVGSLMRGVGISRLTVNSPSGRFEITASRPSVPRRING (SEQ ID NO:132), or an amino acid having at least 70%, 75%, 80%, 85%, 90%, 95%, or 99% sequence identity to SEQ ID NO:132, or fragment thereof.

In some forms, the truncated or cleaved form of a deaminase protein is a truncated or cleaved form of a BE_R1_11 deaminase protein truncated at amino acid Gly41 (BE_R1_11_C_G41), having an amino acid sequence: RNIDIKVNAQTKTHAEADVFQQAKNAKVSADRATLHVDRDLCDACGIKGGVGSLMRGVGI SRLTVNSPSGRFEITASRPSVPRRING (SEQ ID NO:133), or an amino acid having at least 70%, 75%, 80%, 85%, 90%, 95%, or 99% sequence identity to SEQ ID NO:133, or fragment thereof.

In some forms, the cleaved form of a deaminase protein is a cleaved form of a BE_R1_11 deaminase protein cleaved at amino acid Ser70 (BE_R1_11_C_S70), having an amino acid sequence: ADRATLHVDRDLCDACGIKGGVGSLMRGVGISRLTVNSPSGRFEITASRPSVPRRING (SEQ ID NO:150), or an amino acid having at least 70%, 75%, 80%, 85%, 90%, 95%, or 99% sequence identity to SEQ ID NO:150, or fragment thereof.

In some forms, the cleaved form of a deaminase protein is a cleaved form of a BE_R1_11 deaminase protein cleaved at amino acid Gly90 (BE_R1_11_C_G90), having an amino acid sequence: GGVGSLMRGVGISRLTVNSPSGRFEITASRPSVPRRING (SEQ ID NO:134), or an amino acid having at least 70%, 75%, 80%, 85%, 90%, 95%, or 99% sequence identity to SEQ ID NO:134, or fragment thereof.

In some forms, the cleaved form of a deaminase protein is a cleaved form of a BE_R1_1i deaminase protein cleaved at amino acid Gly100 (BE_R1_11_C_G100), having an amino acid sequence: ISRLTVNSPSGRFEITASRPSVPRRING (SEQ ID NO:135), or an amino acid having at least 70%, 75%, 80%, 85%, 90%, 95%, or 99% sequence identity to SEQ ID NO:135, or fragment thereof.

### Combinations of Split BE_R1_11 Deaminase Proteins

In some forms, the truncated or cleaved form of BE_R1_1i deaminase protein lacks deaminase function alone. In some forms, the combination of two or more of the truncated or cleaved form of BE_R1_11 deaminase protein reconstitutes the deaminase function. For example, in some forms, combining one truncated or cleaved form of BE_R1_1i deaminase protein lacking one or more amino acid residues from the amino (NH) terminus, or a fragment from the carboxyl (COOH) terminus of the complete BE_R1_11 deaminase domain becomes functional upon combination or co-localization with one or more truncated or cleaved form of BE_R1_i1 deaminase protein lacking one or more amino acid residues from the carboxyl (COOH) terminus, or a fragment from the amino (NH) terminus of the complete BE_R1_1i deaminase domain. For example, in some forms, base editors include a split BE_R1_11 deaminase domain having an amino acid sequence of any one of SEQ ID NOS:127-131, where the base editor has reconstituted deaminase activity upon co-localization or combination with another split BE_R1_11 deaminase domain having an amino acid sequence of any one of SEQ ID NOS:132-135, or together with a “dead” form of the BE_R1_11 deaminase domain having an amino acid sequence of SEQ ID NO:122, or an amino acid having at least 70%, 75%, 80%, 85%, 90%, 95%, or 99% sequence identity to SEQ ID NO:122.

### (2) Split BE_R1_12 Deaminase Proteins

In some forms, the truncated or cleaved form of a deaminase protein is a truncated or cleaved form of a BE_R1_12 deaminase protein.

Cleaved amino (NH) fragments of BE_R1_12 In some forms, the truncated or cleaved form of a deaminase protein is a truncated or cleaved BE_R1_12 deaminase protein fragment including amino acid residues at the (NH) terminus resulting from cleavage at a position including any of Gly31, or Gly40, or Gly85, Gly110 or Gly140.

In some forms, the cleaved form of a deaminase protein is cleaved form of a BE_R1_12 deaminase protein cleaved at amino acid Gly31 (BE_R1_12_N_G31), having an amino acid sequence: FSKAESGYIEIQRFRRILNMPRYSLTNGRTG (SEQ ID NO:136), or an amino acid having at least 70%, 75%, 80%, 85%, 90%, 95%, or 99% sequence identity to SEQ ID NO:136, or fragment thereof.

In some forms, the cleaved form of a deaminase protein is cleaved form of a BE_R1_12 deaminase protein cleaved at amino acid Gly40 (BE_R1_12_N_G40), having an amino acid sequence: FSKAESGYIEIQRFRRILNMPRYSLTNGRTGTVARVEVNG (SEQ ID NO:137), or an amino acid having at least 70%, 75%, 80%, 85%, 90%, 95%, or 99% sequence identity to SEQ ID NO:137, or fragment thereof.

In some forms, the cleaved form of a deaminase protein is cleaved form of a BE_R1_12 deaminase protein cleaved at amino acid Gly85 (BE_R1_12_N_G85), having an amino acid sequence: FSKAESGYIEIIQRFRRILNMPRYSLTNGRTGTVARVEVNGRRIFGVNTSLIKNSKYAPR DMDLRRRWLREVNWVPPKKNKPNHLG (SEQ ID NO:138), or an amino acid having at least 70%, 75%, 80%, 85%, 90%, 95%, or 99% sequence identity to SEQ ID NO:138, or fragment thereof.

In some forms, the cleaved form of a deaminase protein is cleaved form of a BE_R1_12 deaminase protein cleaved at amino acid Gly110 (BE_R1_12_N_G110), having an amino acid sequence: FSKAESGYIEIIQRFRRILNMPRYSLTNGRTGTVARVEVNGRRIFGVNTSLIKNSKYAPR DMDLRRRWLREVNWVPPKKNKPNHLGHAQSLSHAESHALIRAYERMERLGG (SEQ ID NO:139), or an amino acid having at least 70%, 75%, 80%, 85%, 90%, 95%, or 99% sequence identity to SEQ ID NO:139, or fragment thereof.

In some forms, the cleaved form of a deaminase protein is cleaved form of a BE_R1_12 deaminase protein cleaved at amino acid Gly140 (BE_R1_12_N_G140), having an amino acid sequence: FSKAESGYIEIIQRFRRILNMPRYSLTNGRTGTVARVEVNGRRIFGVNTSLIKNSKYAPR DMDLRRRWLREVNWVPPKKNKPNHLGHAQSLSHAESHALIRAYERMERLGGQLPKKLTMV VDRPTCNICRGEMPALLKRLG (SEQ ID NO:140), or an amino acid having at least 70%, 75%, 80%, 85%, 90%, 95%, or 99% sequence identity to SEQ ID NO:140, or fragment thereof.

Cleaved carboxyl (COOH) fragments of BE_R1_12 In some forms, the cleaved form of a deaminase protein is a cleaved BE_R1_12 deaminase protein fragment including amino acid residues at the carboxyl (COOH) terminus resulting from cleavage at a position including any of Gly31, or Gly40, or Gly85, Gly110 or Gly140.

In some forms, the cleaved form of a deaminase protein is a cleaved form of a BE_R1_12 deaminase protein cleaved at amino acid Gly31 (BE_R1_12_C_G31), having an amino acid sequence: TVARVEVNGRRIFGVNTSLIKNSKYAPRDMDLRRRWLREVNWVPPKKNKPNHLGHAQSLS HAESHALIRAYERMERLGGQLPKKLTMVVDRPTCNICRGEMPALLKRLGIEELTIYSGGR DAIIIKAIK (SEQ ID NO: 141), or an amino acid having at least 70%, 75%, 80%, 85%, 90%, 95%, or 99% sequence identity to SEQ ID NO:141, or fragment thereof.

In some forms, the cleaved form of a deaminase protein is a cleaved form of a BE_R1_12 deaminase protein t cleaved at amino acid Gly40 (BE_R1_12_C_G40), having an amino acid sequence: RRIFGVNTSLIKNSKYAPRDMDLRRRWLREVNWVPPKKNKPNHLGHAQSLSHAESHALIR AYERMERLGGQLPKKLTMVVDRPTCNICRGEMPALLKRLGIEELTIYSGGRDAIIIKAIK (SEQ ID NO:142), or an amino acid having at least 70%, 75%, 80%, 85%, 90%, 95%, or 99% sequence identity to SEQ ID NO:142, or fragment thereof.

In some forms, the cleaved form of a deaminase protein is a cleaved form of a BE_R1_12 deaminase protein cleaved at amino acid Gly85 (BE_R1_12_C_G85), having an amino acid sequence: HAQSLSHAESHALIRAYERMERLGGQLPKKLTMVVDRPTCNICRGEMPALLKRLGIEELT IYSGGRDAIIIKAIK (SEQ ID NO:143), or an amino acid having at least 70%, 75%, 80%, 85%, 90%, 95%, or 99% sequence identity to SEQ ID NO:143, or fragment thereof.

In some forms, the cleaved form of a deaminase protein is a cleaved form of a BE_R1_12 deaminase protein cleaved at amino acid Gly110 (BE_R1_12_C_G110), having an amino acid sequence: QLPKKLTMVVDRPTCNICRGEMPALLKRLGIEELTIYSGGRDAIIIKAIK (SEQ ID NO:144), or an amino acid having at least 70%, 75%, 80%, 85%, 90%, 95%, or 99% sequence identity to SEQ ID NO:144, or fragment thereof.

In some forms, the cleaved form of a deaminase protein is a cleaved form of a BE_R1_12 deaminase protein cleaved at amino acid Gly140 (BE_R1_12_C_G140), having an amino acid sequence: IEELTIYSGGRDAIIIKAIK (SEQ ID NO:145), or an amino acid having at least 70%, 75%, 80%, 85%, 90%, 95%, or 99% sequence identity to SEQ ID NO:145, or fragment thereof.

### Truncated Fragments of BE_R1_12

In some forms, the truncated or cleaved form of a deaminase protein is a truncated or cleaved BE_R1_28 deaminase protein lacking a specific number of contiguous amino acid residues counted from the amino (NH) terminus (i.e., to yield a fragment including the intact carboxyl (COOH) terminus). For example, in some forms, the truncated or cleaved form of a deaminase protein is a truncated or cleaved BE_R1_28 deaminase protein lacking (A) 5 contiguous amino acid residues, or 10, 20, 25, 30, 35, 40, 45, 50, 55, 60, 65, 70, 75, 80, 85, 90, 95, or 100 contiguous amino acid residues counted from the amino (NH) terminus.

In some forms, the truncated form of a deaminase protein is a truncated BE_R1_12 deaminase protein lacking (A) 20 contiguous amino acid residues from the amino (NH) terminus (BE_R1_12_C_A20), having an amino acid sequence: MPRYSLTNGRTGTVARVEVNGRRIFGVNTSLIKNSKYAPRDMDLRRRWLREVNWVPPKKN KPNHLGHAQSLSHAESHALIRAYERMERLGGQLPKKLTMVVDRPTCNICRGEMPALLKRL GIEELTIYSGGRDAIIIKAIK (SEQ ID NO:156), or an amino acid having at least 70%, 75%, 80%, 85%, 90%, 95%, or 99% sequence identity to SEQ ID NO:156, or fragment thereof.

In some forms, the truncated form of a deaminase protein is a truncated BE_R1_12 deaminase protein lacking (A) 25 contiguous amino acid residues from the amino (NH) terminus (BE_R1_12_C_A25), having an amino acid sequence: TNGRTGTVARVEVNGRRIFGVNTSLIKNSKYAPRDMDLRRRWLREVNWVPPKKNKPNHLG HAQSLSHAESHALIRAYERMERLGGQLPKKLTMVVDRPTCNICRGEMPALLKRLGIEELT IYSGGRDAIIIKAIK (SEQ ID NO:157), or an amino acid having at least 70%, 75%, 80%, 85%, 90%, 95%, or 99% sequence identity to SEQ ID NO:157, or fragment thereof.

In some forms, the truncated form of a deaminase protein is a truncated BE_R1_12 deaminase protein lacking (A) 30 contiguous amino acid residues from the Carboxyl (COOH) terminus (BE_R1_12_C_A30), having an amino acid sequence: GTVARVEVNGRRIFGVNTSLIKNSKYAPRDMDLRRRWLREVNWVPPKKNKPNHLGHAQSL SHAESHALIRAYERMERLGGQLPKKLTMVVDRPTCNICRGEMPALLKRLGIEELTIYSGG RDAIIIKAIK (SEQ ID NO:158), or an amino acid having at least 70%, 75%, 80%, 85%, 90%, 95%, or 99% sequence identity to SEQ ID NO:158, or fragment thereof.

In some forms, the truncated form of a deaminase protein is a truncated BE_R1_12 deaminase protein lacking (A) 35 contiguous amino acid residues from the Amino (NH) terminus (BE_R1_12_C_A35), having an amino acid sequence: VEVNGRRIFGVNTSLIKNSKYAPRDMDLRRRWLREVNWVPPKKNKPNHLGHAQSLSHAES HALIRAYERMERLGGQLPKKLTMVVDRPTCNICRGEMPALLKRLGIEELTIYSGGRDAII IKAIK (SEQ ID NO:159), or an amino acid having at least 70%, 75%, 80%, 85%, 90%, 95%, or 99% sequence identity to SEQ ID NO:159, or fragment thereof.

In some forms, the truncated form of a deaminase protein is a truncated BE_R1_12 deaminase protein lacking (A) 40 contiguous amino acid residues from the Amino (NH) terminus (BE_R1_12_C_A40), having an amino acid sequence: RRIFGVNTSLIKNSKYAPRDMDLRRRWLREVNWVPPKKNKPNHLGHAQSLSHAESHALIR AYERMERLGGQLPKKLTMVVDRPTCNICRGEMPALLKRLGIEELTIYSGGRDAIIIKAIK (SEQ ID NO:160), or an amino acid having at least 70%, 75%, 80%, 85%, 90%, 95%, or 99% sequence identity to SEQ ID NO:160, or fragment thereof.

In some forms, the truncated form of a deaminase protein is a truncated BE_R1_12 deaminase protein lacking (A) 45 contiguous amino acid residues from the Amino (NH) terminus (BE_R1_12_C_A45), having an amino acid sequence: VNTSLIKNSKYAPRDMDLRRRWLREVNWVPPKKNKPNHLGHAQSLSHAESHALIRAYERM ERLGGQLPKKLTMVVDRPTCNICRGEMPALLKRLGIEELTIYSGGRDAIIIKAIK (SEQ ID NO:161), or an amino acid having at least 70%, 75%, 80%, 85%, 90%, 95%, or 99% sequence identity to SEQ ID NO:161, or fragment thereof.

In some forms, the truncated form of a deaminase protein is a truncated BE_R1_12 deaminase protein lacking (A) 50 contiguous amino acid residues from the Amino (NH) terminus (BE_R1_12_C_A50), having an amino acid sequence: IKNSKYAPRDMDLRRRWLREVNWVPPKKNKPNHLGHAQSLSHAESHALIRAYERMERLGG QLPKKLTMVVDRPTCNICRGEMPALLKRLGIEELTIYSGGRDAIIIKAIK (SEQ ID NO:162), or an amino acid having at least 70%, 75%, 80%, 85%, 90%, 95%, or 99% sequence identity to SEQ ID NO:162, or fragment thereof.

In some forms, the truncated form of a deaminase protein is a truncated BE_R1_12 deaminase protein lacking (A) 55 contiguous amino acid residues from the Amino (NH) terminus (BE_R1_12_C_A55), having an amino acid sequence: YAPRDMDLRRRWLREVNWVPPKKNKPNHLGHAQSLSHAESHALIRAYERMERLGGQLPKK LTMVVDRPTCNICRGEMPALLKRLGIEELTIYSGGRDAII IKAIK (SEQ ID NO:163), or an amino acid having at least 70%, 75%, 80%, 85%, 90%, 95%, or 99% sequence identity to SEQ ID NO:163, or fragment thereof.

In some forms, the truncated form of a deaminase protein is a truncated BE_R1_12 deaminase protein lacking (A) 60 contiguous amino acid residues from the Amino (NH) terminus (BE_R1_12_C_A60), having an amino acid sequence: MDLRRRWLREVNWVPPKKNKPNHLGHAQSLSHAESHALIRAYERMERLGGQLPKKLTMVV DRPTCNICRGEMPALLKRLGIEELTIYSGGRDAIIIKAIK (SEQ ID NO:164), or an amino acid having at least 70%, 75%, 80%, 85%, 90%, 95%, or 99% sequence identity to SEQ ID NO:164, or fragment thereof.

In some forms, the truncated form of a deaminase protein is a truncated BE_R1_12 deaminase protein lacking (A) 70 contiguous amino acid residues from the Amino (NH) terminus (BE_R1_12_C_A70), having an amino acid sequence: VNWVPPKKNKPNHLGHAQSLSHAESHALIRAYERMERLGGQLPKKLTMVVDRPTCNICRG EMPALLKRLGIEELTIYSGGRDAIIIKAIK (SEQ ID NO:165), or an amino acid having at least 70%, 75%, 80%, 85%, 90%, 95%, or 99% sequence identity to SEQ ID NO:165, or fragment thereof.

In some forms, the truncated form of a deaminase protein is a truncated BE_R1_12 deaminase protein lacking (A) 75 contiguous amino acid residues from the Amino (NH) terminus (BE_R1_12_C_A75), having an amino acid sequence: PKKNKPNHLGHAQSLSHAESHALIRAYERMERLGGQLPKKLTMVVDRPTCNICRGEMPAL LKRLGIEELTIYSGGRDAIIIKAIK (SEQ ID NO:166), or an amino acid having at least 70%, 75%, 80%, 85%, 90%, 95%, or 99% sequence identity to SEQ ID NO:166, or fragment thereof.

In some forms, the truncated form of a deaminase protein is a truncated BE_R1_12 deaminase protein lacking (A) 100 contiguous amino acid residues from the Amino (NH) terminus (BE_R1_12_C_A100), having an amino acid sequence: HALIRAYERMERLGGQLPKKLTMVVDRPTCNICRGEMPALLKRLGIEELTIYSGGRDAII IKAIK (SEQ ID NO:167), or an amino acid having at least 70%, 75%, 80%, 85%, 90%, 95%, or 99% sequence identity to SEQ ID NO:167, or fragment thereof.

Combinations of Split BE-_R1_12 deaminase proteins In some forms, the truncated or cleaved form of BE_R1_12 deaminase protein lacks deaminase function alone. In some forms, the combination of two or more of the truncated or cleaved form of BE_R1_12 deaminase protein reconstitutes the deaminase function. For example, in some forms, combining one truncated or cleaved form of BE_R1_12 deaminase protein lacking one or more amino acid residues from the amino (NH) terminus, or a fragment from the carboxyl (COOH) terminus of the complete BE_R1_12 deaminase domain becomes functional upon combination or co-localization with one or more truncated or cleaved form of BE_R1_12 deaminase protein lacking one or more amino acid residues from the carboxyl (COOH) terminus, or a fragment from the amino (NH) terminus of the complete BE_R1_12 deaminase domain. For example, in some forms, base editors include a split BE_R1_12 deaminase domain having an amino acid sequence of any one of SEQ ID NOS:141-145, where the base editor has reconstituted deaminase activity upon co-localization or combination with another split BE_R1_12 deaminase domain having an amino acid sequence of any one of SEQ ID NOS:136-140, or together with a “dead” form of the BE_R1_12 deaminase domain having an amino acid sequence of SEQ ID NO:124, or an amino acid having at least 70%, 75%, 80%, 85%, 90%, 95%, or 99% sequence identity to SEQ ID NO:124..

In some forms, base editors include a split BE_R1_12 deaminase domain having an amino acid sequence of any one of SEQ ID NOS:146-167, where the base editor has reconstituted deaminase activity upon co-localization or combination with another split BE_R1_12 deaminase domain having an amino acid sequence of any one of SEQ ID NOS:136-140, or together with a “dead” form of the BE_R1_12 deaminase domain having an amino acid sequence of SEQ ID NO:124, or an amino acid having at least 70%, 75%, 80%, 85%, 90%, 95%, or 99% sequence identity to SEQ ID NO:124.

### (3) Split BE_R1_28 Deaminase Proteins

In some forms, the truncated or cleaved form of a deaminase protein is a truncated or cleaved form of a BE_R1_28 deaminase protein.

### Cleaved Amino (NH) Fragments of BE_R1_28

In some forms, the truncated or cleaved form of a deaminase protein is a truncated or cleaved BE_R1_28 deaminase protein fragment including amino acid residues at the (NH) terminus resulting from cleavage at a position including any of Gly33, or Gly51, or Lys71, Gly101 or Gly126.

In some forms, the cleaved form of a deaminase protein is cleaved form of a BE_R1_12 deaminase protein cleaved at amino acid Gly33 (BE_R1_28_NG33), having an amino acid sequence: GVGGAITATVGSTAGAAGRAAARAPSLPAYAGG (SEQ ID NO:146), or an amino acid having at least 70%, 75%, 80%, 85%, 90%, 95%, or 99% sequence identity to SEQ ID NO:146, or fragment thereof.

In some forms, the truncated or cleaved form of a deaminase protein is a truncated or cleaved form of a BE_R1_28 deaminase protein truncated at amino acid Gly51 (BE_R1_28_N_G51), having an amino acid sequence: GVGGAITATVGSTAGAAGRAAARAPSLPAYAGGKTSGVLRTTAGDTALLSG (SEQ ID NO:147), or an amino acid having at least 70%, 75%, 80%, 85%, 90%, 95%, or 99% sequence identity to SEQ ID NO:147, or fragment thereof.

In some forms, the cleaved form of a deaminase protein is cleaved form of a BE_R1_28 deaminase protein cleaved at amino acid Lys71 (BE_R1_28_NK71), having an amino acid sequence: GVGGAITATVGSTAGAAGRAAARAPSLPAYAGGKTSGVLRTTAGDTALLSGYKGPSASMP RGTPGMNGRIK (SEQ ID NO:148), or an amino acid having at least 70%, 75%, 80%, 85%, 90%, 95%, or 99% sequence identity to SEQ ID NO:148, or fragment thereof.

In some forms, the cleaved form of a deaminase protein is cleaved form of a BE_R1_28 deaminase protein cleaved at amino acid Gly101 (BE_R1_28_N_G101), having an amino acid sequence: GVGGAITATVGSTAGAAGRAAARAPSLPAYAGGKTSGVLRTTAGDTALLSGYKGPSASMP RGTPGMNGRIKSHVEAHAAAVMREQGMKEGTLYINRVPCSG (SEQ ID NO:149), or an amino acid having at least 70%, 75%, 80%, 85%, 90%, 95%, or 99% sequence identity to SEQ ID NO:149, or fragment thereof.

### Cleaved Carboxyl (COOH) Fragments of BE_R1_28

In some forms, the truncated or cleaved form of a deaminase protein is a truncated or cleaved BE_R1_28 deaminase protein fragment including amino acid residues at the carboxyl (COOH) terminus resulting from cleavage at a position including any of Gly33, or Gly51, or Lys71, Gly101 or Gly126.

In some forms, the cleaved form of a deaminase protein is cleaved form of a BE_R1_28 deaminase protein cleaved at amino acid Gly33 terminus (BE_R1_28_C_G33), having an amino acid sequence: KTSGVLRTTAGDTALLSGYKGPSASMPRGTPGMNGRIKSHVEAHAAAVMREQGMKEGTLY INRVPCSGATGCDAMLPRMLPPDAHLRVVGPNGYDQVFVGL (SEQ ID NO:151), or an amino acid having at least 70%, 75%, 80%, 85%, 90%, 95%, or 99% sequence identity to SEQ ID NO:151, or fragment thereof.

In some forms, the cleaved form of a deaminase protein is cleaved form of a BE_R1_28 deaminase protein cleaved at amino acid Gly51 (BE_R1_28_CG51), having an amino acid sequence: YKGPSASMPRGTPGMNGRIKSHVEAHAAAVMREQGMKEGTLYINRVPCSGATGCDAMLPR MLPPDAHLRVVGPNGYDQVFVGL (SEQ ID NO:152), or an amino acid having at least 70%, 75%, 80%, 85%, 90%, 95%, or 99% sequence identity to SEQ ID NO:152, or fragment thereof.

In some forms, the cleaved form of a deaminase protein is cleaved form of a BE_R1_28 deaminase protein cleaved at amino acid Lys71 (BE_R1_28_CK71), having an amino acid sequence: SHVEAHAAAVMREQGMKEGTLYINRVPCSGATGCDAMLPRMLPPDAHLRVVGPNGYDQVF VGL (SEQ ID NO:153), or an amino acid having at least 70%, 75%, 80%, 85%, 90%, 95%, or 99% sequence identity to SEQ ID NO:153, or fragment thereof.

In some forms, the cleaved form of a deaminase protein is cleaved form of a BE_R1_28 deaminase protein cleaved at amino acid Gly101 (BE_R1_28_C_G101), having an amino acid sequence: ATGCDAMLPRMLPPDAHLRVVGPNGYDQVFVGL (SEQ ID NO:154), or an amino acid having at least 70%, 75%, 80%, 85%, 90%, 95%, or 99% sequence identity to SEQ ID NO:154, or fragment thereof.

In some forms, the cleaved form of a deaminase protein is cleaved form of a BE_R1_28 deaminase protein cleaved at amino acid Gly126 (BE_R1_28_C_G126), having an amino acid sequence: YDQVFVGL (SEQ ID NO:155), or an amino acid having at least 70%, 75%, 80%, 85%, 90%, 95%, or 99% sequence identity to SEQ ID NO:155, or fragment thereof.

Combinations of Split BE_R1_28 deaminase proteins In some forms, the truncated or cleaved form of BE_R1_28 deaminase protein lacks deaminase function alone. In some forms, the combination of two or more of the truncated or cleaved form of BE_R1_28 deaminase protein reconstitutes the deaminase function. For example, in some forms, combining one truncated or cleaved form of BE_R1_28 deaminase protein lacking one or more amino acid residues from the amino (NH) terminus, or a fragment from the carboxyl (COOH) terminus of the complete BE_R1_28 deaminase domain becomes functional upon combination or co-localization with one or more truncated or cleaved form of BE_R1_28 deaminase protein lacking one or more amino acid residues from the carboxyl (COOH) terminus, or a fragment from the amino (NH) terminus of the complete BE_R1_28 deaminase domain. For example, in some forms, base editors include a split BE_R1_28 deaminase domain having an amino acid sequence of any one of SEQ ID NOS:151-155, where the base editor has reconstituted deaminase activity upon co-localization or combination with another split BE_R1_28 deaminase domain having an amino acid sequence of any one of SEQ ID NOS:146-149, or together with a “dead” form of the BE_R1_12 deaminase domain having an amino acid sequence of SEQ ID NO:123, or an amino acid having at least 70%, 75%, 80%, 85%, 90%, 95%, or 99% sequence identity to SEQ ID NO:123.

### (4) Split BE_R1_41 Deaminase Proteins

In some forms, the truncated or cleaved form of a deaminase protein is a truncated or cleaved form of a BE_R1_41 deaminase protein.

### Cleaved Amino (NH) Fragments of BE_R1_41

In some forms, the truncated or cleaved form of a deaminase protein is a cleaved BE_R1_41 deaminase protein fragment including amino acid residues at the amino (NH) terminus resulting from cleavage at a position including any of Gly33, or Gly43, or Gly69, or Gly108.

In some forms, the cleaved form of a deaminase protein is cleaved form of a BE_R1_41 deaminase protein cleaved at amino acid Gly33 (BE_R1_41_NG33), having an amino acid sequence: GSYTLGSYQISAPQLPAYNGQTVGTFYYVNGAG (SEQ ID NO:168), or an amino acid having at least 70%, 75%, 80%, 85%, 90%, 95%, or 99% sequence identity to SEQ ID NO:168, or fragment thereof.

In some forms, the truncated or cleaved form of a deaminase protein is a truncated or cleaved form of a BE_R1_41 deaminase protein truncated at amino acid Gly43 (BE_R1_41_N_G43), having an amino acid sequence: GSYTLGSYQISAPQLPAYNGQTVGTFYYVNGAGGLESRTFSSG (SEQ ID NO:169), or an amino acid having at least 70%, 75%, 80%, 85%, 90%, 95%, or 99% sequence identity to SEQ ID NO:169, or fragment thereof.

In some forms, the cleaved form of a deaminase protein is cleaved form of a BE_R1_41 deaminase protein cleaved at amino acid Gly69 (BE_R1_41_NG69), having an amino acid sequence: GSYTLGSYQISAPQLPAYNGQTVGTFYYVNGAGGLESRTFSSGGPTPYPNYANAGHVEGQ SALFMRDNG (SEQ ID NO:170), or an amino acid having at least 70%, 75%, 80%, 85%, 90%, 95%, or 99% sequence identity to SEQ ID NO:170, or fragment thereof.

In some forms, the cleaved form of a deaminase protein is cleaved form of a BE_R1_41 deaminase protein cleaved at amino acid Gly108 (BE_R1_41_N_G108), having an amino acid sequence: GSYTLGSYQISAPQLPAYNGQTVGTFYYVNGAGGLESRTFSSGGPTPYPNYANAGHVEGQ SALFMRDNGISDGLVFHNNPEGTCGFCVNMTETLLPENSKLTVVPPEG (SEQ ID NO:171), or an amino acid having at least 70%, 75%, 80%, 85%, 90%, 95%, or 99% sequence identity to SEQ ID NO:171, or fragment thereof.

### Cleaved Carboxyl (COOH) Fragments of BE_R1_41

In some forms, the truncated or cleaved form of a deaminase protein is a truncated or cleaved BE_R1_41 deaminase protein fragment including amino acid residues at the (COOH) terminus resulting from cleavage at a position including any of Gly33, or Gly43, or Gly69, or Gly108.

In some forms, the cleaved form of a deaminase protein is cleaved form of a BE_R1_41 deaminase protein cleaved at amino acid Gly33 terminus (BE_R1_41_C_G33), having an amino acid sequence: GLESRTFSSGGPTPYPNYANAGHVEGQSALFMRDNGISDGLVFHNNPEGTCGFCVNMTET LLPENSKLTVVPPEGAIPVKRGATGETRTFTGNSKSPKSPVKGEC (SEQ ID NO: 172), or an amino acid having at least 70%, 75%, 80%, 85%, 90%, 95%, or 99% sequence identity to SEQ ID NO:172, or fragment thereof.

In some forms, the cleaved form of a deaminase protein is cleaved form of a BE_R1_41 deaminase protein cleaved at amino acid Gly43 (BE_R1_41_C_G43), having an amino acid sequence: GPTPYPNYANAGHVEGQSALFMRDNGISDGLVFHNNPEGTCGFCVNMTETLLPENSKLTV VPPEGAIPVKRGATGETRTFTGNSKSPKSPVKGEC (SEQ ID NO:173), or an amino acid having at least 70%, 75%, 80%, 85%, 90%, 95%, or 99% sequence identity to SEQ ID NO:173, or fragment thereof.

In some forms, the cleaved form of a deaminase protein is cleaved form of a BE_R1_41 deaminase protein cleaved at amino acid Lys71 (BE_R1_41_C_G69), having an amino acid sequence: DNGISDGLVFHNNPEGTCGFCVNMTETLLPENSKLTVVPPEGAIPVKRGATGETRTFTGN SKSPKSPVKGEC (SEQ ID NO:174), or an amino acid having at least 70%, 75%, 80%, 85%, 90%, 95%, or 99% sequence identity to SEQ ID NO:174, or fragment thereof.

In some forms, the cleaved form of a deaminase protein is cleaved form of a BE_R1_41 deaminase protein cleaved at amino acid Gly108 (BE_R1_28_C_G108), having an amino acid sequence: AIPVKRGATGETRTFTGNSKSPKSPVKGEC (SEQ ID NO:175), or an amino acid having at least 70%, 75%, 80%, 85%, 90%, 95%, or 99% sequence identity to SEQ ID NO:175, or fragment thereof.

### Combinations of Split BE-_R1_41 Deaminase Proteins

In some forms, the truncated or cleaved form of BE_R1_41 deaminase protein lacks deaminase function alone. In some forms, the combination of two or more of the truncated or cleaved form of BE_R1_41 deaminase protein reconstitutes the deaminase function. For example, in some forms, combining one truncated or cleaved form of BE_R1_41 deaminase protein lacking one or more amino acid residues from the amino (NH) terminus, or a fragment from the carboxyl (COOH) terminus of the complete BE_R1_41 deaminase domain becomes functional upon combination or co-localization with one or more truncated or cleaved form of BE_R1_41 deaminase protein lacking one or more amino acid residues from the carboxyl (COOH) terminus, or a fragment from the amino (NH) terminus of the complete BE_R1_41 deaminase domain. For example, in some forms, base editors include a split BE_R1_41 deaminase domain having an amino acid sequence of any one of SEQ ID NOS:168-172, where the base editor has reconstituted deaminase activity upon co-localization or combination with another split BE_R1_41 deaminase domain having an amino acid sequence of any one of SEQ ID NOS:173-175, or together with a “dead” form of the BE_R1_12 deaminase domain having an amino acid sequence of SEQ ID NO:123, or an amino acid having at least 70%, 75%, 80%, 85%, 90%, 95%, or 99% sequence identity to SEQ ID NO:123.

### (5) Split BE_R4_21 Deaminase Proteins

In some forms, the truncated or cleaved form of a deaminase protein is a truncated or cleaved form of a BE_R4_21 deaminase protein.

### Cleaved Amino (NH) Fragments of BE_R4_21

In some forms, the truncated or cleaved form of a deaminase protein is a cleaved BE_R4_21 deaminase protein fragment including amino acid residues at the amino (NH) terminus resulting from cleavage at a position including any of Ser62, or Gly127.

In some forms, the cleaved form of a deaminase protein is cleaved form of a BE_R4_21 deaminase protein cleaved at amino acid Ser62 (BE_R4_21_N_S62), having an amino acid sequence: GGSAVVGAGVVATGAKAVTTGKSLSESQATLSVAQRLLATIGEEGKTAGVLELDGELIPL VS (SEQ ID NO:176), or an amino acid having at least 70%, 75%, 80%, 85%, 90%, 95%, or 99% sequence identity to SEQ ID NO:176, or fragment thereof.

In some forms, the truncated or cleaved form of a deaminase protein is a truncated or cleaved form of a BE_R4_21 deaminase protein truncated at amino acid Gly127 (BE_R4_21_N_G127), having an amino acid sequence: GGSAVVGAGVVATGAKAVTTGKSLSESQATLSVAQRLLATIGEEGKTAGVLELDGELIPL VSGKSSLPNYAASGHVEGQAALIMRDRGATSGRLLIDNPSGICGYCKSQVATLLPENATL QVGTPLG (SEQ ID NO:177), or an amino acid having at least 70%, 75%, 80%, 85%, 90%, 95%, or 99% sequence identity to SEQ ID NO:177, or fragment thereof.

### Cleaved Carboxyl (COOH) Fragments of BE_R4_21

In some forms, the truncated or cleaved form of a deaminase protein is a truncated or cleaved BE_R4_21 deaminase protein fragment including amino acid residues at the (COOH) terminus resulting from cleavage at a position including any of Ser62, or Gly127.

In some forms, the cleaved form of a deaminase protein is cleaved form of a BE_R4_21 deaminase protein cleaved at amino acid Ser62 terminus (BE_R4_21_C_S62), having an amino acid sequence: GKSSLPNYAASGHVEGQAALIMRDRGATSGRLLIDNPSGICGYCKSQVATLLPENATLQV GTPLGTVTPSSRWSASRTFTGNDRDPKPWPR (SEQ ID NO:178), or an amino acid having at least 70%, 75%, 80%, 85%, 90%, 95%, or 99% sequence identity to SEQ ID NO:178, or fragment thereof.

In some forms, the cleaved form of a deaminase protein is cleaved form of a BE_R4_21 deaminase protein cleaved at amino acid Gly127 (BE_R4_21_C_G127), having an amino acid sequence: TVTPSSRWSASRTFTGNDRDPKPWPR (SEQ ID NO:179), or an amino acid having at least 70%, 75%, 80%, 85%, 90%, 95%, or 99% sequence identity to SEQ ID NO:179, or fragment thereof.

### Combinations of Split BE_R4_21 Deaminase Proteins

In some forms, the truncated or cleaved form of BE_R4_21 deaminase protein lacks deaminase function alone. In some forms, the combination of two or more of the truncated or cleaved form of BE_R4_21 deaminase protein reconstitutes the deaminase function. For example, in some forms, combining one truncated or cleaved form of BE_R4_21 deaminase protein lacking one or more amino acid residues from the amino (NH) terminus, or a fragment from the carboxyl (COOH) terminus of the complete BE_R4_21 deaminase domain becomes functional upon combination or co-localization with one or more truncated or cleaved form of BE_R4_21 deaminase protein lacking one or more amino acid residues from the carboxyl (COOH) terminus, or a fragment from the amino (NH) terminus of the complete BE_R4_21 deaminase domain. For example, in some forms, base editors include a split BE_R4_21 deaminase domain having an amino acid sequence of any one of SEQ ID NOS:176-177, where the base editor has reconstituted deaminase activity upon co-localization or combination with another split BE_R4_21 deaminase domain having an amino acid sequence of any one of SEQ ID NOS:178-179, or together with a “dead” form of the BE_R4_21 deaminase domain having an amino acid sequence of SEQ ID NO:125, or an amino acid having at least 70%, 75%, 80%, 85%, 90%, 95%, or 99% sequence identity to SEQ ID NO:125.

### (6) Split BE_R2_11 Deaminase Proteins

In some forms, the truncated or cleaved form of a deaminase protein is a truncated or cleaved form of a BE_R2_11 deaminase protein.

### Truncated Fragments of BE_R2_11

In some forms, the truncated or cleaved form of a deaminase protein is a fragment of the BE_R2_11 deaminase protein including amino acid residues resulting from truncation of 54 or 39 contiguous amino acid residues from the amino (NH) terminus.

In some forms, the cleaved form of a deaminase protein is truncated form of a BE_R2_11 deaminase protein resulting from removal of 54 residues from the amino (NH) terminus (BE_R2_11_A54), having an amino acid sequence: HYDKVRKELGTSAEVPGHAEGVAFNKAYQVRKNTGTKGGNAVLYVDKIPCVMCKPGIATL MRSAKVDHLDLHYLQDGKMHHVQYVRNPDTDAVYNPFSGKWTKPSKKK (SEQ ID NO:180), or an amino acid having at least 70%, 75%, 80%, 85%, 90%, 95%, or 99% sequence identity to SEQ ID NO:180, or fragment thereof.

In some forms, the cleaved form of a deaminase protein is truncated form of a BE_R2_11 deaminase protein resulting from removal of 39 residues from the amino (NH) terminus (BE_R2_11_A39), having an amino acid sequence: KWVTKGKTSNYTDKAHYDKVRKELGTSAEVPGHAEGVAFNKAYQVRKNTGTKGGNAVLYV DKIPCVMCKPGIATLMRSAKVDHLDLHYLQDGKMHHVQYVRNPDTDAVYNPFSGKWTKPS KKK (SEQ ID NO:181), or an amino acid having at least 70%, 75%, 80%, 85%, 90%, 95%, or 99% sequence identity to SEQ ID NO:181, or fragment thereof.

### Combinations of Split BE_R2_11 Deaminase Proteins

In some forms, the truncated or cleaved form of BE_R2_11 deaminase protein lacks deaminase function alone. In some forms, the combination of two or more of the truncated or cleaved form of BE_R2_11 deaminase protein reconstitutes the deaminase function. For example, in some forms, combining one truncated or cleaved form of BE_R2_11 deaminase protein lacking one or more amino acid residues from the amino (NH) terminus, or a fragment from the carboxyl (COOH) terminus of the complete BE_R2_11 deaminase domain becomes functional upon combination or co-localization with one or more truncated or cleaved form of BE_R2_11 deaminase protein lacking one or more amino acid residues from the carboxyl (COOH) terminus, or a fragment from the amino (NH) terminus of the complete BE_R2_11 deaminase domain. For example, in some forms, base editors include a split BE_R2_11 deaminase domain having an amino acid sequence of SEQ ID NO:180 or 181, where the base editor has reconstituted deaminase activity upon co-localization or combination with another split BE_R2_11 deaminase domain having an amino acid sequence of SEQ ID NOS:180-181, or together with a “dead” form of the BE_R2_11 deaminase domain having an amino acid sequence of SEQ ID NO:126, or an amino acid having at least 70%, 75%, 80%, 85%, 90%, 95%, or 99% sequence identity to SEQ ID NO:126.

### 2. Functional Domains

The base editors typically include one or more functional domains. Functional domains include programmable DNA binding domains/targeting domains, nucleases, and other domains. In some forms, the functional domain is a targeting domain. In some forms, the targeting domain can recognize and/or bind to a specific target sequence in a nucleic acid (e.g., DNA or RNA sequence). Thus, in some forms, the targeting domain is a DNA and/or RNA binding protein or domain, such as a TALE, CRISPR-Cas9, Cfp1, or Zinc finger. Accordingly, in some forms, the base editor is a targeted base editor that includes a deaminase domain and one or more targeting domains (e.g., DNA binding protein or domain), wherein each targeting domain specifically binds to a target sequence.

A base editor can include any number of functional domains so as long as it retains desired activity (e.g., deaminase activity). For example, a base editor can include a range of 1-5 functional domains. In some forms, a base editor includes 1, 2, 3, 4, 5 or more functional (e.g., targeting) domains. In some forms, a base editor includes a deaminase domain and one functional domain. In some forms, a base editor includes a deaminase domain and two functional domains. In some forms, a base editor includes a deaminase domain and three functional domains. In some forms, a targeted base editor includes a deaminase domain and one targeting domain. In some forms, a targeted base editor includes a deaminase domain and two targeting domains. In some forms, a targeted base editor includes a deaminase domain and three targeting domains.

The one or more functional domains and the deaminase domain can be arranged in any orientation within the base editor. For example, the deaminase domain can be at the N- or C-terminus of the base editor. In some forms, the base editor conforms to the following architecture/structure:
NH₂[deaminase domain]-[functional domain]COOH; or
NH₂[functional domain]-[deaminase domain]COOH
   wherein NH₂ is the N-terminus of the base editor, and COOH is the C-terminus of the base editor. Preferably, the functional domain is a targeting domain. In some forms, the “-” used in the general architecture above indicates the presence of an optional linker.

In some forms, the base editors disclosed herein do not include a linker. In some forms, a linker is present between one or more of the domains or proteins within the base editor (e.g., between a deaminase domain and a first functional (e.g., targeting) domain and/or a second functional domain). In some forms, the deaminase domain and the functional (e.g., targeting) domain are fused via any appropriate linker known in the art, for example, any of the linkers provided below in the subsection entitled “Linkers.” In some forms, the various domains or components forming the base editor are fused via a linker that includes from about 1-200 amino acids, inclusive. In some forms, the linker includes from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100to 200, or 150 to 200 amino acids.

In particular forms, disclosed is a targeted base editor that includes any of the deaminase domains disclosed herein and a targeting domain, wherein the targeting domain specifically binds to a base editor target sequence. Preferably, the targeting domain is or includes a TALE, CRISPR-Cas effector protein (e.g., Cas9, Cfp1), or Zinc finger protein or domain. For example, in cases where the targeting domain is or includes a CRISPR-Cas effector protein (e.g., Cas9, Cfp1), the base editor target sequence can be the same as or include the protospacer sequence.

The base editor target sequence can be present in a target nucleic acid within any distance of the target nucleotide sequence of the deaminase domain that supports deamination of the target nucleotide sequence. A preferred design principle for the disclosed targeted base editors is to select the base editor target sequence (and targeting domain) and linkage of the deaminase domain and targeting domain such that the targeting domain binds the target nucleic acid in proximity to the instance of the target nucleotide sequence in the target nucleic acid intended to be deaminated. This proximity should be such that, for the given target base editor and target nucleic acid, the deaminase domain can deaminate the intended instance of the target nucleotide sequence in the target nucleic acid. For example, the base editor target sequence can be present in a target nucleic acid within 1-100, 20-80, 40-60, 10-50, 20-40, 1-10, 1-20, 10-20, or 5-10 nucleotides of an instance of the target nucleotide sequence of the deaminase domain. In some forms, the base editor target sequence is present in a target nucleic acid within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, or 90-100 nucleotides of an instance of the target nucleotide sequence of the deaminase domain. In preferred forms, the base editor target sequence is selected to be present in a target nucleic acid within 20 nucleotides of an instance of the target nucleotide sequence of the deaminase domain. Preferably, the instance of the target nucleotide sequence is selected to be base edited by the targeted base editor.

In some forms, the instance of the target nucleotide sequence is the only instance of the target nucleotide sequence in the target nucleic acid. In some cases, multiple instances (e.g., 2, 3, 4, 5, or more) of the target nucleotide sequence are present in the target nucleic acid. Thus, in some forms, the specific instance of the multiple instances of the target nucleotide that is selected to be base edited by the targeted base editor can be described or specified based on the distance from the targeted base editor target sequence (e.g., as the only instance within a specified distance from the target base editor target sequence).

For example, in some forms, the instance of the target nucleotide sequence (in the target nucleic acid) that is selected to be base edited is the only instance of the target nucleotide sequence of the deaminase domain within 1-100, 20-80, 40-60, 10-50, 20-40, 1-10, 1-20, 10-20, or 5-10 nucleotides of the base editor target sequence. In some forms, the instance of the target nucleotide sequence (in the target nucleic acid) that is selected to be base edited is the only instance of the target nucleotide sequence of the deaminase domain within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, or 90-100 nucleotides of the base editor target sequence. In some forms, the instance of the target nucleotide sequence (in the target nucleic acid) that is selected to be base edited is the only instance of the target nucleotide sequence of the deaminase domain within 20 nucleotides of the base editor target sequence.

However, independently of this “only instance” distance, the instance of the target nucleotide sequence (in the target nucleic acid) that is selected to be base edited can be any distance from the selected base editor target sequence (so long as it is less than or equal to the “only instance” distance specified). For example, the instance of the target nucleotide sequence (in the target nucleic acid) that is selected to be base edited can be the only instance of the target nucleotide sequence of the deaminase domain within 20 nucleotides of the base editor target sequence, while this instance of the target nucleotide sequence that is selected to be base edited is itself within 20 nucleotides or less of the base editor target sequence. More generally, in some forms, the instance of the target nucleotide sequence (in the target nucleic acid) that is selected to be base edited can be the only instance of the target nucleotide sequence of the deaminase domain within 1-100, 20-80, 40-60, 10-50, 20-40, 1-10, 1-20, 10-20, or 5-10 nucleotides of the base editor target sequence, while this instance of the target nucleotide sequence that is selected to be base edited is itself within 1-100, 20-80, 40-60, 10-50, 20-40, 1-10, 1-20, 10-20, or 5-10 nucleotides or less of the base editor target sequence. Thus, in some forms, the instance of the target nucleotide sequence (in the target nucleic acid) that is selected to be base edited can be the only instance of the target nucleotide sequence of the deaminase domain within 20 nucleotides of the base editor target sequence, while this instance of the target nucleotide sequence that is selected to be base edited is itself within 20, 19, 18, 17, 16, 15, 14, 13, 12, 11, 10, 9, 8, 7, 6, 5, 4, 3, 2, or 1 nucleotides of the base editor target sequence.

In some forms, multiple instances (e.g., 2, 3, 4, 5, or more) of the base editor target sequence are present in the target nucleic acid. Thus, in some forms, the selected base editor target sequence can be described or specified based on the distance from the instance of the target nucleotide sequence that is the selected to be base edited by the targeted base editor (e.g., as the only base editor target sequence in the target nucleic acid that is within a specified distance of the instance of target nucleotide sequence selected to be base edited). For example, in some forms, the base editor target sequence within 1-100, 20-80, 40-60, 10-50, 20-40, 1-10, 1-20, 10-20, or 5-10 nucleotides of the target nucleotide sequence (in the target nucleic acid) that is selected to be base edited is the only base editor target sequence in the target nucleic acid that is within 1-100, 20-80, 40-60, 10-50, 20-40, 1-10, 1-20, 10-20, or 5-10 nucleotides of the target nucleotide sequence that is selected to be base edited. In some forms, the base editor target sequence within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, or 90-100 nucleotides of the target nucleotide sequence (in the target nucleic acid) that is selected to be base edited is the only base editor target sequence in the target nucleic acid that is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, or 90-100 nucleotides of the target nucleotide sequence that is selected to be base edited. In some forms, the base editor target sequence within 20 nucleotides of the target nucleotide sequence (in the target nucleic acid) that is selected to be base edited is the only base editor target sequence in the target nucleic acid that is within 20 nucleotides of the target nucleotide sequence that is selected to be base edited.

In some forms, the base editor target sequence within 1-100, 20-80, 40-60, 10-50, 20-40, 1-10, 1-20, 10-20, or 5-10 nucleotides of the target nucleotide sequence (in the target nucleic acid) that is selected to be base edited is the only base editor target sequence in the target nucleic acid that is within 1-100, 20-80, 40-60, 10-50, 20-40, 1-10, 1-20, 10-20, or 5-10 nucleotides of any instance of the target nucleotide sequence. In some forms, the base editor target sequence within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, or 90-100 nucleotides of the target nucleotide sequence (in the target nucleic acid) that is selected to be base edited is the only base editor target sequence in the target nucleic acid that is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, or 90-100 nucleotides of any instance of the target nucleotide sequence. In some forms, the base editor target sequence within 20 nucleotides of the target nucleotide sequence (in the target nucleic acid) that is selected to be base edited is the only base editor target sequence in the target nucleic acid that is within 20 nucleotides of any instance of the target nucleotide sequence.

In some forms, the instance of the target nucleotide sequence in the target nucleic acid (e.g., selected to be base edited by the targeted base editor) is the only instance of the target nucleotide sequence of the deaminase domain within 20 nucleotides of the base editor target sequence in the target nucleic acid within 20 nucleotides of the instance of the target nucleotide sequence. In some forms, the instance of the target nucleotide sequence in the target nucleic acid (e.g., selected to be base edited by the targeted base editor) is the only instance of the target nucleotide sequence of the deaminase domain within 1-100, 20-80, 40-60, 10-50, 20-40, 1-10, 1-20, 10-20, or 5-10 nucleotides of the base editor target sequence in the target nucleic acid within 1-100, 20-80, 40-60, 10-50, 20-40, 1-10, 1-20, 10-20, or 5-10 nucleotides of the instance of the target nucleotide sequence. In some forms, the instance of the target nucleotide sequence in the target nucleic acid (e.g., selected to be base edited by the targeted base editor) is the only instance of the target nucleotide sequence of the deaminase domain within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, or 90-100 nucleotides of the base editor target sequence in the target nucleic acid within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, or 90-100 nucleotides of the instance of the target nucleotide sequence.

In any of the foregoing, the base editor target sequence can be in nuclear DNA or mitochondrial DNA. In some preferred forms, the base editor target sequence is present in mitochondrial DNA.

i. Programmable DNA Binding Protein

In some forms, the base editors include at least one programmable DNA binding protein. In some forms, the base editors include more than a single programmable DNA binding protein. For example, in some forms, the base editors include a first and a second programmable DNA binding protein. In some forms, the first and/or second programmable DNA binding protein are the same. In other forms, the first and/or second programmable DNA binding protein are different. Exemplary first and/or second programmable DNA binding proteins include a Cas domain (e.g., Cas9), a nickase, a zinc-finger protein and a TALE protein. Therefore, in some forms the base editor includes a heterodimer having first and second monomers, the first monomer including: a Cas domain, a nickase, a zinc-finger protein or a TALE protein; and an N-terminal or C-terminal fragment of a cleaved double-stranded DNA deaminase, and a second monomer including: a Cas domain, a nickase, a zinc-finger protein or a TALE protein; and a second programmable DNA binding protein and an N-terminal or C-terminal fragment of a cleaved double-stranded DNA deaminase, whereby dimerization of the first and second monomers reconstitutes the double-stranded DNA deaminase activity. Exemplary Cas domains include Cas9, Casl2e, Casl2d, Casl2a, Casl2bl, Cas13a, Casl2c, and Argonaute.

ii. Exemplary Functional Domains

In some forms, the base editors include one or more functional domains that are programmable DNA binding factors, such as programmable DNA binding proteins. The terms “programmable DNA binding protein,” “pDNA binding protein,” “pDNA binding protein domain” or “pDNAbp” refer to any protein that localizes to and binds a specific target DNA nucleotide sequence (e.g. a gene locus of a genome). This term embraces RNA-programmable proteins, which associate (e.g. form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. The term also embraces proteins which bind directly to nucleotide sequence in an amino acid-programmable manner, e.g., zinc finger proteins and TALE proteins. Exemplary RNA-programmable proteins are CRISPR-Cas9 proteins, as well as Cas9 equivalents, homologs, orthologs, or paralogs, whether naturally occurring or non-naturally occurring (e.g. engineered or modified), and may include a Cas9 equivalent from any type of CRISPR system (e.g. type II, V, VI), including Cpf1 (a typeV CRISPR-Cas systems), C2cl (a type V CRISPR-Cas system), C2c2 (a type VI CRISPR-Cas system), C2c3 (a type V CRISPR-Cas system), dCas9, GeoCas9, CjCas9, Cas12a, Casl2b, Cas12c, Casl2d, Cas12g, Cas12h, Cas12i, Cas13d, Cas14, Argonaute, and nCas9. Further Cas equivalents are described in Makarova et al., “C2c2 is a single-component programmable RNAguided RNA-targeting CRISPR effector,” Science 2016; 353(6299), the contents of which are incorporated herein by reference.

### a. Zinc Fingers

In some forms, the targeted base editor includes one or more zinc finger proteins or zinc finger DNA-binding domains as the one or more targeting domains. Custom-designed base editors that combine deaminase domains with zinc finger domains offer a general and efficient way to introduce targeted (site-specific) base edits into the genome.

Zinc fingers are part of a large superfamily of protein domains that can bind to DNA. Zinc fingers are among the most common DNA-binding motifs found in eukaryotes. It is estimated that there are 500 zinc finger proteins encoded by the yeast genome and that perhaps 1% of all mammalian genes encode zinc finger containing proteins. A zinc finger consists of two antiparallel R strands, and an a helix. The zinc ion is crucial for the stability of this domain type—in the absence of the metal ion the domain unfolds as it is too small to have a hydrophobic core. The structure of each individual finger is highly conserved and consists of about 30 amino acid residues, constructed as a ββα fold and held together by the zinc ion. The α-helix occurs at the C-terminal part of the finger, while the β-sheet occurs at the N-terminal part.

Zinc finger proteins are classified according to the number and position of the cysteine and histidine residues available for zinc coordination. The CCHH class, typified by the Xenopus transcription factor IIIA, is the largest. These proteins contain two or more fingers in tandem repeats. In contrast, the steroid receptors contain only cysteine residues that form two types of zinc-coordinated structures with four (C4) and five (C5) cysteines. Another class of zinc fingers contains the CCHC fingers. The CCHC fingers, which are found in Drosophila, and in mammalian and retroviral proteins, display the consensus sequence C-N₂-C—N₄-H—N₄—C(SEQ ID NO:28). A configuration of CCHC finger, of the C-N₅-C—N₁₂-H—N₄—C(SEQ ID NO:29) type, is found in the neural zinc finger factor/myelin transcription factor family. Finally, several yeast transcription factors such as GAL4 and CHA4 contain an atypical C6 zinc finger structure that coordinates two zinc ions. Zinc fingers are usually found in multiple copies (up to 37) per protein. These copies can be organized in a tandem array, forming a single cluster or multiple clusters, or they can be dispersed throughout the protein.

Each zinc finger motif is typically considered to recognize and bind to a three-base pair sequence and as such, a protein including more zinc fingers targets a longer sequence and therefore has a greater specificity and affinity to the target site. In some forms, individual zinc-finger domains bind to 3 bp subsites, and arrays of fingers can bind extended 9 or 12 bp sequence targets.

The zinc finger DNA-binding domain, which can, in principle, be designed to target any genomic location of interest, can be a tandem array of Cys2His2 zinc fingers, each of which generally recognizes three to four nucleotides in the target DNA sequence. The Cys2His2 domain has a general structure: Phe (sometimes Tyr)-Cys-(2 to 4 amino acids)-Cys-(3 amino acids)-Phe(sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long. The zinc finger proteins bind to zinc and form structural domains that bind the major groove of the DNA double helix. Variations of key amino acids in each DNA-binding finger contribute to binding affinity and specificity.

The published literature describes many different publicly available zinc-finger engineering methods which can be broadly grouped into two general categories: (1) modular assembly methods in which individual fingers with pre-characterized specificities are joined together in order to design a protein which binds to a specific DNA sequence or (2) selection-based methods which require multiple large randomized libraries (e.g., selection of desirable mutants from a library of randomized zinc fingers using phage display can generate DNA-specific binding domains).

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

Much research has revealed that a key requirement for constructing high-quality, multi-finger domains is accounting for the context-dependent activities of individual finger domains within the longer array. The Oligomerized Pool ENgineering (OPEN) method for constructing multi-finger domains addresses the context-dependent activities of individual zinc fingers but is also robust and relatively easier to perform than previously described methods. See International Patent Application Publication No. WO 2009/146179, which is hereby incorporated by reference in its entirety. OPEN is scalable and can be used to generate high quality multi-finger domains for a very large number of different target sites in parallel. OPEN is enabled by the construction of a large archive of zinc-finger pools designed to bind various DNA sequences. To date, OPEN has been used to generate multi-finger domains for over 500 different target sites that function well in a bacterial cell-based assays.

Zinc finger nucleases (ZFNs) that include a DNA-binding domain derived from a zinc-finger protein linked to a cleavage domain (such as the Type IIS enzyme Fokl) are typically used to induce targeted (site-specific) DNA mutations (e.g., deletions) via double stranded DNA breaks that are repaired by non-homologous end joining (NHEJ). The targeted base editors disclosed herein can be used in an analogous manner, except that a deaminase domain is used instead of the cleavage domain, resulting in targeted base editing of DNA as compared to DNA cleavage. Thus, methods for engineering base editors containing one or more zinc finger proteins or DNA-binding domains are apparent and can be adapted from those known in the art for producing ZFNs.

ZFNs function as dimers with each monomer containing a non-specific cleavage domain fused to an array of artificial zinc fingers engineered to bind a target DNA sequence of interest. Thus, in some forms, the disclosed targeted base editors can also function as dimers that bind to base editor target sequences flanking (e.g., upstream and downstream) a target nucleotide sequence of the deaminase domain. This is especially useful when the deaminase domains (of the base editor) are split into two distinct portions. Thus, in some forms, the N-terminal portion of the deaminase domain is linked to a first zinc finger domain while the C-terminal portion of the deaminase domain is linked to a second zinc finger domain. The two zinc finger domains and/or the base editor target sequences bound by the zinc finger domains can, but need not be, the same. The zinc finger domains can be designed and selected such that the two zinc finger-deaminase domain molecules are optimally spaced on a target nucleic acid so that they dimerize. In some forms, such a split targeted base editor is only capable of deaminating a target nucleotide sequence when the subcomponents are combined (e.g., co-expressed or co-introduced) and dimerize.

Zinc fingers are structurally diverse and exhibit a wide range of functions, from DNA- or RNA-binding to protein-protein interactions and membrane association. There are more than 40 types of zinc fingers annotated in UniProtKB. The most frequent are the C2H2-type, the CCHC-type, the PHD-type and the RING-type. Examples include UniProtKB Accession Nos. Q7Z142, P55197, Q9P2R3, Q9P2G1, Q9P2S6, Q8IUH5, P19811, Q92793, P36406, 095081, and Q9ULV3.

In some forms, the zinc finger protein is (Q7Z142-1) having an amino acid sequence: MPDFTIIQPDRKFDAAAVAGIFVRSSTSSSFPSASSYIAAKKRKNVDNTSTRKPYSYKDR KRKNTEEIRNIKKKLFMDLGIVRTNCGIDNEKQDREKAMKRKVTETIVTTYCELCEQNFS SSKMLLLHRGKVHNTPYIECHLCMKLFSQTIQFNRHMKTHYGPNAKIYVQCELCDRQFKD KQSLRTHWDVSHGSGDNQAVLA (SEQ ID NO:72), or an amino acid having at least 70%, 75%, 80%, 85%, 90%, 95%, or 99% sequence identity to SEQ ID NO:72, or fragment thereof.

Zinc Fingers that recognize the mitochondrial hND DNA region In some forms, the zinc finger protein is a left hand side (L) zinc finger (ZF) protein. In some forms, the left hand side zinc finger protein is a ZF that recognizes the hND1 DNA sequence. In some forms, the left hand side zinc finger protein that recognizes the hND1 DNA sequence is (ZF_hND-L1) having an amino acid sequence: MEPGEKPYKCPECGKSFSTSGSLVRHQRTHTGEKPYKCPECGKSFSDCRDLARHQRTHTG EKPYKCPECGKSFSQNSTLTEHQRTHTGEKPYKCPECGKSFSERSHLREHQRTHTGKKTS (SEQ ID NO:74), or an amino acid having at least 70%, 75%, 80%, 85%, 90%, 95%, or 99% sequence identity to SEQ ID NO:74, or fragment thereof.

In some forms, the left hand side zinc finger protein that recognizes the hND1 DNA sequence is (ZF_hND-L2) having an amino acid sequence: MEPGEKPYKCPECGKSFSRNDTLTEHQRTHTGEKPYKCPECGKSFSREDNLHTHQRTHTG EKPYKCPECGKSFSDCRDLARHQRTHTGEKPYKCPECGKSFSQNSTLTEHQRTHTGEKPY KCPECGKSFSTKNSLTEHQRTHTGKKTS (SEQ ID NO:75), or an amino acid having at least 70%, 75%, 80%, 85%, 90%, 95%, or 99% sequence identity to SEQ ID NO:75, or fragment thereof.

In some forms, the left hand side zinc finger protein that recognizes the hND1 DNA sequence is (ZF_hND-L3) having an amino acid sequence: MEPGEKPYKCPECGKSFSDPGHLVRHQRTHTGEKPYKCPECGKSFSQNSTLTEHQRTHTG EKPYKCPECGKSFSRSDKLTEHQRTHTGEKPYKCPECGKSFSQRANLRAHQRTHTGKKTS (SEQ ID NO:76), or an amino acid having at least 70%, 75%, 80%, 85%, 90%, 95%, or 99% sequence identity to SEQ ID NO:76, or fragment thereof.

In some forms, the left hand side zinc finger protein that recognizes the hND1 DNA sequence is (ZF_hND-L4) having an amino acid sequence: MEPGEKPYKCPECGKSFSQLAHLRAHQRTHTGEKPYKCPECGKSFSTSGELVRHQRTHTG EKPYKCPECGKSFSREDNLHTHQRTHTGEKPYKCPECGKSFSDPGHLVRHQRTHTGEKPY KCPECGKSFSDSGNLRVHQRTHTGKKTS (SEQ ID NO:77), or an amino acid having at least 70%, 75%, 80%, 85%, 90%, 95%, or 99% sequence identity to SEQ ID NO:77, or fragment thereof.

In some forms, the zinc finger protein is a right hand side (R) zinc finger (ZF) protein. In some forms, the right hand side zinc finger protein is a ZF that recognizes the hND1 DNA sequence. In some forms, the right hand side zinc finger protein that recognizes the hND1 DNA sequence is: MEPGEKPYKCPECGKSFSTKNSLTEHQRTHTGEKPYKCPECGKSFSSKKALTEHQRTHTG EKPYKCPECGKSFSTSGELVRHQRTHTGEKPYKCPECGKSFSTSGNLVRHQRTHTGKKTS (SEQ ID NO:78), or an amino acid having at least 70%, 75%, 80%, 85%, 90%, 95%, or 99% sequence identity to SEQ ID NO:78, or fragment thereof.

In some forms, the right hand side zinc finger protein that recognizes the hND1 DNA sequence is (ZF_hND-R2) having an amino acid sequence: MEPGEKPYKCPECGKSFSTSGNLVRHQRTHTGEKPYKCPECGKSFSTKNSLTEHQRTHTG EKPYKCPECGKSFSSKKALTEHQRTHTGEKPYKCPECGKSFSTSGELVRHQRTHTGEKPY KCPECGKSFSTSGNLVRHQRTHTGKKTS (SEQ ID NO:79), or an amino acid having at least 70%, 75%, 80%, 85%, 90%, 95%, or 99% sequence identity to SEQ ID NO:79, or fragment thereof.

In some forms, the right hand side zinc finger protein that recognizes the hND1 DNA sequence is (ZF_hND-R3) having an amino acid sequence: MEPGEKPYKCPECGKSFSTSGNLTEHQRTHTGEKPYKCPECGKSFSRSDNLVRHQRTHTG EKPYKCPECGKSFSTSGHLVRHQRTHTGEKPYKCPECGKSFSRADNLTEHQRTHTGKKTS (SEQ ID NO:80), or an amino acid having at least 70%, 75%, 80%, 85%, 90%, 95%, or 99% sequence identity to SEQ ID NO:80, or fragment thereof.

In some forms, the right hand side zinc finger protein that recognizes the hND1 DNA sequence is (ZF_hND-R4) having an amino acid sequence: MEPGEKPYKCPECGKSFSTSGNLTEHQRTHTGEKPYKCPECGKSFSRSDNLVRHQRTHTG EKPYKCPECGKSFSTSGHLVRHQRTHTGEKPYKCPECGKSFSRADNLTEHQRTHTGEKPY KCPECGKSFSTSGNLVRHQRTHTGKKTS (SEQ ID NO:81), or an amino acid having at least 70%, 75%, 80%, 85%, 90%, 95%, or 99% sequence identity to SEQ ID NO:81, or fragment thereof.

Zinc Fingers that Recognize the Mitochondrial mCOXJ DNA Region

In some forms, the left hand side zinc finger protein is a ZF that recognizes the mCOX DNA sequence. In some forms, the left hand side zinc finger protein that recognizes the mCOX DNA sequence is (ZF_mCOX1-L1) having an amino acid sequence: MEPGEKPYKCPECGKSFSHKNALQNHQRTHTGEKPYKCPECGKSFSTSGNLTEHQRTHTG EKPYKCPECGKSFSTSGNLTEHQRTHTGEKPYKCPECGKSFSHTGHLLEHQRTHTGEKPY KCPECGKSFSTTGALTEHQRTHTGKKTS (SEQ ID NO:82), or an amino acid having at least 70%, 75%, 80%, 85%, 90%, 95%, or 99% sequence identity to SEQ ID NO:82, or fragment thereof.

In some forms, the left hand side zinc finger protein is a ZF that recognizes the mCOX1 DNA sequence. In some forms, the left hand side zinc finger protein that recognizes the mCOX1 DNA sequence is (ZF_mCOX1-L2) having an amino acid sequence: MEPGEKPYKCPECGKSFSSRRTCRAHQRTHTGEKPYKCPECGKSFSHKNALQNHQRTHTG EKPYKCPECGKSFSTSGNLTEHQRTHTGEKPYKCPECGKSFSTSGNLTEHQRTHTGEKPY KCPECGKSFSHTGHLLEHQRTHTGKKTS (SEQ ID NO:83), or an amino acid having at least 70%, 75%, 80%, 85%, 90%, 95%, or 99% sequence identity to SEQ ID NO:83, or fragment thereof.

In some forms, the left hand side zinc finger protein that recognizes the mCOX1 DNA sequence is (ZF_mCOX1-L3) having an amino acid sequence: MEPGEKPYKCPECGKSFSRSDHLTNHQRTHTGEKPYKCPECGKSFSSRRTCRAHQRTHTG EKPYKCPECGKSFSHKNALQNHQRTHTGEKPYKCPECGKSFSTSGNLTEHQRTHTGEKPY KCPECGKSFSTSGNLTEHQRTHTGKKTS (SEQ ID NO:84), or an amino acid having at least 70%, 75%, 80%, 85%, 90%, 95%, or 99% sequence identity to SEQ ID NO:84, or fragment thereof.

In some forms, the left hand side zinc finger protein that recognizes the mCOX1 DNA sequence is (ZF_mCOX1-L4) having an amino acid sequence: MEPGEKPYKCPECGKSFSERSHLREHQRTHTGEKPYKCPECGKSFSRSDHLTNHQRTHTG EKPYKCPECGKSFSSRRTCRAHQRTHTGEKPYKCPECGKSFSHKNALQNHQRTHTGEKPY KCPECGKSFSTSGNLTEHQRTHTGKKTS (SEQ ID NO:85), or an amino acid having at least 70%, 75%, 80%, 85%, 90%, 95%, or 99% sequence identity to SEQ ID NO:85, or fragment thereof.

In some forms, the left hand side zinc finger protein that recognizes the mCOX1 DNA sequence is (ZF_mCOX1-L5) having an amino acid sequence: MEPGEKPYKCPECGKSFSRRDELNVHQRTHTGEKPYKCPECGKSFSRRDELNVHQRTHTG EKPYKCPECGKSFSTTGNLTVHQRTHTGEKPYKCPECGKSFSRTDTLRDHQRTHTGEKPY KCPECGKSFSTKNSLTEHQRTHTGKKTS (SEQ ID NO:86), or an amino acid having at least 70%, 75%, 80%, 85%, 90%, 95%, or 99% sequence identity to SEQ ID NO:86, or fragment thereof.

In some forms, the right hand side zinc finger protein that recognizes the mCOX1 DNA sequence is (ZF_mCOX1-R1) having an amino acid sequence: MEPGEKPYKCPECGKSFSQLAHLRAHQRTHTGEKPYKCPECGKSFSQRAHLERHQRTHTG EKPYKCPECGKSFSRSDNLVRHQRTHTGEKPYKCPECGKSFSTSGSLVRHQRTHTGEKPY KCPECGKSFSTTGNLTVHQRTHTGKKTS (SEQ ID NO:87), or an amino acid having at least 70%, 75%, 80%, 85%, 90%, 95%, or 99% sequence identity to SEQ ID NO:87, or fragment thereof.

In some forms, the right hand side zinc finger protein that recognizes the mCOX1 DNA sequence is (ZF_mCOX1-R2) having an amino acid sequence: MEPGEKPYKCPECGKSFSRRDELNVHQRTHTGEKPYKCPECGKSFSQLAHLRAHQRTHTG EKPYKCPECGKSFSQRAHLERHQRTHTGEKPYKCPECGKSFSRSDNLVRHQRTHTGEKPY KCPECGKSFSTSGSLVRHQRTHTGKKTS (SEQ ID NO:88), or an amino acid having at least 70%, 75%, 80%, 85%, 90%, 95%, or 99% sequence identity to SEQ ID NO:88, or fragment thereof.

In some forms, the right hand side zinc finger protein that recognizes the mCOX1 DNA sequence is (ZF_mCOX1-R3) having an amino acid sequence: MEPGEKPYKCPECGKSFSRRDELNVHQRTHTGEKPYKCPECGKSFSTSGSLVRHQRTHTG EKPYKCPECGKSFSTTGNLTVHQRTHTGEKPYKCPECGKSFSRKDNLKNHQRTHTGEKPY KCPECGKSFSRSDKLVRHQRTHTGKKTS (SEQ ID NO:89), or an amino acid having at least 70%, 75%, 80%, 85%, 90%, 95%, or 99% sequence identity to SEQ ID NO:89, or fragment thereof.

b. Transcription Activator-Like (TAL) Effectors

In some forms, the targeted base editor includes one or more transcription activator-like (TAL) effectors as the one or more targeting domains. Custom-designed base editors that combine deaminase domains with TAL effectors offer a general and efficient way to introduce targeted (site-specific) base edits into the genome.

TAL effectors are proteins of plant pathogenic bacteria that are injected by the pathogen into the plant cell, where they travel to the nucleus and function as transcription factors to turn on specific plant genes. The modular DNA recognition domain of transcription activator-like effectors (TALEs) was originally found in natural transcription factors encoded by pathogenic bacteria of the genus Xanthomonas and more recently in Ralstonia solanacearum. Xanthomonas TALEs are the most widely used in the genome engineering field. The primary amino acid sequence of a TAL effector dictates the nucleotide sequence to which it binds. Thus, target sites can be predicted for TAL effectors, and TAL effectors also can be engineered and generated for the purpose of binding to particular nucleotide sequences, such as base editor target sequences as described herein.

Each module within the TAL effector DNA binding domain contains a conserved stretch of typically 34 residues that mediates the interaction with a single nucleotide via a di-residue in positions 12 and 13, called the ‘repeat variable di-residues’ (RVDs). Modules with different specificities can be fused into tailored arrays without the context-dependency issues that represent the major limitation for the generation of zinc-finger arrays. Hence, this simple ‘one module to one nucleotide’ cypher makes the generation of TALEs with novel specificities rapid and affordable.

The TAL effector DNA-binding domain is a tandem array of amino acid repeats, each about 34 residues long. The repeats are very similar to each other; typically they differ principally at two positions (amino acids 12 and 13, called the repeat variable residue, or RVD). Each RVD specifies preferential binding to one of the four possible nucleotides, meaning that each TALE repeat binds to a single base pair, though the NN RVD is known to bind adenines in addition to guanine. Non-limiting examples of RVDs and their corresponding target nucleotides are shown below in Table 1. See also, International Patent Application Publication No. WO 2010/079430, which is hereby incorporated by reference in its entirety.

TABLE 1
Exemplary RVDs and their corresponding target nucleotides.

| RVD | Nucleotide |
|---|---|
| HD | C |
| NG | T |
| NI | A |
| NN | G or A |
| NS | A or C or G |
| HG | T |
| IG | T |

Natural TALEs have a strict requirement for the presence of a T at the beginning of their target site (TO rule), a specificity that is dictated by the TALE N-terminal domain. Engineered TALE N-terminal domains have been described that relax this specificity and allow targeting sequences that start with other nucleotides (Lamb, B. M., Mercer, A. C., & Barbas III, C. F. (2013). Directed evolution of the TALE N-terminal domain for recognition of all 5′ bases. Nucleic acids research, 41(21), 9779-9785).

TAL effector DNA binding is mechanistically less well understood than that of zinc-finger proteins, but their seemingly simpler code is beneficial for programmable, site-specific DNA binding. TALEs also have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). US Published Application No. 2011/0145940, which discloses TAL effectors and methods of using them to modify DNA. Miller et al. Nature Biotechnol 29: 143 (2011) reported making transcription activator-like effector nucleases (TALENs) for site-specific nuclease architecture by linking TAL truncation variants to the catalytic domain of Fokl nuclease. The resulting TALENs were shown to induce gene modification in immortalized human cells. General design principles for TALE binding domains can be found in, for example, WO 2011/072246, which is hereby incorporated by reference in its entirety.

A sequence-specific TALE can recognize a particular sequence within a preselected target nucleic acid (e.g., present on chromosomal or mitochondrial DNA). Thus, in some forms, a target nucleotide sequence can be scanned for TALE recognition sites, and a particular TALE can be selected based on the target sequence. In other forms, a TALE can be engineered to target a particular sequence. Sequence-specific TAL effectors that contain a plurality of DNA binding repeats that, in combination, bind to a base editor target sequence can be designed. As described herein, TAL effectors include a number of imperfect repeats that determine the specificity with which they interact with DNA. Each repeat binds to a single base, depending on the particular di-amino acid sequence at residues 12 and 13 of the repeat. Thus, by engineering the repeats within a TAL effector (e.g., using standard techniques known in the art), particular DNA sites can be targeted.

Similar to ZFNs, some TALENs contain endonucleases (e.g., Fokl) that only function as dimers, which can be capitalized upon to enhance the target specificity of the TAL effector. For example, in some cases each Fokl monomer can be fused to a TAL effector sequence that recognizes a different DNA target sequence, and only when the two recognition sites are in close proximity do the inactive monomers come together to create a functional TALEN. The targeted base editors disclosed herein can be used in an analogous manner, except that a deaminase domain is used instead of the endonuclease (e.g., Fokl), resulting in targeted base editing of DNA as compared to DNA cleavage. Thus, methods for engineering base editors containing one or more TAL effectors are apparent and can be adapted from those known in the art for producing TALENs.

As discussed above when zinc fingers are used as the targeting domain(s) of base editors, a disclosed targeted base editor containing a TAL effector as the targeting domain can also function as a dimer in some forms. Thus, in some forms, the disclosed targeted base editors can function as dimers that bind to base editor target sequences flanking (e.g., upstream and downstream) a target nucleotide sequence of the deaminase domain. This is especially useful when the deaminase domains (of the base editor) are split into two distinct portions. Thus, in some forms, the N-terminal portion of the deaminase domain is linked to a first TAL effector while the C-terminal portion of the deaminase domain is linked to a second TAL effector. The two TAL effectors and/or the base editor target sequences bound by the TAL effectors can, but need not be, the same. The TAL effectors can be designed and selected such that the two TALE-deaminase domain molecules are optimally spaced on a target nucleic acid so that they dimerize. In some forms, such a split targeted base editor is only capable of deaminating a target nucleotide sequence when the subcomponents are combined (e.g., co-expressed or co-introduced) and dimerize.

In some forms, the TALE protein is a left hand side (L) TALE protein, or a right hand side (R) TALE protein. In some forms, the TALE protein is a TALE that recognizes the hND1 DNA sequence.

TALEs that Recognize the hND DNA Region

In some forms, the left hand side TALE protein that recognizes the hND1 DNA sequence is (TALE_hND-L1) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNGGGKQALETVQRLLPVLCQAHGLTPQQVVAIASNIGGKQALETVQRLLPVLCQAHG LTPEQVVAIASNNGGKQALETVQALLPVLCQAHGLTPEQVVAIASHDGGKQALETVQALL PVLCQAHGLTPEQVVAIASHDGGKQALETVQALLPVLCQAHGLTPEQVVAIASNGGGKQA LETVORLLPVLCQAHGLTPEQVVAIASNIGGKQALETVQALLPVLCQAHGLTPEQVVAIA SNNGGKQALETVQRLLPVLCQAHGLTPEQVVAIASHDGGKQALETVQALLPVLCQAHGLT PEQVVAIASHDGGKQALETVQALLPVLCQAHGLTPEQVVAIASNNGGKQALETVQALLPV LCQAHGLTPEQVVAIASNGGGKQALETVQRLLPVLCQAHGLTPQQVVAIASNGGGKQALE TVQRLLPVLCQAHGLTPQQVVAIASNGGGRPALESIVAQLSRPDPALAALTNDHLVALAC LGGRPALDAVKKGLG (SEQ ID NO:90), or an amino acid having at least 70%, 75%, 80%, 85%, 90%, 95%, or 99% sequence identity to SEQ ID NO:90, or fragment thereof.

In some forms, the right hand side TALE protein that recognizes the hND1 DNA sequence is (TALE_hND-R1) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNNGGKQALETVQALLPVLCQAHGLTPQQVVA IASNIGGKQALETVQRLLPVLCQAHGLTPQQVVAIASNNGGKQALETVQRLLPVLCQAHG LTPEQVVAIASNGGGKQALETVQALLPVLCQAHGLTPEQVVAIASNGGGKQALETVQALL PVLCQAHGLTPEQVVAIASNGGGKQALETVQALLPVLCQAHGLTPEQVVAIASNNGGKQA LETVQRLLPVLCQAHGLTPEQVVAIASNIGGKQALETVQALLPVLCQAHGLTPEQVVAIA SNGGGKQALETVQRLLPVLCQAHGLTPEQVVAIASNNGGKQALETVQALLPVLCQAHGLT PEQVVAIASHDGGKQALETVQALLPVLCQAHGLTPEQVVAIASNGGGKQALETVQALLPV LCQAHGLTPEQVVAIASHDGGKQALETVQRLLPVLCQAHGLTPQQVVAIASNIGGKQALE TVQRLLPVLCQAHGLTPEQVVAIASHDGGKQALETVQALLPVLCQAHGLTPQQVVAIASH DGGKQALETVQRLLPVLCQAHGLTPQQVVAIASHDGGKQALETVQRLLPVLCQAHGLTPQ QVVAIASNGGGRPALESIVAQLSRPDPALAALTNDHLVALACLGGRPALDAVKKGLG (SEQ ID NO:91), or an amino acid having at least 70%, 75%, 80%, 85%, 90%, 95%, or 99% sequence identity to SEQ ID NO:91, or fragment thereof.

In some forms, the TALE protein is a TALE that recognizes the mND6 DNA sequence. In some forms, the left hand side TALE protein that recognizes the mND6 DNA sequence is (TALE_mND6-L1) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNIGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNGGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASHDGGKQALETVQALLPVLCQAHGLTPEQVVAIA SNGGGKQALETVQRLLPVLCQAHGLTPEQVVAIASNIGGKQALETVQALLPVLCQAHGLT PEQVVAIASNIGGKQALETVQALLPVLCQAHGLTPEQVVAIASHDGGKQALETVQALLPV LCHAHGLTPEQVVAIASNIGGKQALETVQRLLPVLCQAHGLTPEQVVAIASNGGGKQALE TVQALLPVLCQAHGLTPQQVVAIASNGGGRPALESIVAQLSRPDPALAALTNDHLVALAC LGGRPALDAVKKGLG (SEQ ID NO:92), or an amino acid having at least 70%, 75%, 80%, 85%, 90%, 95%, or 99% sequence identity to SEQ ID NO:92, or fragment thereof.

In some forms, the right hand side TALE protein that recognizes the mND6 DNA sequence is (TALE_mND6-R1) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNIGGKQALETVQALL PVLCQAHGLTPEQVVAIASNGGGKQALETVQALLPVLCQAHGLTPEQVVAIASNIGGKQA LETVQRLLPVLCQAHGLTPEQVVAIASNNGGKQALETVQALLPVLCQAHGLTPEQVVAIA SHDGGKQALETVQRLLPVLCQAHGLTPEQVVAIASHDGGKQALETVQALLPVLCQAHGLT PEQVVAIASNGGGKQALETVQALLPVLCQAHGLTPEQVVAIASNGGGKQALETVQALLPV LCHAHGLTPEQVVAIASHDGGKQALETVQRLLPVLCQAHGLTPEQVVAIASHDGGKQALE TVQALLPVLCQAHGLTPQQVVAIASNGGGRPALESIVAQLSRPDPALAALTNDHLVALAC LGGRPALDAVKKGLG (SEQ ID NO:93), or an amino acid having at least 70%, 75%, 80%, 85%, 90%, 95%, or 99% sequence identity to SEQ ID NO:93, or fragment thereof.

In some forms, the right hand side TALE protein that recognizes the mND6 DNA sequence is (TALE_mND6-R2) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNIGGKQALETVQALLPVLCQAHGLTPQQVVA IASNIGGKQALETVQRLLPVLCQAHGLTPQQVVAIASNIGGKQALETVQRLLPVLCQAHG LTPEQVVAIASHDGGKQALETVQALLPVLCQAHGLTPEQVVAIASHDGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASHDGGKQALETVQALLPVLCQAHGLTPEQVVAIA SNIGGKQALETVQRLLPVLCQAHGLTPEQVVAIASNGGGKQALETVQALLPVLCQAHGLT PEQVVAIASNIGGKQALETVQALLPVLCQAHGLTPEQVVAIASNIGGKQALETVQALLPV LCHAHGLTPEQVVAIASNIGGKQALETVQRLLPVLCQAHGLTPQQVVAIASNGGGRPALE SIVAQLSRPDPALAALTNDHLVALACLGGRPALDAVKKGLGGSAIPVKRGATGETKVFTG NSNSPKSPTKGGC (SEQ ID NO:94), or an amino acid having at least 70%, 75%, 80%, 85%, 90%, 95%, or 99% sequence identity to SEQ ID NO:94, or fragment thereof.

In some forms, the TALE protein is a TALE that recognizes the mND1 DNA sequence. In some forms, the left hand side TALE protein that recognizes the mND1 DNA sequence is (TALE_mND1-L1) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNIGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNGGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASHDGGKQALETVQALLPVLCQAHGLTPEQVVAIA SNGGGKQALETVQRLLPVLCQAHGLTPEQVVAIASNIGGKQALETVQALLPVLCQAHGLT PEQVVAIASNIGGKQALETVQALLPVLCQAHGLTPEQVVAIASHDGGKQALETVQALLPV LCHAHGLTPEQVVAIASNIGGKQALETVQRLLPVLCQAHGLTPEQVVAIASNGGGKQALE TVQALLPVLCQAHGLTPQQVVAIASNGGGRPALESIVAQLSRPDPALAALTNDHLVALAC LGGRPALDAVKKGLG (SEQ ID NO:95), or an amino acid having at least 70%, 75%, 80%, 85%, 90%, 95%, or 99% sequence identity to SEQ ID NO:95, or fragment thereof.

In some forms, the left hand side TALE protein that recognizes the mND1 DNA sequence is (TALE_mND1-L2) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNIGGKQALETVQALL PVLCQAHGLTPEQVVAIASNGGGKQALETVQALLPVLCQAHGLTPEQVVAIASNIGGKQA LETVQRLLPVLCQAHGLTPEQVVAIASNNGGKQALETVQALLPVLCQAHGLTPEQVVAIA SHDGGKQALETVQRLLPVLCQAHGLTPEQVVAIASHDGGKQALETVQALLPVLCQAHGLT PEQVVAIASNGGGKQALETVQALLPVLCQAHGLTPEQVVAIASNGGGKQALETVQALLPV LCHAHGLTPEQVVAIASHDGGKQALETVQRLLPVLCQAHGLTPEQVVAIASHDGGKQALE TVQALLPVLCQAHGLTPQQVVAIASNGGGRPALESIVAQLSRPDPALAALTNDHLVALAC LGGRPALDAVKKGLG (SEQ ID NO:96), or an amino acid having at least 70%, 75%, 80%, 85%, 90%, 95%, or 99% sequence identity to SEQ ID NO:96, or fragment thereof.

In some forms, the TALE protein is a TALE that recognizes the h11 DNA sequence. In some forms, TALE protein that recognizes the h11 DNA sequence is (TALE_h11) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNIGGKQALETVQRLLPVLCQAHGLTPQQVVAIASNGGGKQALETVQRLLPVLCQAHG LTPEQVVAIASNIGGKQALETVQALLPVLCQAHGLTPEQVVAIASNGGGKQALETVQALL PVLCQAHGLTPEQVVAIASNGGGKQALETVQALLPVLCQAHGLTPEQVVAIASNNGGKQA LETVQRLLPVLCQAHGLTPEQVVAIASHDGGKQALETVQALLPVLCQAHGLTPEQVVAIA SNGGGKQALETVQRLLPVLCQAHGLTPEQVVAIASNGGGKQALETVQALLPVLCQAHGLT PEQVVAIASHDGGKQALETVQALLPVLCQAHGLTPEQVVAIASHDGGKQALETVQALLPV LCHAHGLTPEQVVAIASNNGGKQALETVQRLLPVLCQAHGLTPQQVVAIASNGGGRPALE SIVAQLSRPDPALAALTNDHLVALACLGGRPALDAVKKGLG (SEQ ID NO:97), or an amino acid having at least 70%, 75%, 80%, 85%, 90%, 95%, or 99% sequence identity to SEQ ID NO:97, or fragment thereof.

In some forms, the TALE protein is a TALE that recognizes the h12 DNA sequence. In some forms, TALE protein that recognizes the h12 DNA sequence is (TALE_h12) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNIGGKQALETVQRLLPVLCQAHGLTPQQVVAIASNIGGKQALETVQRLLPVLCQAHG LTPEQVVAIASNIGGKQALETVQALLPVLCQAHGLTPEQVVAIASNNGGKQALETVQALL PVLCQAHGLTPEQVVAIASNGGGKQALETVQALLPVLCQAHGLTPEQVVAIASNIGGKQA LETVQRLLPVLCQAHGLTPEQVVAIASNGGGKQALETVQALLPVLCQAHGLTPEQVVAIA SNGGGKQALETVQRLLPVLCQAHGLTPEQVVAIASNGGGKQALETVQALLPVLCQAHGLT PEQVVAIASNIGGKQALETVQALLPVLCQAHGLTPEQVVAIASNNGGKQALETVQALLPV LCHAHGLTPEQVVAIASHDGGKQALETVQRLLPVLCQAHGLTPQQVVAIASNGGGRPALE SIVAQLSRPDPALAALTNDHLVALACLGGRPALDAVKKGLG (SEQ ID NO:98), or an amino acid having at least 70%, 75%, 80%, 85%, 90%, 95%, or 99% sequence identity to SEQ ID NO:98, or fragment thereof.

In some forms, the TALE protein is a TALE that recognizes the mCOX1 DNA sequence. In some forms, the left hand side TALE protein that recognizes the mCOX1 DNA sequence is (TALE_mCOX1-L1) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNIGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASNIGGKQALETVQALLPVLCQAHGLTPEQVVAIA SNGGGKQALETVQRLLPVLCQAHGLTPEQVVAIASNIGGKQALETVQALLPVLCQAHGLT PEQVVAIASNGGGKQALETVQALLPVLCQAHGLTPQQVVAIASNGGGKQALETVQRLLPV LCQAHGLTPQQVVAIASHDGGKQALETVQRLLPVLCQAHGLTPEQVVAIASNNGGKQALE TVQALLPVLCQAHGLTPEQVVAIASNGGGKQALETVQALLPVLCQAHGLTPEQVVAIASN IGGKQALETVQALLPVLCQAHGLTPEQVVAIASNNGGKQALETVQRLLPVLCQAHGLTPE QVVAIASNNGGKQALETVQALLPVLCQAHGLTPEQVVAIASNIGGKQALETVQRLLPVLC QAHGLTPQQVVAIASNNGGRPALESIVAQLSRPDPALAALTNDHLVALACLGGRPALDAV KKGLG (SEQ ID NO:99), or an amino acid having at least 70%, 75%, 80%, 85%, 90%, 95%, or 99% sequence identity to SEQ ID NO:99, or fragment thereof.

In some forms, the left hand side TALE protein that recognizes the mCOX1 DNA sequence is (TALE_mCOX1-L2) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNIGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASNIGGKQALETVQALLPVLCQAHGLTPEQVVAIA SNGGGKQALETVQRLLPVLCQAHGLTPEQVVAIASNIGGKQALETVQALLPVLCQAHGLT PEQVVAIASNGGGKQALETVQALLPVLCQAHGLTPQQVVAIASNGGGKQALETVQRLLPV LCQAHGLTPQQVVAIASHDGGKQALETVQRLLPVLCQAHGLTPEQVVAIASNNGGKQALE TVQALLPVLCQAHGLTPEQVVAIASNGGGKQALETVQALLPVLCQAHGLTPEQVVAIASN IGGKQALETVQALLPVLCQAHGLTPEQVVAIASNNGGKQALETVQRLLPVLCQAHGLTPE QVVAIASNNGGKQALETVQALLPVLCQAHGLTPQQVVAIASNIGGRPALESIVAQLSRPD PALAALTNDHLVALACLGGRPALDAVKKGLG (SEQ ID NO:100), or an amino acid having at least 70%, 75%, 80%, 85%, 90%, 95%, or 99% sequence identity to SEQ ID NO:100, or fragment thereof.

In some forms, the left hand side TALE protein that recognizes the mCOX1 DNA sequence is (TALE_mCOX1-L3) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNIGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASNIGGKQALETVQALLPVLCQAHGLTPEQVVAIA SNGGGKQALETVQRLLPVLCQAHGLTPEQVVAIASNIGGKQALETVQALLPVLCQAHGLT PEQVVAIASNGGGKQALETVQALLPVLCQAHGLTPQQVVAIASNGGGKQALETVQRLLPV LCQAHGLTPQQVVAIASHDGGKQALETVQRLLPVLCQAHGLTPEQVVAIASNNGGKQALE TVQALLPVLCQAHGLTPEQVVAIASNGGGKQALETVQALLPVLCQAHGLTPEQVVAIASN IGGKQALETVQALLPVLCQAHGLTPEQVVAIASNNGGKQALETVQRLLPVLCQAHGLTPQ QVVAIASNNGGRPALESIVAQLSRPDPALAALTNDHLVALACLGGRPALDAVKKGLG (SEQ ID NO:101), or an amino acid having at least 70%, 75%, 80%, 85%, 90%, 95%, or 99% sequence identity to SEQ ID NO:101, or fragment thereof.

In some forms, the left hand side TALE protein that recognizes the mCOX1 DNA sequence is (TALE_mCOX1-L4) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNIGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASNIGGKQALETVQALLPVLCQAHGLTPEQVVAIA SNGGGKQALETVQRLLPVLCQAHGLTPEQVVAIASNIGGKQALETVQALLPVLCQAHGLT PEQVVAIASNGGGKQALETVQALLPVLCQAHGLTPQQVVAIASNGGGKQALETVQRLLPV LCQAHGLTPQQVVAIASHDGGKQALETVQRLLPVLCQAHGLTPEQVVAIASNNGGKQALE TVQALLPVLCQAHGLTPQQVVAIASNGGGRPALESIVAQLSRPDPALAALTNDHLVALAC LGGRPALDAVKKGLG (SEQ ID NO:102), or an amino acid having at least 70%, 75%, 80%, 85%, 90%, 95%, or 99% sequence identity to SEQ ID NO:102, or fragment thereof.

In some forms, the left hand side TALE protein that recognizes the mCOX1 DNA sequence is (TALE_mCOX1-L5) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASNNGGKQALETVQRLLPVLCQAHGLTPQQVVAIASHDGGKQALETVQRLLPVLCQAHG LTPEQVVAIASHDGGKQALETVQALLPVLCQAHGLTPEQVVAIASNIGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASNIGGKQALETVQALLPVLCQAHGLTPEQVVAIA SNGGGKQALETVQRLLPVLCQAHGLTPEQVVAIASNIGGKQALETVQALLPVLCQAHGLT PQQVVAIASNGGGRPALESIVAQLSRPDPALAALTNDHLVALACLGGRPALDAVKKGLG (SEQ ID NO:103), or an amino acid having at least 70%, 75%, 80%, 85%, 90%, 95%, or 99% sequence identity to SEQ ID NO:103, or fragment thereof.

In some forms, the left hand side TALE protein that recognizes the mCOX1 DNA sequence is (TALE_mCOX1-L6) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASHDGGKQALETVQALLPVLCQAHGLTPQQVVA IASHDGGKQALETVQRLLPVLCQAHGLTPQQVVAIASNGGGKQALETVQRLLPVLCQAHG LTPEQVVAIASNIGGKQALETVQALLPVLCQAHGLTPEQVVAIASHDGGKQALETVQALL PVLCQAHGLTPEQVVAIASNNGGKQALETVQALLPVLCQAHGLTPEQVVAIASNIGGKQA LETVQRLLPVLCQAHGLTPEQVVAIASNIGGKQALETVQALLPVLCQAHGLTPEQVVAIA SNGGGKQALETVQRLLPVLCQAHGLTPEQVVAIASNIGGKQALETVQALLPVLCQAHGLT PEQVVAIASNGGGKQALETVQALLPVLCQAHGLTPEQVVAIASNNGGKQALETVQALLPV LCHAHGLTPEQVVAIASNIGGKQALETVQRLLPVLCQAHGLTPQQVVAIASNGGGRPALE SIVAQLSRPDPALAALTNDHLVALACLGGRPALDAVKKGLG (SEQ ID NO:104), or an amino acid having at least 70%, 75%, 80%, 85%, 90%, 95%, or 99% sequence identity to SEQ ID NO:104, or fragment thereof.

In some forms, the left hand side TALE protein that recognizes the mCOX1 DNA sequence is (TALE_mCOX1-L7) having an amino acid sequence: DIADLRTLGYSQQQQEKIKPKVRSTVAQHHEALVGHGFTHAHIVALSQHPAALGTVAVKY QDMIAALPEATHEAIVGVGKSRSGARALEALLTVAGELRGPPLQLDTGQLLKIAKRGGVT AVEAVHAWRNALTGAPLNLTPEQVVAIASHDGGKQALETVQALLPVLCQAHGLTPEQVVA IASHDGGKQALETVQALLPVLCQAHGLTPEQVVAIASHDGGKQALETVQALLPVLCHAHG LTPEQVVAIASNIGGKQALETVQRLLPVLCQAHGLTPEQVVAIASHDGGKQALETVQALL PVLCQAHGLTPEQVVAIASNGGGKQALETVQALLPVLCQAHGLTPQQVVAIASNGGGKQA LETVQRLLPVLCQAHGLTPEQVVAIASHDGGKQALETVQALLPVLCQAHGLTPQQVVAIA SNNGGKQALETVQRLLPVLCQAHGLTPQQVVAIASHDGGKQALETVQRLLPVLCQAHGLT PEQVVAIASHDGGKQALETVQALLPVLCQAHGLTPEQVVAIASNIGGKQALETVQALLPV LCQAHGLTPEQVVAIASNGGGKQALETVQALLPVLCQAHGLTPEQVVAIASHDGGKQALE TVQRLLPVLCQAHGLTPEQVVAIASNIGGKQALETVQALLPVLCQAHGLTPEQVVAIASN GGGKQALETVQRLLPVLCQAHGLTPEQVVAIASNIGGKQALETVQALLPVLCQAHGLTPQ QVVAIASNGGGRPALESIVAQLSRPDPALAALTNDHLVALACLGGRPALDAVKKGLG (SEQ ID NO:105), or an amino acid having at least 70%, 75%, 80%, 85%, 90%, 95%, or 99% sequence identity to SEQ ID NO:105, or fragment thereof.

In some forms, the left hand side TALE protein that recognizes the mCOX1 DNA sequence is (TALE_mCOX1-L7) having an amino acid sequence: DIADLRTLGYSQQQQEKIKPKVRSTVAQHHEALVGHGFTHAHIVALSQHPAALGTVAVKY QDMIAALPEATHEAIVGVGKYHGARALEALLTVAGELRGPPLQLDTGQLLKIAKRGGVTA VEAVHAWRNALTGAPLNLTPEQVVAIASHDGGKQALETVQALLPVLCQAHGLTPEQVVAI ASHDGGKQALETVQALLPVLCQAHGLTPEQVVAIASHDGGKQALETVQALLPVLCHAHGL TPEQVVAIASNIGGKQALETVQRLLPVLCQAHGLTPEQVVAIASHDGGKQALETVQALLP VLCQAHGLTPEQVVAIASNGGGKQALETVQALLPVLCQAHGLTPQQVVAIASNGGGKQAL ETVQRLLPVLCQAHGLTPEQVVAIASHDGGKQALETVQALLPVLCQAHGLTPQQVVAIAS NNGGKQALETVQRLLPVLCQAHGLTPQQVVAIASHDGGKQALETVQRLLPVLCQAHGLTP EQVVAIASHDGGKQALETVQALLPVLCQAHGLTPEQVVAIASNIGGKQALETVQALLPVL CQAHGLTPEQVVAIASNGGGKQALETVQALLPVLCQAHGLTPEQVVAIASHDGGKQALET VQRLLPVLCQAHGLTPEQVVAIASNIGGKQALETVQALLPVLCQAHGLTPEQVVAIASNG GGKQALETVQRLLPVLCQAHGLTPEQVVAIASNIGGKQALETVQALLPVLCQAHGLTPQQ VVAIASNGGGRPALESIVAQLSRPDPALAALTNDHLVALACLGGRPALDAVKKGLG (SEQ ID NO:106), or an amino acid having at least 70%, 75%, 80%, 85%, 90%, 95%, or 99% sequence identity to SEQ ID NO:106, or fragment thereof.

In some forms, the right hand side TALE protein that recognizes the mCOX1 DNA sequence is (TALE_mCOX1-R1) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNNGGKQALETVQALLPVLCQAHGLTPQQVVA IASNIGGKQALETVQRLLPVLCQAHGLTPQQVVAIASNNGGKQALETVQRLLPVLCQAHG LTPEQVVAIASNNGGKQALETVQALLPVLCQAHGLTPEQVVAIASNNGGKQALETVQALL PVLCQAHGLTPEQVVAIASNIGGKQALETVQALLPVLCQAHGLTPEQVVAIASNIGGKQA LETVQRLLPVLCQAHGLTPEQVVAIASNNGGKQALETVQALLPVLCQAHGLTPEQVVAIA SNIGGKQALETVQRLLPVLCQAHGLTPEQVVAIASNIGGKQALETVQALLPVLCQAHGLT PEQVVAIASNGGGKQALETVQALLPVLCQAHGLTPQQVVAIASNNGGKQALETVQRLLPV LCQAHGLTPQQVVAIASNGGGKQALETVQRLLPVLCQAHGLTPEQVVAIASNGGGKQALE TVQALLPVLCQAHGLTPEQVVAIASNIGGKQALETVQALLPVLCQAHGLTPEQVVAIASN GGGKQALETVQALLPVLCQAHGLTPEQVVAIASNNGGKQALETVQRLLPVLCQAHGLTPQ QVVAIASNGGGRPALESIVAQLSRPDPALAALTNDHLVALACLGGRPALDAVKKGLG (SEQ ID NO:108), or an amino acid having at least 70%, 75%, 80%, 85%, 90%, 95%, or 99% sequence identity to SEQ ID NO:108, or fragment thereof.

In some forms, the right hand side TALE protein that recognizes the mCOX1 DNA sequence is (TALE_mCOX1-R2) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNNGGKQALETVQALLPVLCQAHGLTPQQVVA IASNIGGKQALETVQRLLPVLCQAHGLTPQQVVAIASNNGGKQALETVQRLLPVLCQAHG LTPEQVVAIASNNGGKQALETVQALLPVLCQAHGLTPEQVVAIASNNGGKQALETVQALL PVLCQAHGLTPEQVVAIASNIGGKQALETVQALLPVLCQAHGLTPEQVVAIASNIGGKQA LETVQRLLPVLCQAHGLTPEQVVAIASNNGGKQALETVQALLPVLCQAHGLTPEQVVAIA SNIGGKQALETVQRLLPVLCQAHGLTPEQVVAIASNIGGKQALETVQALLPVLCQAHGLT PEQVVAIASNGGGKQALETVQALLPVLCQAHGLTPQQVVAIASNNGGKQALETVQRLLPV LCQAHGLTPQQVVAIASNGGGKQALETVQRLLPVLCQAHGLTPEQVVAIASNGGGKQALE TVQALLPVLCQAHGLTPQQVVAIASNIGGRPALESIVAQLSRPDPALAALTNDHLVALAC LGGRPALDAVKKGLG (SEQ ID NO:109), or an amino acid having at least 70%, 75%, 80%, 85%, 90%, 95%, or 99% sequence identity to SEQ ID NO:109, or fragment thereof.

In some forms, the right hand side TALE protein that recognizes the mCOX1 DNA sequence is (TALE_mCOX1-R3) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNNGGKQALETVQALLPVLCQAHGLTPQQVVA IASNIGGKQALETVQRLLPVLCQAHGLTPQQVVAIASNNGGKQALETVQRLLPVLCQAHG LTPEQVVAIASNNGGKQALETVQALLPVLCQAHGLTPEQVVAIASNNGGKQALETVQALL PVLCQAHGLTPEQVVAIASNIGGKQALETVQALLPVLCQAHGLTPEQVVAIASNIGGKQA LETVQRLLPVLCQAHGLTPEQVVAIASNNGGKQALETVQALLPVLCQAHGLTPEQVVAIA SNIGGKQALETVQRLLPVLCQAHGLTPEQVVAIASNIGGKQALETVQALLPVLCQAHGLT PEQVVAIASNGGGKQALETVQALLPVLCQAHGLTPQQVVAIASNNGGKQALETVQRLLPV LCQAHGLTPQQVVAIASNGGGKQALETVQRLLPVLCQAHGLTPQQVVAIASNGGGRPALE SIVAQLSRPDPALAALTNDHLVALACLGGRPALDAVKKGLG (SEQ ID NO:110), or an amino acid having at least 70%, 75%, 80%, 85%, 90%, 95%, or 99% sequence identity to SEQ ID NO:110, or fragment thereof.

In some forms, the right hand side TALE protein that recognizes the mCOX1 DNA sequence is (TALE_mCOX1-R4) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNNGGKQALETVQALLPVLCQAHGLTPQQVVA IASNIGGKQALETVQRLLPVLCQAHGLTPQQVVAIASNNGGKQALETVQRLLPVLCQAHG LTPEQVVAIASNNGGKQALETVQALLPVLCQAHGLTPEQVVAIASNNGGKQALETVQALL PVLCQAHGLTPEQVVAIASNIGGKQALETVQALLPVLCQAHGLTPEQVVAIASNIGGKQA LETVQRLLPVLCQAHGLTPEQVVAIASNNGGKQALETVQALLPVLCQAHGLTPEQVVAIA SNIGGKQALETVQRLLPVLCQAHGLTPEQVVAIASNIGGKQALETVQALLPVLCQAHGLT PEQVVAIASNGGGKQALETVQALLPVLCQAHGLTPQQVVAIASNNGGKQALETVQRLLPV LCQAHGLTPQQVVAIASNGGGRPALESIVAQLSRPDPALAALTNDHLVALACLGGRPALD AVKKGLG (SEQ ID NO:111), or an amino acid having at least 70%, 75%, 80%, 85%, 90%, 95%, or 99% sequence identity to SEQ ID NO:111, or fragment thereof.

In some forms, the right hand side TALE protein that recognizes the mCOX1 DNA sequence is (TALE_mCOX1-R5) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNNGGKQALETVQALLPVLCQAHGLTPQQVVA IASNIGGKQALETVQRLLPVLCQAHGLTPQQVVAIASNNGGKQALETVQRLLPVLCQAHG LTPEQVVAIASNNGGKQALETVQALLPVLCQAHGLTPEQVVAIASNNGGKQALETVQALL PVLCQAHGLTPEQVVAIASNIGGKQALETVQALLPVLCQAHGLTPEQVVAIASNIGGKQA LETVQRLLPVLCQAHGLTPEQVVAIASNNGGKQALETVQALLPVLCQAHGLTPEQVVAIA SNIGGKQALETVQRLLPVLCQAHGLTPEQVVAIASNIGGKQALETVQALLPVLCQAHGLT PQQVVAIASNGGGRPALESIVAQLSRPDPALAALTNDHLVALACLGGRPALDAVKKGLG (SEQ ID NO:112), or an amino acid having at least 70%, 75%, 80%, 85%, 90%, 95%, or 99% sequence identity to SEQ ID NO:112, or fragment thereof.

In some forms, the right hand side TALE protein that recognizes the mCOX1 DNA sequence is (TALE_mCOX1-R6) having an amino acid sequence: DIADLRTLGYSQQQQEKIKPKVRSTVAQHHEALVGHGFTHAHIVALSQHPAALGTVAVKY QDMIAALPEATHEAIVGVGKQWSGARALEALLTVAGELRGPPLQLDTGQLLKIAKRGGVT AVEAVHAWRNALTGAPLNLTPEQVVAIASNIGGKQALETVQALLPVLCQAHGLTPQQVVA IASNIGGKQALETVQRLLPVLCQAHGLTPQQVVAIASHDGGKQALETVQRLLPVLCQAHG LTPEQVVAIASNIGGKQALETVQALLPVLCQAHGLTPEQVVAIASNGGGKQALETVQALL PVLCQAHGLTPEQVVAIASNGGGKQALETVQALLPVLCQAHGLTPEQVVAIASHDGGKQA LETVQRLLPVLCQAHGLTPEQVVAIASNGGGKQALETVQALLPVLCQAHGLTPEQVVAIA SNGGGKQALETVQRLLPVLCQAHGLTPEQVVAIASHDGGKQALETVQALLPVLCQAHGLT PEQVVAIASHDGGKQALETVQALLPVLCQAHGLTPEQVVAIASHDGGKQALETVQALLPV LCHAHGLTPQQVVAIASNGGGRPALESIVAQLSRPDPALAALTNDHLVALACLGGRPALD AVKKGLG (SEQ ID NO:113), or an amino acid having at least 70%, 75%, 80%, 85%, 90%, 95%, or 99% sequence identity to SEQ ID NO:113, or fragment thereof.

In some forms, the TALE protein recognizes the NT(G) DNA sequence (TALE_NT(G)) and has an amino acid sequence: DIADLRTLGYSQQQQEKIKPKVRSTVAQHHEALVGHGFTHAHIVALSQHPAALGTVAVKY QDMIAALPEATHEAIVGVGKSRSGARALEALLTVAGELRGPPLQLDTGQLLKIAKRGGVT AVEAVHAWRNALTGAPLN (SEQ ID NO:114), or an amino acid having at least 70%, 75%, 80%, 85%, 90%, 95%, or 99% sequence identity to SEQ ID NO:114, or fragment thereof.

In some forms, the TALE protein recognizes the NT(bN) DNA sequence (TALE_NT(bN)) and has an amino acid sequence: DIADLRTLGYSQQQQEKIKPKVRSTVAQHHEALVGHGFTHAHIVALSQHPAALGTVAVKY QDMIAALPEATHEAIVGVGKYHGARALEALLTVAGELRGPPLQLDTGQLLKIAKRGGVTA VEAVHAWRNALTGAPLN (SEQ ID NO:115), or an amino acid having at least 70%, 75%, 80%, 85%, 90%, 95%, or 99% sequence identity to SEQ ID NO:115, or fragment thereof. c. BAT proteins In some forms, the DNA binding protein is a TALE-like (e.g., BAT) protein.

Unlike TALEs, natural BATs do not follow a TO rule and have a relaxed specificity at their N-terminal domain, thus they can be designed to bind to targets with any starting nucleotides. In some forms, the BAT protein is a left hand side BAT protein, or a right hand side BAT protein. In some forms, the BAT protein is a left hand side BAT protein that recognizes the hND1 DNA sequence. In some forms, the left hand side BAT protein that recognizes the hND1 DNA sequence is (BAT_hND1-L) having an amino acid sequence: STAFVDQDKQMANRLNLSPLERSKIEKQYGGATTLAFISNKQNELAQILSRADILKIASY DCAAHALQAVLDCGPMLGKRGFSQSDIVKIAGNGGGAQALQAVLDLESMLGKRGFSRDDI AKMAGHDGGAQTLQAVLDLESAFRERGFSQADIVKIAGNGGGAQALYSVLDVEPTLGKRG FSRADIVKIAGNIGGAQALHTVLDLEPALGKRGFSRIDIVKIAANNGGAQALHAVLDLGP TLRECGFSQATIAKIAGHDGGAQALQMVLDLGPALGKRGFSQATIAKIAGHDGGAQALQT VLDLEPALCERGFGQATIAKMAGNGGGAQALQTVLDLEPALRKRDFRQADIIKIAGNIGG AQALQAVIEHGPTLRQHGFNLADIVKMAGNNGGAQALQAVLDLKPVLDEHGFSQADIVKI AGHDGGTQALHAVLDLERMLGERGFSRADIVNVAGHDGGAQALKAVLEHEATLNERGFSR ADIVKIAGNNGGAQALKAVLEHEATLDERGFSRADIVNVAGNGGGAQALKAVLEHEATLN ERGFNLTDIVEMAANGGGAQALKAVLEHGPTLRQRGLSLIDIVEIAGNGGGAQALKAVLK YGPVLMQAGRSNEEIVHVAARRGGAGRIRKMVAPLLERQ (SEQ ID NO:116), or an amino acid having at least 70%, 75%, 80%, 85%, 90%, 95%, or 99% sequence identity to SEQ ID NO:106, or fragment thereof.

In some forms, the BAT protein is a right hand side BAT protein that recognizes the hND1 DNA sequence. In some forms, the right hand side BAT protein that recognizes the hND1 DNA sequence is (BAT_hND1-R) having an amino acid sequence: STAFVDQDKQMANRLNLSPLERSKIEKQYGGATTLAFISNKQNELAQILSRADILKIASY DCAAHALQAVLDCGPMLGKRGFSQSDIVKIAGNNGGAQALQAVLDLESMLGKRGFSRDDI AKMAGNGGGAQTLQAVLDLESAFRERGFSQADIVKIAGNGGGAQALYSVLDVEPTLGKRG FSRADIVKIAGNGGGAQALHTVLDLEPALGKRGFSRIDIVKIAANNGGAQALHAVLDLGP TLRECGFSQATIAKIAGNIGGAQALQMVLDLGPALGKRGFSQATIAKIAGNGGGAQALQT VLDLEPALCERGFGQATIAKMAGNNGGAQALQTVLDLEPALRKRDFRQADIIKIAGHDGG AQALQAVIEHGPTLRQHGFNLADIVKMAGNGGGAQALQAVLDLKPVLDEHGFSQADIVKI AGHDGGTQALHAVLDLERMLGERGFSRADIVNVAGNIGGAQALKAVLEHEATLNERGFSR ADIVKIAGHDGGAQALKAVLEHEATLDERGFSRADIVNVAGHDGGAQALKAVLEHEATLN ERGFNLTDIVEMAAHDGGAQALKAVLEHGPTLRQRGLSLIDIVEIAGNGGGAQALKAVLK YGPVLMQAGRSNEEIVHVAARRGGAGRIRKMVAPLLERQ (SEQ ID NO:117), or an amino acid having at least 70%, 75%, 80%, 85%, 90%, 95%, or 99% sequence identity to SEO ID NO:117, or fragment thereof.

In some forms, the BAT protein is a left hand side BAT protein that recognizes the mCOX1 DNA sequence. In some forms, the left hand side BAT protein that recognizes the mCOX1 DNA sequence is (BAT_mCOX1-L) having an amino acid sequence: STAFVDQDKQMANRLNLSPLERSKIEKQYGGATTLAFISNKQNELAQILSRADILKIASY DCAAHALQAVLDCGPMLGKRGFSQSDIVKIAGHDGGAQALQAVLDLESMLGKRGFSRDDI AKMAGNIGGAQTLQAVLDLESAFRERGFSQADIVKIAGHDGGAQALYSVLDVEPTLGKRG FSRADIVKIAGNGGGAQALHTVLDLEPALGKRGFSRIDIVKIAANGGGAQALHAVLDLGP TLRECGFSQATIAKIAGHDGGAQALQMVLDLGPALGKRGFSQATIAKIAGNNGGAQALQT VLDLEPALCERGFGQATIAKMAGHDGGAQALQTVLDLEPALRKRDFRQADIIKIAGHDGG AQALQAVIEHGPTLRQHGFNLADIVKMAGNIGGAQALQAVLDLKPVLDEHGFSQADIVKI AGNGGGTQALHAVLDLERMLGERGFSRADIVNVAGHDGGAQALKAVLEHEATLNERGFSR ADIVKIAGNIGGAQALKAVLEHEATLDERGFSRADIVNVAGNGGGAQALKAVLEHEATLN ERGFNLTDIVEMAANIGGAQALKAVLEHGPTLRQRGLSLIDIVEIAGNGGGAQALKAVLK YGPVLMQAGRSNEEIVHVAARRGGAGRIRKMVAPLLERQ (SEQ ID NO:118), or an amino acid having at least 70%, 75%, 80%, 85%, 90%, 95%, or 99% sequence identity to SEQ ID NO:118, or fragment thereof.

In some forms, the BAT protein is a right hand side BAT protein that recognizes the mCOX1 DNA sequence. In some forms, the right hand side BAT protein that recognizes the mCOX1 DNA sequence is (BAT_mCOX1-R) having an amino acid sequence: STAFVDQDKQMANRLNLSPLERSKIEKQYGGATTLAFISNKQNELAQILSRADILKIASY DCAAHALQAVLDCGPMLGKRGFSQSDIVKIAGNGGGAQALQAVLDLESMLGKRGFSRDDI AKMAGNGGGAQTLQAVLDLESAFRERGFSQADIVKIAGNNGGAQALYSVLDVEPTLGKRG FSRADIVKIAGNIGGAQALHTVLDLEPALGKRGFSRIDIVKIAANNGGAQALHAVLDLGP TLRECGFSQATIAKIAGNNGGAQALQMVLDLGPALGKRGFSQATIAKIAGNNGGAQALQT VLDLEPALCERGFGQATIAKMAGNIGGAQALQTVLDLEPALRKRDFRQADIIKIAGNIGG AQALQAVIEHGPTLRQHGFNLADIVKMAGNNGGAQALQAVLDLKPVLDEHGFSQADIVKI AGNIGGTQALHAVLDLERMLGERGFSRADIVNVAGNIGGAQALKAVLEHEATLNERGFSR ADIVKIAGNGGGAQALKAVLEHEATLDERGFSRADIVNVAGNNGGAQALKAVLEHEATLN ERGFNLTDIVEMAANGGGAQALKAVLEHGPTLRQRGLSLIDIVEIAGNGGGAQALKAVLK YGPVLMQAGRSNEEIVHVAARRGGAGRIRKMVAPLLERQ (SEQ ID NO:119), or an amino acid having at least 70%, 75%, 80%, 85%, 90%, 95%, or 99% sequence identity to SEQ ID NO:119, or fragment thereof.

In some forms, the BAT protein is a left hand side BAT protein that recognizes the mND6 DNA sequence. In some forms, the left hand side BAT protein that recognizes the mND6 DNA sequence is (BAT_mND6-L) having an amino acid sequence: STAFVDQDKQMANRLNLSPLERSKIEKQYGGATTLAFISNKQNELAQILSRADILKIASY DCAAHALQAVLDCGPMLGKRGFSQSDIVKIAGNGGGAQALQAVLDLESMLGKRGFSRDDI AKMAGHDGGAQTLQAVLDLESAFRERGFSQADIVKIAGNGGGAQALYSVLDVEPTLGKRG FSRADIVKIAGNGGGAQALHTVLDLEPALGKRGFSRIDIVKIAANNGGAQALHAVLDLGP TLRECGFSQATIAKIAGNNGGAQALQMVLDLGPALGKRGFSQATIAKIAGNNGGAQALQT VLDLEPALCERGFGQATIAKMAGNGGGAQALQTVLDLEPALRKRDFRQADIIKIAGNGGG AQALQAVIEHGPTLRQHGFNLADIVKMAGNIGGAQALQAVLDLKPVLDEHGFSQADIVKI AGNNGGTQALHAVLDLERMLGERGFSRADIVNVAGHDGGAQALKAVLEHEATLNERGFSR ADIVKIAGNIGGAQALKAVLEHEATLDERGFSRADIVNVAGNGGGAQALKAVLEHEATLN ERGFNLTDIVEMAANGGGAQALKAVLEHGPTLRQRGLSLIDIVEIAGNIGGAQALKAVLK YGPVLMQAGRSNEEIVHVAARRGGAGRIRKMVAPLLERQ (SEQ ID NO:120, or an amino acid having at least 70%, 75%, 80%, 85%, 90%, 95%, or 99% sequence identity to SEQ ID NO:120, or fragment thereof.

In some forms, the BAT protein is a right hand side BAT protein that recognizes the mND6 DNA sequence. In some forms, the right hand side BAT protein that recognizes the mND6 DNA sequence is (BAT_mND6-R) having an amino acid sequence: STAFVDQDKQMANRLNLSPLERSKIEKQYGGATTLAFISNKQNELAQILSRADILKIASY DCAAHALQAVLDCGPMLGKRGFSQSDIVKIAGNGGGAQALQAVLDLESMLGKRGFSRDDI AKMAGNIGGAQTLQAVLDLESAFRERGFSQADIVKIAGNIGGAQALYSVLDVEPTLGKRG FSRADIVKIAGNIGGAQALHTVLDLEPALGKRGFSRIDIVKIAAHDGGAQALHAVLDLGP TLRECGFSQATIAKIAGHDGGAQALQMVLDLGPALGKRGFSQATIAKIAGNGGGAQALQT VLDLEPALCERGFGQATIAKMAGNIGGAQALQTVLDLEPALRKRDFRQADIIKIAGNIGG AQALQAVIEHGPTLRQHGFNLADIVKMAGNIGGAQALQAVLDLKPVLDEHGFSQADIVKI AGHDGGTQALHAVLDLERMLGERGFSRADIVNVAGHDGGAQALKAVLEHEATLNERGFSR ADIVKIAGNGGGAQALKAVLEHEATLDERGFSRADIVNVAGHDGGAQALKAVLEHEATLN ERGFNLTDIVEMAAHDGGAQALKAVLEHGPTLRQRGLSLIDIVEIAGNIGGAQALKAVLK YGPVLMQAGRSNEEIVHVAARRGGAGRIRKMVAPLLERQ (SEQ ID NO:121, or an amino acid having at least 70%, 75%, 80%, 85%, 90%, 95%, or 99% sequence identity to SEQ ID NO:121, or fragment thereof.

d. CRISPR-Cas Effector Proteins

In some forms, the targeted base editor includes one or more Crispr-Cas effector proteins as the one or more targeting domains. An advantage of the CRISPR-Cas system is that it does not require the generation of customized proteins to target specific sequences, but rather, a single Cas protein can be programmed by guide molecules to recognize a specific nucleic acid target. In other words the Crispr-Cas effector protein can be recruited to a specific nucleic acid target locus of interest using said guide molecule.

Preferably, the CRISPR-Cas effector protein is considered to substantially lack all DNA cleavage activity (e.g., when the DNA cleavage activity of the mutated enzyme is about no more than 25%, 10%, 5%, 1%, 0.1%, 0.01%, or less of the DNA cleavage activity of the non-mutated form of the enzyme). An example can be when the DNA cleavage activity of the mutated form is nil or negligible as compared with the non-mutated form. In such forms, the CRISPR-Cas protein is used as a generic DNA binding protein.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). Methods of preparing compositions for use in genome editing using the CRISPR/Cas systems are described in detail in WO 2013/176772 and WO 2014/018423, which are specifically incorporated by reference herein in their entireties.

As used herein, the term “Cas” generally refers to an effector protein of a CRISPR-Cas system or complex. The term “Cas” may be used interchangeably with the terms “CRISPR” protein, “CRISPR-Cas protein,” “CRISPR effector,” CRISPR-Cas effector,” “CRISPR enzyme,” “CRISPR-Cas enzyme” and the like, unless otherwise apparent. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some forms, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the ‘target sequence’ and the tracrRNA is often referred to as the ‘scaffold’.

The Crispr-Cas effector protein may be without limitation a type II, type V, or type VI Cas effector protein.

Non-limiting examples of Crispr-Cas effector proteins include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologues thereof, or modified versions thereof. In some forms, the unmodified CRISPR enzyme has DNA cleavage activity. Preferably, the Crispr-Cas effector protein is mutated with respect to a corresponding wild-type enzyme such that the mutated CRISPR enzyme lacks the ability to cleave one or both strands of a target polynucleotide containing a target sequence.

### (1) Cas9

In some forms, the Type II CRISPR enzyme is a Cas9 enzyme such as disclosed in International Patent Application Publication No. WO/2014/093595. In some forms, the Cas9 enzyme is S. pneumoniae, S. pyogenes or S. thermophilus Cas9, and may include mutated Cas9 derived from these organisms. The enzyme may be a Cas9 homolog or ortholog. Additional orthologs include, for example, Cas9 enzymes from Corynebacter diptheriae, Eubacterium ventriosum, Streptococcus pasteurianus, Lactobacillus farciminis, Sphaeroachaeta globus, Azospirillum B510, Gluconacetobacter diazotrophicus, Neisseria cinereal, Roseburia intestinalis, Parvibaculum lavamentivorans, Staphylococcus aureus, Nitratifractor salsuginis DSM 16511, Camplyobacter lari CF89-12, and Streptococcus thermophilus LMD-9.

In some forms, the Cas9 effector protein and orthologs thereof may be modified for enhanced function. For example, improved target specificity of a CRISPR-Cas9 system may be accomplished by approaches that include, but are not limited to, designing and preparing guide RNAs having optimal activity, selecting Cas9 enzymes of a specific length, truncating the Cas9 enzyme making it smaller in length than the corresponding wild-type Cas9 enzyme by truncating the nucleic acid molecules coding therefor and generating chimeric Cas9 enzymes wherein different parts of the enzyme are swapped or exchanged between different orthologs to arrive at chimeric enzymes having tailored specificity.

A Cas9 enzyme may comprise one or more mutations and may be used as a generic DNA binding protein with or without fusion to or being operably linked to a functional domain. The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains. Preferred examples of suitable mutations are the catalytic residue(s) in the N-term RuvC I domain of Cas9 or the catalytic residue(s) in the internal HNH domain. In some forms, the Cas9 is (or is derived from) the Streptococcus pyogenes Cas9 (SpCas9). In such forms, preferred mutations are at any or all of positions 10, 762, 840, 854, 863 and/or 986 of SpCas9 or corresponding positions in other Cas9 orthologs with reference to the position numbering of SpCas9 (which may be ascertained for instance by standard sequence comparison tools, e.g. ClustalW or MegAlign by Lasergene 10 suite). In particular, any or all of the following mutations are preferred in SpCas9: D10A, E762A, H840A, N854A, N863A and/or D986A; as well as conservative substitution for any of the replacement amino acids is also envisaged. The same mutations (or conservative substitutions of these mutations) at corresponding positions with reference to the position numbering of SpCas9 in other Cas9 orthologs are also preferred. Particularly preferred are D10 and H840 in SpCas9. However, in other Cas9s, residues corresponding to SpCas9 D10 and H840 are also preferred. These are advantageous as when singly mutated they provide nickase activity and when both mutations are present the Cas9 is converted into a catalytically null mutant which is useful for generic DNA binding.

In some example forms, the Cas9 protein may comprise an inducible dimer, or comprises or consists essentially of or consists of an inducible heterodimer. In some forms, the first half or a first portion or a first fragment of the inducible heterodimer is or comprises or consists of or consists essentially of an FKBP, optionally FKBP12. In some forms, of the inducible CRISPR-Cas system, the second half or a second portion or a second fragment of the inducible heterodimer is or comprises or consists of or consists essentially of FRB. The arrangement of the first CRISPR enzyme fusion construct may comprise or consist of or consist essentially of N′ terminal Cas9 part-FRB-NES. The arrangement of the first CRISPR enzyme fusion construct may also comprise or consists of or consists essentially of NES-N′ terminal Cas9 part-FRB-NES. The arrangement of the second CRISPR enzyme fusion construct may comprise, or consists essentially of, or consists of C′ terminal Cas9 part-FKBP-NLS. The arrangement of the second CRISPR enzyme fusion construct may comprise or consists of or consists essentially of NLS-C′ terminal Cas9 part-FKBP-NLS. There may be a linker that separates the Cas9 part from the half or portion or fragment of the inducible dimer. The inducer energy source may comprise, or consists essentially of, or consists of rapamycin. The inducible dimer may be an inducible homodimer. In some forms, in inducible CRISPR-Cas system, the CRISPR enzyme is Cas9, e.g., SpCas9 or SaCas9. In some forms of inducible CRISPR-Cas system, the Cas9 is split into two parts at any one of the following split points, according or with reference to SpCas9: a split position between 202A/203S; a split position between 255F/256D; a split position between 310E/311I; a split position between 534R/535K; a split position between 572E/573C; a split position between 713S/714G; a split position between 1003L/104E; a split position between 1054G/1055E; a split position between 1114N/1115S; a split position between 1152K/1153S; a split position between 1245K/1246G; or a split between 1098 and 1099.

In some forms, chimeric Cas9 proteins are used. Chimeric Cas9 proteins are proteins that comprise fragments that originate from different Cas9 orthologs. For instance, the N-terminal of a first Cas9 ortholog may be fused with the C-terminal of a second Cas9 ortholog to generate a resultant Cas9 chimeric protein. These chimeric Cas9 proteins may have a higher specificity or a higher efficiency than the original specificity or efficiency of either of the individual Cas9 enzymes from which the chimeric protein was generated. These chimeric proteins may also comprise one or more mutations or may be linked to one or more functional domains.

Also suitable are Cas9 proteins that have different PAM specificities. Typically, Cas9 proteins, such as Cas9 from S. pyogenes (spCas9), require a canonical NGG PAM sequence to bind a particular nucleic acid region. In some forms, the base editor may need to be placed at a precise location, for example where a target base is placed within a 4 base region (e.g., a “deamination window”), which is approximately 15 bases upstream of the PAM. See Komor, A. C., et al., Nature 533, 420-424 (2016), the entire contents of which are hereby incorporated by reference. Accordingly, in some forms, the base editor may contain a Cas9 protein that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B P., et al., Nature 523, 481-485 (2015); and Kleinstiver, B P., et al., Nature Biotechnology 33, 1293-1298 (2015); the entire contents of each are hereby incorporated by reference.

In preferred forms, the CRISPR enzyme is a deadCas (dCas), which is a CRISPR enzyme having a diminished nuclease activity. For example, the nuclease activity can be diminished by at least 97% or 100% (i.e., no more than 3% and advantageously 0% nuclease activity) as compared with the CRISPR enzyme not having any mutations. In some forms, dCas can be a deadCas9 (dCas9). In some forms, the dCas9 can comprise at least one mutation or two or more mutations. In some forms, the at least one mutation can be at position H840 (or at the corresponding position in any corresponding ortholog). In some forms, the two or more mutations can comprise mutations at two or more of the positions D10, E762, H840, N854, N863, or D986 according to SpCas9 protein (or corresponding positions in any corresponding ortholog), at position N580 according to SaCas9 protein (or corresponding positions in any corresponding ortholog).

### (2) Cas12a (Cpf1)

In some forms, the CRISPR effector is a class 2, type V CRISPR effector. In some forms, the CRISPR effector is a class 2, type V-A; class 2, type V-B; class 2, type V-C; class 2, type V-U; class 2, type V-U1; class 2, type V-U2; class 2, type V-U3; class 2, type V-U4; or class 2, type V-U5 CRISPR effector.

In some forms, the CRISPR effector is Cas12a (Cpf1). Cas12s effector proteins include effector proteins derived from an organism from a genus including Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In some forms, the effector protein (e.g., a Cpf1) comprises an effector protein (e.g., a Cpf1) from an organism from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii.

The effector protein may comprise a chimeric effector protein including a first fragment from a first effector protein (e.g., a Cpf1) ortholog and a second fragment from a second effector (e.g., a Cpf1) protein ortholog, and wherein the first and second effector protein orthologs are different. Cpf1 effector proteins may be modified, e.g., an engineered or non-naturally-occurring effector protein or Cpf1. In some forms, the modification may comprise mutation of one or more amino acid residues of the effector protein. The one or more mutations may be in one or more catalytically active domains of the effector protein.

The effector protein may have reduced or abolished nuclease activity compared with an effector protein lacking said one or more mutations. In preferred forms, the one or more mutations may comprise two mutations. The effector protein may not direct cleavage of one or other DNA or RNA strand at the target locus of interest. In preferred forms, the Cpf1 effector protein is an FnCpf1 effector protein. In preferred forms, the one or more modified or mutated amino acid residues are D917A, E1006A or D1255A with reference to the amino acid position numbering of the FnCpf1 effector protein. In further preferred forms, the one or more mutated amino acid residues are D908A, E993A, and D1263A with reference to the amino acid positions in AsCpf1 or LbD832A, E925A, D947A, and D1180A with reference to the amino acid positions in LbCpf1.

In some forms, one or more mutations of the two or more mutations can be in a catalytically active domain of the effector protein including a RuvC domain. In some forms, the RuvC domain may comprise a RuvCI, RuvCII or RuvCIII domain, or a catalytically active domain which is homologous to a RuvCI, RuvCII or RuvCIII domain. Additional Cas12a enzymes that may be delivered used the compositions disclosed herein are discussed in International Patent Application Nos. WO/2016/205711, WO/2017/106657, and WO/2017/172682.

In some forms, a protospacer adjacent motif (PAM) or PAM-like motif directs binding of the effector protein complex to the target locus of interest. In some forms, the PAM is 5′ TTN, where N is A/C/G or T and the effector protein is FnCpf1p. In some forms, the PAM is 5′ TTTV, where V is A/C or G and the effector protein is AsCpf1, LbCpf1 or PaCpf1p. In some forms, the PAM is 5′ TTN, where N is A/C/G or T, the effector protein is FnCpf1p, and the PAM is located upstream of the 5′ end of the protospacer. In some forms, the PAM is 5′ CTA, where the effector protein is FnCpf1p, and the PAM is located upstream of the 5′ end of the protospacer or the target locus.

e. Base Excision Repair Inhibitors

In some forms, the targeted base editor further includes a base excision repair (BER) inhibitor. Base excision repair corrects small base lesions that do not significantly distort the DNA helix structure. Such damage typically results from deamination, oxidation, or methylation. BER takes place in nuclei, as well as in mitochondria, largely using different isoforms of proteins or genetically distant proteins. BER is initiated by a DNA glycosylase that recognizes and removes the damaged base, leaving an abasic site which is further processed by short-patch repair or long-patch repair. At least 11 distinct mammalian DNA glycosylases are known, each recognizing a few related lesions, frequently with some overlap in specificities.

The DNA-repair (e.g., BER) response to the presence of mismatches (e.g., I:T; U:G) caused by the deamination of a target nucleotide by a disclosed deaminase or base editor, may lead to a decrease in efficiency of a completing a desired base edit in cells. Thus, inhibitors of BER can inhibit or reduce undesirable BER activity that can revert the DNA to its original state.

For example, deamination of adenine results in the formation of hypoxanthine (herein represented as “I” for inosine, the nucleoside formed from hypoxanthine). A BER response to the presence of I:T pairing may be responsible for a decrease in base editing efficiency in cells. Alkyladenine DNA glycosylase (also known as DNA-3-methyladenine glycosylase, 3-alkyladenine DNA glycosylase, or N-methylpurine DNA glycosylase) catalyzes removal of hypoxanthine from DNA in cells, which may initiate base excision repair, resulting in reversion of the I:T pair to a A:T pair.

Thus in some forms, the BER inhibitor is an inhibitor of alkyladenine DNA glycosylase (e.g., human alkyladenine DNA glycosylase). In some forms, the BER inhibitor is a polypeptide inhibitor. In some forms, the BER inhibitor is a protein that binds hypoxanthine (e.g., in DNA). In some forms, the BER inhibitor is a catalytically inactive alkyladenine DNA glycosylase protein or binding domain thereof. In some forms, the BER inhibitor is a catalytically inactive alkyladenine DNA glycosylase protein or binding domain thereof that does not excise hypoxanthine from the DNA. Other proteins that are capable of inhibiting (e.g., sterically blocking) an alkyladenine DNA glycosylase base-excision repair enzyme are also suitable. Additionally, any proteins that block or inhibit base-excision repair are also useful.

Deamination of cytosine results in the formation of uracil (“U”). A BER response to the presence of U:G pairing may be responsible for a decrease in base editing efficiency in cells. At least four different human DNA glycosylases may remove uracil and thus initiate base excision repair, resulting in reversion of the U:G pair to a C:G pair. These enzymes, referred to as uracil-DNA glycosylases (UDGs), include UNG, SMUG1, TDG and MBD4.

Thus in some forms, the BER inhibitor is a uracil glycosylase inhibitor (“UGI”). Preferably, the UGI is a peptide or protein that is capable of inhibiting a uracil-DNA glycosylase base-excision repair enzyme, such as those listed above. The term “uracil glycosylase inhibitor” or “UGI,” as used herein, refers to a protein that is capable of inhibiting a uracil-DNA glycosylase base-excision repair enzyme. In some forms, a UGI domain includes a wild-type UGI or a UGI as set forth in SEQ ID NO:21. In some forms, the UGI proteins provided herein include fragments of UGI and proteins homologous to a UGI or a UGI fragment. For example, in some forms, a UGI domain includes a fragment of the amino acid sequence set forth in SEQ ID NO: 21. In some forms, the UGI comprises the following amino acid sequence or a fragment thereof: MTNLSDIIEKETGKQLVIQESILMLPEEVEEVIGNKPESDILVHTAYDESTDENVMLLTS DAPEYKPWALVIQDSNGENKIKML (SEQ ID NO:21). In some forms, a UGI comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to the amino acid sequence as set forth in SEQ ID NO:21. In some forms, a UGI is a protein that binds single-stranded DNA (e.g., a Erwinia tasmaniensis single-stranded binding protein). In some forms, a UGI inhibitor is a protein that binds uracil (e.g., uracil in DNA). In some forms, a uracil glycosylase inhibitor is a catalytically inactive uracil DNA-glycosylase (e.g., a UDG that does not excise uracil from the DNA). Other suitable UGI are known in the art and include, for example, those described in Wang et al., J. Biol. Chem. 264:1163-1171 (1989); Lundquist et al., J. Biol. Chem. 272:21408-21419 (1997); Ravishankar et al., Nucleic Acids Res. 26:4880-4887 (1998); Putnam et al., J. Mol. Biol. 287:331-346 (1999), and U.S. 2019/0093099, the entire contents of each are incorporated herein by reference. Therefore, in some forms, the base editor includes a canonical UGI amino acid sequence that is:

(SEQ ID NO: 70)
INLSDIIEKETGKQLVIQESILMLPEEVEEVIGNKPESDILVHTAYDES
TDENVMLLTSDAPEYKPWALVIQDSNGENKIKML.

Without wishing to be bound by any particular theory, base excision repair may be inhibited by molecules that bind the edited strand, block the edited base, inhibit alkyladenine DNA glycosylase, inhibit uracil DNA glycosylase(s), inhibit base excision repair, protect the edited base, and/or promote fixing of the non-edited strand. It is believed that the use of the BER inhibitor can increase the editing efficiency of an deaminase or base editor thereof that is capable of effecting an A to G base edit or a C to T base edit.

In some forms, a base editor additionally including a BER inhibitor conforms to the following architecture/structure:
NH₂[deaminase domain]-[functional domain]-[BER inhibitor]COOH;
NH₂[deaminase domain]-[BER inhibitor]-[functional domain]COOH;
NH₂[BER inhibitor]-[deaminase domain]-[functional domain]COOH;
NH₂[BER inhibitor]-[functional domain]-[deaminase domain]COOH
NH₂[functional domain]-[deaminase domain]-[BER inhibitor]COOH
NH₂[functional domain]-[BER inhibitor]-[deaminase domain]COOH
   wherein NH₂ is the N-terminus of the base editor, COOH is the C-terminus of the base editor, and “-” indicates the presence of an optional linker. Preferably, the functional domain is a targeting domain, for example a DNA binding protein or domain, such as a zinc finger, TAL effector, or Crispr-Cas effector.

### 4. Linkers

A linker may be used to fuse or join any of the domains described herein. Generally, such linkers have no specific biological activity other than to join or to preserve some minimum distance or other spatial relationship between the domains. However, in certain forms, the linker may be selected to influence some property of the linker and/or the linked components such as the folding, flexibility, net charge, or hydrophobicity of the linker. In particular forms, a base editor contains one or more linkers to separate the deaminase domain and functional (e.g., targeting) domain by a distance sufficient to ensure that each domain retains its required functional property.

Typically, the linker is positioned between, or flanked by, two groups, molecules, or other moieties and connected to each one via a covalent bond, thus connecting the two. The linker may be as simple as a covalent bond, or it may be a polymeric linker many atoms in length. The linker can be an amino acid or a plurality of amino acids (e.g., a peptide or protein). In preferred forms, the linker contains amino acids. In some forms, the linker is preferably a peptide. Preferred peptide linker sequences adopt a flexible extended conformation and do not exhibit a propensity for developing an ordered secondary structure. Preferably, the linker comprises amino acids. Typical amino acids in flexible linkers include Gly (G), Asn (N) and Ser (S). Accordingly, in particular forms, the linker contains a combination of one or more of Gly (G), Asn (N) and Ser (S) amino acids. Other near neutral amino acids, such as Thr (T) and Ala (A), also may be used in the linker sequence.

In some forms, the linker can be 2-200 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also suitable. GlySer linkers such as GS, GGS, GGGS (SEQ ID NO:23) or GSG can be used in repeats of 3, 4, 5, 6, 7, 9, 12 or more, to provide suitable lengths. Suitable linkers include, without limitation, (GGGS)n (SEQ ID NO:23), (SGGS)n (SEQ ID NO:24), (GGGGS)n (SEQ ID NO:25), (EAAAK)n (SEQ ID NO:26), (G)n, (GGS)n, SGSETPGTSESATPES (SEQ ID NO:27; referred to as the XTEN linker), and (XP)n, or a combination of any of these, wherein n is independently an integer between 1 and 30, and wherein X is any amino acid. In some forms, n is 1, 2, 3, 4, 5, 6, 7, 8, 9, 10, 11, 12, 13, 14, or 15. In some forms, N- and C-terminal NLSs can also function as linkers (e.g., PKKKRKVEASSPKKRKVEAS; SEQ ID NO:30).

In other forms, the linker is not peptide-like. The linker can be an organic molecule, group, polymer, or chemical moiety. In certain forms, the linker is a covalent bond (e.g., a carbon-carbon bond, disulfide bond, carbon-heteroatom bond, etc.). In some forms, the linker is a carbon-nitrogen bond of an amide linkage. In some forms, the linker is a cyclic or acyclic, substituted or unsubstituted, branched or unbranched aliphatic or heteroaliphatic linker. In some forms, the linker is polymeric (e.g., polyethylene, polyethylene glycol, polyamide, polyester, etc.). In some forms, the linker includes a monomer, dimer, or polymer of aminoalkanoic acid. In some forms, the linker includes an aminoalkanoic acid (e.g., glycine, ethanoic acid, alanine, beta-alanine, 3-aminopropanoic acid, 4-aminobutanoic acid, 5-pentanoic acid, etc.). In some forms, the linker includes a monomer, dimer, or polymer of aminohexanoic acid (Ahx). In some forms, the linker is based on a carbocyclic moiety (e.g., cyclopentane, cyclohexane), a polyethylene glycol moiety (PEG), or an aryl or heteroaryl moiety. In some forms, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Exemplary linkers are also disclosed in Maratea et al. (1985), Gene 40: 39-46; Murphy et al. (1986) Proc. Natl. Acad. Sci. USA 83: 8258-62; U.S. Pat. Nos. 4,935,233; and 4,751,180.

i. Coiled-Coil Linkers

In some forms, a deaminase, split deaminase domain, base editor, targeting domain, or other disclosed domain, protein or polypeptide can be fused to or operably linked to linkers which include but are not limited to a protein having a coiled-coil configuration.

In some forms, the coiled-coil linker, has a sequence that pairs with another coiled-coil linker. For example, in some forms two or more different coiled-coil linkers co-localize to provide a more rigid conformation that can restrict and guide the position of a base editor on a target DNA strand. For example, in some forms, a base editor includes a split deaminase protein domain bound to a first coiled-coil linker and a second split deaminase domain bound to a second coiled coil linker. The co-localization of the coiled-coil domains provides a more rigid linker to guide the position of the co-localized deaminase domains on a target DNA strand. In some forms, a first coiled coil linker includes the amino acid sequence: GGGSGGSGEIAALEAKNAALKAEIAALEAKIAALKAGY (SEQ ID NO:184). In other forms, the coiled coil includes an amino acid sequence that is about 50%, 55%, 60%, 65%, 70%, 75%, 80%, 85%, 90% or 95% identity to SEQ ID NO: 184.

In some forms, a second coiled coil linker includes the amino acid sequence: GGSGGSYKIAALKAENAALEAKIAALKAEIAALEAGC (SEQ ID NO:185). In other forms, the coiled coil includes an amino acid sequence that is about 50%, 55%, 60%, 65%, 70%, 75%, 80%, 85%, 90% or 95% identity to SEQ ID NO: 185.

Typically, the first coiled coil linker pairs with the second coiled coil linker upon co-localization.

### 5. Other Domains and Modifications

The deaminase, base editor, targeting domain, or other disclosed domain, protein or polypeptide may be modified in various ways. In some forms, the modification(s) may render the protein or peptides more stable (e.g., resistant to degradation in vivo) or more capable of penetrating into cells or subcellular compartments, or other desirable characteristic as will be appreciated by one skilled in the art. Such modifications include, without limitation, chemical modification, N terminus modification, C terminus modification, peptide bond modification, backbone modifications, residue modification, D-amino acids, or non-natural amino acids or others. In some forms, one or more modifications may be used simultaneously. In preferred forms, the deaminases, base editors, targeting domains, or other disclosed domains, proteins or polypeptides are stabilized against proteolysis. For example, the stability and activity of peptides can be improved by protecting some of the peptide bonds with N-methylation or C-methylation. It is believed that modifications, such as amidation, also enhance the stability of peptides to peptidases.

The modifications may or may not cause an altered functionality. By means of example, and in particular with reference to deaminase or base editor, modifications which do not result in an altered functionality include for instance codon optimization for expression into a particular host, or providing the deaminase or base editor with a particular marker or epitope tag (e.g., for visualization and/or isolation or purification).

In some forms, a deaminase, base editor, targeting domain, or other disclosed domain, protein or polypeptide can be fused to or operably linked to domains which include but are not limited to a transcriptional activator, transcriptional repressor, a recombinase, a transposase, a histone remodeler, a DNA methyltransferase, a cryptochrome, a light inducible/controllable domain, or a chemically inducible/controllable domain.

i. Nuclear Localization Sequences

In some forms, the deaminase, base editor, targeting domain, or other disclosed domain, protein or polypeptide can include or be associated with one or more (e.g., two or more, three or more, or four or more) nuclear localization sequences (NLSs). Any convenient NLS can be used. Examples include Class 1 and Class 2 “monopartite NLSs,” as well as NLSs of Classes 3-5 (Kosugi et al., J Biol Chem. 284(1):478-485 (2009)). In some cases, an NLS has the formula: (K/R)(K/R)X₁₀₋₁₂(K/R)₃₋₅. In some cases, an NLS has the formula: K(K/R)X(K/R) (SEQ ID NO:31). The NLS(s) can be placed at the N- or C-termini of the deaminase, base editor, targeting domain, or other disclosed domain, protein or polypeptide. In some instances, it is advantageous to position the NLS at the N-terminus.

Examples of NLSs that can be used include: T-ag NLS (PKKKRKV; SEQ ID NO:32), T-Ag-derived NLS (PKKKRKVEDPYC-SV40; SEQ ID NO:33), NLS SV40 (PKKKRKVGPKKKRKVGPKKKRKVGPKKKRKVGC; SEQ ID NO:34), CYGRKKRRQRRR-N-terminal cysteine of cysteine-TAT (SEQ ID NO:35), CSIPPEVKFNKPFVYLI (SEQ ID NO:36), DRQIKIWFQNRRMKVVKK (SEQ ID NO:37), PKKKRKVEDPYC-C-term cysteine of an SV40 T-Ag-derived NLS (SEQ ID NO:38), and cMyc NLS (PAAKRVKLD; SEQ ID NO:39). Other useful NLSs are described in Kosugi et al., J Biol Chem. 284(1):478-485 (2009).

ii. Mitochondrial Localization Sequences

The deaminase, base editor, targeting domain, or other disclosed domain, protein or polypeptide, can include or be associated with one or more (e.g., two or more, three or more, or four or more) mitochondrial targeting sequences (MTSs), or mitochondrial targeting sequences (MTS). Any convenient mitochondrial localization sequence can be used. Examples of mitochondrial localization sequences include: PEDEIWLPEPESVDVPAKPISTSSMMM (SEQ ID NO:22), a mitochondrial localization sequence of SDHB, mono/di/triphenylphosphonium or other phosphoniums, VAMP 1A, VAMP 1B, the 67 N-terminal amino acids of DGAT2, and the 20 N-terminal amino acids of Bax. The MTS(s) can be placed at the N- or C-termini of the deaminase, base editor, targeting domain, or other disclosed domain, protein or polypeptide.

a. MTS Derived from Cox8

In some forms, the mitochondrial targeting sequences (MTS) is derived from Cox8. In some forms, the mitochondrial localization sequence derived from Cox8, a mitochondrial cytochrome c oxidase subunit VIII. In some forms, a mitochondrial localization sequence derived from COX8 includes the amino acid sequence: MSVLTPLLLRGLTGSARRLPVPRAKIHSL (SEQ ID NO: 69). In other forms, the mitochondrial localization sequence derived from COX8 includes an amino acid sequence that is about 50%, 55%, 60%, 65%, 70%, 75%, 80%, 85%, 90% or 95% identity to SEQ ID NO: 69.

In other forms, a mitochondrial localization sequence derived from Cox8 includes the amino acid sequence: SVLTPLLLRSLTGSARRLMVPRAQVHSK (SEQ ID NO: 183). In other forms, the mitochondrial localization sequence derived from Cox8 includes an amino acid sequence that is about 50%, 55%, 60%, 65%, 70%, 75%, 80%, 85%, 90% or 95% identity to SEQ ID NO:183.

b. MTS Derived from SOD2

In some forms, the mitochondrial targeting sequences (MTS) is derived from SOD2. In some forms, a mitochondrial localization sequence derived from SOD2 includes the amino acid sequence: MLSRAVCGTSRQLAPVLGYLGSRQKHSLPD (SEQ ID NO: 71). In other forms, the mitochondrial localization sequence derived from SOD2 includes an amino acid sequence that is about 50%, 55%, 60%, 65%, 70%, 75%, 80%, 85%, 90% or 95% identity to SEQ ID NO: 71. In other forms, a mitochondrial localization sequence derived from SOD2 includes the amino acid sequence: LCRAACSTGRRLGPVAGAAGSRHKHSLPD (SEQ ID NO: 182). In other forms, the mitochondrial localization sequence derived from SOD2 includes an amino acid sequence that is about 50%, 55%, 60%, 65%, 70%, 75%, 80%, 85%, 90% or 95% identity to SEQ ID NO:182.

c. I-Tev I Nuclease

In some forms, the base editors include one or more nucleases, such as the small, sequence-tolerant monomeric nuclease domain from the homing endonuclease I-Tev (I-TevI enzyme; Kleinstiver, et al., G3 GenesIGenomeslGenetics, Volume 4, Issue 6, 1 Jun. 2014, Pages 1155-1165, https://doi.org/10.1534/g3.114.011445). The additional specificity of the I-TevI nuclease domain has the potential to reduce cleavage at off-target sites, because the required cleavage motif may not be found within the vicinity of sites that result from promiscuous DNA binding. In some forms, I-Tev I nuclease can be used as a nickase to misguide the mitochondrial repair system and direct the repair toward desired outcome (i.e., edited target)

In some forms, the targeted base editor includes one or more I-TEVI domains. In some forms the I-TEVI domain has an amino acid sequence of: KSGIYQIKNTLNNKVYVGSAKDFEKRWKRHFKDLEKGCHSSIKLQRSFNKHGNVFECSILEEIPYEKDLIIE RENFWIKELNSKINGYNIADATFGDTCSTHPLKEEIIKKRSETVKAKMLKLGPDGRKALYSKPGSKNGRWNP ETHKFCKCGVRIQTSAYTCSKCRNRSGENNSFFNHKHS (SEQ ID NO:186), or an amino acid having at least 70%, 75%, 80%, 85%, 90%, 95%, or 99% sequence identity to SEQ ID NO:186, or fragment thereof.

### d. 2A Self-Cleaving Peptides

In some forms, the targeted base editor further includes a 2A peptide motif. 2A self-cleaving peptides, or 2A peptides, is a class of 18-22 aa-long peptides, which can induce ribosomal skipping during translation of a protein in a cell. These peptides share a core sequence motif of DxExNPGP, and are found in a wide range of viral families. They help generating polyproteins by causing the ribosome to fail at making a peptide bond.

The members of 2A peptides are named after the virus in which they have been first described. For example, F2A, the first described 2A peptide, is derived from foot-and-mouth disease virus. The name “2A” itself comes from the gene numbering scheme of this virus. Exemplary 2A peptides for use in the base editors include P2A, E2A, F2A, and T2A. In some forms, the 2A peptide has an amino acid sequence ATNFSLLKQAGDVEENPGP (SEQ ID NO: 187), or an amino acid having at least 70%, 75%, 80%, 85%, 90%, 95%, or 99% sequence identity to SEQ ID NO:187, or fragment thereof.

e. IRES

In some forms, the targeted base editor further includes an IRES motif. An internal ribosome entry site, abbreviated IRES, is an RNA element that allows for translation initiation in a cap-independent manner, as part of the greater process of protein synthesis. In eukaryotic translation, initiation typically occurs at the 5′ end of mRNA molecules, since 5′ cap recognition is required for the assembly of the initiation complex. The location for IRES elements is often in the 5′UTR, but can also occur elsewhere in mRNAs. The IRES can be used to express polycistronic proteins with defined stop codons in intended eukaryotic cells, while avoiding toxicity observed when in case of P2A peptide when cloning the dsDNA specific deaminases in E. coli. The IRES design is used to make a single-AAV base editors (using ZFs as DNA binding domains) where all the required components are packaged into a single AAV vector which is then used to successfully edit mitochondrial genomes in human cell lines.

In some forms, when the split deaminase domains or base editors are to be delivered via a vector, such as a viral vector, the base editors include one or more IRES domains. In some forms the IRES domain has a nucleic acid sequence: GAGGGCCCGGAAACCTGGCCCTGTCTTCTTGACGAGCATTCCTAGGGGTCTTTCCCCTCT CGCCAAAGGAATGCAAGGTCTGTTGAATGTCGTGAAGGAAGCAGTTCCTCTGGAAGCTTC TTGAAGACAAACAACGTCTGTAGCGACCCTTTGCAGGCAGCGGAACCCCCCACCTGGCGA CAGGTGCCTCTGCGGCCAAAAGCCACGTGTATAAGATACACCTGCAAAGGCGGCACAACC CCAGTGCCACGTTGTGAGTTGGATAGTTGTGGAAAGAGTCAAATGGCTCACCTCAAGCGT ATTCAACAAGGGGCTGAAGGATGCCCAGAAGGTACCCCATTGTATGGGATCTGATCTGGG GCCTCGGTGCACATGCTTTACATGTGTTTAGTCGAGGTTAAAAAACGTCTAGGCCCCCCG AACCACGGGGACGTGGTTTTCCTTTGAAAAACACGATGATAA (SEQ ID NO:188), or a nucleic acid having at least 70%, 75%, 80%, 85%, 90%, 95%, or 99% sequence identity to SEQ ID NO:188, or fragment thereof.

f. CBh Promoter

In some forms, the targeted base editor further includes a Promoter for recombinant adeno-associated virus-mediated gene expression. In some forms, the promoter sequence is a CBh promoter.

In some forms, the CBh promoter has a nucleic acid sequence: CGTTACATAACTTACGGTAAATGGCCCGCCTGGCTGACCGCCCAACGACCCCCGC CCATTGACGTCAATAATGACGTATGTTCCCATAGTAACGCCAATAGGGACTTTCCATTGA CGTCAATGGGTGGAGTATTTACGGTAAACTGCCCACTTGGCAGTACATCAAGTGTATCAT ATGCCAAGTACGCCCCCTATTGACGTCAATGACGGTAAATGGCCCGCCTGGCATTATGCC CAGTACATGACCTTATGGGACTTTCCTACTTGGCAGTACATCTACGTATTAGTCATCGCT ATTACCATGGTCGAGGTGAGCCCCACGTTCTGCTTCACTCTCCCCATCTCCCCCCCCTCC CCACCCCCAATTTTGTATTTATTTATTTTTTAATTATTTTGTGCAGCGATGGGGGCGGGG GGGGGGGGGGGGCGCGCGCCAGGCGGGGCGGGGCGGGGCGAGGGGCGGGGCGGGGCGAGG CGGAGAGGTGCGGCGGCAGCCAATCAGAGCGGCGCGCTCCGAAAGTTTCCTTTTATGGCG AGGCGGCGGCGGCGGCGGCCCTATAAAAAGCGAAGCGCGCGGCGGGCGGGAGTCGCTGCG CGCTGCCTTCGCCCCGTGCCCCGCTCCGCCGCCGCCTCGCGCCGCCCGCCCCGGCTCTGA CTGACCGCGTTACTCCCACAGGTGAGCGGGCGGGACGGCCCTTCTCCTCCGGGCTGTAAT TAGCTGAGCAAGAGGTAAGGGTTTAAGGGATGGTTGGTTGGTGGGGTATTAATGTTTAAT TACCTGGAGCACCTGCCTGAAATCACTTTTTTTCAGGTTGG (SEQ ID NO:189), or a nucleic acid having at least 70%, 75%, 80%, 85%, 90%, 95%, or 99% sequence identity to SEO ID NO:189, or fragment thereof.

g. Polyadenylation Motif

In some forms, the targeted base editor further includes a poly adenylation motif for recombinant adeno-associated virus-mediated gene expression. Exemplary poly adenylation motifs include those from SV40, hGH, BGH, and rbGlob. In some forms, the poly adenylation motif is from BGH, having a nucleic acid sequence: CTGTGCCTTCTAGTTGCCAGCCATCTGTTGTTTGCCCCTCCCCCGTGCCTTCCTTGACCC TGGAAGGTGCCACTCCCACTGTCCTTTCCTAATAAAATGAGGAAATTGCATCGCATTGTC TGAGTAGGTGTCATTCTATTCTGGGGGGTGGGGTGGGGCAGGACAGCAAGGGGGAGGATT GGGAAGACAATAGCAGGCATGCTGGGGATGCGGTGGGCTCTATGG (SEQ ID NO:190), or a nucleic acid having at least 70%, 75%, 80%, 85%, 90%, 95%, or 99% sequence identity to SEQ ID NO:190, or fragment thereof.

### 6. Exemplary Base Editor Configurations

In some forms, the targeted base editor includes a first and second portion, wherein the first portion includes
(a) a first split deaminase domain including an amino acid sequence of SEQ ID NO:120, and
(b) a Left hand TALE programmable DNA binding domain; and wherein the second portion comprises
(c) a second split deaminase domain including an amino acid sequence of any one of SEQ ID NOs:156, 158, 160 or 164, and
(d) a Right hand TALE programmable DNA binding domain.

In some forms, the targeted base editor includes a first and second portion, wherein the first portion includes
(a) a first split deaminase domain including an amino acid sequence of SEQ ID NO:169, and
(b) a Left hand TALE programmable DNA binding domain; and wherein the second portion comprises
(c) a second split deaminase domain including an amino acid sequence of any one of SEQ ID NOs:173, or 175, and
(d) a Right hand TALE programmable DNA binding domain.

In some forms, the targeted base editor includes a first and second portion, wherein the first portion includes
(a) a first split deaminase domain including an amino acid sequence of SEQ ID NO:171, and
(b) a Left hand TALE programmable DNA binding domain; and wherein the second portion comprises
(c) a second split deaminase domain including an amino acid sequence of any one of SEQ ID NO:175, and
(d) a Right hand TALE programmable DNA binding domain.

In some forms, the targeted base editor includes a first and second portion, wherein the first portion includes
(a) a first split deaminase domain including an amino acid sequence of SEQ ID NO:169, and
(b) a Left hand BAT programmable DNA binding domain; and wherein the second portion comprises
(c) a second split deaminase domain including an amino acid sequence of any one of SEQ ID NOs:173, or 175, and
(d) a Right hand TALE programmable DNA binding domain.

In some forms, the targeted base editor includes a first and second portion, wherein the first portion includes
(a) a first split deaminase domain including an amino acid sequence of SEQ ID NO:169, and
(b) a first coiled coil domain, and
(c) optionally a Left hand TALE programmable DNA binding domain; and wherein the second portion comprises
(d) a second split deaminase domain including an amino acid sequence of any one of SEQ ID NOs:173, or 175, and
(e) a second coiled coil domain, and
(f) optionally a Right hand TALE programmable DNA binding domain;
wherein the first and second coiled coil domains interact together upon combination of the first and second portions.

Vectors including or expressing the targeted base editors are also described.

In some forms, the vector is an altered adenovirus (AAV) vector, or a Lentivirus vector. Typically, the targeted base editor is encapsulated within the vector.

### 7. Exemplary Base Editor Sequences

In an exemplary form, the base editor is based on the BE_R1_12 deaminase domain, including a first and second portions. In an exemplary form, the base editor includes a first portion having a dead or inactive split BE_R1_12 deaminase domain, and a second portion having a truncated split BE_R1_12 deaminase domain.

In an exemplary form, the base editor includes a first portion, configured as follows:

pCBh-Kozak Start codon-mCox8 MTS-linker-TALE_R_mCox1-linker-dBE_R1_12-linker-UGI-bGH Poly A.

In an exemplary form, the first portion of the BE_R1_12 base editor has the nucleic acid sequence:

(SEQ ID NO: 264)
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATGGTCGAGGTGAGCCCCACGTTCTGCTTCACTCTCCCCATC
TCCCCCCCCTCCCCACCCCCAATTTTGTATTTATTTATTTTTTAATTAT
TTTGTGCAGCGATGGGGGCGGGGGGGGGGGGGGGGCGCGCGCCAGGCGG
GGCGGGGCGGGGCGAGGGGCGGGGCGGGGCGAGGCGGAGAGGTGCGGCG
GCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGT
CGCTGCGCGCTGCCTTCGCCCCGTGCCCCGCTCCGCCGCCGCCTCGCGC
CGCCCGCCCCGGCTCTGACTGACCGCGTTACTCCCACAGGTGAGCGGGC
GGGACGGCCCTTCTCCTCCGGGCTGTAATTAGCTGAGCAAGAGGTAAGG
GTTTAAGGGATGGTTGGTTGGTGGGGTATTAATGTTTAATTACCTGGAG
CACCTGCCTGAAATCACTTTTTTTCAGGTTGGAGCAGAGCTGGTTTAGT
GGATATCTTAAGCCACCATGGCCTCTGTCCTGACGCCACTGCTGCTGAG
GAGCCTGACCGGCTCGGCCCGGCGGCTCATGGTGCCGCGGGCTCAGGTC
CACTCGAAGTCTAGAGATATCGCCGACCTCAGAACCCTGGGTTACAGTC
AGCAGCAACAGGAGAAGATAAAACCTAAGGTGCGCTCCACTGTTGCTCA
ACATCATGAGGCATTGGTGGGCCACGGATTTACACACGCCCATATAGTA
GCCTTGTCCCAACACCCCGCTGCTCTTGGTACTGTTGCTGTAAAATATC
AAGACATGATAGCAGCATTGCCTGAAGCCACTCACGAGGCTATCGTTGG
AGTAGGAAAGTATCATGGGGCTCGCGCACTTGAGGCTTTGCTCACCGTT
GCAGGTGAACTTCGAGGCCCACCTCTTCAGCTCGACACCGGACAATTGC
TCAAGATTGCCAAGCGAGGGGGGGTCACCGCCGTAGAAGCCGTCCATGC
TTGGCGCAACGCACTCACTGGGGCCCCCCTGAACTTAACGCCCGAGCAG
GTGGTTGCTATAGCGTCGCACGATGGCGGTAAGCAAGCCCTTGAAACAG
TTCAGGCCTTGTTACCTGTCTTATGCCAGGCACATGGACTGACTCCTGA
ACAGGTAGTTGCGATTGCCTCACATGACGGAGGTAAACAAGCTTTAGAA
ACAGTGCAGGCTTTGCTCCCGGTTCTTTGTCAGGCGCATGGCTTGACTC
CGGAACAGGTTGTCGCTATTGCTTCACACGATGGGGGTAAACAAGCCCT
CGAAACAGTGCAAGCCCTTTTACCGGTCCTATGCCACGCACACGGTTTG
ACACCAGAACAGGTAGTAGCTATAGCCTCGAATATTGGTGGTAAGCAAG
CCTTAGAGACCGTGCAGCGGTTACTGCCTGTACTGTGTCAAGCTCACGG
GCTTACACCTGAGCAAGTAGTTGCAATAGCAAGTCACGACGGCGGTAAA
CAAGCCTTGGAGACCGTTCAAGCTCTCCTTCCAGTATTGTGTCAAGCAC
ATGGCCTAACTCCCGAGCAGGTAGTGGCTATCGCTAGTAACGGTGGTGG
GAAACAGGCACTAGAGACAGTTCAAGCTCTACTTCCAGTGTTGTGCCAG
GCTCACGGGCTCACACCCCAACAAGTTGTCGCCATCGCCAGTAATGGAG
GTGGAAAGCAGGCCCTCGAAACCGTGCAACGGCTCCTTCCAGTGCTCTG
CCAAGCGCATGGACTTACGCCAGAGCAGGTGGTGGCAATAGCCTCGCAT
GACGGCGGCAAGCAGGCGTTGGAGACCGTCCAAGCATTGCTGCCAGTTT
TATGTCAGGCACATGGTTTAACACCACAACAGGTAGTCGCAATAGCTAG
CAACAATGGCGGAAAACAGGCTCTGGAAACTGTCCAACGATTGCTACCC
GTTCTGTGTCAGGCCCATGGATTGACGCCGCAACAAGTGGTCGCGATTG
CGAGTCACGACGGAGGTAAACAGGCCCTGGAAACGGTGCAGAGACTACT
CCCCGTCCTCTGCCAAGCCCACGGTCTCACGCCTGAGCAGGTAGTAGCG
ATAGCATCTCACGACGGTGGTAAGCAAGCGTTAGAGACAGTACAAGCGT
TACTACCAGTTCTCTGTCAAGCTCATGGGCTAACGCCGGAACAGGTTGT
CGCTATTGCAAGCAACATCGGCGGGAAACAGGCATTAGAGACGGTCCAA
GCGCTGTTGCCCGTACTGTGTCAGGCGCATGGTCTGACACCGGAGCAAG
TTGTGGCCATCGCGTCCAACGGTGGTGGTAAACAGGCATTGGAAACCGT
ACAGGCGCTTTTGCCTGTGCTTTGTCAAGCGCACGGACTTACTCCGGAA
CAGGTAGTGGCGATCGCAAGCCATGATGGAGGAAAACAAGCACTTGAGA
CTGTTCAAAGATTATTGCCAGTGCTATGTCAAGCACACGGTCTTACCCC
AGAACAGGTCGTAGCCATAGCTTCTAATATTGGAGGCAAACAAGCCTTA
GAAACAGTCCAAGCTTTATTACCCGTGTTATGTCAGGCTCACGGCCTCA
CTCCCGAACAAGTCGTTGCCATTGCATCGAACGGCGGTGGAAAGCAAGC
TCTGGAGACGGTACAACGTTTGCTTCCGGTACTTTGCCAGGCACACGGA
TTAACGCCCGAGCAGGTGGTTGCTATAGCGTCGAACATTGGCGGTAAGC
AAGCCCTTGAAACAGTTCAGGCCTTGTTACCTGTCTTATGCCAGGCACA
TGGACTGACGCCTCAGCAAGTAGTGGCTATTGCTTCCAACGGCGGCGGA
CGCCCAGCACTCGAGAGTATCGTAGCACAGCTCAGTCGCCCAGATCCCG
CCTTGGCTGCCCTCACCAATGATCACCTTGTGGCACTCGCTTGCCTTGG
GGGTCGCCCTGCTCTGGATGCAGTTAAGAAAGGCCTAGGCGGCAGCTTC
AGCAAAGCGGAATCTGGGTATATTGAGATACAACGCTTCAGGAGAATTC
TCAACATGCCCCGCTATTCACTTACGAATGGCCGTACTGGTACGGTGGC
GCGTGTGGAGGTAAACGGGCGTCGCATTTTCGGGGTTAATACTTCGTTG
ATTAAGAACTCTAAGTATGCTCCGCGCGACATGGACTTACGCCGCCGTT
GGCTGCGCGAGGTTAACTGGGTGCCCCCAAAAAAAAACAAACCAAACCA
CTTAGGACACGCGCAGAGCCTGTCGCACGCCGCATCCCACGCTTTGATC
CGCGCATACGAACGTATGGAGCGTCTTGGGGGTCAGTTACCAAAGAAAC
TTACTATGGTAGTCGATCGCCCCACCTGCAATATCTGTCGCGGGGAGAT
GCCCGCGCTACTAAAGCGCCTGGGGATTGAAGAACTTACCATCTATTCA
GGTGGCCGCGATGCAATCATCATTAAGGCGATTAAGTCCGGAGGGTCGA
CTAATCTGAGCGACATTATAGAAAAAGAAACAGGTAAGCAGTTGGTCAT
CCAAGAGAGTATTTTGATGCTGCCAGAGGAAGTCGAGGAGGTAATTGGT
AACAAACCAGAGAGTGACATTCTTGTGCATACCGCTTATGACGAGTCAA
CTGACGAGAATGTTATGCTCTTGACCTCTGATGCACCCGAATACAAACC
TTGGGCACTCGTTATCCAGGACAGTAATGGAGAAAATAAAATAAAAATG
TTGTAATGAGCTCGGATCCCTGTGCCTTCTAGTTGCCAGCCATCTGTTG
TTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGG
TGTCATTCTATTCTGGGGGGTGGGGTGGGGCAGGACAGCAAGGGGGAGG
ATTGGGAAGACAATAGCAGGCATGCTGGGGATGCGGTGGGCTCTATGG.

In an exemplary form, the first portion of the BE_R1_12 base editor is a fusion

(SEQ ID NO: 265)
MASVLTPLLLRSLTGSARRLMVPRAQVHSKSRDIADLRTLGYSQQQQEK
IKPKVRSTVAQHHEALVGHGFTHAHIVALSQHPAALGTVAVKYQDMIAA
LPEATHEAIVGVGKYHGARALEALLTVAGELRGPPLQLDTGQLLKIAKR
GGVTAVEAVHAWRNALTGAPLNLTPEQVVAIASHDGGKQALETVQALLP
VLCQAHGLTPEQVVAIASHDGGKQALETVQALLPVLCQAHGLTPEQVVA
IASHDGGKQALETVQALLPVLCHAHGLTPEQVVAIASNIGGKQALETVQ
RLLPVLCQAHGLTPEQVVAIASHDGGKQALETVQALLPVLCQAHGLTPE
QVVAIASNGGGKQALETVQALLPVLCQAHGLTPQQVVAIASNGGGKQAL
ETVQRLLPVLCQAHGLTPEQVVAIASHDGGKQALETVQALLPVLCQAHG
LTPQQVVAIASNNGGKQALETVQRLLPVLCQAHGLTPQQVVAIASHDGG
KQALETVQRLLPVLCQAHGLTPEQVVAIASHDGGKQALETVQALLPVLC
QAHGLTPEQVVAIASNIGGKQALETVQALLPVLCQAHGLTPEQVVAIAS
NGGGKQALETVQALLPVLCQAHGLTPEQVVAIASHDGGKQALETVQRLL
PVLCQAHGLTPEQVVAIASNIGGKQALETVQALLPVLCQAHGLTPEQVV
AIASNGGGKQALETVQRLLPVLCQAHGLTPEQVVAIASNIGGKQALETV
QALLPVLCQAHGLTPQQVVAIASNGGGRPALESIVAQLSRPDPALAALT
NDHLVALACLGGRPALDAVKKGLGGSFSKAESGYIEIQRFRRILNMPRY
SLTNGRTGTVARVEVNGRRIFGVNTSLIKNSKYAPRDMDLRRRWLREVN
WVPPKKNKPNHLGHAQSLSHAASHALIRAYERMERLGGQLPKKLTMVVD
RPTCNICRGEMPALLKRLGIEELTIYSGGRDAIIIKAIKSGGSTNLSDI
IEKETGKQLVIQESILMLPEEVEEVIGNKPESDILVHTAYDESTDENVM
LLTSDAPEYKPWALVIQDSNGENKIKML.

In an exemplary form, the base editor includes a second portion, configured as follows:
pCBh-Kozak-Start codon-mCox8 MTS-linker-BAT_R_mCox1-linker-BE_R1_12(A60)-linker-UGI-Poly A.

In an exemplary form, the second portion of the BE_R1_12 base editor has the nucleic acid sequence:

(SEQ ID NO: 266)
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATGGTCGAGGTGAGCCCCACGTTCTGCTTCACTCTCCCCATC
TCCCCCCCCTCCCCACCCCCAATTTTGTATTTATTTATTTTTTAATTAT
TTTGTGCAGCGATGGGGGCGGGGGGGGGGGGGGGGCGCGCGCCAGGCGG
GGCGGGGCGGGGCGAGGGGCGGGGCGGGGCGAGGCGGAGAGGTGCGGCG
GCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGT
CGCTGCGCGCTGCCTTCGCCCCGTGCCCCGCTCCGCCGCCGCCTCGCGC
CGCCCGCCCCGGCTCTGACTGACCGCGTTACTCCCACAGGTGAGCGGGC
GGGACGGCCCTTCTCCTCCGGGCTGTAATTAGCTGAGCAAGAGGTAAGG
GTTTAAGGGATGGTTGGTTGGTGGGGTATTAATGTTTAATTACCTGGAG
CACCTGCCTGAAATCACTTTTTTTCAGGTTGGAGCAGAGCTGGTTTAGT
GGATATCTTAAGCCACCATGGCCTCTGTCCTGACGCCACTGCTGCTGAG
GAGCCTGACCGGCTCGGCCCGGCGGCTCATGGTGCCGCGGGCTCAGGTC
CACTCGAAGTCTAGATCCACTGCTTTCGTTGATCAGGACAAACAGATGG
CCAACCGTCTGAACCTGTCTCCGCTGGAACGCTCCAAAATCGAGAAACA
GTACGGCGGTGCCACTACCCTGGCCTTCATTTCTAACAAGCAAAATGAA
CTGGCGCAGATCCTGAGCCGCGCGGATATCCTGAAGATCGCGTCTTATG
ATTGCGCGGCACACGCGTTGCAGGCTGTTCTGGATTGCGGCCCGATGCT
GGGCAAGCGTGGCTTTTCCCAATCTGACATCGTCAAGATTGCGGGCAAT
GGTGGCGGTGCCCAGGCTCTGCAGGCAGTTCTGGATCTGGAAAGCATGC
TGGGTAAACGCGGTTTCAGCCGTGATGACATAGCGAAAATGGCAGGTAA
CGGCGGCGGTGCACAAACTCTGCAAGCCGTACTGGATCTGGAGTCCGCG
TTTAGAGAGCGTGGCTTTTCTCAAGCAGACATTGTAAAGATAGCGGGCA
ACAATGGGGGTGCTCAAGCACTATATAGCGTCCTGGACGTAGAGCCGAC
CCTGGGTAAACGTGGTTTCTCACGTGCTGACATCGTGAAGATCGCCGGC
AACATCGGTGGCGCCCAGGCCCTGCACACTGTGCTTGATCTGGAGCCTG
CACTAGGAAAACGAGGATTTTCCCGTATTGACATCGTTAAAATCGCGGC
CAACAATGGTGGCGCGCAAGCATTGCACGCTGTTTTAGACCTGGGTCCG
ACGCTGCGTGAGTGTGGTTTCAGTCAGGCGACCATCGCGAAGATTGCTG
GTAATAATGGAGGAGCACAAGCACTGCAAATGGTACTTGACCTGGGACC
CGCATTAGGCAAAAGGGGCTTCTCCCAGGCAACTATTGCTAAAATTGCT
GGTAACAATGGAGGGGCTCAAGCACTGCAGACCGTTCTTGACCTGGAAC
CGGCTCTGTGCGAGCGTGGTTTTGGCCAAGCAACAATTGCCAAAATGGC
TGGAAATATCGGGGGTGCGCAGGCATTACAAACAGTATTGGATTTAGAA
CCAGCGCTGCGAAAACGAGACTTCAGACAGGCCGATATTATAAAAATTG
CGGGAAATATTGGTGGAGCTCAGGCTCTACAGGCGGTTATTGAACACGG
ACCGACTTTGAGACAACATGGCTTTAACCTGGCGGACATCGTGAAAATG
GCTGGGAACAATGGCGGGGCCCAAGCGCTTCAGGCCGTCTTAGATTTAA
AACCCGTCTTGGATGAGCACGGCTTCAGCCAGGCTGACATCGTCAAAAT
CGCAGGCAATATCGGTGGGACCCAAGCGCTGCATGCGGTGCTGGATTTG
GAGCGTATGCTGGGGGAGCGCGGTTTCAGCAGAGCAGACATCGTGAATG
TGGCGGGAAACATTGGTGGTGCACAGGCTCTAAAGGCGGTATTAGAGCA
TGAAGCTACTCTTAATGAAAGAGGATTCTCCCGCGCCGACATCGTTAAA
ATCGCTGGCAACGGTGGCGGTGCCCAAGCTCTTAAAGCAGTTCTTGAGC
ACGAGGCAACACTGGATGAACGCGGTTTCTCGCGCGCGGATATTGTAAA
TGTTGCCGGGAACAACGGAGGCGCACAGGCGCTGAAAGCAGTGTTGGAA
CACGAGGCGACGTTAAACGAACGTGGGTTTAATCTGACAGACATCGTGG
AGATGGCTGCTAACGGCGGTGGCGCACAGGCATTAAAGGCTGTCCTTGA
GCATGGTCCGACCCTTCGCCAGCGCGGCTTGAGCTTGATTGACATTGTC
GAAATTGCCGGGAATGGCGGAGGAGCACAAGCGTTGAAAGCAGTCTTAA
AGTATGGACCGGTCCTTATGCAGGCCGGCCGTAGTAATGAAGAAATCGT
CCACGTAGCGGCGCGACGTGGTGGAGCAGGTCGTATTCGTAAAATGGTA
GCTCCGCTGCTCGAGCGTCAGGGCCTAGGCGGCAGCATGGACTTGAGGA
GACGCTGGCTGCGGGAGGTGAATTGGGTGCCTCCGAAGAAAAATAAGCC
AAACCACCTGGGCCACGCTCAGTCCCTTTCTCACGCTGAATCTCACGCC
CTGATTAGAGCTTATGAACGCATGGAGCGCCTCGGGGGCCAACTGCCTA
AGAAACTGACAATGGTGGTTGACCGCCCTACTTGTAACATTTGCAGGGG
CGAGATGCCTGCCCTCCTGAAACGCTTGGGCATTGAAGAGCTGACCATC
TACTCCGGCGGGCGCGACGCCATCATTATCAAGGCCATCAAATCCGGAG
GGTCGACTAATCTGAGCGACATTATAGAAAAAGAAACAGGTAAGCAGTT
GGTCATCCAAGAGAGTATTTTGATGCTGCCAGAGGAAGTCGAGGAGGTA
ATTGGTAACAAACCAGAGAGTGACATTCTTGTGCATACCGCTTATGACG
AGTCAACTGACGAGAATGTTATGCTCTTGACCTCTGATGCACCCGAATA
CAAACCTTGGGCACTCGTTATCCAGGACAGTAATGGAGAAAATAAAATA
AAAATGTTGTAATGAGCTCGGATCCCTGTGCCTTCTAGTTGCCAGCCAT
CTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTG
AGTAGGTGTCATTCTATTCTGGGGGGTGGGGTGGGGCAGGACAGCAAGG
GGGAGGATTGGGAAGACAATAGCAGGCATGCTGGGGATGCGGTGGGCTC
TATGG.

In an exemplary form, the second portion of the BE_R1_12 base editor is a fusion

(SEQ ID NO: 267)
MASVLTPLLLRSLTGSARRLMVPRAQVHSKSRSTAFVDQDKQMANRLNL
SPLERSKIEKQYGGATTLAFISNKQNELAQILSRADILKIASYDCAAHA
LQAVLDCGPMLGKRGFSQSDIVKIAGNGGGAQALQAVLDLESMLGKRGF
SRDDIAKMAGNGGGAQTLQAVLDLESAFRERGFSQADIVKIAGNNGGAQ
ALYSVLDVEPTLGKRGFSRADIVKIAGNIGGAQALHTVLDLEPALGKRG
FSRIDIVKIAANNGGAQALHAVLDLGPTLRECGFSQATIAKIAGNNGGA
QALQMVLDLGPALGKRGFSQATIAKIAGNNGGAQALQTVLDLEPALCER
GFGQATIAKMAGNIGGAQALQTVLDLEPALRKRDFRQADIIKIAGNIGG
AQALQAVIEHGPTLRQHGFNLADIVKMAGNNGGAQALQAVLDLKPVLDE
HGFSQADIVKIAGNIGGTQALHAVLDLERMLGERGFSRADIVNVAGNIG
GAQALKAVLEHEATLNERGFSRADIVKIAGNGGGAQALKAVLEHEATLD
ERGFSRADIVNVAGNNGGAQALKAVLEHEATLNERGFNLTDIVEMAANG
GGAQALKAVLEHGPTLRQRGLSLIDIVEIAGNGGGAQALKAVLKYGPVL
MQAGRSNEEIVHVAARRGGAGRIRKMVAPLLERQGLGGSMDLRRRWLRE
VNWVPPKKNKPNHLGHAQSLSHAESHALIRAYERMERLGGQLPKKLTMV
VDRPTCNICRGEMPALLKRLGIEELTIYSGGRDAIIIKAIKSGGSTNLS
DIIEKETGKQLVIQESILMLPEEVEEVIGNKPESDILVHTAYDESTDEN
VMLLTSDAPEYKPWALVIQDSNGENKIKML.

### III. Methods

Disclosed herein are various methods related to the disclosed compositions and reagents (including deaminase domains, base editors, etc.) and their use. For example, disclosed are methods of performing genome modification, deaminating a target nucleic acid, performing nucleic acid (base) editing in vitro or in vivo, identifying methylated nucleotides in a target nucleic acid and generating sequence diversity in a pool of target nucleic acids.

### A. Nucleic Acid Editing

Disclosed are sequence-specific DNA deaminases and targeted base editors that enable the precise or non-targeted editing of DNA both in vitro (e.g., in test tubes) and in vivo (e.g., in living cells). Unlike most of the previously characterized DNA deaminases that are known to be only active on single-stranded DNA (Iyer LM., et al., et al., Nucleic Acids Research 39, 9473-9497 (2011)), deaminases disclosed herein are active on double-stranded DNA (dsDNA) and possess various degrees of sequence specificity. For example, the deaminases and targeted base editors can deaminate dsDNA in certain contexts but not the others. These features make the DNA deaminases and targeted base editors useful for certain applications over base editors that use ssDNA-specific deaminases. For example, leveraging the disclosed dsDNA-specific deaminases, protein-only base editors are made (e.g. by fusing the deaminases to an array of protein-only targeting domains) that do not require any additional RNA or DNA moiety for their functions. These protein-only editors are especially useful for editing DNA species located in cellular compartments to which nucleic delivery is not efficient (e.g. mitochondria and chloroplast), thus sidestepping one of the major limitation of applying RNA-guided base editors for editing the genome of those organelles. Furthermore, due to their sequence specificity, the disclosed base editors can achieve precise genome editing with nucleotide resolution, without introducing mutations in the bystander nucleotides in the vicinity of a given target site. Existing base editors lack nucleotide resolution specificity and could introduce unwanted mutations to by-stander bases within the editing window, but the disclosed base editors equipped with sequence-specific DNA deaminases possess an additional layer of specificity originating from the deaminase domain. This has broad utility in addressing human genetic diseases and other biotechnological applications. For example, a disclosed targeted base editor including a deaminase domain with the desired specificity fused to a programmable DNA-binding domain (e.g., Cas9, Cfp1, TALEs, Zinc Fingers (ZFs), etc.) can be use perform sequence-specific base editing, the specificity of which can be influenced dictated by both the specificity of the DNA-binding domain as well as the deaminase domain.

As a further example, in some forms, when tethered to Cas9 (or another DNA-binding protein), an adenosine deaminase is localized to a gene of interest and catalyzes A to G mutations in the DNA substrate. This base editor can be used to target and revert single nucleotide polymorphisms (SNPs) in disease-relevant genes, which require A to G reversion. This base editor can also be used to target and revert SNPs in disease-relevant genes, which require T to C reversion by mutating the A, opposite of the T, to a G. The T may then be replaced with a C, for example by base excision repair mechanisms, or may be changed in subsequent rounds of DNA replication.

Thus, disclosed is a method of performing nucleic acid editing. In some forms, the method involves bringing into contact a target nucleic acid and a targeted base editor, whereby one or more instances of a target nucleotide sequence within the target nucleic acid is deaminated by the targeted base editor. In some forms, the target nucleic acid is single-stranded DNA or double-stranded DNA. Preferably, the target nucleic acid is double-stranded DNA.

Preferably, a target nucleotide in the target nucleotide sequence is deaminated. By “deaminated” is meant the removal of an amino group from a base (e.g., A, C) in the target nucleotide. Preferably, the removal is catalyzed by a disclosed deaminase via hydrolytic deamination. In some forms of the method, a deaminated nucleotide in the target nucleotide sequence is converted to a thymine or a guanine nucleotide, represented as T and G respectively. In some forms, a C is converted to T. In some forms, an A is converted to G. Typically, such conversion completes a base edit of the target nucleotide sequence. A “base edit” refers to the complete conversion of a nucleotide to another, optionally through an intermediate. For example, deamination of adenine (A) by an adenosine deaminase or base editor thereof results in the formation of hypoxanthine (I), which preferably base pairs with cytosine (C). DNA repair and/or replication machinery repair the I to G, which repair completes the base edit. Thus, a base edit can change an A. T base pair to G.C.

Analogously, deamination of cytosine (C) by a cytosine deaminase or base editor thereof results in the formation of uracil (U), which preferably base pairs with adenosine (A). DNA repair and/or replication machinery subsequently repairs the U to T, which repair completes the base edit. Thus, a base edit can change a C. G base pair to T. A.

Any target nucleotide sequence can be deaminated as long as an appropriate deaminase or base editor thereof is selected. In some forms, the target nucleotide sequence is AC, CC, GC, TC. In any of the foregoing exemplary target nucleotide sequences, in some forms, the last C in the target nucleotide sequence is deaminated by the deaminase or targeted base editor thereof.

In some forms, the intended target nucleotide sequence is edited with an efficiency of at least 1%, 2%, 3%, 4%, 5%, 10%, 15%, 20%, 25%, 30%, 35%, 40%, 45%, or 50%. In some forms, the method causes less than 19%, 18%, 16%, 14%, 12%, 10%, 8%, 6%, 4%, 2%, 1%, 0.5%, 0.2%, or less than 0.1% indel formation. In some forms, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some forms, the ratio of intended point mutation to indel formation is greater than 1:1, 10:1, 50:1, 100:1, 500:1, or 1000:1, or more.

In some forms, the target nucleic acid is nuclear (e.g., chromosomal) DNA. In some forms, the target nucleic acid is organelle genomes (mitochondrial, chloroplast, or plastids). In some forms, the target nucleic acid is outside of the cells, either in the form of purified or unpurified genomic DNA, plasmid, PCR product, or some form of synthetic DNA.

### Mitochondrial Genome Engineering

In some forms, the target nucleic acid is mitochondrial DNA. Thus, in some forms, the instance of the target nucleotide sequence in the mitochondrial DNA that is within a specified distance (e.g., 20 nucleotides) of the base editor target sequence is comprised in the mitochondrial DNA sequence.

The disclosed reagents and compositions, including deaminases and base editors thereof can be used to engineer mitochondrial genomes. This can be used to model mitochondrial genetic diseases (i.e. introduce pathogenic mutations to the mitochondrial genome) or correct pathogenic variants associated with mitochondrial genetic diseases. Due to the absence of efficient mechanisms to deliver guide RNAs (gRNAs) to the mitochondria, RNA-guided genome editing approaches have not been successfully used for engineering of the mitochondrial genome (Gammage PA., et al., Trends Genet., 34(2): 101-110 (2018)). Protein-only DNA binding domains such as TALEs and ZFs fused to ssDNA-specific deaminases cannot efficiently edit a target sequence in mitochondrial DNA since these DNA binding domains, unlike Cas9, do not expose a ssDNA region when bound to DNA. Recently, a dsDNA-specific cytidine deaminase (DddA) was fused to TALE to achieve mitochondrial genome engineering in human cell cultures (Mok et al., 2020). However, due to the context-dependency of this deaminase, only TC-to-TT mutations can be introduced, which corresponds to 4/93 confirmed pathogenic mutations in the MITOMAP database. In contrast, the disclosed deaminases and base editors thereof have expanded sequence specificities and, collectively, can edit cytidines in any sequence context (AC, CC, GC, and TC), allowing correction of 79/93 mitochondrial genetic mutations that cannot be addressed with the existing tools.

Thus, in some forms of the method of nucleic acid editing, the target nucleic acid is in a cell (e.g., in mitochondria). In some forms, the method involves bringing into contact the target nucleic acid and the targeted base editor by facilitating entry of the targeted base editor into the cell. “Facilitating entry” includes bringing the targeted base editor into contact with the cell, where the targeted base editor is formulated or composed to be able to enter the cell. In some forms the cell is in a subject (e.g., an animal). Thus, in some forms, bringing into contact the target nucleic acid and the targeted base editor is accomplished by administering the targeted base editor to the subject (e.g., animal).

Also disclosed is a method of performing mitochondrial genome engineering in vivo by introducing to a cell a targeted cytosine or adenosine deaminase base editor, wherein a target nucleotide sequence within mitochondrial DNA is deaminated by the targeted base editor. In some forms the cell is in a subject (e.g., an animal). In some forms, editing of a target nucleotide or target nucleotide sequence in mitochondrial DNA results in correction of a mutation (e.g., a pathogenic or disease-associated mutation) in mitochondria. Pathogenic or disease-associated mitochondrial mutations are known in the art, some of which are catalogued in the MITOMAP database (http://www.mitomap.org/), a database of human mitochondrial DNA variation. Table 2 provides a non-limiting list of pathogenic mitochondrial mutations.

TABLE 2
Exemplary pathogenic mitochondrial mutations, loci and associated diseases.

| | | | Fixing | | Nucleotide |
|---|---|---|---|---|---|
| Locus | Associated Diseases | Allele | mutation | AA | +/− 3 |
| MT-TF | MELAS/MM & EXIT | m.583G > A | m.583A > G | tRNA Phe | TATGTAG |
| MT-TF | Maternally inherited epilepsy/ | m.616T > C | m.616C > T | tRNA Phe | AAATGTT |
| | kidney disease | | | | |
| MT-RNR1 | DEAF | m.1494C > T | m.1494T > C | 12S rRNA | CACCCTC |
| MT-RNR1 | DEAF; autism spectrum | m.1555A > G | m.1555G > A | 12S rRNA | GAGACAA |
| | intellectual disability; | | | | |
| | possibly antiatherosclerotic | | | | |
| MT-TV | AMDF | m.1606G > A | m.1606A > G | tRNA Val | AGAGTGT |
| MT-TV | MNGIE-like disease/ | m.1630A > G | m.1630G > A | tRNA Val | CCAACTT |
| | MELAS | | | | |
| MT-TV | Leigh Syndrome/HCM/ | m.1644G > A | m.1644A > G | RNA Val | GGAGATT |
| | MELAS | | | | |
| MT-TL1 | MELAS/Leigh Syndrome/ | m.3243A > G | m.3243G > A | tRNA Leu | CAGAGCC |
| | DMDF/MIDD/SNHL/ | | | (UUR) | |
| | CPEO/MM/FSGS/ASD/ | | | | |
| | Cardiac + multi-organ | | | | |
| | dysfunction | | | | |
| MT-TL1 | MM/MELAS/SNHL/ | m.3243A > T | m.3243T > A | tRNA Leu | CAGAGCC |
| | CPEO | | | (UUR) | |
| MT-TL1 | MELAS; possible | m.3256C > T | m.3256T > C | tRNA Leu | TCGCATA |
| | atherosclerosis risk | | | (UUR) | |
| MT-TL1 | MELAS/Myopathy | m.3258T > C | m.3258C > T | tRNA Leu | GCATAAA |
| | | | | (UUR) | |
| MT-TL1 | MMC/MELAS | m.3260A > G | m.3260G > A | tRNA Leu | ATAAAAC |
| | | | | (UUR) | |
| MT-TL1 | PEM/retinal dystrophy in | m.3271delT | m.3271delT | tRNA Leu | AACTTTA |
| | MELAS | | | (UUR) | |
| MT-TL1 | MELAS/DM | m.3271T > C | m.3271C > T | tRNA Leu | AACTTTA |
| | | | | (UUR) | |
| MT-TL1 | Myopathy | m.3280A > G | m.3280G > A | tRNA Leu | GTCAGAG |
| | | | | (UUR) | |
| MT-TL1 | MELAS/Myopathy/ | m.3291T > C | m.3291C > T | tRNA Leu | AATTCCT |
| | Deafness + Cognitive | | | (UUR) | |
| | Impairment | | | | |
| MT-TL1 | MM | m.3302A > G | m.3302G > A | tRNA Leu | TTAACAA |
| | | | | (UUR) | |
| MT-TL1 | MMC | m.3303C > T | m.3303T > C | tRNA Leu | TAACAAC |
| | | | | (UUR) | |
| MT-ND1 | LHON MELAS overlap | m.3376G > A | m.3376A > G | E-K | ACCGAAC |
| MT-ND1 | LHON | m.3460G > A | m.3460A > G | A-T | GACGCCA |
| MT-ND1 | LHON | m.3635G > A | m.3635A > G | S-N | CTAGCCT |
| MT-ND1 | MELAS/Leigh Syndrome/ | m.3697G > A | m.3697A > G | G-S | ATCGGCG |
| | LDYT/BSN | | | | |
| MT-ND1 | LHON | m.3700G > A | m.3700A > G | A-7 | GGCGCAC |
| MT-ND1 | LHON | m.3733G > A | m.3733A > G | E-K | TATGAAG |
| MT-ND1 | Progressive | m.3890G > A | m.3890A > G | R-Q | ACCGAAC |
| | Encephalomyopathy/Leigh | | | | |
| | Syndrome/Optic Atrophy | | | | |
| MT-ND1 | EXIT + myalgia/severe | m.3902_3908, | m.3902_3908, | DLA- | TCGACCT |
| | LA + cardiac/3-MGA | ACCTTGCinv | ACCTTGCinv | GKV | TGCCGA |
| | aciduria | | | | |
| MT-ND1 | LHON/Leigh-like | m.4171C > A | m.4171A > C | L-M | CTCCTAT |
| | phenotype | | | | |
| MT-TI | CPEO/MS | m.4298G > A | m.4298A > G | tRNA Ile | AGAGTAA |
| MT-TI | MICM | m.4300A > G | m.4300G > A | tRNA Ile | AGTAAAT |
| MT-TI | CPEO | m.4308G > A | m.4308A > G | tRNA Ile | ATAGGAG |
| MT-TQ | Encephalopathy/MELAS | m.4332G > A | m.4332A > G | RNA Gln | CTAGGAC |
| MT-TM | Myopathy/MELAS/Leigh | m.4450G > A | m.4450A > G | tRNA Met | TTGGTTA |
| | Syndrome | | | | |
| MT-TW | Mitochondrial myopathy | m.5521G > A | m.5521A > G | tRNA Trp | TTAGGTT |
| MT-TW | Leigh Syndrome | m.5537_5538 | m.5537_5538 | tRNA Trp | CCAAGAG |
| | | insT | insT | | |
| MT-TA | Myopathy | m.5650G > A | m.5650A > G | tRNA Ala | TAAGCCC |
| MT-TN | CPEO + ptosis + proximal | m.5690A > G | m.5690G > A | tRNA Asn | CTTAGTT |
| | myopathy | | | | |
| MT-TN | CPEO/MM | m.5703G > A | m.5703A > G | tRNA Asn | TAAGCAC |
| MT-TN | Multiorgan failure/ | m.5728T > C | m.5728C > T | tRNA Asn | ATCTACT |
| | myopathy | | | | |
| MT-CO1 | SNHL | m.7445A > G | m.7445G > A | Term- | TAGACAA |
| | | | | Term | |
| MT-TS1 | SNHL | m.7445A > G | m.7445G > A | tRNA Ser | TAGACAA |
| precursor | | | | (UCN) | |
| | | | | precursor | |
| MT-TS1 | PEM/AMDF/Motor | m.7471_7472 | m.7471_7472 | tRNA Ser | CCCCAAA |
| | neuron disease-like | insC | insC | (UCN) | |
| MT-TS1 | MM/EXIT | m.7497G > A | m.7497A > G | tRNA Ser | CATGGCC |
| | | | | (UCN) | |
| MT-TS1 | SNHL | m.7510T > C | m.7510C > T | tRNA Ser | ACTTTTT |
| | | | | (UCN) | |
| MT-TS1 | SNHL/Deafness | m.7511T > C | m.7511C > T | tRNA Ser | CTTTTTC |
| | | | | (UCN) | |
| MT-TK | Severe adult-onset | m.8306T > C | m.8306C > T | tRNA Lys | AGCTAAC |
| | multisymptom myopathy/ | | | | |
| | Myoclonic epilepsy | | | | |
| MT-TK | MNGIE/Progressive mito | m.8313G > A | m.8313A > G | tRNA Lys | TTAGCAT |
| | cytopathy | | | | |
| MT-TK | Myopathy/Exercise | m.8340G > A | m.8340A > G | tRNA Lys | TAAGAGA |
| | Intolerance/Eye | | | | |
| | disease + SNHL | | | | |
| MT-TK | MERRF; Other-LD/ | m.8344A > G | m.8344G > A | tRNA Lys | AGAACCA |
| | Depressive mood disorder/ | | | | |
| | leukoencephalopathy/HiCM | | | | |
| MT-TK | MERRF | m.8356T > C | m.8356C > T | tRNA Lys | TCTTTAC |
| MT-TK | MICM + DEAF/MERRF/ | m.8363G > A | m.8363A > G | tRNA Lys | AGTGAAA |
| | Autism/Leigh Syndrome/ | | | | |
| | Ataxia + Lipomas | | | | |
| MT- | Infantile cardiomyopathy | m.8528T > C | m.8528C > T | ATP8: | AAATGAA |
| ATP8/6 | | | | W-R | |
| | | | | ATP6: | |
| | | | | M-T | |
| MT-ATP6 | BSN/Leigh syndrome | m.8851T > C | m.8851C > T | W-R | TTATGAG |
| MT-ATP6 | Mitochondrial myopathy, | m.8969G > A | m.8969A > G | S-N | TCAGCCT |
| | lactic acidosis and | | | | |
| | sideroblastic anemia | | | | |
| | (MLASA)/IgG nephropathy | | | | |
| MT-ATP6 | NARP/Leigh Disease/ | m.8993T > C | m.8993C > T | L-P | CCCTGGC |
| | MILS/other | | | | |
| MT-ATP6 | NARP/Leigh Disease/ | m.8993T > G | m.8993G > T | L-R | CCCTGGC |
| | MILS/other | | | | |
| MT-ATP6 | Ataxia syndromes | m.9035T > C | m.9035C > T | L-P | TACTCAT |
| MT-ATP6 | MIDD, renal insufficiency | m.9155A > G | m.9155G > A | Q-R | TCCAAGC |
| MT-ATP6 | FBSN/Leigh Disease | m.9176T > C | m.9176C > T | L-P | TTCTAGT |
| MT-ATP6 | Leigh Disease/Spastic | m.9176T > G | m.9176G > T | L-R | TTCTAGT |
| | Paraplegia | | | | |
| MT-ATP6 | Leigh Disease/Ataxia | m.9185T > C | m.9185C > T | L-P | GCCTCTA |
| | syndromes/NARP-like | | | | |
| | disease | | | | |
| MT-ATP6 | Encephalopathy/Seizures/ | m.9205_9206 | m.9205_9206 | Ter-M | ACATAAT |
| | Lacticacidemia | delTA | delTA | | |
| MT-TG | PEM | m.10010T > C | m.10010C > T | tRNA Gly | TAGTACC |
| MT-ND3 | Leigh Disease/MELAS | m.10158T > C | m.10158C > T | S-P | AAATCCA |
| MT-ND3 | Leigh Disease/Leigh-like | m.10191T > C | m.10191C > T | S-P | ATATCCC |
| | Disease/ESOC | | | | |
| MT-ND3 | Leigh Disease/Dystonia/ | m.10197G > A | m.10197A > G | A-T | CCCGCCC |
| | Stroke/LDYT | | | | |
| MT-ND4L | LHON | m.10663T > C | m.10663C > T | V-A | TAGTCTT |
| MT-ND4 | Leigh Disease | m.11777C > A | m.11777A > C | R-S | AGTCGCA |
| MT-ND4 | LHON/Progressive | m.11778G > A | m.11778A > G | R-H | GTCGCAT |
| | Dystonia | | | | |
| MT-TH | MERRF-MELAS/ | m.12147G > A | m.12147A > G | tRNA His | ATAGTTT |
| | Encephalopathy | | | | |
| MT-TS2 | DMDF/RP + SNHL | m.12258C > A | m.12258A > C | tRNA Ser | TGGCTTT |
| | | | | (AGY) | |
| MT-TL2 | CPEO | m.12276G > A | m.12276A > G | tRNA Leu | AAGGATA |
| | | | | (CUN) | |
| MT-TL2 | CPEO/ | m.12294G > A | m.12294A > G | tRNA Leu | TTGGTCT |
| | EXIT + Ophthalmoplegia | | | (CUN) | |
| MT-TL2 | CPEO/KSS/possible | m.12315G > A | m.12315A > G | tRNA Leu | TTTGGTG |
| | carotid atherosclerosis risk, | | | (CUN) | |
| | trend toward myocardial | | | | |
| | infarction risk | | | | |
| MT-TL2 | CPEO | m.12316G > A | m.12316A > G | tRNA Leu | TTGGTGC |
| | | | | (CUN) | |
| MT-ND5 | Leigh Disease | m.12706T > C | m.12706C > T | F-L | ATCTTCC |
| MT-ND5 | Optic neuropathy/ | m.13042G > A | m.13042A > G | A-T | TCAGCCA |
| | retinopathy/LD | | | | |
| MT-ND5 | LHON | m.13051G > A | m.13051A > G | G-S | GAAGGCC |
| MT-ND5 | Ataxia + PEO/MELAS, LD, | m.13094T > C | m.13094C > T | V-A | TAGTTGT |
| | LHON, myoclonus, fatigue | | | | |
| MT-ND5 | LHON | m.13379A > C | m.13379C > A | H-P | TCCACAA |
| MT-ND5 | Leigh Disease/MELAS/ | m.13513G > A | m.13513A > G | D-N | AAAGACC |
| | LHON-MELAS Overlap | | | | |
| | Syndrome/negative | | | | |
| | association w Carotid | | | | |
| | Atherosclerosis | | | | |
| MT-ND5 | Leigh Disease/MELAS/ | m.13514A > G | m.13514G > A | D-G | AAGACCA |
| | Ca2+ downregulation | | | | |
| MT-ND6 | LDYT/Leigh Disease/ | m.14459G > A | m.14459A > G | A-V | ATCGCTG |
| | dystonia/carotid | | | | |
| | atherosclerosis risk | | | | |
| MT-ND6 | LHON | m.14482C > A | m.14482A > C | M-I | AACCATC |
| MT-ND6 | LHON | m.14482C > G | m.14482G > C | M-I | AACCATC |
| MT-ND6 | LHON | m.14484T > C | m.14484C > T | M-V | CCATCAT |
| MT-ND6 | Dystonia/Leigh Disease/ | m.14487T > C | m.14487C > T | M-V | TCATTCC |
| | ataxia/ptosis/epilepsy | | | | |
| MT-ND6 | LHON | m.14495A > G | m.14495G > A | L-S | CCTAAAT |
| MT-ND6 | LHON | m.14568C > T | m.14568T > C | G-S | CACCGCT |
| MT-TE | Reversible COX deficiency | m.14674T > C | m.14674C > T | tRNA Glu | CATTATT |
| | myopathy | | | | |
| MT-TE | MM+DMDF/ | m.14709T > C | m.14709C > T | tRNA Glu | ATATGAA |
| | Encephalomyopathy/ | | | | |
| | Dementia + diabetes + ophthal | | | | |
| | moplegia | | | | |
| MT-TE | Encephalomyopathy + | m.14710G > A | m.14710A > G | tRNA Glu | TATGAAA |
| | Retinopathy | | | | |
| MT-CYB | EXIT/Septo-Optic | m.14849T > C | m.14849C > T | S-P | GGCTCAC |
| | Dysplasia | | | | |
| MT-CYB | Multisystem Disorder, EXIT | m.15579A > G | m.15579G > A | Y-C | CCTACAC |

LHON: Leber's hereditary optic neuropathy; MELAS: mitochondrial encephalomyopathy, lactic acidosis, and stroke-like episodes; NARP: neuropathy, ataxia, and retinitis pigmentosa; MILS: maternally inherited Leigh syndrome; MERRF: myoclonic epilepsy with ragged red fibers.

In some forms, a target nucleotide that is deaminated by a disclosed targeted base editor is selected from mutations listed in Table 2. In some forms, a target nucleotide that is deaminated by a disclosed targeted base editor is selected from m.583G>A, m.616T>C, m.1606G>A, m.1644G>A, m.3258T>C, m.3271T>C, m.3460G>A, m.4298G>A, m.5728T>C, m.5650G>A, m.3243A>G, m.8344A>G, m.14459G>A, m.11778G>A, m.14484T>C, m.8993T>C, m.14484T>C, m.3460G>A, ad m.1555A>G. Most preferred are m.3243A>G, m.8344A>G, m.14459G>A, m.11778G>A, m.14484T>C, m.8993T>C, m.14484T>C, m.3460G>A, and m.1555A>G.

Thus, disclosed is a method of addressing a mitochondrial genetic disease by fixing its underlying mutation. The method involves introducing to a cell a targeted cytosine or adenosine deaminase base editor, wherein a target nucleotide sequence within mitochondrial DNA is deaminated by the targeted base editor. In some forms, the deaminated nucleotide in the target nucleotide sequence is converted to a thymine or a guanine nucleotide. The conversion completes a base edit of the target nucleotide sequence. The base edit results in fixing a pathogenic or mitochondrial disease-associated mutation and reverting that mutation back to WT or non-pathogenic form in mitochondrial nucleic acid. Any suitable patient-derived cell may be used, including but not limited to, fibroblasts, lymphocytes, pancreatic cells, muscle cells, neuronal cells, and stem cells, including iPSCs. In some forms, the cell is in a subject (e.g., an animal or human); thus, the base editors can be used as a thereby to fix a pathogenic mutation and underlying disease condition. Given the absence of any reliable technology to introduce precise edits to mitochondrial genome, making cell or animal models for mitochondrial genetic diseases has been challenging. Besides, correction of pathogenic mitochondrial variants to cure mitochondrial diseases (i.e. gene therapy applications), the disclosed base editors can also be used in methods of making cell or animal models for mitochondrial genetic diseases. Such methods enable forward genetics studies of these genetic diseases as well as mitochondrial physiology, and genetic heteroplasmy. Additionally, the disclosed base editors enable forward genetics studies for complex diseases such as cancer, metabolic disorders and aging and could help to unravel role of mitochondrial encoded genes and mutations in these and similar non genetically defined disorders.

Thus, disclosed is a method of making a cell model for a mitochondrial genetic disease. The method involves introducing to a cell a targeted cytosine or adenosine deaminase base editor, wherein a target nucleotide sequence within mitochondrial DNA is deaminated by the targeted base editor. In some forms, the deaminated nucleotide in the target nucleotide sequence is converted to a thymine or a guanine nucleotide. The conversion completes a base edit of the target nucleotide sequence. The base edit results in introduction of a pathogenic or mitochondrial disease-associated mutation in a previously wildtype or non-mutated target mitochondrial nucleic acid. Any suitable cell may be used, including but not limited to, fibroblasts, lymphocytes, pancreatic cells, muscle cells, neuronal cells, and stem cells, including iPSCs. In some forms, the cell is in a subject (e.g., an animal); thus, animal models of mitochondrial diseases can be made thereby.

Exemplary wildtype mitochondrial DNA target nucleotide sequences which can undergo a base edit to generate a pathogenic mutation for disease modeling can be selected from Table 2 and include, without limitation, CACcCTC, GAGaCAA, CAGaGCC, TCGcATA, GTCaGAG, TAAcAAC, AGTaAAT, TAGaCAA, CACcGCT, and AGAaCCA, wherein the target nucleotide that is edited to generate the pathogenic mutation is in lowercase.

The various reagents and compositions to be used in methods of nucleic acid editing can be introduced to a cell or subject by a variety of means known in the art. For example, the deaminase, targeted base editor, or other reagents can be delivered in various forms, such as combinations of DNA, RNA, protein, or combinations thereof. For example, a base editor may be delivered as a DNA-coding polynucleotide or an RNA-coding polynucleotide or as a protein. In cases where the base editor comprises a Crispr-Cas effector protein as the targeting domain, an appropriate guide RNA or crRNA may be delivered as a DNA-coding polynucleotide or an RNA. All possible combinations are envisioned, including mixed forms of delivery.

In some forms, the methods comprise delivering one or more polynucleotides, such as or one or more vectors, one or more transcripts thereof, and/or one or proteins transcribed therefrom, to a host cell. Suitable vectors for introducing or providing the nucleic acid editing reagents into a cell include, without limitation, plasmids and viral vectors derived from, for example, bacteriophages, baculoviruses, retroviruses (such as lentiviruses), adenoviruses, poxviruses, Epstein-Barr viruses, and adeno-associated viruses (AAV). The viral vector can be derived from a DNA virus (e.g., dsDNA or ssDNA virus) or an RNA virus (e.g., an ssRNA virus), or it could be a virus-like particle (VLP). Numerous vectors and expression systems are commercially available from commercial vendors including Addgene, Novagen (Madison, WI), Clontech (Palo Alto, CA), Stratagene (La Jolla, CA), and Invitrogen/Life Technologies (Carlsbad, CA). Advantageous vectors include lentiviruses and adeno-associated viruses, and subtypes of such vectors can also be selected for targeting particular types of cells.

The nucleic acid editing reagents (e.g., base editor) can be introduced to a cell by a variety of viral or non-viral techniques. The reagents can be provided in a viral vector (e.g., a retrovirus such as a lentivirus, adenovirus, poxvirus, Epstein-Barr virus, adeno-associated virus (AAV), virus-like particle (VLP), etc.). Non-viral approaches such as physical and/or chemical methods can also be used, including, but not limited to cationic liposomes and polymers, exosomes, DNA nanoclew, gene gun, microinjection, electroporation, nucleofection, particle bombardment, ultrasound utilization, magnetofection, and conjugation to cell penetrating peptides. Such methods are described for example, in Nayerossadat N., et al., Adv. Biomed. Res., 1:27 (2012) and Lino C A, et al., Drug Deliv., 25(1):1234-1257 (2018). A skilled artisan, based on known delivery methods in the art in context of their respective advantages and disadvantages would be able to determine an optimal method.

In some forms, the deaminase or base editor thereof can be introduced to the cell via an mRNA that encodes the deaminase or base editor. The mRNA can contain modifications such as N6-methyladenosine (m6A), 5-methylcytosine (m5C), pseudouridine (ψ), N1-methylpseudouridine (me1ψ), and 5-methoxyuridine (5moU); a 5′ cap; a poly(A) tail; one or more nuclear localization signals; or combinations thereof. The mRNA can be codon optimized for expression in a eukaryotic cell and can be introduced to the cell via electroporation, transfection, and/or nanoparticle mediated delivery. The deaminase or base editor can also be introduced via a viral vector that encodes the RNA-guided endonuclease, or direct electroporation of the deaminase or base editor protein, or base editor protein-RNA complex.

The nucleic acid editing reagents can each individually be contained in a composition and introduced to a cell individually or collectively. Alternatively, these components can be provided in a single composition for introduction to a cell.

### B. Identifying Modified Nucleotides

Methods for identifying the presence and/or position of nucleotide modifications (i.e. epigenetic marks) in a target nucleic acid are also provided.

Epigenetic sequencing is typically used to identify and localize modifications to nucleotides in the genome via DNA sequencing. While a variety of modifications exist, the most prevalent and consequential are 5-methylCytosine (5-mC) and 5-hydroxymethylCytosine (5-hmC). The main technique used to identify these epigenetic modifications is bisulfite sequencing (Raiber E A., et al., Nat Rev Chem 1, 0069 (2017)). In this approach, extracted genomes are treated with the chemical bisulfite, which converts all unmodified Cytosines to uracil. During sequencing, these are read as “T.” While this technique is widely adopted, it results in the chemical destruction of 99% of DNA molecules used. In addition, it results in sequencing errors since the conversion of all unmodified C's to U's skews the distribution of bases. Furthermore, the conversion is not 100%, resulting in potential misidentification of modified cytosines. A newly developed approach by New England Biolabs (NEB), replaces the harsh chemical treatment of bisulfite with APOBEC: a ssDNA-specific enzyme which analogously converts Cytosines to Uracils (https://www.neb.com/tools-and-resources/feature-articles/enzymatic-methyl-seq-the-next-generation-of-methylome-analysis). However, APOBEC also deaminates 5mC and 5hmC, making it impossible to differentiate between cytosine and its modified forms. In order to detect 5mC and 5hmC, this method also utilizes TET2 and an Oxidation Enhancer, which enzymatically modifies 5mC and 5hmC to forms that are not substrates for APOBEC. The TET2 enzyme converts 5mC to 5caC and the Oxidation Enhancer converts 5hmC to SghmC. Ultimately, cytosines are sequenced as Thymines and 5mC and 5hmC are sequenced as cytosines, thereby protecting the integrity of the original 5mC and 5hmC sequence information. While this is an improvement, it still skews the distribution of bases, making standard genome sequencing challenging. The requirement for using TET2 and Oxidation Enhancer and the presence of DNA in ssDNA form as the substrate for APOBEC, makes the process limited, complicated and inefficient.

A significant improvement to bisulfite sequencing is the recently developed TET-assisted pyridine borane sequencing (TAPS) (Liu Y., et al., Nat Biotechnol 37, 424-429 (2019)). This method uses a combination of enzymatic and chemical treatments to convert 5-mC and 5-hmC to U. TAPS is less harsh than bisulfite sequencing and mitigates sequencing artifacts that arise from skewed base distributions. However, its main limitation is its inability to distinguish 5-mC from 5-hmC.

The disclosed deaminases and base editors thereof are active on dsDNA and can detect (or be evolved to detect) methylation (5mC and 5hmC) or other modifications on DNA, thus greatly facilitating and improving the existing epigenetic sequencing workflows and opening up new frontiers for detecting epigenetic marks beyond methylation by sequencing. The epigenetic marker identifications can be used for various R&D and diagnostics applications, including detection of cancer and many other diseases, and provide an additional information layer to genomic data.

Thus, methods for determining the presence and/or position of epigenetic marks are provided. In some forms, the methods involve determining the presence and/or position of modified nucleotides (e.g., 5mC and 5hmC) in DNA. An exemplary method includes bringing into contact a target nucleic acid and a deaminase domain, wherein the target nucleic acid is double-stranded cytosine-methylated DNA and sequencing the target nucleic acid to identify methylated cytosine nucleotides in the target nucleic acid. Preferably, the deaminase domain can deaminate double-stranded DNA and possess differential activity (e.g. different deamination rates) on non-methylated cytidine and various forms of cytidine modifications (e.g., mC and hmC). In some forms, the deaminase domain and target nucleic acid are incubated for a period of time and under conditions suitable for the deaminase domain to deaminate the target nucleic acid. In some forms, the deaminase domain deaminates substantially only non-methylated cytosine nucleotides in the target nucleic acid. In some forms, the methylated nucleotide on the DNA substrate are first converted to oxidized forms (e.g. caC and fC) using TET2 and BGT enzyme treatment (via methods that are known in the prior art) before treating with dsCDAs to allow better differentiation between methylated and non-methylated cytidines. In some forms, substantially all (or majority) of the non-methylated cytosine nucleotides in the target nucleic acid are deaminated by the deaminase domain. Upon sequencing the deaminated target nucleic acid, methylated cytosine nucleotides in the target nucleic acid are identified (they are sequenced as cytosines). In addition, unmodified cytosines in the in the target nucleic can be identified since they are sequenced as thymines. Appropriate methods for sequencing nucleic acids are known in the art. Various types of sequencing can be performed including targeted sequencing, whole genome sequencing, or whole exome sequencing. Single-end or paired-end sequencing of the nucleic acid sample may be performed.

Suitable sequencing methods include, but are not limited to, sanger sequencing high-throughput sequencing, pyrosequencing, sequencing-by-synthesis, single-molecule sequencing, nanopore sequencing (e.g., MinION), semiconductor sequencing, sequencing-by-ligation, sequencing-by-hybridization, Digital Gene Expression (Helicos), Next generation sequencing (e.g., Roche 454, Solexa platforms such as HiSeq2000, and SOLiD), Single Molecule Sequencing by Synthesis (SMSS)(Helicos), massively-parallel sequencing, Clonal Single Molecule Array (Solexa), Single Molecule Real Time sequencing (SMRT), shotgun sequencing, Maxim-Gilbert sequencing, primer walking, sequencing using PacBio, SOLiD, Ion Torrent, or Nanopore platforms and any other sequencing methods known in the art.

In some forms, the deaminase domain deaminates at least 50%, 60%, 70%, 75%, 80%, 85%, 90%, 95%, 96%, 97%, 98%, 99%, or 99.5% of the non-methylated cytosine nucleotides in the target nucleic acid. In some forms, the deaminase domain deaminates 50-100%, 50-90%, 50-80%, 60-100%, 60-90%, 60-80%, 70-100%, 70-90%, 70-80%, 80-100%, 80-95%, 80-90%, 90-100%, 90-95%, 95-100%, or 95-99.5% of the non-methylated cytosine nucleotides in the target nucleic acid. Preferably, the deaminase domain deaminates 90% or more (e.g., 95%, 96%, 97%, 98%, 99%, 99.5%, or more) of the non-methylated cytosine nucleotides in the target nucleic acid.

In some forms the deaminase is a dsDNA specific cytosine deaminase, and preferably, a substantially non-sequence specific cytosine deaminase. For example, the deaminase domain may have a preference for, but is not limited to, deaminating a specific target nucleotide sequence. In some forms, a mixture of dsDNA specific deaminases can be used to minimize sequence bias imposed by any individual deaminase and deaminate non-methylated cytosines independent of their sequence context.

Different dsDNA-specific deaminases (dsCDAs) show different activities on cytidine and its various modifications (i.e. epigenetic marks. 5mC, 5hmC, 5fC, 5caC). This feature can be leveraged to differentially mark various epigenetic marks (cytidine modifications) which can then be read by sequencing methods. This method offers an enzymatic alternative to bisulfite sequencing, and address shortcoming and technical limitations associated with bisulfite treatment of DNA, thus minimizing generating better quality results. As set forth in the Examples, it has been shown that deaminases are more active on non-methylated cytidines [(m)C], but not on methylated cytidines (5mC and 5hmC). In addition, the editing efficiency (C-to-T conversion) was higher on non-methylated dC residues, suggesting that dsCDAs act differentially on non-methylated and methylated DNA. It was found that 5hmC and 5mC were more resistant to deamination when protected by glucosylation and oxidation.

### C. Generating Sequence Diversity

Random mutagenesis encompasses a set of techniques that generate sequence diversity and library of closely related variants to explore gene and protein function. Common among these methods is Error-prone PCR (Wilson D S and Keefe A D., Curr Protoc Mol Biol. 2001; PMID: 18265275), where an error-prone polymerase, or another mutator enzyme, is used to diversify/amplify a gene of interest and introduce random mutations that can impact the function of the gene. Despite its utility, error-prone PCR is biased in the types of mutations it is able to produce. Another approach is DNA-shuffling (Joern J. M. (2003) DNA Shuffling. In: Arnold F. H., Georgiou G. (eds). Methods in Molecular Biology™, vol 231. Humana Press. internet site doi.org/10.1385/1-59259-395-X:85), where short sequences between two similar genes are randomly shuffled to yield a library of variant genes. The main limitation of this approach is the requirement for the two genes to have significant sequence similarity. In another approach, a transposase is used to randomly insert a short segment of DNA into a gene (Cartman S T and Minton N P, Appl Environ Microbiol., 76(4):1103-9 (2010)). While less commonly used, tranposase based approaches suffer from requirements on their insertion sites. Finally, random mutations can be used via the use of chemicals such as ethyl methanesulfonate (EMS), which primarily makes modifications of guanosine nucleotides. Chemical mutagenesis approaches often require in vivo DNA repair mechanisms and only make modifications to guanosines, limiting the diversity of sequences that can be generated.

The disclosed dsDNA-specific deaminases can be used to introduce random mutations with tunable efficiency into a DNA molecule of interest, thus facilitating and streamlining directed evolution workflows for optimizing various genetically encoded biomolecules (e.g., antibodies, aptamers, etc.). Thus, methods for randomly mutating a pool of DNA sequences are provided. Methods for generating sequence diversity in a pool of target nucleic acids are also provided. In such methods, the deaminase is preferably, a substantially non-sequence specific deaminase or a mixture of sequence-specific deaminases that collectively can edit a target sequence with minimal context dependency. For example, the deaminase domain may have a preference for, but is not limited to, deaminating a specific target nucleotide sequence, or multiple deaminases with distinct specificity are used concurrently.

In some forms, such methods involve bringing into contact a deaminase domain and a plurality of copies of a target nucleic acid for a time and under conditions that results in deamination of the target nucleic acid. In some forms, the method effects deamination of an average of 0.1 to 5.0 nucleotides per copy of the target nucleic acid. In some forms, the method effects deamination of an average of about 0.1, 0.5, 1.0, 1.5, 2.0, 2.5, 3.0, 3.5, 4.0, 4.5, or 5.0 nucleotides per copy of the target nucleic acid. Preferably, the target nucleic acid is double-stranded DNA and the deaminase domain can deaminate double-stranded DNA.

In some forms, the copies of the target nucleic acid are in vitro. Thus, the deaminated nucleotides in the copies of the target nucleic acid can be converted to a thymine or a guanine nucleotide via an in vitro reaction.

In some forms, the method further includes subjecting the deaminated copies of the target nucleic acid to a selection or screen procedure, that could be conducted in vivo or in vitro. Selection or screening methods directly eliminate unwanted variants through applying certain selective pressure to the library of target nucleic acids. Suitable selection procedures include, without limitation, mRNA display, ribosome display, and SELEX (in vitro), or in vivo cell based selection methods (the latter requires cloning the diversified DNA fragment into a suitable vector before introducing to the cells).

In some forms, the deaminated nucleotides in the copies of the target nucleic acid are converted to a thymine or a guanine nucleotide, wherein the conversion completes one or more base edits of some or all of the copies of target nucleic acid.

In some forms, the deaminated nucleotides in the copies of the target nucleic acid can be converted to a thymine or a guanine nucleotide by incubating the copies of the target nucleic acid in cells. Thus, in some forms, the copies of the target nucleic acid are in cells, and the deaminase domain and the copies of a target nucleic acid are brought into contact by facilitating entry of the deaminase domain into the cells (e.g., through electroporation of mRNA or protein, transfection with an expression vector, transformation, etc.).

In some forms, the deaminase domain is an isolated deaminase domain. In some forms, the deaminase domain is fused to a targeting domain (e.g., DNA binding domain, Transcription factor, DNA or RNA polymerase (e.g. an orthogonal RNA polymerase such as T7 RNA polymerase in human cells), other replication and transcription accessory factors, etc.) so that the deaminase domain is preferentially co-localized with the targeting domain on the DNA sequence that is occupied by the targeting domain (e.g. DNA binding domain target site, transcription factor target site, the entire genome in the case of DNA polymerase fusion, the promoter and genes transcribed by RNA polymerase fusion, etc.). This approach could be used to identify binding sites of transcription factors or other DNA interacting proteins in high-throughput (as an alternative to ChIP-Seq) by fusing the dsDNA specific deaminase to transcription factor(s) or other DNA interacting domain of interest and introducing the fusion to the cells, where the interactions of the domain of interest with DNA are uniquely marked by the deaminase in the form of C to T mutations, which can then be detected by whole genome sequencing.

In other forms, the approach could be used to continuously diversify a locus of interest inside the cells with high efficiency, e.g. by fusing the deaminase domain to DNA interacting domains. The choice of DNA interacting domains can be made so that the mutations are generated across the genome (e.g. a deaminase domain is fused DNA polymerase or an accessory protein to DNA polymerase can be used). Alternatively, only a defined segment of a genome or plasmid can be targeted (e.g. the deaminase domain is fused to an RNA polymerase to target regions defined by the promoters for that polymerase. The deaminase can be fused to an orthogonal DNA polymerase such as T7 RNA polymerase in a host that doesn't naturally encode T7 promoter. A DNA segment of interest can be placed in front of T7 and expressed in the given host to continuously diversify that segment of interest without diversifying the rest of the genome. Such continuous in vivo diversification strategies could be used for continuous evolution of traits of interest of cellular barcoding applications. The use of dsDNA-specific deaminase as opposed to ssDNA-specific deaminases would result in higher editing efficiencies in these applications. For example T7 RNA polymerases fused to ssDNA-specific deaminases have been described before, but the efficiency of editing with such designs have been limited to <1% without applying selections, likely because the ssDNA substrate (i.e. transcription bubble) that is generated transiently during transcription is buried within the polymerase and not readily accessible to ssDNA-specific deaminase (see webpage nature.com/articles/s41467-021-21876-z and internet site pubs.acs.org/doi/10.1021/jacs.8b04001). The dsDNA-specific deaminase can readily access their preferred substrate (dsDNA) as the polymerase passes along its transcriptional cassettes, thus achieving higher editing efficiencies than ssDNA-specific deaminase that could only act on the exposed ssNDA, a feature that is desirable for continuous in vivo evolution and cellular barcoding applications.

In some forms, the cells are in an animal. Thus, in some forms, the deaminase domain is administered to the animal to bring it into contact with the copies of a target nucleic acid.

In some forms when the copies of the target nucleic acid are in cells, the deaminase domain is encoded by an expression vector in the cells. Thus, in some forms, expressing the deaminase domain in the cells (e.g., transiently) results in bringing the deaminase domain into contact with the copies of a target nucleic acid.

In an exemplary method, dsDNA of interest (e.g., a gene encoding a protein of interest) is treated with the dsDNA-specific deaminase to create a library of variants of the gene of interest which can then be subjected to various directed evolution strategies (e.g., ribosome display) or other selection/screening-based methods. As set forth in the Examples, C-to-T editing was observed at the upstream of the gRNA binding site, demonstrating successful targeted editing in the defined target region.

### IV. Kits

The disclosed reagents, materials, and compositions as well as other materials can be packaged together in any suitable combination as a kit useful for performing, or aiding in the performance of, the disclosed methods. It is useful if the components in a given kit are designed and adapted for use together in the disclosed method.

In some forms, the kits can include, for example, one or more nucleic acid constructs including a nucleotide sequence encoding a deaminase domain or a base editor.

The kit may include expression vectors including such polynucleotides. In other forms, the kits may include a deaminase protein or base editor thereof in a suitable buffer. The kits can additionally or alternatively include cells expressing a deaminase domain or base editor thereof.

In some forms, the kits include reagents for performing deamination assays and/or analyzing gene expression. For example, the kits can include PCR reagents, sequencing reagents, flow cytometry reagents, primers, and combinations thereof. Preferably, the kits include instructional materials. The instructional material can include a publication, a recording, a diagram, or any other medium of expression which can be used to communicate the usefulness of the compositions and methods of the kit. For example, the instructional material may provide instructions for methods using the kit components, such as performing targeted nucleic acid editing in vitro or in vivo.

### V. Methods for Identifying and Characterizing Deaminase Enzyme Domains

Methods for identifying deaminase domains that are active on double stranded DNA (dsDNA) and determining their editing context specificity are also described. The methods systematically characterize deaminase domains available in the genomics and metagenomics databases. In some forms, the methods include one or more steps to identify one or more representative deaminase domains from one or more of the deaminase protein family. In some forms, the methods identify deaminase domains in the Cytidine deaminase-like (CDA) superfamily within one or more genomics and metagenomics databases. Exemplary genomics and metagenomics databases include the internet resource pfam database, available on the world-wide web a//pfam.xfam.org/clan/CDA. The protein functions in the pfam database are generally annotated computationally. The gene domains that are identified in the database(s) are synthesized, for example, using commercially available gene synthesizing services.

The methods include one or more steps to express the genes, for example, using an in vitro transcription/translation system. The methods include steps to characterize the activity of the synthesized, expressed deaminase domains. Typically, the methods include one or more steps to characterize the deaminases, for example, to determine their strand-bias and sequence specificity function on ssDNA and dsDNA substrates using one or more assays. Exemplary assays include DNA sequencing, and/or deamination assays.

Exemplary sequencing assays include (i) expressing a given CDA domain by in vitro translation; (ii) adding a dsDNA plasmid to the in vitro translation reaction; followed by (iii) incubation for a period of time under suitable conditions for deaminase activity; and (iv) sequence analysis of the resulting DNA product to determine deaminase activity.

Exemplary conditions include: incubation at 37 C temperature for two hour; inactivating the reaction by briefly heating to 95 C; amplification of residual DNA product, for example, by PCR; and sequencing to identify DNA integrity. Exemplary sequencing techniques include Next-Generation-Sequencing (NGS) and Sanger sequencing. In some forms, where the methods identify active deaminase domains, the methods include one or more steps to identify analogous deaminase domains in genetically-associated subfamilies of protein genes within the same or different genomics and metagenomics databases. For example, in some forms, the methods repeat the screen in subfamilies that were found to contain active dsDNA-specific CDAs in the first screen which led to identification of one or more dsCDAs. The method also includes identifying signature motifs that are present in the identified dsCDAs and absent in the non-active dsCDAs. These signature motifs can be used to identify additional dsDNA in databases.

Similar approach could be used to quickly characterize other RNA and DNA modifying/processing enzymes from genomic and metagenomic databases.

The disclosed compositions and methods can be further understood through the following numbered paragraphs.

1. An isolated deaminase domain, wherein the deaminase domain can deaminate double-stranded DNA, wherein the deaminase domain has greater deaminase activity on double-stranded DNA comprising a target nucleotide sequence as compared to the deaminase activity of the deaminase domain on double-stranded DNA that does not comprise the target nucleotide sequence,
wherein the target nucleotides are each individually fully or partially defined and are in a fixed sequential relationship to each other, and
wherein the deaminase domain is not the deaminase domain of DddA from Burkholderia cenocepacia.

2. The deaminase domain of paragraph 1, wherein the target nucleotide sequence comprises two or more target nucleotides,
wherein the target nucleotides are each individually fully or partially defined and are in a fixed sequential relationship to each other.

3. The deaminase domain of paragraph 1 or 2, wherein the target nucleotides are GC, AC, or CC.

4. The deaminase domain of any one of paragraphs 1-3, wherein the deaminase domain comprises two portions,
wherein the deaminase domain is only capable of deaminating when the two portions are combined together.

5. The deaminase domain of any one of paragraphs 1-4, wherein the deaminase domain can deaminate cytosine nucleotides.

6. The deaminase domain of one of paragraphs 1-5, wherein the target nucleotide sequence is AC.

7. The deaminase domain of one of paragraphs 1-5, wherein the target nucleotide sequence is CC.

8. The deaminase domain of one of paragraphs 1-5, wherein the target nucleotide sequence is GC.

9. The deaminase domain of paragraph 1 or 4, wherein the target nucleotide sequence is TC.

10. The deaminase domain of any one of paragraphs 1-9, wherein deaminase domain comprises an amino acid sequence of any one of SEQ ID NOs:1-4, 9, 11, 14-16, or 40-67, or a fragment or variant thereof.

11. The deaminase domain of paragraph 10, wherein the deaminase domain comprises BE_R1_41, having an amino acid sequence of SEQ ID NO:4, or an amino acid having at least 70%, 75%, 80%, 85%, 90%, 95%, or 99% sequence identity to SEQ ID NO:4, or fragment thereof.

12. The deaminase domain of paragraph 11, wherein the deaminase domain comprises BE_R1_11, having an amino acid sequence of SEQ ID NO:1, or an amino acid having at least 70%, 75%, 80%, 85%, 90%, 95%, or 99% sequence identity to SEQ ID NO:1, or fragment thereof.

13. The deaminase domain of paragraph 11, wherein the deaminase domain comprises BE_R1_12, having an amino acid sequence of SEQ ID NO:2, or an amino acid having at least 70%, 75%, 80%, 85%, 90%, 95%, or 99% sequence identity to SEQ ID NO:2, or fragment thereof.

14. The deaminase domain of paragraph 11, wherein the deaminase domain comprises BE_R1_28, having an amino acid sequence of SEQ ID NO:3, or an amino acid having at least 70%, 75%, 80%, 85%, 90%, 95%, or 99% sequence identity to SEQ ID NO:3, or fragment thereof.

15. A targeted base editor comprising the deaminase domain of any one of paragraphs 1-14 and a targeting domain, wherein the targeting domain specifically binds to a base editor target sequence.

16. The targeted base editor of paragraph 15, wherein the targeting domain comprises a TALE, BAT, CRISPR-Cas9, Cfp1, or Zinc finger.

17. The targeted base editor of paragraph 15 or 16, wherein the base editor target sequence is selected to be present in a target nucleic acid within 20 nucleotides of an instance of the target nucleotide sequence of the deaminase domain, wherein the instance of the target nucleotide sequence is selected to be base edited by the targeted base editor.

18. The targeted base editor of paragraph 17, wherein the base editor target sequence within 20 nucleotides of the instance of the target nucleotide sequence selected to be base edited by the targeted base editor is the only base editor target sequence in the target nucleic acid that is within 20 nucleotides of any instance of target nucleotide sequence.

19. The targeted base editor of paragraph 17 or 18, wherein the instance of the target nucleotide sequence in the target nucleic acid is the only instance of the target nucleotide sequence of the deaminase domain within 20 nucleotides of the base editor target sequence in the target nucleic acid within 20 nucleotides of the instance of the target nucleotide sequence.

20. The targeted base editor of any one of paragraphs 15-19, wherein the base editor target sequence is present in a mitochondrial DNA, or a chloroplast DNA, or plastid DNA.

21. The targeted base editor of any one of paragraphs 15-20, wherein the base editor comprises two portions,
wherein the first portion includes a first split deaminase domain, and wherein the second portion comprises a second split deaminase domain.

22. The targeted base editor of paragraph 21, wherein the first portion comprises a split deaminase domain comprising an amino acid sequence of any one of SEQ ID NOs:122-181, and
wherein the second portion comprises a split deaminase domain comprising an amino acid sequence of any one of SEQ ID Nos:127-181, and
wherein the first and second split deaminase domains are inactive alone but are capable of deamination when brought into proximity together.

23. The targeted base editor of any one of paragraphs 21-22, wherein the first split deaminase domain comprises an amino acid sequence of any one of SEQ ID Nos:122-126.

24. The targeted base editor of any one of paragraphs 21-22, wherein both the first and second split deaminase domains comprises a wild-type deaminase domain active site.

25. The targeted base editor of any one of paragraphs 21-24, wherein the first and second split deaminase domains each comprise a fragment or variant of BE_R1_11.

26. The targeted base editor of paragraph 25, wherein the first split deaminase domain comprises any one of SEQ ID NOs:122, or 127-135, or 150, and wherein the second split deaminase domain comprises any one of SEQ ID NOs:127-135 or 150.

27. The targeted base editor of paragraph 25, wherein the first split deaminase domain comprises SEQ ID NO:122, and
wherein the second split deaminase domain comprises any one of SEQ ID NOs:127-134 or 150.

28. The targeted base editor of paragraph 25, wherein the first split deaminase domain comprises SEQ ID NO:129, and
wherein the second split deaminase domain comprises SEQ ID NO:150.

29. The targeted base editor of any one of paragraphs 21 to 24, wherein the first and second split deaminase domains each comprise a fragment or variant of BE_R1_12.

30. The targeted base editor of paragraph 29, wherein the first split deaminase domain comprises any one of SEQ ID NOs:124, or 136-140, or 156-167, and
wherein the second split deaminase domain comprises any one of SEQ ID NOs:136-140, or 156-167.

31. The targeted base editor of paragraph 29 or 30, wherein the first split deaminase domain comprises SEQ ID NO:124, and wherein the second split deaminase domain comprises any one of SEQ ID NOs:156-166

32. The targeted base editor of paragraph 29 or 30, wherein the first split deaminase domain comprises SEQ ID NO:137, and
wherein the second split deaminase domain comprises SEQ ID NO:142.

33. The targeted base editor of paragraph 29 or 30, wherein the first split deaminase domain comprises SEQ ID NO:139, and
wherein the second split deaminase domain comprises SEQ ID NO:144.

34. The targeted base editor of paragraph 22, wherein the first and second split deaminase domains each comprise a fragment or variant of BE_R1_41.

35. The targeted base editor of paragraph 34, wherein the first split deaminase domain comprises any one of SEQ ID NOs:168-171, and
wherein the second split deaminase domain comprises any one of SEQ ID Nos: 172-175.

36. The targeted base editor of any one of paragraphs 34-35, wherein the first split deaminase domain comprises SEQ ID NO:168, and
wherein the second split deaminase domain comprises SEQ ID NO:173

37. The targeted base editor of paragraph 34-35, wherein the first split deaminase domain comprises SEQ ID NO:171, and
wherein the second split deaminase domain comprises SEQ ID NO:175.

38. The targeted base editor of paragraph 34, wherein the first split deaminase domain comprises SEQ ID NO:171, and
wherein the second split deaminase domain comprises SEQ ID NO:173.

39. The targeted base editor of any one of paragraphs 21 to 24, wherein the first and second split deaminase domains each comprise a fragment or variant of BE_R1_28.

40. The targeted base editor of paragraph 39, wherein the first split deaminase domain comprises any one of SEQ ID NOs:123, or 146-149, or 151-155, and
wherein the second split deaminase domain comprises any one of SEQ ID NOs:146-149, or 151-155.

41. The targeted base editor of paragraph 39 or 40, wherein the first split deaminase domain comprises SEQ ID NO:123, and
wherein the second split deaminase domain comprises any one of SEQ ID NOs:149, or 151-153.

42. The targeted base editor of any one of paragraphs 21 to 24, wherein the first and second split deaminase domains each comprise a fragment or variant of BE_R4_21.

43. The targeted base editor of paragraph 42, wherein the first split deaminase domain comprises any one of SEQ ID NOs:125, or 176-177, and
wherein the second split deaminase domain comprises any one of SEQ ID NOs:176-177.

44. The targeted base editor of paragraph 42, wherein the first split deaminase domain comprises SEQ ID NO:125, and
wherein the second split deaminase domain comprises SEQ ID NO:177.

45. The targeted base editor of paragraph 42, wherein the first split deaminase domain comprises SEQ ID NO:176, and
wherein the second split deaminase domain comprises SEQ ID NO:177.

46. The targeted base editor of any one of paragraphs 21 to 24, wherein the first and second split deaminase domains each comprise a fragment or variant of BE_R2_11.

47. The targeted base editor of paragraph 46, wherein the first split deaminase domain comprises any one of SEQ ID NOs:126, or 180-181, and
wherein the second split deaminase domain comprises any one of SEQ ID NOs:180-181.

48. The targeted base editor of paragraph 42, wherein the first split deaminase domain comprises SEQ ID NO:125, and
wherein the second split deaminase domain comprises any one of SEQ ID NOs:180-181.

49. The targeted base editor of paragraph 42, wherein the first split deaminase domain comprises SEQ ID NO:180, and
wherein the second split deaminase domain comprises SEQ ID NO:181.

50. The targeted base editor of any one of paragraphs 22 to 49, wherein the first, or the second portion, or both the first and second portions comprises a programmable DNA binding domain selected from the group consisting of a TALE, BAT, CRISPR-Cas9, Cfp1, or Zinc finger.

51. The targeted base editor of paragraph 50, wherein one programmable DNA binding domain is a TALE selected from the group consisting of a Left hand side TALE and a Right hand side TALE.

52. The targeted base editor of paragraph 50 or 51, wherein one programmable DNA binding domain is a Left hand side TALE comprising an amino acid sequence of any one of SEQ ID NOs:90, 92, 95, 97-106.

53. The targeted base editor of any one of paragraphs 50-52, wherein one programmable DNA binding domain is a Right hand side TALE comprising an amino acid sequence of any one of SEQ ID NOs:91, 93-94, 96, 108-113.

54. The targeted base editor of any one of paragraphs 50-53, wherein one or more programmable DNA binding domain is TALE that binds to mitochondrial mND1 DNA, having an amino acid sequence comprising any one of SEQ ID NOS:95-96.

55. The targeted base editor of any one of paragraphs 50-54, wherein one programmable DNA binding domain is a Right hand side TALE that binds to mitochondrial mND1 DNA, having an amino acid sequence comprising SEQ ID NO:96.

56. The targeted base editor of any one of paragraphs 54 or 55, wherein one programmable DNA binding domain is a Left hand side TALE that binds to mitochondrial hND1 DNA, having an amino acid sequence comprising SEQ ID NO:95.

57. The targeted base editor of paragraph 51, wherein one or more programmable DNA binding domain is TALE that binds to mitochondrial mCOX1 DNA, having an amino acid sequence comprising any one of SEQ ID NOs:99-106, or 108-113.

58. The targeted base editor of paragraph 57, wherein one programmable DNA binding domain is a Right hand side TALE that binds to mitochondrial mCOX1 DNA, having an amino acid sequence comprising any one of SEQ ID NOs:108-113.

59. The targeted base editor of any one of paragraphs 57 or 58, wherein one programmable DNA binding domain is a Left hand side TALE that binds to mitochondrial mCOX1 DNA, having an amino acid sequence comprising any one of SEQ ID NOs:90-106.

60. The targeted base editor of paragraph 50, wherein one or more programmable DNA binding domain is TALE that binds to h12 DNA, having an amino acid sequence comprising SEQ ID NO:98

61. The targeted base editor of paragraph 50, wherein one programmable DNA binding domain is a TALE with NT(G)N-terminal domain, having an amino acid sequence comprising SEQ ID NO:114.

62. The targeted base editor of any one of paragraphs 50, wherein one programmable DNA binding domain is a TALE with NT(bn)N-terminal domain, having an amino acid sequence comprising SEQ ID NO:115.

63. The targeted base editor of paragraph 51, wherein one or more programmable DNA binding domain is TALE that binds to the mitochondrial ND6 DNA, having an amino acid sequence comprising any one of SEQ ID NOs:92-94.

64. The targeted base editor of paragraph 63, wherein one programmable DNA binding domain is a Right hand side TALE that binds to the mitochondrial ND6 DNA, having an amino acid sequence comprising any one of SEQ ID NOs:93-94.

65. The targeted base editor of any one of paragraphs 63 or 64, wherein one programmable DNA binding domain is a Left hand side TALE that binds to mitochondrial mND6 DNA, having an amino acid sequence comprising SEQ ID NO:92.

66. The targeted base editor of paragraph 51, wherein one or more programmable DNA binding domain is TALE that binds to mitochondrial hND DNA, having an amino acid sequence comprising any one of SEQ ID NOs:90-91.

67. The targeted base editor of paragraph 66, wherein one programmable DNA binding domain is a Right hand side TALE that binds to mitochondrial hND DNA, having an amino acid sequence comprising SEQ ID NO:90.

68. The targeted base editor of any one of paragraphs 66 or 67, wherein one programmable DNA binding domain is a Left hand side TALE that binds to mitochondrial hND DNA, having an amino acid sequence comprising SEQ ID NO:91.

69. The targeted base editor of paragraph 50, wherein one programmable DNA binding domain is a TALE that binds to h11 DNA, having an amino acid sequence comprising SEQ ID NO:97.

70. The targeted base editor of any one of paragraphs 50-69, wherein one or both of the first and second portions independently comprise a zinc finger programmable DNA binding domain.

71. The targeted base editor of any one of paragraphs 50-70, wherein one programmable DNA binding domain is a zinc finger selected from the group consisting of a Left hand side zinc finger and a Right hand side zinc finger.

72. The targeted base editor of any one of paragraphs 50 or 57 or 70-71, wherein one programmable DNA binding domain is a zinc finger that binds to mCOX1 DNA, having an amino acid sequence comprising any one of SEQ ID NOs:82-89.

73. The targeted base editor of any one of paragraphs 50, or 70-72, wherein one programmable DNA binding domain is a Right hand side zinc finger that binds to mCOX1 DNA, having an amino acid sequence of any one of SEQ ID NOS:82-86, or 87-89.

74. The targeted base editor of any one of paragraphs 50 or 70-73, wherein one programmable DNA binding domain is a Left hand side zinc finger that binds to mCOX1 DNA, having an amino acid sequence comprising any one of SEQ ID NOs:82-86.

75. The targeted base editor of paragraphs 50, or 66, or 70-71, wherein one programmable DNA binding domain is a zinc finger that binds to hND DNA, having an amino acid sequence comprising any one of SEQ ID NOs:74-81.

76. The targeted base editor of any one of paragraphs 50 or 70 or 74-75, wherein one programmable DNA binding domain is a Right hand side zinc finger that binds to hND DNA, having an amino acid sequence of any one of SEQ ID NOs:78-81.

77. The targeted base editor of any one of paragraphs 50 or 70, or 74-76, wherein one programmable DNA binding domain is a Left hand side zinc finger that binds to hND DNA, having an amino acid sequence comprising any one of SEQ ID NOs:74-77.

78. The targeted base editor of any one of paragraphs 50-77, wherein one or both of the first and second portions independently comprise a BAT programmable DNA binding domain.

79. The targeted base editor of paragraph 50-78, wherein one programmable DNA binding domain is a BAT selected from the group consisting of a Left hand side BAT and a Right hand side BAT.

80. The targeted base editor of any one of paragraphs 50 or 57 or 72, wherein one programmable DNA binding domain is a BAT that binds to mCOX1 DNA, having an amino acid sequence comprising any one of SEQ ID NOs:118-119.

81. The targeted base editor of any one of paragraphs 50, or 57, or 70, or 72, or 80, wherein one programmable DNA binding domain is a Right hand side BAT that binds to mCOX1 DNA, having an amino acid sequence of any one of SEQ ID NO:119.

82. The targeted base editor of any one of paragraphs 50, or 57, or 70, or 72, or 80-81 wherein one programmable DNA binding domain is a Left hand side BAT that binds to mCOX1 DNA, having an amino acid sequence comprising any one of SEQ ID NO:118.

83. The targeted base editor of paragraphs 50, or 70, or 63, or, 78-79 wherein one programmable DNA binding domain is a BAT that binds to ND6 DNA, having an amino acid sequence comprising any one of SEQ ID NOs:120-121.

84. The targeted base editor of any one of paragraphs 50, or 70, or 63, or, 78-79, or 83, wherein one programmable DNA binding domain is a Right hand side BAT that binds to hND DNA, having an amino acid sequence of any one of SEQ ID NO:121.

85. The targeted base editor of any one of paragraphs 50, or 70, or 63, or, 78-79, or 83-84, wherein one programmable DNA binding domain is a Left hand side BAT that binds to hND DNA, having an amino acid sequence comprising any one of SEQ ID NO:120.

86. The targeted base editor of any one of paragraphs 21-22, wherein the first portion comprises
(a) a first split deaminase domain comprising an amino acid sequence of SEQ ID NO:120, and
(b) a Left hand TALE programmable DNA binding domain; and wherein the second portion comprises
(c) a second split deaminase domain comprising an amino acid sequence of any one of SEQ ID NOs:156, 158, 160 or 164, and
(d) a Right hand TALE programmable DNA binding domain.

87. The targeted base editor of any one of paragraphs 21-22, wherein the first portion comprises
(a) a first split deaminase domain comprising an amino acid sequence of SEQ ID NO:169, and
(b) a Left hand TALE programmable DNA binding domain; and wherein the second portion comprises
(c) a second split deaminase domain comprising an amino acid sequence of any one of SEQ ID NOs:173, or 175, and
(d) a Right hand TALE programmable DNA binding domain.

88. The targeted base editor of any one of paragraphs 21-22, wherein the first portion comprises
(a) a first split deaminase domain comprising an amino acid sequence of SEQ ID NO:171, and
(b) a Left hand TALE programmable DNA binding domain; and
   wherein the second portion comprises
(c) a second split deaminase domain comprising an amino acid sequence of any one of SEQ ID NO:175, and
(d) a Right hand TALE programmable DNA binding domain.

89. The targeted base editor of any one of paragraphs 21-22, wherein the first portion comprises
(a) a first split deaminase domain comprising an amino acid sequence of SEQ ID NO:169, and
(b) a Left hand BAT programmable DNA binding domain; and
   wherein the second portion comprises
(c) a second split deaminase domain comprising an amino acid sequence of any one of SEQ ID NOs:173, or 175, and
(d) a Right hand TALE programmable DNA binding domain.

90. The targeted base editor of any one of paragraphs 21-22, wherein the first portion comprises
(a) a first split deaminase domain comprising an amino acid sequence of SEQ ID NO:169, and
(b) a first coiled coil domain, and
(c) optionally a Left hand TALE programmable DNA binding domain; and
   wherein the second portion comprises
(d) a second split deaminase domain comprising an amino acid sequence of any one of SEQ ID NOs:173, or 175, and
(e) a second coiled coil domain, and
(f) optionally a Right hand TALE programmable DNA binding domain;
   wherein the first and second coiled coil domains interact together upon combination of the first and second portions.

91. The targeted base editor of any one of paragraphs 22-91, wherein one or both of the first and second portions comprises at least one linker.

92. The targeted base editor of any one of paragraphs 50-90, wherein one or both of the first and second portions comprises at least one linker, and
wherein the linker is positioned between the programmable DNA binding domain and the split deaminase domain.

93. The targeted base editor of any one of paragraph 92, wherein both of the first and second portions comprise a linker between the programmable DNA binding domain and the split deaminase domain.

94. The targeted base editor of any one of any one of paragraphs 91-93, wherein the linker is between 2 and 200 amino acids in length.

95. The targeted base editor of paragraphs 94, wherein the linker is between 2 and 16 amino acids in length.

96. The targeted base editor of any one of paragraph 91-95, wherein the linker comprises an amino acid sequence of any of GS, GSG, GSS, or SEQ ID NOS:23-27 or 30.

97. The targeted base editor of any one of paragraphs 50-96, wherein the base editor is configured such that the target nucleic acid is between 9 and 11 base pairs from a programmable binding domain binding site on a target DNA strand.

98. The targeted base editor of any one of paragraphs 50-97, wherein the distance between two binding sites of two programmable binding domains on a target DNA strand is between 12 and 22 base pairs.

99. The targeted base editor of paragraph 98, wherein the distance between two binding sites of two programmable binding domains on a target DNA strand is between 14 and 19 base pairs.

100. The targeted base editor of any one of paragraphs 22-99, wherein at least one of the first and second portions comprises a cellular targeting moiety.

101. The targeted base editor of paragraph 100, wherein both of the first and second portions comprises a cellular targeting moiety.

102. The targeted base editor of paragraph 101, wherein both of the first and second portions comprise the same cellular targeting moiety.

103. The targeted base editor of any one of paragraphs 100-102, wherein cellular targeting moiety is selected from the group consisting of a mitochondrial targeting sequence (MTS), and a nuclear localization sequence (NLS).

104. The targeted base editor of paragraph 103, wherein the NLS comprises an amino acid sequence of any one of SEQ ID NOs:34-39.

105. The targeted base editor of paragraph 104, wherein the MTS comprises an amino acid sequence of any one of SEQ ID NOs:22, 69, 71, 182 or 183.

106. The targeted base editor of any one of paragraphs 22-105, wherein at least one of the first and second portions comprises a base excision repair inhibitor.

107. The targeted base editor of paragraph 106, wherein the base excision repair inhibitor is a mammalian DNA glycosylase inhibitor.

108. The targeted base editor of paragraph 106 or 107, wherein the base excision repair inhibitor is a uracil glycosylase inhibitor.

109. The targeted base editor of any one of paragraphs 106-108, wherein the base excision repair inhibitor has an amino acid sequence comprising any one of SEQ ID NO:21 or 70.

110. A method comprising
bringing into contact a target nucleic acid and a targeted base editor of any one of paragraphs 17-109, wherein the target nucleic acid is double-stranded DNA, whereby the instance of the target nucleotide sequence is deaminated by the targeted base editor.

111. The method of paragraph 110, wherein the deaminated nucleotide in the target nucleotide sequence is converted to a thymine or a guanine nucleotide, wherein the conversion completes a base edit of the target nucleotide sequence.

112. The method of paragraph 110 or 111, wherein the target nucleic acid is mitochondrial DNA.

113. The method of any one of paragraphs 110-112, wherein the target nucleotide sequence is AC.

114. The method of any one of paragraphs 110-112, wherein the target nucleotide sequence is CC.

115. The method of any one of paragraphs 110-112, wherein the target nucleotide sequence is GC.

116. The method of any one of paragraphs 110-112, wherein the target nucleotide sequence is TC.

117. The method of any one of paragraphs 110-116, wherein the last C in the target nucleotide sequence is deaminated by the targeted base editor.

118. The method of any one of paragraphs 110-117, wherein the instance of the target nucleotide sequence in the target DNA is within 20 nucleotides of the base editor target sequence.

119. The method of any one of paragraphs 110-118, wherein the target nucleic acid is in a cell, wherein bringing into contact the target nucleic acid and the targeted base editor is accomplished by facilitating entry of the targeted base editor into the cell.

120. The method of paragraph 119, wherein the cell is in an animal, wherein bringing into contact the target nucleic acid and the targeted base editor is accomplished by administering the targeted base editor to the animal.

121. A method comprising:
bringing into contact a target nucleic acid and one or more deaminase domain, wherein the target nucleic acid is double-stranded cytosine-methylated DNA, wherein the deaminase domain can deaminate double-stranded DNA, wherein the deaminase domain deaminates substantially only non-methylated cytosine nucleotides in the target nucleic acid,
wherein substantially all of the non-methylated cytosine nucleotides in the target nucleic acid are deaminated by the deaminase domain; and
sequencing the deaminated target nucleic acid, whereby methylated cytosine nucleotides in the target nucleic acid are identified.

122. The method of paragraph 121, wherein the deaminase domain deaminates 90% or more of the non-methylated cytosine nucleotides in the target nucleic acid.

123. A method comprising:
bringing into contact a deaminase domain and a plurality of copies of a target nucleic acid for a time and under conditions that results in deamination of an average of 0.1 to 5.0 nucleotides per copy of the target nucleic acid,
wherein the target nucleic acid is double-stranded DNA, wherein the deaminase domain can deaminate double-stranded DNA.

124. The method of paragraph 123, wherein the copies of the target nucleic acid are in vitro.

125. The method of paragraph 124, wherein the deaminated nucleotides in the copies of the target nucleic acid are converted to a thymine or a guanine nucleotide via an in vitro reaction.

126. The method any one of paragraphs 121-125 further comprising subjecting the deaminated copies of the target nucleic acid to a selection procedure.

127. The method of paragraph 126, wherein the selection procedure comprises mRNA display, ribosome display, or SELEX, or cell-based selection assays.

128. The method of any one of paragraphs 125-127, wherein the deaminated nucleotides in the copies of the target nucleic acid are converted to a thymine or a guanine nucleotide, wherein the conversion completes one or more base edits of some or all of the copies of target nucleic acid.

129. The method of paragraph 123, wherein the deaminated nucleotides in the copies of the target nucleic acid are converted to a thymine or a guanine nucleotide by incubating the copies of the target nucleic acid in cells followed by a DNA replication/amplification step.

130. The method of paragraph 123, wherein the copies of the target nucleic acid are in cells, wherein bringing into contact the deaminase domain and the copies of a target nucleic acid is accomplished by facilitating entry of the deaminase domain into the cells.

131. The method of paragraph 130, wherein the cells are in an animal, wherein bringing into contact the deaminase domain and the copies of a target nucleic acid is accomplished by administering the deaminase domain to the animal.

132. The method of paragraph 130, wherein the copies of the target nucleic acid are in cells, wherein the deaminase domain is encoded by a transgenic expression construct in the cells, wherein bringing into contact the deaminase domain and the copies of a target nucleic acid is accomplished by transiently expressing the deaminase domain in the cells.

133. A method of treating or preventing a mitochondrial genetic disease in a subject by editing one or more nucleic acids in mitochondrial DNA in a cell of the subject, comprising
introducing to the cell the targeted cytosine deaminase base editor of any one of paragraphs 1-110,
wherein a target nucleic acid within mitochondrial DNA is deaminated by the targeted base editor.

134. The method of paragraph 133, wherein the deaminated nucleotide in the target nucleotide sequence is converted to a thymine or a guanine nucleotide.

135. The method of any one of paragraphs 133-134, wherein one or more nucleic acids in the mitochondrial DNA is edited to a non-pathogenic form.

136. The method of any one of paragraphs 133-135, wherein the deaminated nucleotide is at a position selected from m.583G>A, m.616T>C, m.1606G>A, m.1644G>A, m.3258T>C, m.3271T>C, m.3460G>A, m.4298G>A, m.5728T>C, m.5650G>A, m.3243A>G, m.8344A>G, m.14459G>A, m.11778G>A, m.14484T>C, m.8993T>C, m.14484T>C, m.3460G>A, and m.1555A>G.

137. The method of any one of paragraphs 133-136, wherein the cell is selected from the group consisting of a fibroblast, lymphocyte, pancreatic cell, muscle cell, neuronal cell, and a stem cell.

138. A vector comprising or expressing the targeted base editor of any one of paragraphs 22-110.

139. The vector of paragraph 138, wherein the vector is an altered adenovirus (AAV) vector, a Lentivirus vector, or a virus-like particle (VLP).

140. The vector of paragraph 138 or 139, wherein the targeted base editor is encapsulated within the vector.

141. The method of any one of paragraphs 120, or 129-137, wherein the deaminase domain comprises a targeted base editor within a vector.

142. The targeted base editor of any one of paragraphs 22 to 49, wherein the first and second portions each comprise a programmable DNA binding domain independently selected from the group consisting of a TALE, BAT, CRISPR-Cas9, Cfp1, and Zinc finger.

143. The targeted base editor of paragraph 50/142, wherein the first portion is a TALE and the second portion is a TALE, wherein the first portion is a TALE and the second portion is a BAT, wherein the first portion is a TALE and the second portion is a Zinc finger, wherein the first portion is a TALE and the second portion is a CRISPR-Cas9, wherein the first portion is a TALE and the second portion is a Cfp1, wherein the first portion is a BAT and the second portion is a TALE, wherein the first portion is a BAT and the second portion is a BAT, wherein the first portion is a BAT and the second portion is a Zinc finger, wherein the first portion is a BAT and the second portion is a CRISPR-Cas9, wherein the first portion is a BAT and the second portion is a Cfp1, wherein the first portion is a Zinc finger and the second portion is a TALE, wherein the first portion is a Zinc finger and the second portion is a BAT, wherein the first portion is a Zinc finger and the second portion is a Zinc finger, wherein the first portion is a Zinc finger and the second portion is a CRISPR-Cas9, wherein the first portion is a Zinc finger and the second portion is a Cfp1, wherein the first portion is a CRISPR-Cas9 and the second portion is a TALE, wherein the first portion is a CRISPR-Cas9 and the second portion is a BAT, wherein the first portion is a CRISPR-Cas9 and the second portion is a Zinc finger, wherein the first portion is a CRISPR-Cas9 and the second portion is a CRISPR-Cas9, wherein the first portion is a CRISPR-Cas9 and the second portion is a Cfp1, wherein the first portion is a Cfp1 and the second portion is a TALE, wherein the first portion is a Cfp1 and the second portion is a BAT, wherein the first portion is a Cfp1 and the second portion is a Zinc finger, wherein the first portion is a Cfp1 and the second portion is a CRISPR-Cas9, or wherein the first portion is a Cfp1 and the second portion is a Cfp1.

144. A method of editing one or more nucleic acids in mitochondrial DNA in a mitochondrion or chloroplast DNA in a chloroplast, comprising
introducing to the mitochondrion or the chloroplast the targeted cytosine deaminase base editor of any one of paragraphs 1-110,
wherein a target nucleic acid within mitochondrial or chloroplast DNA is deaminated by the targeted base editor.

145. The method of paragraph 144, wherein the mitochondrion or the chloroplast is in vitro.

146. The deaminase domain of paragraph 1 or 2, wherein the target nucleotides each exhibit a context specificity defined by the deaminase probability sequence logo at a defined editing threshold.

The present invention will be further understood by reference to the following non-limiting examples.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### Example 1: Generation and Identification of Cytosine Deaminase Domains Active on ssDNA and/or dsDNA

### Materials and Methods

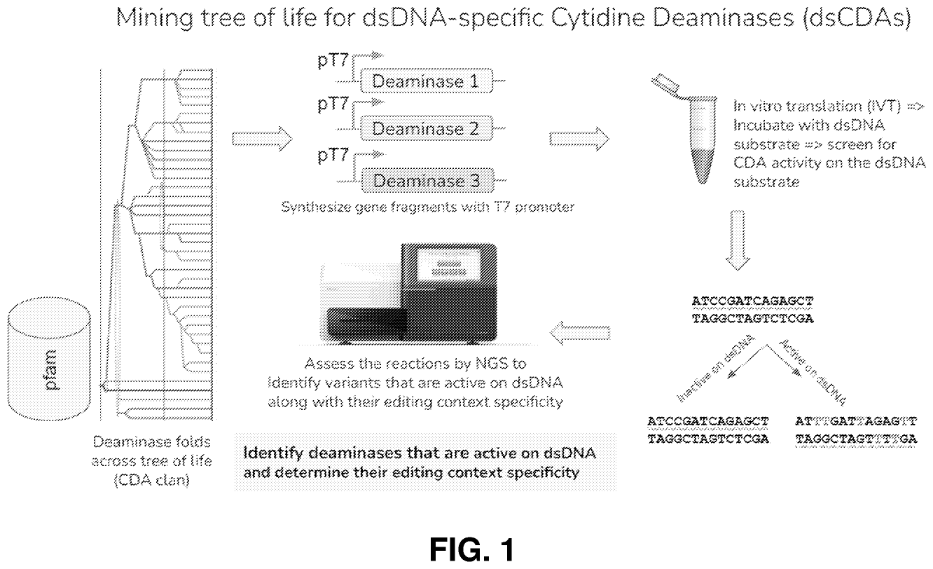

Systematic characterization of various putative deaminase domains available in the genomics and metagenomics databases was performed to assess the activity of deaminase proteins and base editors. Multiple representative domains from each deaminase protein family of the Cytidine deaminase-like (CDA) clan available on the pfam database (https://pfam.xfam.org/clan/CDA, the protein functions in this database are generally annotated computationally) were chosen. The sequences encoding these protein domains were synthesized using commercial synthesis resources and expressed using a cell-free in vitro transcription/translation system. Generally, the domains/polypeptides identified by the screen are part of natural proteins, however, only sequences corresponding to isolated deaminase domains were synthesized using the GBLOCK™ gene fragment synthesis system (IDT). A synthetic in vitro system was found to be effective to assess the activity of these enzymes, since it was found that dsDNA specific deaminases are toxic when expressed in cells as they can introduce unwanted mutations across the genome. The system enabled efficient in vitro assessment of base-editor activity which are usually assessed within the living cell context. Subsequently, the activity of the deaminase domains on ssDNA and dsDNA substrates was assessed using various assays (DNA sequencing or deamination assay) to determine their strand-bias and sequence specificity. An overview of this method is illustrated in FIG. 1.

For the sequencing assay, dsDNA plasmid was added to the in vitro translation reaction expressing a given CDA domain and incubated for two hours at 37 C. Incubating double stranded DNA substrate (e.g. plasmid or PCR amplicon) with the In Vitro Translation (IVT)-expressed protein can identify high levels of deamination (C-to-T or G-to-A) mutations that can be detected by PCR amplification (using a dU permissive polymerase such as Q5U or Kapa U+ polymerase) followed by NGS high-throughput sequencing or Sanger sequencing of the amplified DNA. Subsequently, the reaction was inactivated by briefly heating up the samples at 95 C, the substrate was PCR amplified and sequenced (either with NGS or Sanger sequencing). Additional rounds of screen (R2-R4) were performed in subfamilies that were found to contain active dsDNA-specific CDAs in the first round (MafB19 and SCP1201 deaminases) which led to identification of additional dsCDAs.

For the deaminase assay, a USER (Uracil-Specific Excision Reagent) Enzyme-based assay for deamination was employed to test the activity of various deaminase domains on the substrates. The assay works on the principle that deamination of the cytosine target residue results in conversion of the target cytosine to a uracil. The USER Enzyme excises the uracil base and cleaves the DNA backbone at that position, cutting the DNA substrate into two shorter fragments. The DNA substrate can be labeled on one end with a dye, e.g., with a FAM label. Upon deamination, excision, and cleavage of the strand, the substrate can be subjected to electrophoresis, and the substrate and any fragment released from it can be visualized by detecting the label. dsDNA substrates (A(15)XA(15)) were used as the substrates, where X is one of the sequences shown as the substrate (e.g., substrate called AC corresponds to [A(15)]AC[A(15)]).

FAM-labeled ssDNA or dsDNA substrates containing dC in various contexts were used. After incubation with the in vitro translated domain, USER enzyme was added to cleave off the deaminated substrates. The substrate cleavage was analyzed by running the reactions on denaturing TBE-Urea gels.

To systematically determine the context specificity of the identified dsDNA specific deaminases in free-floating form, activity against a synthetic substrate encoding all the possible triplet nucleotides (NNN) in the IVT system was tested and their activity read out by Illumina sequencing. Sites (corresponding to cytidines) with editing frequency >50% were identified from NGS data, and the nucleotides flanking the edited cytidines were extracted and used to make sequence logo representing the editing contexts for each deaminase. The sequence for the dsDNA substrate used in this experiment was:

(SEQ ID NO: 73)
TAATAATTATATTATTATTTTAAATTAATTATTTAACCGTGGTGCGCGG
GGTCGCCCAGCAATAGTATAGGTTGTCGAGTATGAAGGGTCTAAAAGAT
TTTAAGACACCTTACGGACGAAGAGTTTCTCTCTTAGTCCCCTGATCTG
CAGAACCCAGGATATCAAGCACATTTCACTTCACGTGTTTTGATGAAAC
TATACATCACCCGCGCCACAGGCGCTGTGCGGTTTATAATATATTATAA
TTTATATTTATATTAAATT

The substrate was appended with AT-only adapters to facilitate downstream amplification for NGS library prep.

### Results

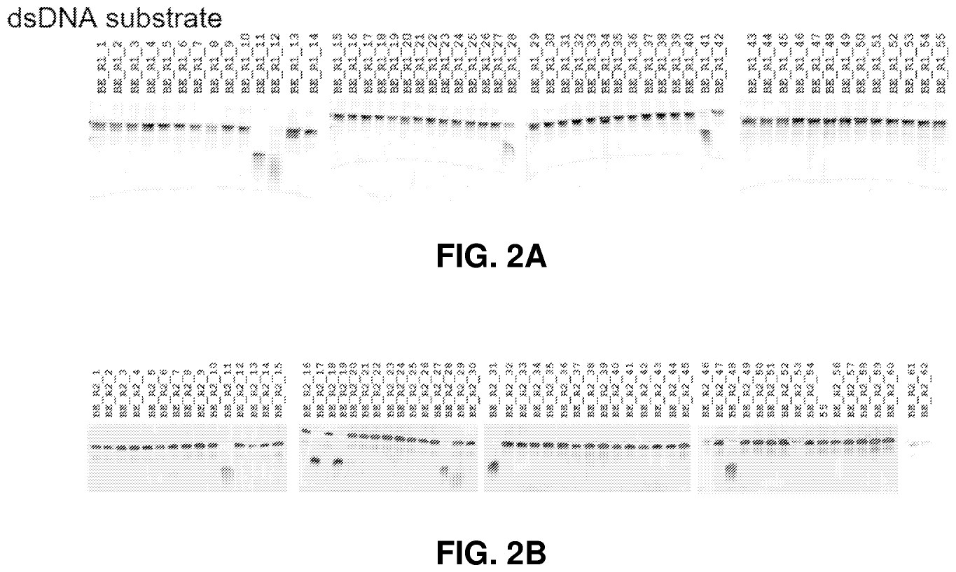

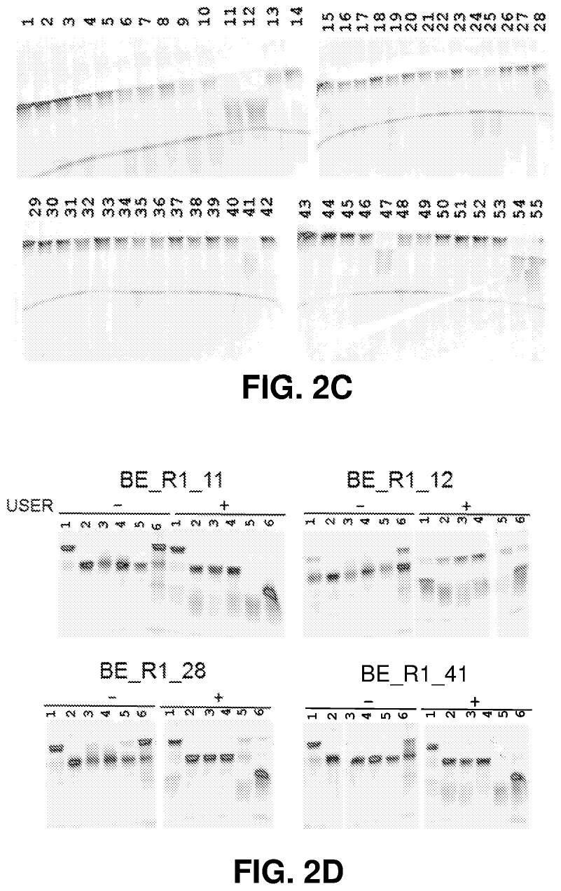

Activity of the deaminase domains on ssDNA and dsDNA was detected by a deamination assay. In a first screen, genes encoding 55 different deaminases were expressed in vitro, and their activity on ssDNA and dsDNA substrates (A(15)ACCGCTCA(15); SEQ ID NO:39) were determined (Table 3). Cleavage events observed after electrophoresis indicate activity of the specified deaminase on the indicated substrate (FIGS. 2A-2C). It was observed that the deaminases BE11 (SEQ ID NO:1), BE12 (SEQ ID NO:2), BE28 (SEQ ID NO:3), and BE41 (SEQ ID NO:4) were active on both dsDNA and ssDNA, whereas BE47 (SEQ ID NO:5), BE54 (SEQ ID NO:6), and BE55 (SEQ ID NO:7) were active on ssDNA (FIGS. 2A, 2C).

Inspired by these results, additional deaminase domains from the protein families that the above-identified active dsDNA-specific deaminases belong to (specifically MafB19-deam and SCP1201-deam families) were further screened. The second screen determined the activity of the additional deaminase domains by deaminase assay, including those high activity on dsDNA: BE_R2_18 (SEQ ID NO:11), BE_R2_27, BE_R2_29 (SEQ ID NO:14), BE_R2_31 (SEQ ID NO:15), and BE_R2_48 (SEQ ID NO:16); BE_R2_11 (SEQ ID NO:9), 19 (SEQ ID NO:45), 28 (SEQ ID NO:48), while BE_R2_7 (SEQ ID NO:8), BE_R2_17 (SEQ ID NO:10), and BE_R2_26 (SEQ ID NO:12) exhibited lower activity on dsDNA (FIG. 2B).This resulted in the identification of additional deaminase domains active on dsDNA, with showing high activity on dsDNA. Additional rounds of screens of potential dsDNA specific deaminases were performed (rounds R3 and R4). Results of biochemical characterization and sequence details for the identified domains are summarized in Table 3.

It was then investigated whether the identified dsDNA-specific deaminase domains possessed some level of sequence specificity. Different substrates containing dC in various contexts were used in the deaminase assay, including dsDNA substrates (A(15)XA(15)) were used as the substrates, where X is one of the sequences shown as the substrate (e.g., substrate called AC corresponds to [A(15)]AC[A(15)]). The dsDNA substrates used included:

1.
(SEQ ID NO: 268)
AAAAAAAAAAAAAAATGCGCCAAAAAAAAAAAAAAA
2.
(SEQ ID NO: 269)
AAAAAAAAAAAAAAAACAAAAAAAAAAAAAAA
3.
(SEQ ID NO: 270)
AAAAAAAAAAAAAAACCAAAAAAAAAAAAAAA
4.
(SEQ ID NO: 271)
AAAAAAAAAAAAAAAGCAAAAAAAAAAAAAAA
5.
(SEQ ID NO: 272)
AAAAAAAAAAAAAAATCAAAAAAAAAAAAAAA
6.
(SEQ ID NO: 273)
AAAAAAAAAAAAAAAACCCCTCAAAAAAAAAAAAAAA

The only known dsDNA-specific deaminase (dddA, a recently described deaminase from bacterial toxins) was used as a positive control.

Different deaminase domains showed different levels of activity on different substrates, indicating that the enzymes possess some level of sequence specificity (FIG. 2D). Based on these results (FIG. 2D), the following sequence specificities or preferences for the isolated deaminase were observed:
BE_R1_11: TC-specific. AC- and GC-specific to lesser extent
BE_R1_12: AC- and GC-specific. CC specific to lesser extent
BE_R1_28: TC-specific (context-specificity is more strict than BE_R1_11 and BE_R1_41)
BE_R1_41: TC-specific. AC- and CC-specific to lesser extent.

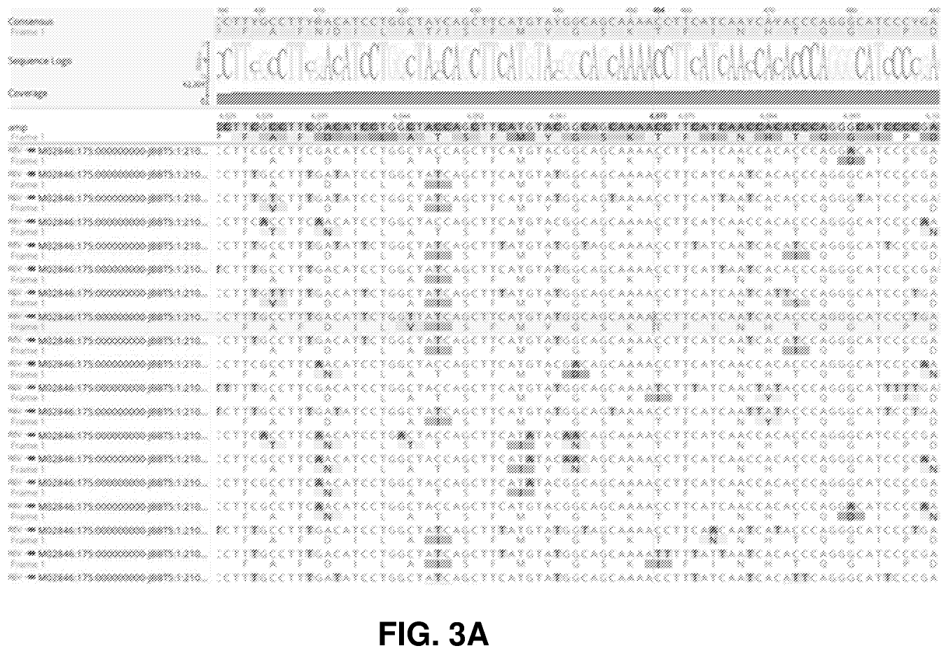

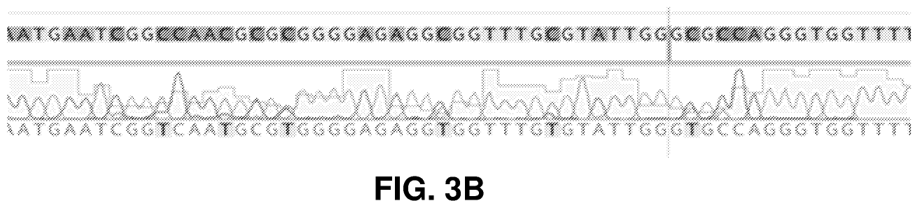

Next, DNA deamination events were assayed by sequencing. The sequencing results demonstrated that the deaminases were highly active on dsDNA, and possess some level of sequence specificity, and these enzymes deaminate dC in various contexts with various efficiencies (FIGS. 3A-3B).

The NGS data was used to determine the sequence specificity of the identified dsDNA-specific deaminases. In brief, dsDNA plasmid substrates were incubated with the in vitro translated deaminases. Subsequently, the substrate was PCR amplified and Illumina adapters and barcodes were added with a second round of PCR. SNP variants with indicated editing frequencies were identified, and a sequence frequency logo for each level of editing efficiency (25% or 50% edited sites) was determined (FIGS. 4A-4B). These results demonstrate that the identified deaminases have distinct substrate specificities and can collectively allow to edit any cytidines in any given context (NCN). Depending on the target sequence context, deaminases with more relaxed or stringent sequence specificity can be selected from the identified deaminase panel.

Due to their activity on dsDNA, the identified deaminases could be toxic when expressed in living cells if their activity is not somehow contained. In natural systems, activity of these proteins are contained at the transcriptional or translational level, or by sequestration to specific cell compartments or by co-expression of inhibitory proteins (such as the case in toxin-antitoxin systems). Splitting toxic proteins into inactive halves has been used previously to express toxic proteins such as FokI (endonuclease) and DddA (DNA deaminase). When co-expressed, the inactive halves can reconstitute the active form of the protein. By controlling the localization of the two halves, one can ensure that the fully functional form of the protein only reconstitute in a desired compartment/location (e.g., a desired DNA sequence) and off-target activity of the toxic protein on the rest of genome is minimized.

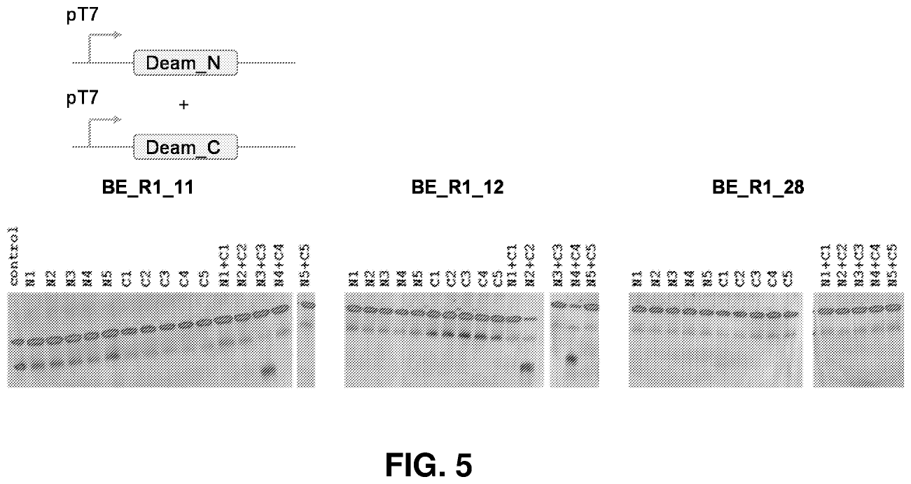

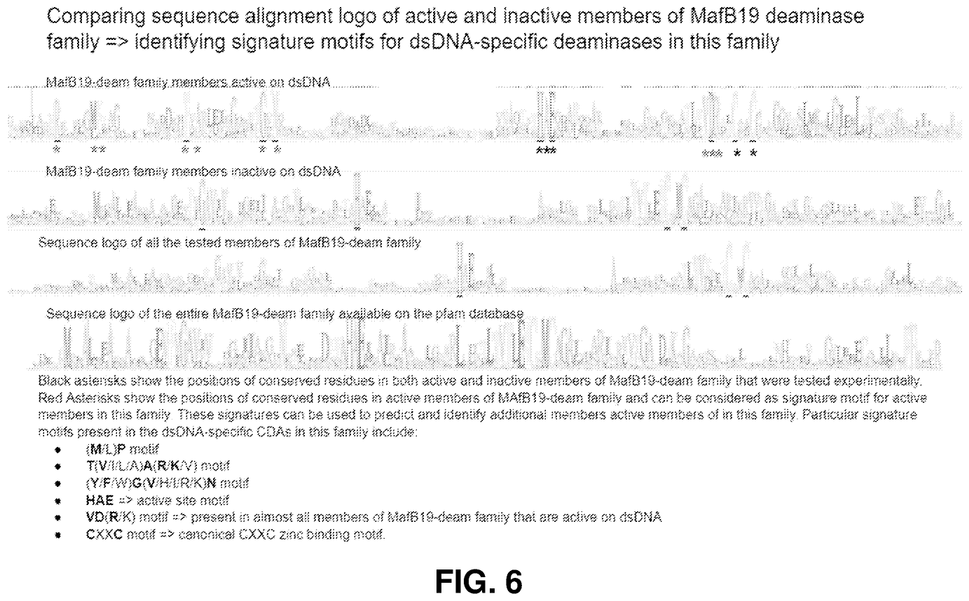

With this in mind, split versions of the identified deaminases were created in order to use them for in vivo applications without imposing toxicity to cells. The identified deaminases were split at different positions along their encoding gene (to make various N- and C-terminal halves of the proteins), and their activity (as individual halves or when complementary halves were combined) was assessed with the deaminase assay. As shown in FIG. 5, some of the split forms showed activity when mixed with their complementary halves (BE11: N3+C3, BE12: N2+C2, BE12: N4+C4).

Comparative genomics was performed across the sequences of the identified cytidine deaminase domains having dsDNA activity (also referred to as “dsCDA”).

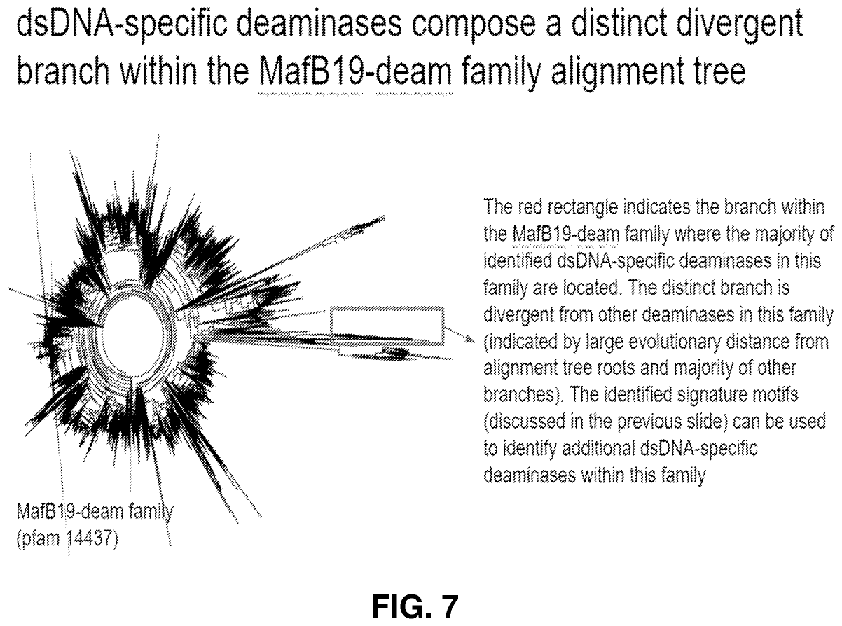

Majority of the identified deaminases belonged to the two main families (MafB19 and SCP1201) within the CDA Clan. FIG. 7A shows the sequence alignment logo and signature motifs identified for members of MafB19 family that are active on dsCDAs, those that inactive on dsDNA, as well as the entire MafB19 family.

Particular conserved residues (i.e., signature motifs) were present in the dsDNA-specific CDAs in the MafB19-deam family that tested experimentally but were absent in the non-active members of this family. These signatures can be used to predict and identify additional members active members of in this family and include:
(M/L)P motif
T(V/I/L/A)A(R/K/V) motif
(Y/F/W)G(V/H/I/R/K)N motif
HAE=>active site motif
VD(R/K) motif=>present in almost all members of MafB19-deam family that are active on dsDNA
CXXC motif=>canonical CXXC zinc binding motif.

The identified signature motifs can be used to identify additional dsDNA-specific deaminases within this family.

A branch within the MafB19-deam family where the majority of identified dsDNA-specific deaminases in this family are located was identified (FIG. 7B). The distinct branch is divergent from other deaminases in this family (indicated by large evolutionary distance from alignment tree roots and majority of other branches).

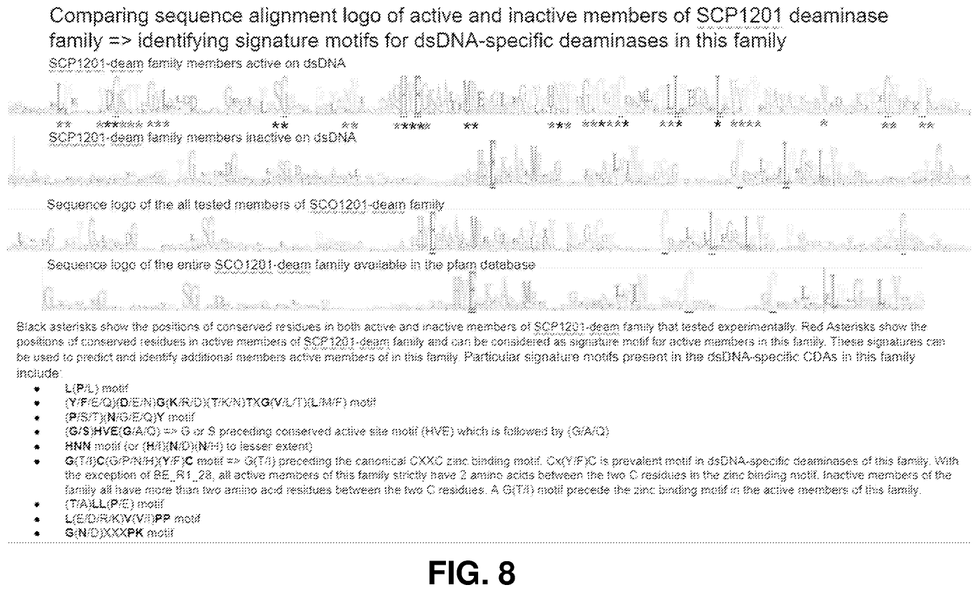

Similar analysis was performed for the SCP1201-deam protein family (FIG. 8). Particular signature motifs present in the dsDNA-specific CDAs in the SCP1201-deam family that tested experimentally include:
L(P/L) motif;
(Y/F/E/Q)(D/E/N)G(K/R/D)(T/K/N)TXG(V/L/T)(L/M/F) motif;
(P/S/T)(N/G/E/Q)Y motif;
(G/S)HVE(G/A/Q)=>G or S preceding conserved active site motif (HVE) which is followed by (G/A/Q);
HNN motif (or (H/I)(N/D)(N/H) to lesser extent);
G(T/I)C(G/P/N/H)(Y/F)C motif=>G(T/I) preceding the canonical CXXC zinc binding motif;
Cx(Y/F)C is prevalent motif in dsDNA-specific deaminases of this family. With the exception of BE_R1_28, all active members of this family strictly have 2 amino acids between the two C residues in the zinc binding motif. Inactive members of the family all have more than two amino acid residues between the two C residues. A G(T/I) motif precede the zinc binding motif in the active members of this family.
(T/A)LL(P/E) motif;
L(E/D/R/K)V(V/I)PP motif; and
G(N/D)XXXPK motif.

The identified signature motifs can be used to identify additional dsDNA-specific deaminases within each family.

To further characterize the dsDNA/deaminase interaction, predictive structural models of Deaminases bound to dsDNA were calculated.

The predicted structure of BE12 docked on dsDNA was calculated as an exemplary representative of MafB19-deam family. The positions corresponding to the signature residues for MafB19-deam family were determined. The deaminase seem to bind to dsDNA by interacting with both the minor and major grooves of DNA. The conserved/signature motifs cluster around the enzyme active site (HAE) and the DNA binding sites. The signature motifs (specially VDR and G(V/H/I/R/K)N motifs seem to stabilize the interaction of the deaminase with dsDNA. The R residue in the VDR motif directly interacts with the dsDNA backbone, and could participate in unwinding of the double-strand DNA either by a protrusion or base flipping mechanism).

The predicted structure of BE41 docked on dsDNA was also calculated as an exemplary representative of SCP1201-deam family. The positions corresponding to the signature residues for SCP1201-deam family were determined. The deaminase seem to bind to dsDNA by interacting with both the minor and major grooves of DNA. The conserved/signature motifs cluster around the enzyme active site (HAE) and the DNA binding sites. The signature motifs (specially (Y/F/E/Q)(D/E/N)G(K/Q/T)(T/K)TXG(V/L/T)(L/M/F), (P/S/T)(N/G/E/Q)Y, SG, and HNN motifs seem to stabilize the interaction of the deaminase with dsDNA).

TABLE 3
Identities and sequences for the identified dsDNA-specific CDA domains

| | Deaminase Domain Sequence extracted | | | | | Activity | | |
|---|---|---|---|---|---|---|---|---|
| Name used | from the protein indicated by | SEQ | Uniprot ID # | | Activity on ssDNA | | Activity on dsDNA | |
| in this | the corresponding Uniprot | ID | (Accession | Protein | deaminase | based on | deaminase | based on |
| study | ID# and tested experimentally | NO: | number) | Family | assay | NGS | assay | NGS |
| BE_R1_10 | MEMGTRSLPQETEYMREALKEAEKAYALGETPI | 40 | A0A3P2ALZ1_ | MafB19- | N | Y (weak | N | Y (weak |
| | GCVIVWRGEIIGRGYNRRAIDKSVLAHAEITAI | | 9FIRM | deam | | activity) | | activity) |
| | AEAERYLADWRLEEATLYVTLEPCPMCAGAIVQ | | | | | | | |
| | ARVGRVVYATANLKAGSAGTVIDMMHVAGFNHQ | | | | | | | |
| | VEVVGGILEKECTDLLKRFFRELRAEKDKPYPP | | | | | | | |
| | K | | | | | | | |
| BE_R1_11 | TKSANSGGAAKDLAKYRERQGMPRAGSADDAHT | 1 | A0A1Y5Y1M1_ | MafB19- | Y | Y | Y | Y |
| | AARLDVGGRSFYGHNAHGRNIDIKVNAQTKTHA | | KIBAR | deam | | | | |
| | EADVFQQAKNAKVSADRATLHVDRDLCDACGIK | | | | | | | |
| | GGVGSLMRGVGISRLTVNSPSGRFEITASRPSV | | | | | | | |
| | PRRING | | | | | | | |
| BE_R1_12 | FSKAESGYIEIQRFRRILNMPRYSLINGRTGTV | 2 | A0A2T4Z6L8_ | MafB19- | Y | Y | Y | Y |
| | ARVEVNGRRIFGVNTSLIKNSKYAPRDMDLRRR | | 9BACL | deam | | | | |
| | WLREVNWVPPKKNKPNHLGHAQSLSHAESHALI | | | | | | | |
| | RAYERMERLGGQLPKKLTMVVDRPTCNICRGEM | | | | | | | |
| | PALLKRLGIEELTIYSGGRDAIIIKAIK | | | | | | | |
| BE_R1_15 | EVQARLNGLAAEARQGLPPNKGNVAVAEINIPE | 41 | A0A433SEU4_ | BURPS668_ | N | Y (weak | N | Y (weak |
| | LADQPFITKAFSGYQTDKDGFVGKPSGNVDTWA | | 9BURK | 1122 | | activity) | | activity) |
| | LQPQKSSPEFIGGPGAYFRDVDTEFKILENLAQ | | | | | | | |
| | KLGPNTNATGTVNLISEKVVCPSCTTVIMQFRE | | | | | | | |
| | RYPNIQLNIFTRD | | | | | | | |
| BE_R1_21 | INYAKENGITGGRNVAVFEYIDLNGKIQTIIKA | 42 | A0A3P2A0L6_ | XOO_ | N | Y (weak | N | Y (weak |
| | SERGKGHAERLIAMELQNKGIPNSNVTRIYSEL | | 9NEIS | 2897-deam | | activity) | | activity) |
| | EPCSAPGGYCSNMIKYGSPNGLGPYSNAKVTYS | | | | | | | |
| | FSYGGNPHNAEAARQGVDALRKAREQQKR | | | | | | | |
| BE_R1_28 | GVGGAITATVGSTAGAAGRAAARAPSLPAYAGG | 3 | A0A0K1EKV1_ | SCP1201- | Y | Y | Y | Y |
| | KTSGVLRTTAGDTALLSGYKGPSASMPRGTPGM | | CHOCO | deam | | | | |
| | NGRIKSHVEAHAAAVMREQGMKEGTLYINRVPC | | | | | | | |
| | SGATGCDAMLPRMLPPDAHLRVVGPNGYDQVFV | | | | | | | |
| | GLPD | | | | | | | |
| BE_R1_41 | DPIGLMGGLNLYQYAPNSIAWTDWWGLAGSYTL | 4 | C5ALM7_ | SCP1201- | Y | Y | Y | Y |
| | GSYQISAPQLPAYNGQTVGTFYYVNGAGGLESR | | BURGB | deam | | | | |
| | TFSSGGPTPYPNYANAGHVEGQSALFMRDNGIS | | | | | | | |
| | DGLVFHNNPEGTCGFCVNMTETLLPENSKLTVV | | | | | | | |
| | PPEGAIPVKRGATGETRTFTGNSKSPKSPVKGE | | | | | | | |
| | C | | | | | | | |
| BE_R2_1 | GGTPSCSTTLDGLVPTDALEEFATRAYTQEEGA | 43 | A0A0F6W299_ | MafB19- | ND | Y (weak | N | Y (weak |
| | CSGYYVVGSANSARVEGVLTACDATTTSVGNEW | | 9DELT | deam | | activity) | | activity) |
| | REEAGTTRACQLFGWPGAIPESVEIDRARCRLA | | | | | | | |
| | EQDWARLQQRREDCGLPPRTLVPNDGHTVAILT | | | | | | | |
| | TPGEDEITGLNGRIGGAQPYRARAVEEGTCPPP | | | | | | | |
| | LTRTYGEDATRYRGAGPTHCHAEGDALEQLSVL | | | | | | | |
| | RMREPGTPGAGDPRQGATGGRTTGSAELIVDRD | | | | | | | |
| | PCAMSCAPRGVDRMRSIAGLEELIVRSPQGTRR | | | | | | | |
| | YADGLPETGVPLD | | | | | | | |
| BE_R2_3 | GRLGSEVGEGVLAARPADGHTIKVTESGRIIRC | 44 | A0A0N9HXW6_ | MafB19- | ND | Y (weak | Y (weak | Y (weak |
| | SRCDDILDLLDEYRAVFADNPGYVERLGRIEDL | | 9PSEU | deam | | activity) | activity) | activity) |
| | ADAARKARKAKNPNASQLADQAADDAAALLRDV | | | | | | | |
| | RTSAQARGNLAREGQPLSGAGRLPAEVVQPISP | | | | | | | |
| | ARIQEGLNSLAAQRVQRGLPPAGSATDVSTVCR | | | | | | | |
| | LDIGGESFYGVNAHHTTMDLHVNAQTATHAEGQ | | | | | | | |
| | AFQLGARSLPASRETRAVLYVDRELCRACGDFG | | | | | | | |
| | GVESMAKQLGLLQLDVYTPNGLALTLDFAGR | | | | | | | |
| BE_R2_7 | MPPAGSETDKSTIAKLEISGQNFFGINSGSNPN | 8 | A0A1U7ISE2_ | MafB19- | ND | Y (weak | Y (weak | Y (weak |
| | PRQITFNVNPITKTHAEADAFQQAADVGIRGGK | | 9CYAN | deam | | activity) | activity) | activity) |
| | ARLIVDRDLCAACGIRGGVNSMAWQLGIEELEI | | | | | | | |
| | ITPSVSKTIAVKPPNRRRQ | | | | | | | |
| BE_R2_11 | SQFDNVRKDMGLPARIGDDDPYTTSVLRIDGHE | 9 | A0A2T4Z7P2_ | MafB19- | ND | Y | Y | Y |
| | YWGKNGKWVTKGKTSNYTDKAHYDKVRKELGTS | | 9BACL | deam | | | | |
| | AEVPGHAEGVAFNKAYQVRKNTGTKGGNAVLYV | | | | | | | |
| | DKIPCVMCKPGIATLMRSAKVDHLDLHYLQDGK | | | | | | | |
| | MHHVQYVRNPDTDAVYNPFSGKWTKPSKKK | | | | | | | |
| BE_R2_17 | GRLKKDERVYRNAHQPFRLQNQYYDEETGLHYN | 10 | D2ZY33_ | MafB19- | ND | Y | Y | Y |
| | LMRYYEPEAGRFVNQDPIGLLGGDNLYWFAPNA | | NEIMU | deam | | | | |
| | AMWLDPWGLAVVDAIFEMQGHTFTGTNPLDRNP | | | | | | | |
| | RISSPIQGLSAVNNDKFKMHAEIDAMTQAHDKG | | | | | | | |
| | LRGGKGVLKIKGKNACSYCKGDIKKMALKLDLD | | | | | | | |
| | ELEVHNHDGTVHKFSKGDLKPVKKGGKGWKKPK | | | | | | | |
| | KSKKPGAC | | | | | | | |
| BE_R2_18 | RAPEAIQTLRDSYGTDLLGRPLLGDSDTVAHGI | 11 | A0A0A8K6F0_ | MafB19- | ND | Y (weak | N | Y (weak |
| | VDGETFMGVNSGAIVEYSQRDLNDAKRALIPLV | | 9RHIZ | deam | | activity) | | activity) |
| | RKRPDIMSTHNIGQRPNDALFHAESTVLLRAAR | | | | | | | |
| | ANDGTLSGKVIDITVDRPICSSCKKVLPLIGQE | | | | | | | |
| | LGNPIVRFTEPSGRVRTMHNGEWKDQD | | | | | | | |
| BE_R2_19 | GSYASPDPLGLEAAPNNHAYVANPATAADPTGL | 45 | A0A1I4B7X1_ | MafB19- | ND | Y | Y | Y |
| | IPCDVADDLAAYRQRQGMPVAGSAEDAHTAARL | | 9PSEU | deam | | | | |
| | DVDGQSFYGRNGHGMDIDIRANAQTKTHAEAQA | | | | | | | |
| | FQEAKNAGVSGKIGTLYVDRDFCRACGPNGGVG | | | | | | | |
| | SLMRGLGLERLEVHTPSGRYTIDATKRPSIPVP | | | | | | | |
| | WSEG | | | | | | | |
| BE_R2_20 | MPVAGSVDDKHTAAKLIFGDNEYYGHNGHGMQD | 46 | A0A1M7DT37_ | MafB19- | ND | Y (weak | Y (weak | Y (weak |
| | EVKGAFSVNAQTATHAEGLAFYNAKTSGVEGTS | | 9FIRM | deam | | activity) | activity) | activity) |
| | ATLITDRPACASCGYYGGIRSMAKDMGINDLTV | | | | | | | |
| | VSPNNAPITFNPQVKPIPNPFPKPVPKTIR | | | | | | | |
| BE_R2_21 | GLAGGEKPYAYVGNPAQAVDPLGLAGCEDPWKI | 47 | A0AIN6MQY7_ | MafB19- | ND | Y (weak | N | Y (weak |
| | VDRFRRSKNKMEPLGDRIPGAIDKDGLHTVAFF | | 9GAMM | deam | | activity) | | activity) |
| | EMNGRRVFGVNSGTLYKKDKALGKQWNEKIDYL | | | | | | | |
| | TKEEKGTSAFHAEGHALMRAHKKFGGVMPKEIT | | | | | | | |
| | MYVDRVTCNHCERFLPALMKEMGIEKLKLFSKN | | | | | | | |
| | GTSSVLHAAR | | | | | | | |
| BE_R2_28 | GSNGAIYSDVAAAQKAATTASRIGENDLATFRV | 48 | B9JGM2_ | MafB19- | ND | Y | Y | Y |
| | QLGLPPAGTAADKSTLAVIEINGQKIYGVNAHG | | AGRRK | deam | | | | |
| | QPVSGVNAISSTHAEIDALNQIKQQGIDVSGQN | | | | | | | |
| | LTLYVDRTPCAACGINGGIRSMVEQLGLKQLTV | | | | | | | |
| | VGPDGPMIVTPR | | | | | | | |
| BE_R2_29 | GALDNLAQTVTVADNATPSSADIFAEIAKSGDN | 14 | D2QYF9_ | MafB19- | ND | Y (weak | Y (weak | Y (weak |
| | ASQSTVDTFTDLAKSLDEAPPLDQSNAPNRTPW | | PIRSD | deam | | activity) | activity) | activity) |
| | DTIDHFRSHKQGMAELGDAIPVKGDKLGTVAFV | | | | | | | |
| | EIEGSKVFGVNSTALVDDADKALGRMWRDRLGF | | | | | | | |
| | NSGQAQALFHGEAHSLMRAYEKFSGKLPKDLTL | | | | | | | |
| | YVDRLTCGPCQGALPDLMKAMGIERLKIVIKSG | | | | | | | |
| | RVGEISGGVFRWLE | | | | | | | |
| BE_R2_31 | GGGTVTVSSTASAQVYATAQTEVEVTKKTKELA | 15 | G8SI56_ACTS5 | MafB19- | ND | Y | Y | Y |
| | AEQQQAQAYQCPVTGKACTGDPFNDLAAFRKRQ | | | deam | | | | |
| | GMPEAGTDADKDTAARLDVGGQIFYGRNGKGKV | | | | | | | |
| | TDIPVNAYTRDHAEGDVFQQAKNAKITADRAVM | | | | | | | |
| | YVDRPLCDGCGAYGGVGSLLRGTGIKEVVVVAP | | | | | | | |
| | NGRFLITAARPSTPQPLD | | | | | | | |
| BE_R4_4 | DKVADDVVEDAAKAIKGGSSSINLPEYDGKTTH | 49 | WP_216577045 | SCP1201- | ND | Y | ND | Y |
| | GVLVLDDGTQVPFSSGNANPNYKNYIPASHVEG | | | deam | | | | |
| | KSAIYMRENGINNGTVFHNNTDGTCPYCDKMLP | | | | | | | |
| | TLLEEGSTLTVVPPANANAPKPSWVDTVKTYIG | | | | | | | |
| | NDKIPKKPK | | | | | | | |
| BE_R4_6 | MSLPEYDGTTTHGVLVLDDGTQIGFTSGNGDPR | 50 | A0A7G9FZY2_ | SCP1201- | ND | Y | ND | Y |
| | YTNYRNNGHVEQKSALYMRENNISNATVYHNNT | | 9FIRM | deam | | | | |
| | NGTCGYCNTMTATFLPEGATLTVVPPENAVANN | | | | | | | |
| | SRAIDYVKTYTGTSNDPKISPRYKGN | | | | | | | |
| BE_R4_7 | MSITDRLAKQKEKQDNTNIIDNRPKLPDYDGKT | 51 | A0A7X7XYI6_ | SCP1201- | ND | Y | ND | Y |
| | THGILVTPNSEHIPFSSGNPNPNYKNYIPASHV | | CLOSP | deam | | | | |
| | EGKSAIYMRENGITSGTIYYNNTDGTCPYCDKM | | | | | | | |
| | LSTLLEEGSVLEVIPPINAKAPKPSWVDKPKTY | | | | | | | |
| | IGNNKVPKPNK | | | | | | | |
| BE_R4_10 | ELPPYDGKTTYGVLILDDGKQYSFNSGKPAPIY | 52 | MBR1615955.1 | SCP1201- | ND | Y | ND | Y |
| | RNYIPASHVEGKAAIYMRENKIQSGTVYHNNTD | | | deam | | | | |
| | GTCPYCDKMLPTLLEKDSTLKVVPPQNATSSKK | | | | | | | |
| | GWITNEKIYIGNDKIPKT | | | | | | | |
| BE_R4_12 | TDEFKLAYEQLKDIEQAYEYANIDKDKIDIPDF | 53 | MGYP000605828529 | SCP1201- | ND | Y | ND | Y |
| | DGKITWGILVLEDGTCITFSSGNANPMFNHYIP | | | deam | | | | |
| | ASHAEGKAAIYMRQKGIKHGVIFHNNTDGTCPY | | | | | | | |
| | CNTMLPILLEENSTLIVVPPINAVAKKRGWIDK | | | | | | | |
| | IKIYTGNNKIPKIN | | | | | | | |
| BE_R4_13 | GASGAAGHGLSTTGKNVLGHFEPTPTTPQGTSS | 54 | WP_021798742 | SCP1201- | ND | Y | ND | Y |
| | DTIAEMLNSASQPGRTAGVLDIDGELTPLISGR | | | deam | | | | |
| | PSLPNYIASGHVEGQAAMIMRQQQVQSATVYHD | | | | | | | |
| | NPNGTCGYCYSQLPILLPEGAALDVVPPAGTVP | | | | | | | |
| | PSNRWHNGGPSFIGNSSEPKPWPR | | | | | | | |
| BE_R4_14 | SHYAEEYKQLLKDIDTKREAEEAALLREAYPSM | 55 | WP_059988487 | SCP1201- | ND | Y | ND | Y |
| | EGATLPPFDGKTTIGLMFYTDASGQYQVKKLFS | | | deam | | | | |
| | GEKVLSNYDATGHVEGKAALIMRNEKITEAVVM | | | | | | | |
| | HNHPSGTCNYCDKQVETLLPKNATLRVIPPENA | | | | | | | |
| | KAPTSYWNDQPTTYRGDGKDPKAPSKK | | | | | | | |
| BE_R4_15 | ASASPSTNSAGSSGKNVRLPRDYASELPEYDGK | 56 | WP_082507154 | SCP1201- | ND | Y | ND | Y |
| | TTYGVLVTNEGKVIQLRSGGKEVPYSGYKAVSA | | | deam | | | | |
| | SHVEGKAAIWIRENASSGGTVYHNNTTGTCGYC | | | | | | | |
| | NSQVKALLPEGVELKIVPPANAVARNSQAKAIP | | | | | | | |
| | TINVGNATQPGRKP | | | | | | | |
| BE_R4_16 | KPEALKDAREPKTKPPHNRVHQDPNTSWNPNNY | 57 | WP_112210906 | CYT_DCMP_ | ND | Y | ND | Y |
| | PDTPSGQLPAYDGKNTLGRIEIDGEIYHVKNGK | | | DEAMINASES_ | | | | |
| | GQPGETLKTDPTVKAGAVSPSHAEGHAVAIMKE | | | 1 | | | | |
| | TGTKEAVLDINHPTGPCGFCDKVLENMLPEGSK | | | | | | | |
| | LTVNWPNGSQVFTGNSK | | | | | | | |
| BE_R4_17 | SHYAKEYKQLLADIDALAEAREDALLREQFPSM | 58 | WP_133186147 | SCP1201- | ND | Y | ND | Y |
| | DAVTLPPFDGKTTIGYMFYTDANGQYHVRKLYS | | | deam | | | | |
| | GGKVLSNYDSSGHVEGMAALIMRKGRITEAVVM | | | | | | | |
| | HNHPSGTCHYCNGQVETLLPKNAKLKVIPPANA | | | | | | | |
| | KAPTKYWYDQPVDYLGNSNDPKPPS | | | | | | | |
| BE_R4_18 | GGSAVVGGGIAATGAKALTTGKKLTESPGTLNA | 59 | WP_157869269 | SCP1201- | ND | Y | ND | Y |
| | AQRLLASIGEEGKTAGVLEVDGALFPLVSGKSV | | | deam | | | | |
| | LPNYAASGHVEGQAALLMQGMGATNGRLLIDNP | | | | | | | |
| | NGICGYCTSQVPTLLPENAVLEVGTPLGTVTPS | | | | | | | |
| | ARWSASKPFIGNDREPKPWPR | | | | | | | |
| BE_R4_19 | IGKVGKLRFAPKVESAESMLRSLSQEGKTAGVL | 60 | WP_165946289 | SCP1201- | ND | Y | ND | Y |
| | DINGELIPLVSGTSSLKNYAASGHVEGQAALIM | | | deam | | | | |
| | RERGVASARLIIDNPSGICGYCRSQVPTLLPAG | | | | | | | |
| | ATLEVTTPRGTVPPTARWSNGKTFVGNENDPKP | | | | | | | |
| | WPR | | | | | | | |
| BE_R4_20 | LEDKIDYDDLVRKREKAREDLLEAEKRLREEEI | 61 | WP_174422267 | SCP1201- | ND | Y | ND | Y |
| | RAKYPTPEEAQLPPYDGDTTYALMYYTDEHGKS | | | deam | | | | |
| | HVVELSSGGADDEHSNYAAAGHTEGQAAVIMRQ | | | | | | | |
| | RKITSAVVVHNNTDGTCPFCVAHLPTLLPSGAE | | | | | | | |
| | LRVVPPRSAKAKKPGWIDVSKTFEGNARKPLDN | | | | | | | |
| | KNKKST | | | | | | | |
| BE_R4_21 | GGSAVVGAGVVATGAKAVTTGKSLSESQATLSV | 62 | WP_189594293 | SCP1201- | ND | Y | ND | Y |
| | AQRLLATIGEEGKTAGVLELDGELIPLVSGKSS | | | deam | | | | |
| | LPNYAASGHVEGQAALIMRDRGATSGRLLIDNP | | | | | | | |
| | SGICGYCKSQVATLLPENATLQVGTPLGTVTPS | | | | | | | |
| | SRWSASRIFTGNDRDPKPWPR | | | | | | | |
| BE_R4_22 | DSAVDRLEQELEKLDVRNFFEDESETESGSSSI | 63 | MGYP000498443267 | SCP1201- | ND | Y | ND | Y |
| | NLPEYDGKTTHGVLVLDDGTQVPFSSGNANPNY | | | deam | | | | |
| | KNYIPASHVEGKSAIYMRENGINNGTVFHNNTD | | | | | | | |
| | GTCPYCDKMLPTLLDEGSTLTVVPPTNASAPKP | | | | | | | |
| | SWVDTVKTYIGNDKIPKKPK | | | | | | | |
| BE_R4_23 | SGYDSQYPCKEEMSAGAGESGRKTISLPEYDGT | 64 | WP_195441564 | SCP1201- | ND | Y | ND | Y |
| | TTHGVLVLDDGTQIGFTSGNGDPRYTNYRNNGH | | | deam | | | | |
| | VEQKSALYMRENNISNATVYHNNINGTCGYCNT | | | | | | | |
| | MTATFLPEGATLTVVPPENAVANNSRAIDYVKT | | | | | | | |
| | YTGTSNDPKISPRYKGN | | | | | | | |
| BE_R4_24 | ASPAVGTNAAGSSGKNVRMPRDYASELPEYDGK | 65 | WP_211232061 | SCP1201- | ND | Y | ND | Y |
| | TTHGVLVTNEGKVIQLRSGGKEEPYTGYKAVSA | | | deam | | | | |
| | SHVEGKAAIWIRENGSSGGTVYHNNTTGTCGYC | | | | | | | |
| | NSQVKALLPEGVELKIVPPTNAVAKNAQARAVP | | | | | | | |
| | TINVGNGTQPGRKQK | | | | | | | |
| BE_R4_25 | YVGENGVWVHNASSEYGEVPELPEFNGKKTEGV | 66 | MGYP000402883179 | SCP1201- | ND | Y | ND | Y |
| | FRTADGKEIKFESGGSTEYKNPSASHAEGKAAI | | | deam | | | | |
| | YMRENGIKEGTVFHNNPNGTCNYCDKGLATLLP | | | | | | | |
| | EGARLTVVPPIGAVAPNKYWVDVPKTYTGNGNL | | | | | | | |
| | PSMK | | | | | | | |
| BE_R4_26 | HVGKCRLLVHNANCNQEKPVLPKYDGKTTEGVM | 67 | MGYP000186340475 | SCP1201- | ND | Y | ND | Y |
| | VTPDGKQISFKSGNSSTPSYPQYKAQSASHVEG | | | deam | | | | |
| | KAALYMRENGINEATVFHNNPNGTCGFCDRQVP | | | | | | | |
| | ALLPKGAKLTVVPPSNSVANNVRAIPVPKTYIG | | | | | | | |
| | NSTVPKIK | | | | | | | |

### Example 2: Generation and Identification of Protein-Only Base Editors for Mitochondrial Genome Engineering

Mitochondrial genetic diseases caused by mutations in the mitochondrial genome are a class of devastating human diseases that are currently incurable, due to lack of technologies that allow precise editing of these mutations. Majority of these mutations (78 out of 93 confirmed pathogenic mutations) are in the form of single point mutations and can be potentially fixed by base editing, however, due to lack of efficient mechanisms for delivery of nucleic acids to mitochondria, existing RNA-guided technologies like those based on CRISPR have not been successfully applied to mitochondria. The main limitation with the use of CRISPR and any editing system that relies on a DNA (e.g., a template) or RNA (e.g. guide RNA) moiety for editing is the lack of mechanisms that can be used to shuttle those moieties across the mitochondrial double membrane into mitochondrial lumen. Although there have been reports claiming successful editing of mitochondrial genome using RNA-guided system (e.g. CRISPR-Cas9), they have remained controversial and not reproducible. The evidence provided in most of these studies are indirect (e.g., qPCR) rather than showing direct evidence of editing (sequencing the edited loci).

In the absence of precise genome editors (which mainly rely on RNA-guided proteins like CRISPR-Cas9), programmable protein-only nucleases (mitochondrial Zinc Finger Nucleases (mitoZFNs), mitochondrial TALE-Nucleases (mitoTALENs), and mitochondrial Restriction Enzymes (mitoRE)) have been leveraged to shift the level of mitochondrial genome heteroplasmy in cell cultures/patient derived samples/animal models. All of these approaches rely on a fusion of a (split) nuclease with programmable DNA binding domains. The DNA binding domain (ZF, TALE, RE) is designed in a way that it can bind to the mutated copy (but not the WT copy) of the mitochondrial genome with high affinity, and thus preferentially binds to and cleaves the mutated copy of mitochondrial genome, thus shifting the heteroplasmy toward the desired (wt) allele. This approach is only applicable to diseases that have significant levels of heteroplasmy (both wt and mutated allele are present at considerable amount) and not is not currently very effective in addressing the disease.

Due to their activity on dsDNA, full length dsDNA-specific deaminaes are toxic when expressed in the cells (it can introduce global mutations across the genome). To manage the toxicity, recent studies used a strategy that was previously used in case of FokI nuclease in TALENs and ZFNs and other toxic domains, namely split the toxic protein into two halves. They then fused each deaminase halve to a TALE domain appended with mitochondrial targeting peptide and UGI (which blocks repair machinery).

Similar to TALEN approach, TALE binding sites were designed at both sides of the target sites. Once bound to their targets, they bring the two deaminase halves together and form a functional cytidine deaminase that can deaminate cytidines in the vicinity of deaminase binding site.

A main limitation of the recent approach based on dsDNA specific DddA described by Mok et al. is, however, its narrow context-specificity. Due to the context specificity of DddA (which can only edit cytidines in TC contexts, as shown in the above sequence logo from Mok et al paper), the published base editor can only edit Cytidines that precedes with a Thymine which accounts for 4/93 confirmed pathogenic mutations in humans.

By leveraging a panel of dsDNA-specific deaminases, a suite of protein-only base editors that can edit cytidines in any contexts (NCN: ACN, CCN, GCN, TCN) with high efficiency was developed. In addition, engineering rules that allow tuning the window of activity of the deaminase on the target region and used those principles to engineer efficiently and precisely edit different dsDNA substrate in vitro and in vivo (nuclear or mitochondrial genomes) have been developed. Due to limitations of CRISPR-based methods for delivery of guide RNA to mitochondria, as well as limited context specificity of dddA-based approach, the base editors described herein enable base editing in a broader sequence context and are especially suited for mitochondrial genome engineering applications as well as base editing in other membranous organelles.

Site-Specific Deamination of dsDNA by Fusing dsDNA-Specific Cytidine Deaminases to Programmable DNA Binding Domains

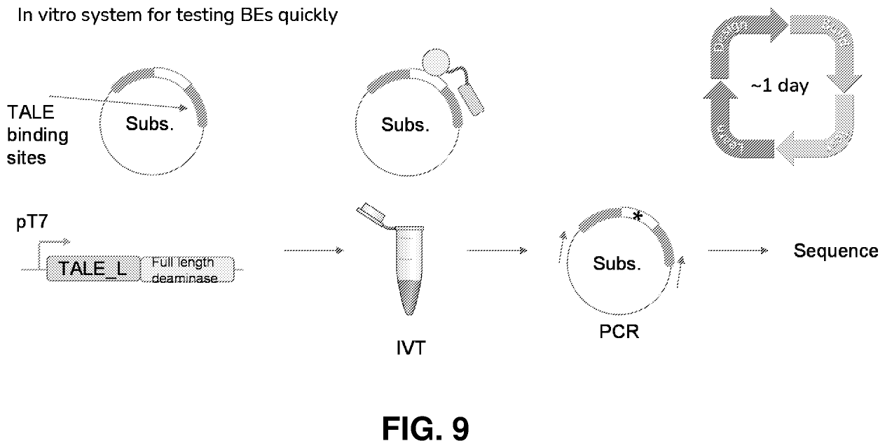

Gene editing experiments usually are performed in cells which could take days and weeks for each round of experiments. To reduce this time, and to avoid toxicity issues that may arise from using base editors, initial experiments were set up an in vitro system based on in vitro transcription/translation (IVT) system (previously used to identify novel dsDNA-specific deaminases) to quickly test performance of gene editors and base editors in vitro (FIG. 9).

Briefly, the base editor were made by cloning the deaminase domains downstream of designer TALE. The entire cassette was cloned downstream of a T7 promoter and used as template in the IVT reaction. The target (encoding binding sites for DNA binding domains of interest, e.g. designer TALEs) were cloned on plasmids which was then used as dsDNA substrate in the IVT reaction. Upon expression in the IVT system, the base editor protein (e.g., TALE deaminase fusion) binds to its target on the substrate plasmid and introduce edits to the target plasmid. The substrate plasmid was then PCR amplified and the position/frequency of edits are determined by either sequencing or T7 endonuclease assay.

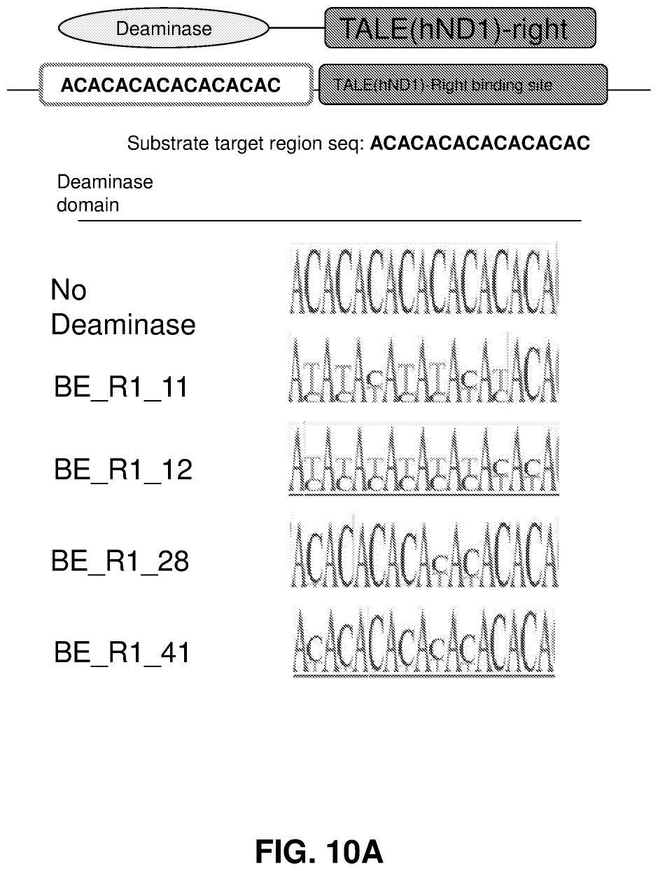

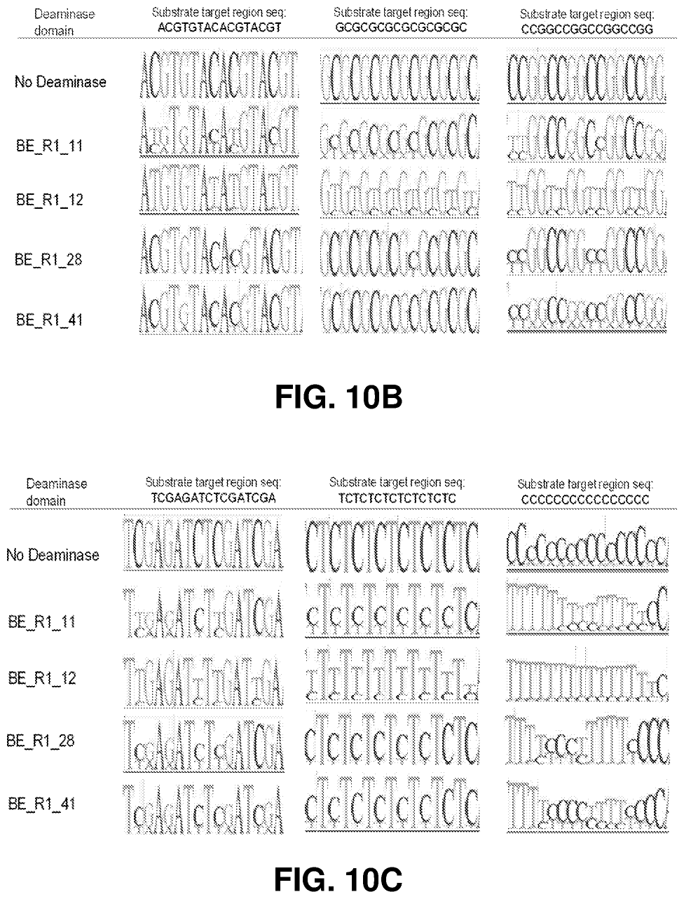

The activity of TALE-full-length deaminase fusions for a subset of the identified dsDNA specific deaminases was tested with different substrates with different sequence contexts. The deaminases were active on all the possible dinucleotide contexts (AC, CC, GC, TC) and different fusions showed different window of activity and editing efficiencies on different substrates (FIGS. 10A-10B).

Interestingly, a 10 bp period in editing window was observed. The editing was more pronounced in some substrates (e.g. polyC or poly TC) than others. Optimal editing window happens periodically (with 10 bp period which corresponds to one double helix turn). This suggests the deaminase only has access to one side of the double helix. Periodic window is less pronounced in TALE_BE_R1_11 and TALE_BE_R1_12 either because these deaminases are too active or, the linker between the TALE and deaminase core is too flexible. This is consistent with and supports the structure prediction models predicting that the deaminase interacts with both minor and major grooves of DNA. When fused to TALE, the movement of the deaminase will be restricted from one side, thus the deaminase will have better access to one side of the double helix vs. the other.

A predicted model for TALE-deaminase fusion bound to DNA (using BE_R1_41 as an example of dsDNA-specific CDA) was calculated. The model suggested that deaminase when fused to TALE has preferentially access to one side of the double helix. The requirement for interacting with major and minor grooves of DNA dictates the ˜10 bps period window of activity observed in these experiments.

### Split Base Editor Designs

Gene editing experiments usually are performed in cells which could take days and weeks for each round of experiments. To reduce this time, and to avoid toxicity issues that may arise from using base editors, initial experiments used an in vitro system based on in vitro transcription/translation (IVT) system (which was previously used to identify novel dsDNA-specific deaminases) to quickly test performance of gene editors and base editors in vitro. The base editor halves were made by cloning the deaminase split domains downstream of designer TALEs (called TALE_Left and TALE_Right). The entire cassette was cloned downstream of T7 promoter and used as template in the IVT reaction. The target (encoding binding sites for DNA binding domains of interest, e.g. designer TALEs) were cloned on plasmids which was then used as dsDNA substrate in the IVT reaction. Upon expression in the IVT system, the base editor protein (e.g., TALE deaminase) binds to its target on the substrate plasmid and introduce edits to the target plasmid. The substrate plasmid is then PCR amplified and the position/frequency of edits are determined by either sequencing or T7 endo nuclease assay.

In the absence of structural data for the identified deaminases, split deaminase proteins were designed by using the SPELL webtool, which predicts positions in proteins that could potentially result in functional protein upon assembly. split forms were tested by co-expressing the predicted split halves in the IVT system followed by deamination assay. A few designs including BE_R1_11 (N3+C3) and BE_R1_12 (N2+C2 and N4+C4) showed some levels of activity (no activity were detected when either of the split halves were expressed individually). However, the activity of these split variants was significantly less than the full length deaminase, and did not result in significant editing of target region when fused to TALE DNA binding domain (FIG. 5).

The initial attempts to create split deaminase TALE fusion for MafB19-deam family implied possibilities for other requirement for activity of these deaminase and inspired us to come up with alternative approaches for making split proteins. When designing split proteins, the goal is to find a position within protein of interest that once the protein is split into two halves at that position the protein halves do not retain activity, but the activity is reconstituted once the two halves are come together under certain conditions. The first attempt to design split CDA proteins without having structure data using existing tools failed and a new and more universal approach for making split proteins was sought without prior knowledge about protein structure. Rather than splitting the protein into N-ter and C-ter halves as being done traditionally, we devised an approach that involves complementing an inactive (dead) copy of a full length protein with a truncated copy of the protein that does not retain the activity by itself. The enzymatic activity is reconstituted once the dead copy of the enzyme and the truncated copy of the enzyme are colocalized. The colocalization can be achieved, for example, by fusing the two moieties to DNA binding domains with juxtaposing binding sites on a DNA molecule.

BE_R1_12 was used for initial studies (which showed strong activity when expressed as full length deaminase) fused to TALE DNA binding domains to demonstrate this conditional protein colocalization and activation concept.

First, a “dead” (inactive) BE_R1_12 (dead BE12 or dBE12) protein was made by mutating the conserved Glutamic Acid (E residue in the HAE motif, which is predicted to be the active site of the enzyme based on homology with known cytidine deaminase such as APOBEC and AID) to Alanine.

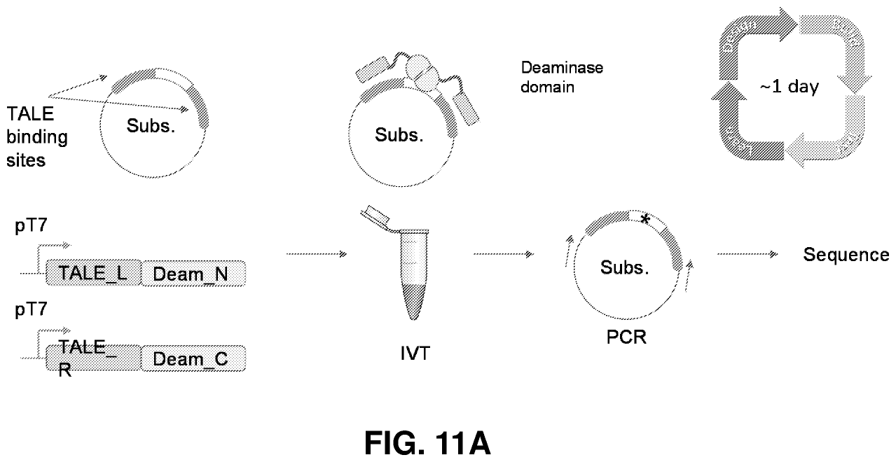

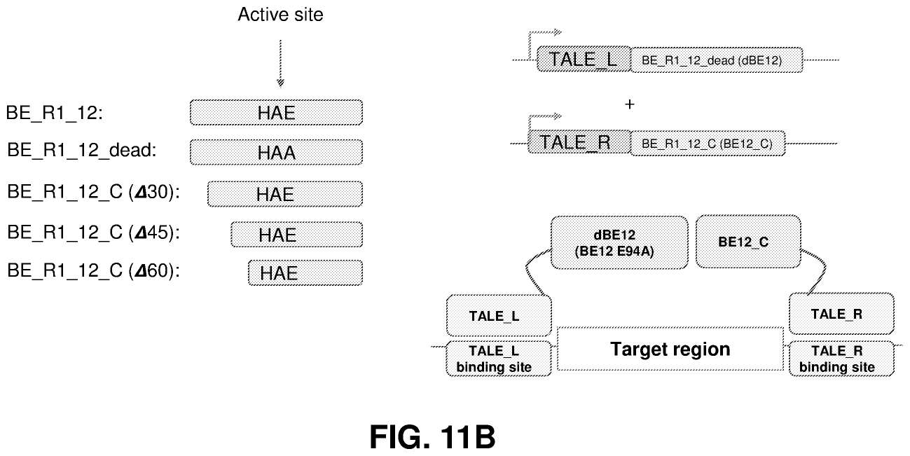

The dead copy of deaminase was fused to TALE_Left (TALE_L) domain that binds to the left side of target region in the substrate plasmid. The full length active BE_R1_12 was also sequentially truncated from N-ter every 5 amino acids (the truncated domain still retained the HAE active site). The truncated domains were fused to TALE_Right (TALE_R) domain that binds to the opposite side of the target region across TALE_L binding site. The two TALE-deaminase fusion halves were tested individually or in combination in the IVT system. Unlike the traditional approach for split protein design, this new approach doesn't require information about protein structure, and potentially allow making functional split proteins that become active in dimeric form but not monomeric form (FIG. 11).

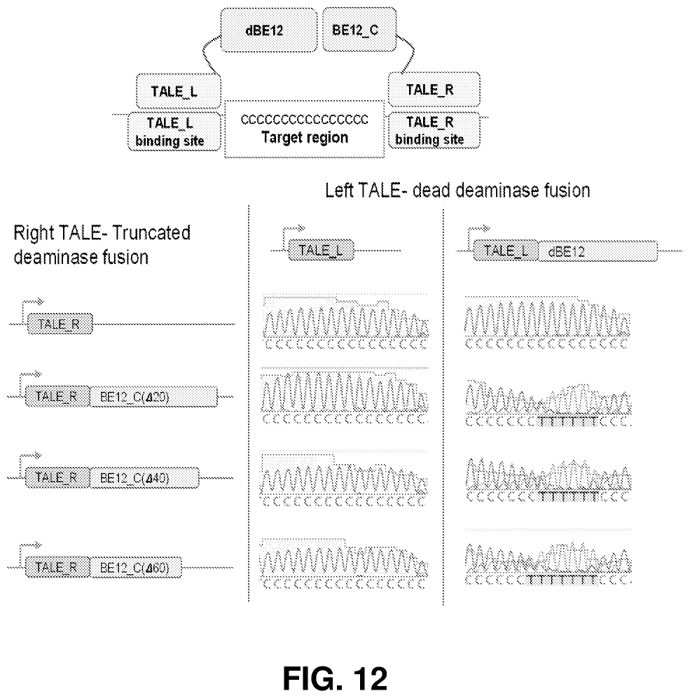

The Split TALE-BE_R1_12 base editor was incubated with treating a polyC containing substrate flanked by TALE binding sites and the outcome of base editing was read out by Sanger sequencing. The TALE_R_truncated_BE12 fusions as well as the TALE-dead_BE12 fusions are inactive on the dsDNA polyC containing substrate. However, when both the TALE_R_truncated_BE12 and TALE_L_dead_BE12 are added, deaminase activity is reconstituted at the vicinity of the TALE binding sites, leading to the efficient editing of the cytidines in the Target region (FIGS. 12-13). Unlike dddA which can only efficiently edit cytidines in TC context, split BE12 base editor can efficiently edit all the possible contexts (AC, CC, GC, TC), and thus acts as a context-independent base editor. In this design, the maximum window of activity is toward the middle of target region.

### Example 3: Additional Split Base Editor Architectures (Making Highly Efficient Split Base Editor with 2× Deaminase Active Sites Instead of Lx Active Site)

An additional approach was devised for making split base editors where instead of one copy of the active site, two copies of the active sites are localized to the target region, leading to higher on-target activity. To achieve this, instead of using a single split site, two different split sites were used on both sides of the deaminase active site. The split sites are chosen in a way that none of the individual fragments lead to enzymatic activity, but they can complement each other once fused to TALE and localized on the target region upon binding of TALEs to their target. When using the bigger fragments of each split site, this approach could give 2× copies of the active site (HVE) on the target, instead of 1× in the traditional approach leading to higher editing activity.

### Cleaved (Split) Fragments of BE41

This approach was demonstrated by making split fragments of BE41 (a protein belonging to the SCP1201-deam family, and a homolog of dddA, for which protein structure and split sites have been identified before). Based on homology, positions G43 and G108 in BE41 were identified as potential split sites. The N-ter and C-ter fragments were then fused into TALE_R and TALE_L DNA binding domains and expressed them individual or in combination (N-ter+C-ter fragments) in the IVT system. A plasmid containing 16 bps poly C flanked by TALE binding sites was used as substrate (all positions across the target region in the poly C substrate can potentially get edited, thus allowing to better quantify and visualize editing activity/efficiency across the target region). Interestingly, the position of split site affected the window of activity (positions within the target region that are edited, shown by red curve on top of sanger chromatograms) of the base editor. The window of activity for the combinations that contained C_G43 fragments was between positions 6-13 of the 16 bps target region, whereas the window of activity when C_G108 fragment was used was at positions 8-15.

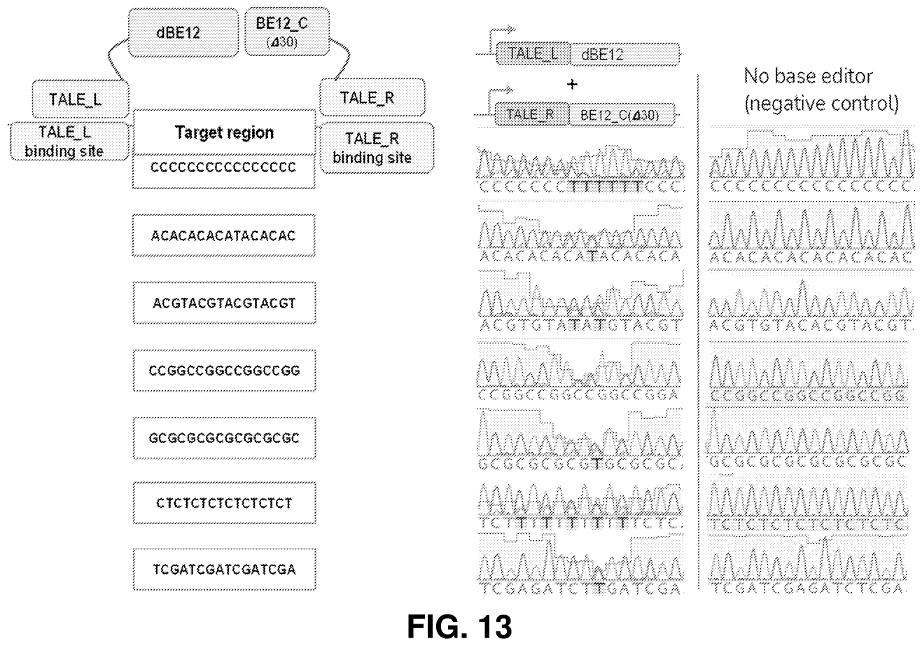

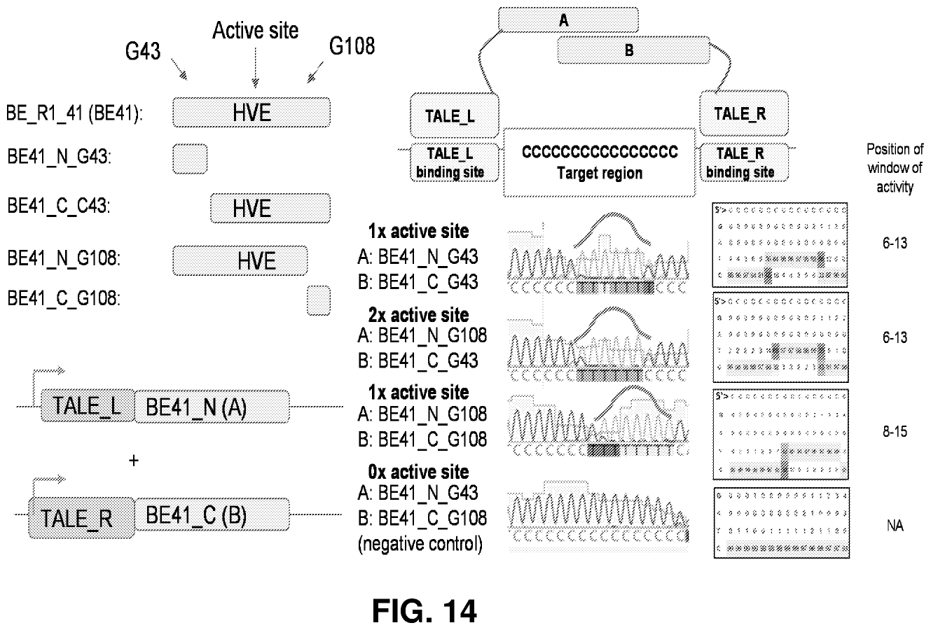

The 2 bps shift in the window of activity in the C_G108 vs C_G43 combinations is likely due to the shorter length (and thus reduced flexibility) of the C_ter fragment in C_G108 fragment. This future can be used to tune the window of activity of this class of base editors. This experiment demonstrates that the position of split site in a deaminase affects the window of activity of base editors and can be leveraged to tune window of activity of this class of base editors. Designing additional split sites for the deaminase proteins can help to further tune window of activity of the base editors when needed (FIG. 14).

### BE41_N_G108+BE41_C_43 Combination (2× Active Site Split Design)

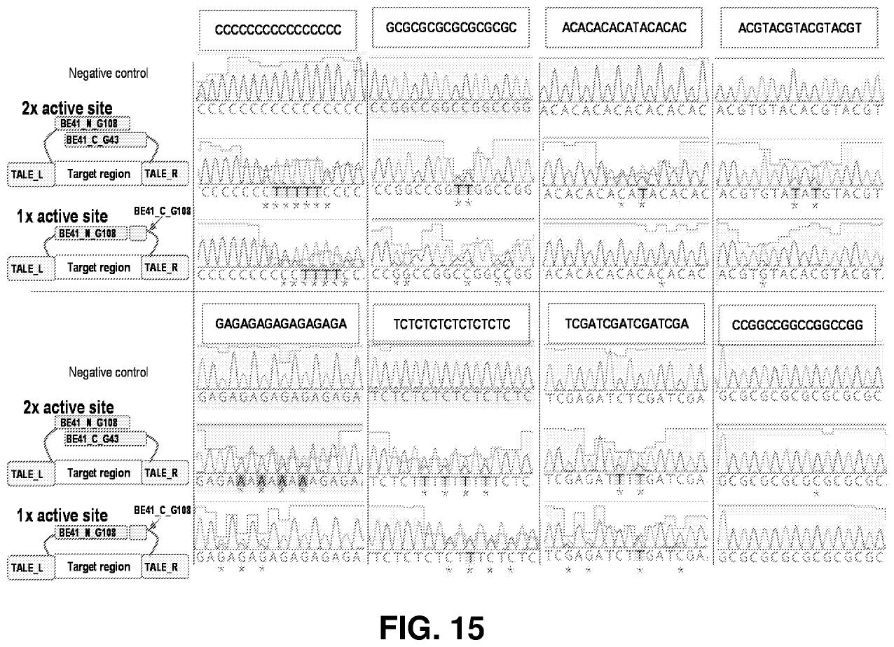

BE41_N_G108+BE41_C_43 combination (2× active site split design) result in higher editing efficiencies than BE41_N_G108+BE41_C_G108. The 1× active site combination is active on TC and CC contexts, but not on AC or GC contexts. The design with 2× active site is relatively more active on TC and CC context, and also is somewhat active on AC context, and slightly on GC context. The maximal activity is observed in the middle of the activity window. For 2× active site design, the maximal activity is observed at positions 9-11 (in a 16 bps target region) and drops by distance from the center. The maximal activity for 1× active site design is observed at positions 11-13 (in a 16 bps target region) and drops by distance. The red asterisks indicate positions of edit sites. The relative heights of the peaks at positions corresponding to the asterisks indicate the editing efficiency (C to T conversion on the forward strand (shown) or G to A conversion (C to T conversion on the reverse complement strand))(FIG. 15).

### 2× Active Site BE41 Base Editor Design

2× active site BE41 base editor design shows higher activity than 1× BE41 base editor. Both BE41 base editor architectures is proficient in editing in CC and TC contexts that falls within their corresponding window of activity. BE41 prefers Poly C over Poly TC context. 1× active site BE41 base editor struggles on AC contexts.

BE41 base editor can deaminate cytidines on the reverse strand, resulting in G-to-A mutation on the forward strand after PCR amplification. The window of activity on the reverse strand is the opposite side of the window on the forward strand.

Unlike BE12 base editors that could edit cytidine in any context, BE41 base editors struggle with editing cytosine in GC contexts and to lesser extent on AC context. Some degree of editing is observed in these contexts at the position corresponding to the maximal window of activity (10 bps away from Left-side TALE in the case of 2× active site design, and 12 bps away from Left-side TALE in the case of 1× active site design).

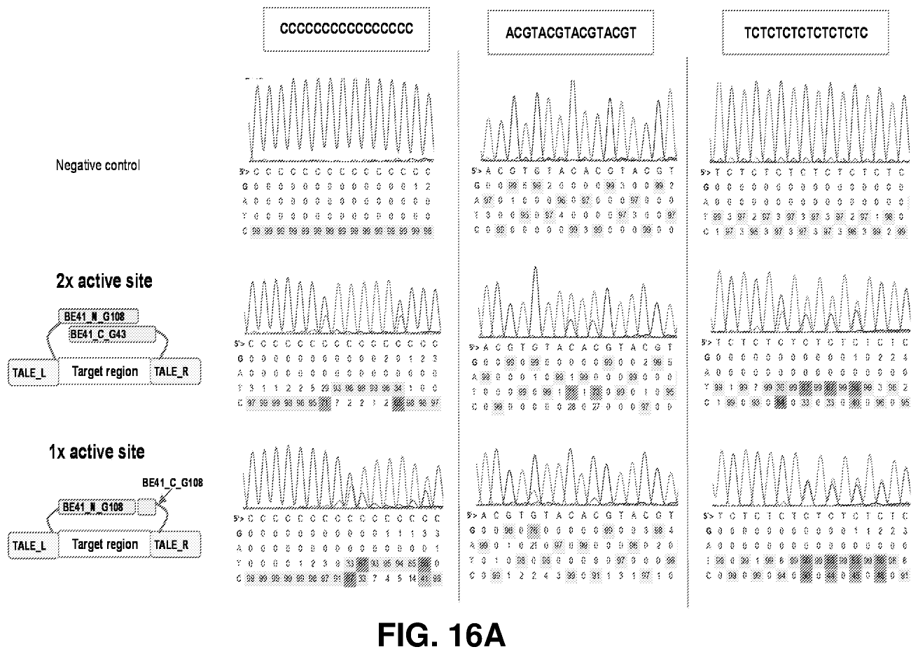

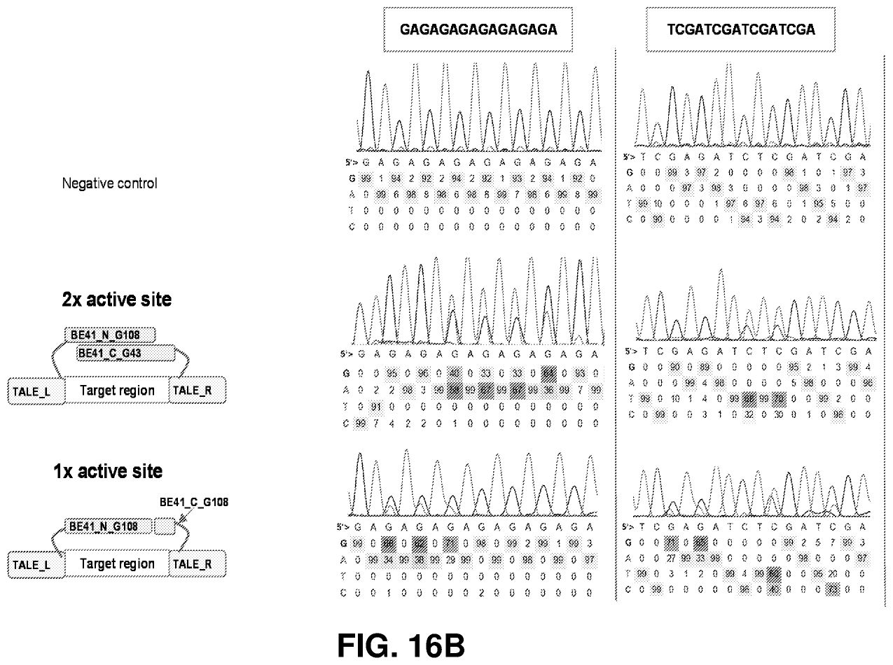

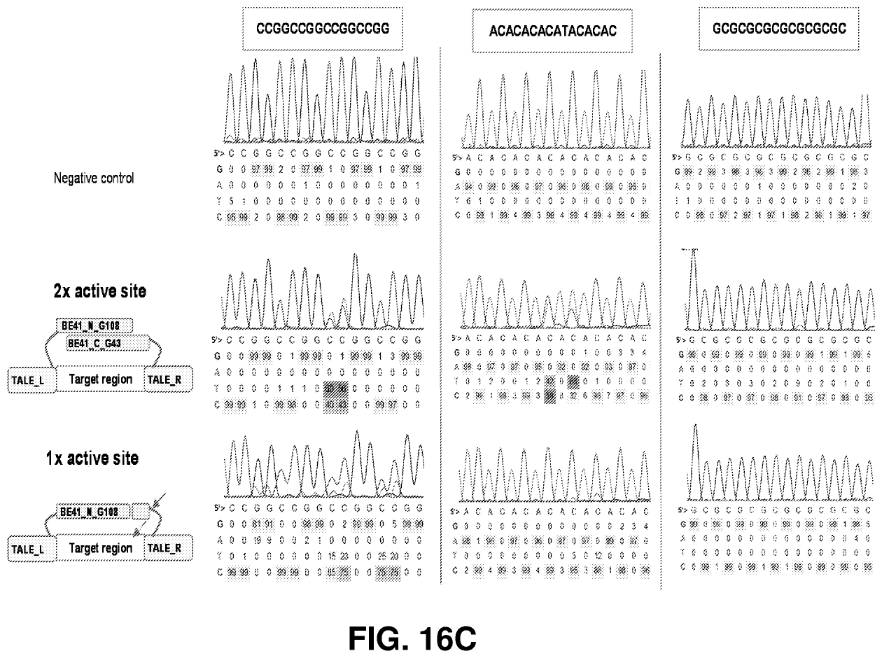

### (FIGS. 16A-C).

### Example 4: Base Editors Window of Activity: Factors Affecting Window of Activity and how to Tune them

It was determined that swapping the deaminase split halves affects editing efficiency but doesn't change the position of window of activity significantly. It was established that the directionality of DNA in the target region is important.

Swapping the deaminase halves between TALE_Right and TALE_Left doesn't change the position of window of activity, that is to say, for this specific deaminase (BE41), the cytosines on the right side (but not the left side) of the forward strand within the target region are preferentially edited, independent of the orientation of the deaminase split halves. Fusing the smaller fragment to the TALE with a binding site closer to the window of activity (the Right TALE in this case) leads to higher efficiency, likely because better spatial accommodation of the large and small fragments in respect to the window of activity. This is a counter intuitive observation; however it can be explained by the finding that the deaminase interacts with and bind to dsDNA through both minor and major groove of DNA. This binding requirement is needed for deamination activity, and restricts the window of activity of base editor. Since the each turn of dsDNA helix is 10 bps, within the 16 bps target region used in this experiment, only one minor and major groove pair is accessible to the deaminase for binding, and thus only a half a turn of the forward strand satisfies the deaminase binding requirement and is effectively deaminated.

### Structural Modelling of Split Base Editors

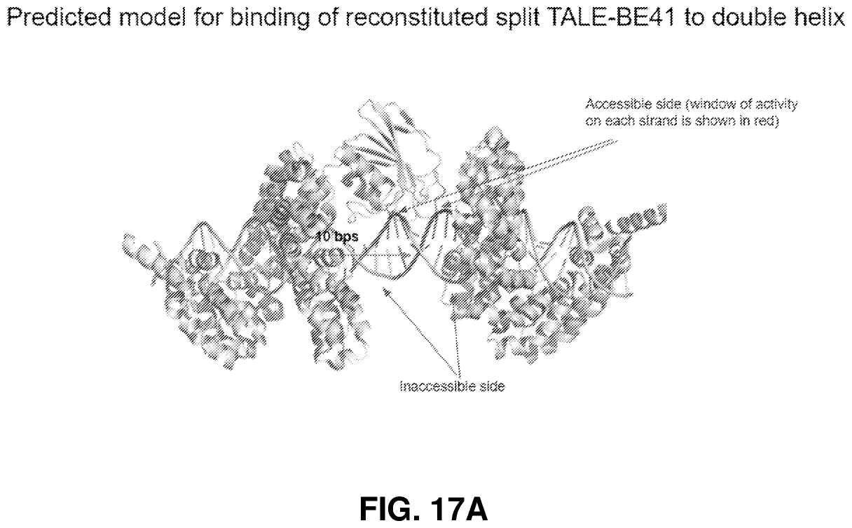

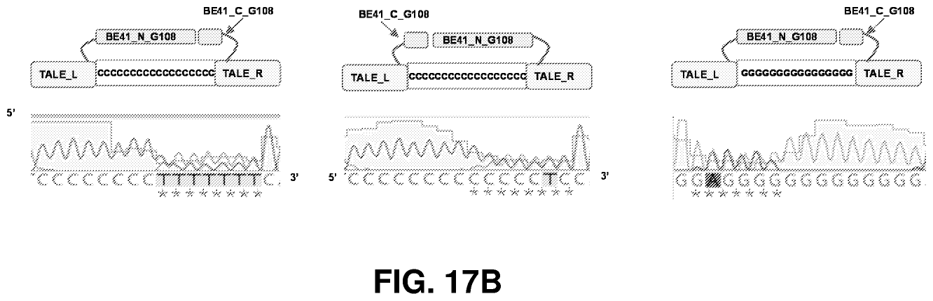

A computational structural model was calculated to model binding of reconstituted split TALE-BE41 to the DNA double helix (FIG. 17A). This model predicted that cytidines on the reverse strand should also be accessible to the deaminase and subject to deamination, which was verified to be true by using a PolyG substrate instead of PolyC. When PolyG substrate used instead of PolyC, the positions in the first half of the target region were deaminated (Cs on the reverse strand), further confirming the proposed model

### (FIG. 17B).

These findings suggest that that this class of base editors that leverage dsDNA specific deaminases possess a periodic window of activity with a asymmetric phase on forward and reverse strand.

Based on this model, the position of deaminase active site relative to the accessible side of DNA (i.e. accessible minor and major grooves of DNA within the target region) would affect the position of window of activity. The position of split site would affect the relative position of active site relative to DNA. The data indicated that changing the flexibility and length of the linker could affect the position of enzyme active site with respect to the accessible side of DNA, and hence affect the editing window and efficiency. The body of deaminase itself could, therefore, act as a linker and affect the accessibility of the deaminase to dsDNA. These findings are valuable in tuning the window of activity and minimizing mutation of bystander residues by this class of base editors.

### Tuning Window of Activity of Base Editors

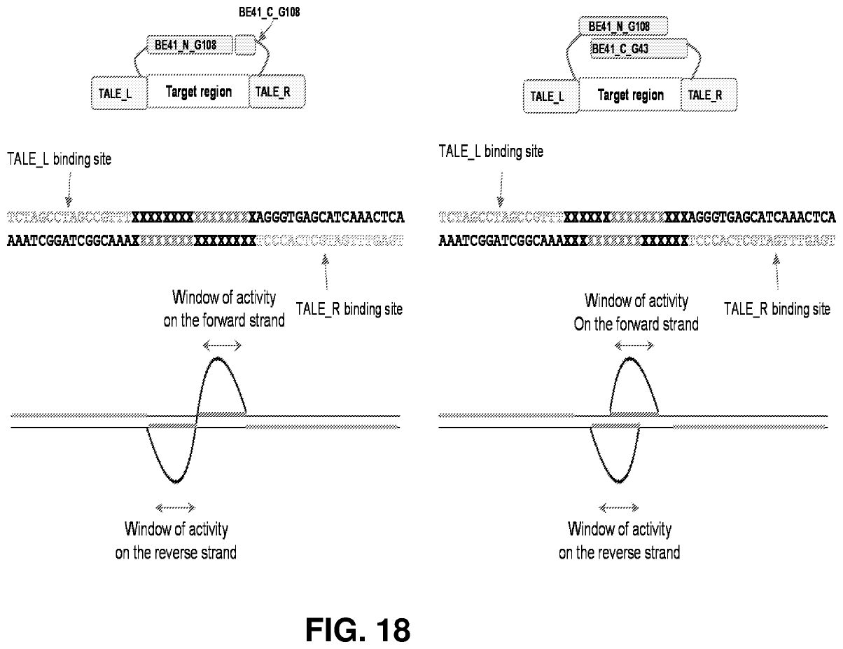

The window of activity for split BE41 base editors that is based on the computational model and data is depicted in (FIG. 18). The TALE binding sites and positions corresponding to the window of activity for each strand of DNA are indicated. The window of activity could change based on the nature of deaminase, the position of split sites, the type of linker being used, etc. However, when the deaminase binding requires interaction with minor and major grooves of DNA, a periodic and asymmetric activity window is expected.

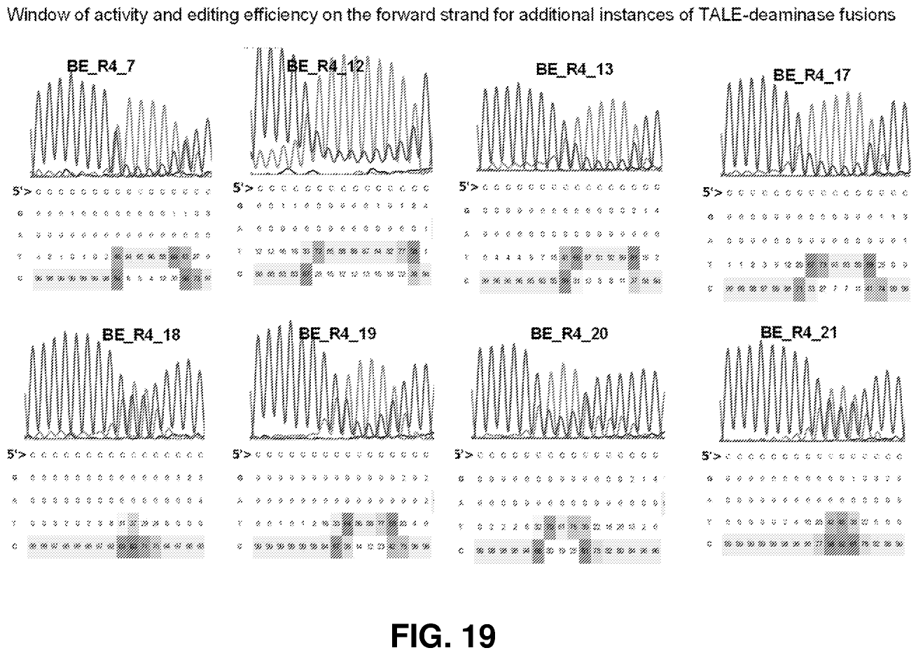

Base editors made using different dsCDA showed different window of activity and editing efficiency on a given substrate (FIG. 19), further indicating that different deaminases have different windows of activity.

### Effect of the Distance Between the DNA Binding Domains

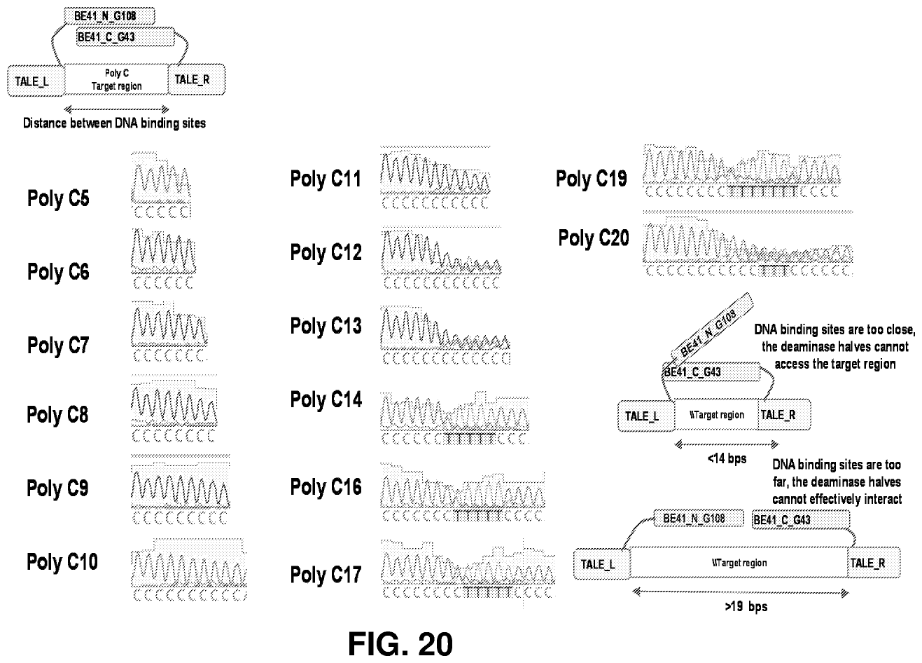

An optimal distance between the DNA binding sites are needed to ensure efficient editing. In the case of split BE41 deaminase, this distance is between 14-19 base pairs. If the target region (the distance between the two DNA binding sites) is <14 bps, the deaminase will not have enough space to fit in the target region and access the minor and major grooves of DNA in the right orientation. On the other hand, if the target region is >19 bps, the editing efficiency drops, likely as a result of the distance between two deaminase become too far, and their interactions (and thus editing efficiency) becomes dependent on molecular movements of dsDNA and other factors. The optimal distance between DNA binding sites represents the optimal distance that the two deaminase halves can efficiently interact. This optimal distance could vary based on the nature of deaminase and DNA binding domains, the linker connecting those domains, and the position of the split site in the deaminase domain (FIG. 20).

### Example 5: Nature of the DNA Binding Domain/Linker Affects Base Editor Window of Activity

To further confirm the model, the TALE DNA binding domain was replaced with BAT DNA binding domains (a recently described TALE-like DNA binding domain with the same DNA binding code as TALE) targeted to the same DNA sequence. Although the BAT repeats use the same RDV code as TALEs (A:NI, C:HD, G:NN, T:NG), the N- and C-terminus of TALE and BATs are different. Unlike TALEs that follow a TO rule (TALE binding site needs to strictly start with a T), BAT N-terminal domain is more flexible for binding and the BAT binding site can start by any of the four nucleotides. C-terminus of BAT is non-homologous to TALE and shorter (30 aa in BATa vs. 41 aa in TALEs used in this experiment).

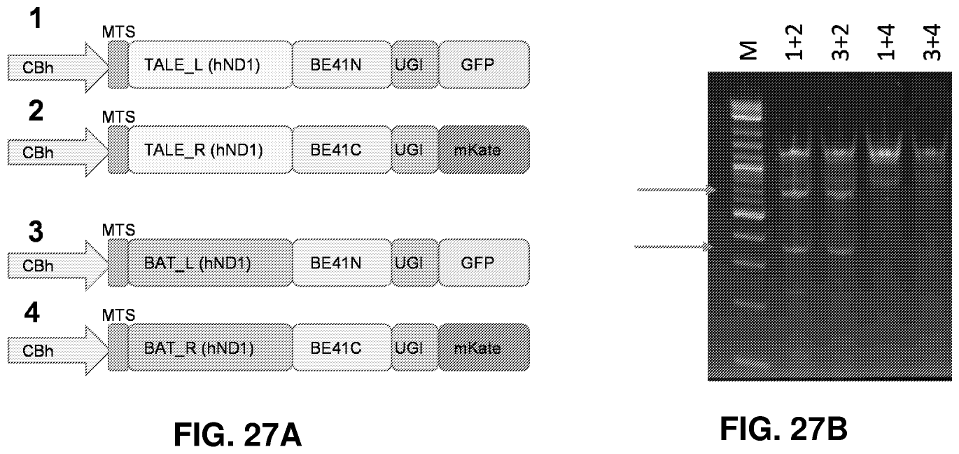

Replacing one of the TALE domains with synonymous BAT resulted in a shorter window of activity, with the window of activity shifting toward the TALE domain (FIGS. 27A-B). The shorter window of activity suggests that the active deaminase is reconstituted on a shorter span on the double helix, because of the less flexibility and/or shorter length of the BAT C-ter. Replacing both TALE domains with synonymous BATs completely abolished the base editing activity, likely because the shorter C-ter domains of BATs were not long/flexible enough to allow interaction of the deaminase halves. The activity of BAT-TALE pairs was further verified by expressing the constructs in HEK293 cells and assessing the outcome of editing by T7 endonuclease assay (FIG. 27B).

This experiment demonstrated two main points:
i) BATs (and likely other TALE-like proteins) can be used as an alternative to TALEs in this class of base editors; and
ii) the window of activity is dependent on the type of DNA binding domain fused to deaminase domain and can be tuned by changing the sequence/length of the linker between the deaminase halves and DNA binding domains.

The C-ter domain of the deaminase domain should be considered as part of the linker, since its flexibility and length would contribute to the interaction of the deaminase halves with each other and with the DNA. This insight is useful in tuning the window of activity of base editors and narrowing down the window to avoid mutations of bystander C-residues residues in the target region.

Effect of the Distance Between DNA Binding Sites with TALE/BAT DNA Binding Domain Pairs

The nature of DNA Binding domain affects the window of activity of base editors. In the case of BE41 when TALEs are used as both Left and Right DNA binding domains, wider window of activity with efficient editing is achieved: Replacing the Left-side TALE with synonymous BAT domain resulted in efficient editing with a narrower window of activity; Replacing the Right-side TALE with BAT resulted in smaller window of activity but that comes with cost of lower editing efficiency.

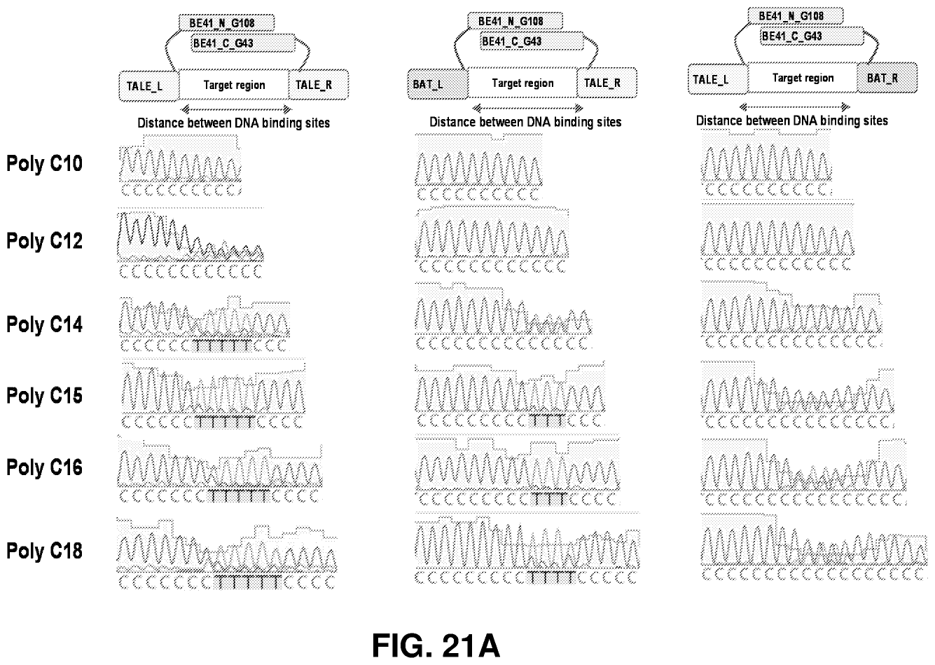

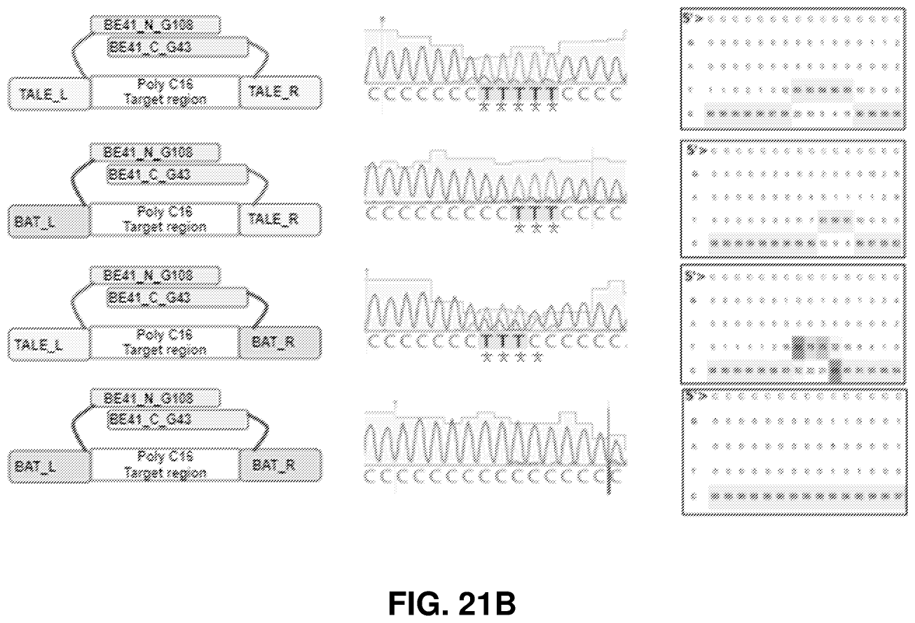

These data show that the nature of DNA binding domain (i.e. the nature of DNA binding domain and deaminase linker, e.g. C-ter domain of the DNA binding domain) is an important factor in design of this class of base editors and would affect the window of activity and editing efficiency, likely through restricting the area within the target region where active deaminase can be effectively reconstituted. This feature is an important design factor in this class of base editors and one parameter that, based on the requirements (e.g. fixing a pathogenic mutation) can be tuned to achieve wider or narrower window of activity and modulating editing efficiency. Tuning editing window is important to avoid off-targets (bystander C residues) within the target region. (FIG. 21B)

### Example 6: Expanding the Window of Activity of Base Editor by Relaxing Deaminase Movement

Whether the lack of flexibility imposed by DNA binding domains restrict the reconstitution of active deaminase and access of deaminase to DNA double helix was assessed. Potentially, relaxing the interaction could facilitate the access of deaminase to DNA and extends the window of activity.

To test this hypothesis, complementary coiled-coiled domains were appended to the end of split deaminase with or without TALE fusions and tested the activity of these modified base editors. As shown in FIG. 22, replacing or removing one of the TALEs in the presence of Coiled-coil led to extended window of activity, demonstrating that relaxing one of the deaminase halves by removing its attached DNA binding domain could help extending the deaminase window of activity toward the removed TALE direction (i.e. removing Right-side TALE leads to extension of the window of activity to the right, and removing the Left-side TALE leads to extension of the window of activity toward Left).

Removing both TALEs simultaneously resulted in a drop of editing below the limit of detection, as expected, due to loss of specificity. These results demonstrate that the editing window is constrained by the restrictions imposed by TALEs on the deaminase halves.

### Example 7: Tuning On-Target Activity and Minimizing Bystander Off-Targets by Moving Window of Activity of the Base Editor

When installing a mutation by base editing, it is often desired to minimize mutation of bystander Cs in the vicinity of the target region, while maximizing editing efficiency of targeted C residues.

Having identified the rules that define the window of activity of Mt-CBE base editors, base editors that can install a mutation corresponding to fixing a pathogenic mitochondrial mutation (mCox1 V421A in mouse mitochondria, corresponding to converting C6589 to T) were designed that minimize off-target mutation of the bystander C residue (C6593).

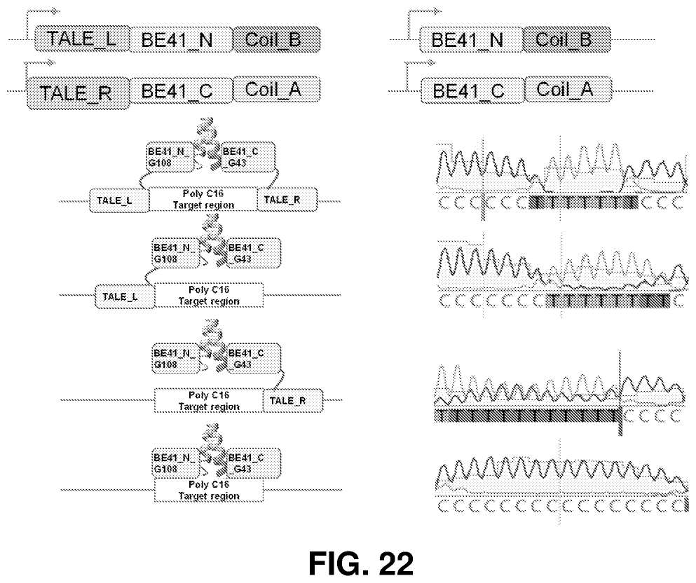

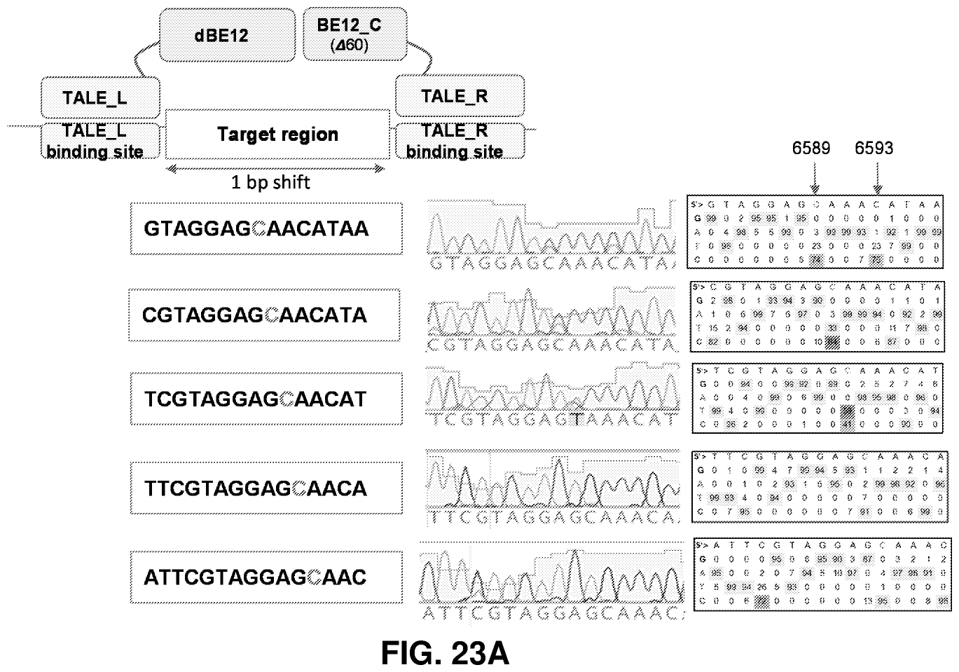

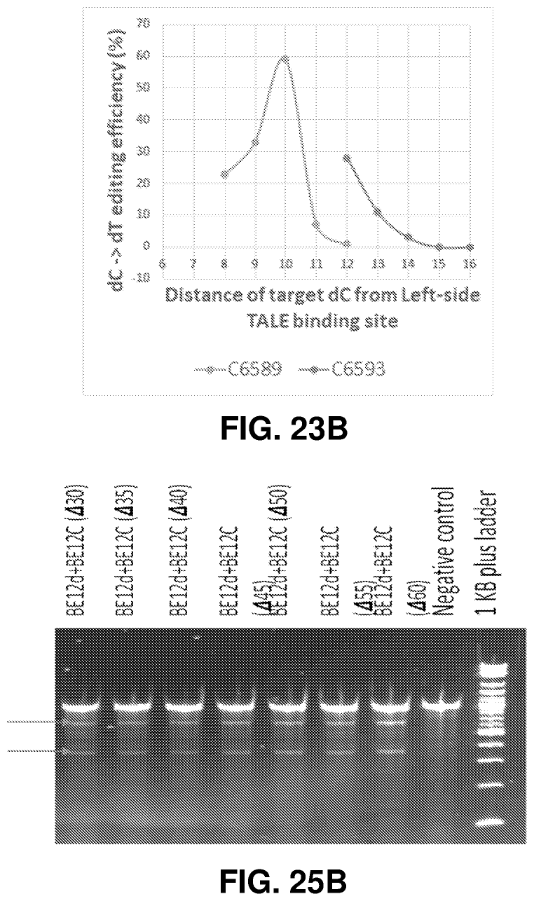

To this end, multiple plasmid substrates encoding the mCox1 target region with 1 bp shift were prepared. The C6589 residue precedes with G residue (GC context), so the BE12 base editor was chosen which was previously demonstrated to edit Cytidines within GC context (note: dddA has no activity on GC containing substrate). By sliding the target region within two non-variable binding sites the position of targeted base within the window of activity of base editors was assessed and optimized without the need to make new base editors that bind different DNA sequences. As shown in FIGS. 23A-23B, the maximal on target editing of C6589 occurs when this C residue is 10 bps (corresponding to 1 turn of double helix) away from the Left-side TALE binding site, indicating that in this base editor architecture the deaminase has better access to dsDNA at this position. The activity drops as the target reside move away from position 10 in both direction, although the drop is sharper when the target residue is moves toward right. The same trend is observed in the case of C6593, and deamination activity goes below the limit of detection as this residue passes position 14 within the target window.

The data:
i) demonstrated efficient and targeted editing of C residues within GC context in the context of a pathogenic mutation;
ii) depict the window of activity of BE12 base editor and a method to tune that window of activity; and
iii) offer a base editing architecture for editing pathogenic C6593 mutation and minimized off-targets by placing the target base 10 base pairs away from the Left-side TALE binding site.

Similar target sliding approaches can optimize the editing efficiency of other base editors and minimize bystander off-targets for other base editors, without the need to make multiple DNA binding domains and base editor.

### Summary: Base Editor Design

Different parameters that affect base editing window of activity and editing efficiency include:

### 1. The Nature of DNA Binding Domain.

It has been established that different types of programmable dsDNA-specific DNA binding domains (including TALEs, ZFs and BATs) can be used to provide specificity in making these base editors.

It has also been established that the nature of DNA binding domain affects the position and span of window of activity. Given that the dsDNA specific deaminases currently have some inherent limitations (e.g., ZFs cannot be designed for all possible targets, TALEs and ZFs and possibly BATs bind to some targets better than the others, etc.), for any given target some optimization regarding the nature of the deaminase may be required to optimize the performance of the base editor.

2. The Nature of dsDNA Specific Deaminase.

The nature of the deaminase domain used affects the sequence context within which cytidine bases can get edited. Previously published dddA deaminase data indicates the dddA deaminase can only edit Cs within TC context (Mok et al.).

The data presented here characterize various deaminases that can edit cytidines within various contexts. This panel of deaminases collectively can be used to edit cytidines in any possible context (AC, CC, GC, TC). One can choose a deaminase that allow maximal on-target editing and minimal off-target editing for a given target. It has also been demonstrated that the nature of deaminase also affects the position and span of window of activity on either forward or reverse strand.

### 3. The Position/Nature of Split Site

The data demonstrate that the position of split site affects (the position and span of) the window of activity of base editor on both forward and reverse strand. Different split position can be used to tune window of activity of the deaminase. Two designs for making split base editors have been devised and provided:

i. A first design strategy involves fusing a “dead”/inactive, full-length copy of deaminase to one DNA binding domain, and fusing a truncated copy of the deaminase with intact active site to the other copy of DNA binding domain (BE12 was used as proof of concept). None of the two copies of deaminase (dead or truncated) are active (individually or as fusions to DNA binding domain). However, when they are brought up together on the target DNA, they can complement each other and reconstitute the deaminase activity (this general design can be used for making split version other enzymes as well, without knowledge about their structure). In this design, the dead copy of the enzyme (which contains a deactivated active site) complements the structural elements for the truncated copy of the enzyme (that have a functional active site but lacks one or more necessary structural elements). This approach can be used for making split proteins that require dimerization for their activity as well.

ii. A second design strategy includes the bigger fragments obtained from two separate split sites of a single protein (BE41 was used as proof of concept). None of the two fragments (i.e. N- and C-ter truncated, overlapping fragments) are active individually, but they reconstitute the enzymatic activity once brought on the target by the DNA binding domains. In this design, each fragment complements the structural motif the other fragment is lacking, and since there are two active sites co-localized on the target, higher enzymatic activity is achieved.

The approaches (i) and (ii) described above are structural data agnostic and can be applied without access to the protein structure and could allow making split proteins that require dimer or multimer formation for their activity. These are as opposed to traditional approach where the protein is split at a single site to non-overlapping N- and C-termini. To design split proteins with the traditional approach often structural data are needed. More importantly, only one copy of the protein can be reconstituted effectively on the target, thus proteins that require dimerization or multimerization cannot be turned into split version using the traditional approach.

### 4. The Nature of the Linker

It has been demonstrated that the length and nature of the linker can affect the position and span of the window of activity by permitting/restricting the area on the dsDNA where the deaminase activity can be reconstituted along the double helix.

It should be noted that the non-essential sequences that may exist in the DNA binding domain and deaminase domain and are immediately attached to the linker should be considered as an extension of the linker. For example, naturally occurring TALEs and TALE-like proteins can tolerate truncations in their C-ter domain without affecting their binding affinity. The non-essential amino acids that are part of the body of DNA binding domain or deaminase domain should be considered as an extension to the linker, and their composition (length/flexibility) could serve as a parameter that can be tuned to tune the editing efficiency and window of activity of the base editor.

### 5. The Distance Between the DNA Binding Domains

Another parameter that affects the position of window of activity on the target region is distance separating the DNA binding factors. It has been demonstrated that to achieve optimal activity the distance between the two binding sites needs to be within a certain range: if the distance is too short, minimal/no editing would occur, likely because the deaminase halves to dsDNA is sterically hindered; on the other hand, if the distance is too far, the efficient concentration of the deaminase halves drops, and the interaction of the deaminase halves becomes less efficient.

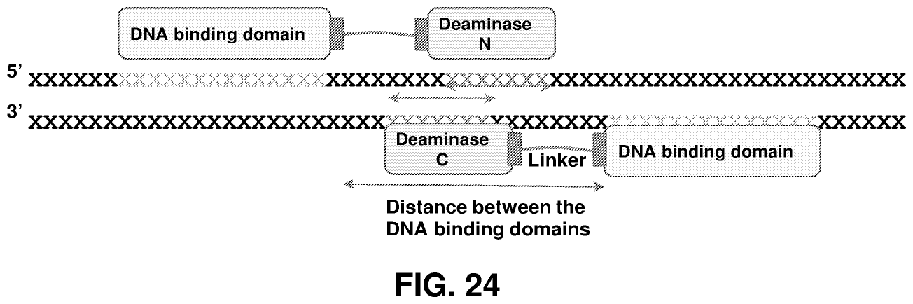

For the tested base editor designs, the optimal window of activity was found to be between 14-20 bps. The optimal distance could be slightly different when different types of DNA binding domains/deaminases/linkers are used. It may be that minimum one turn of dsDNA (10 bps) distance is needed to achieve efficient editing, since below that range the access of the deaminase to dsDNA would be sterically hindered (FIG. 24).

### Example 8: Editing Mitochondrial Genome Using Mt-CBE Base Editors

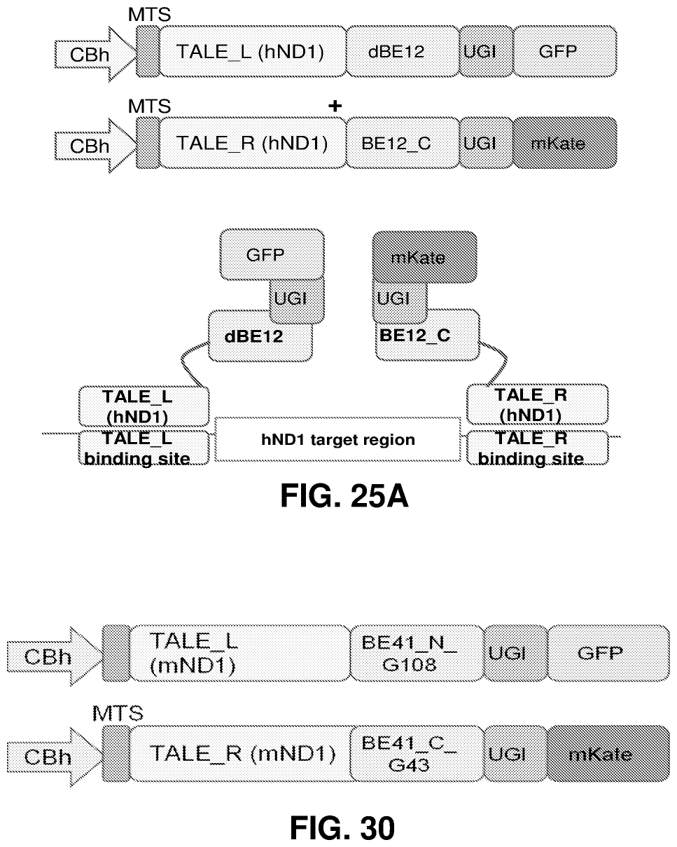

To demonstrate activity of split BE12 base editors, the TALE-split deaminase fusions targeting mitochondrial hND1 gene were fused to UGI (to limit the activity of mitochondrial uracil DNA glycosylase) and GFP (in the case of Left-side TALE fusion) and mKate (in the case of right TALE fusion), and constructs were co-transfected to HEK293T cell line. The cells were harvested after 3 days and the editing outcome was assessed by T7 endonuclease assay (FIGS. 25A-25B).

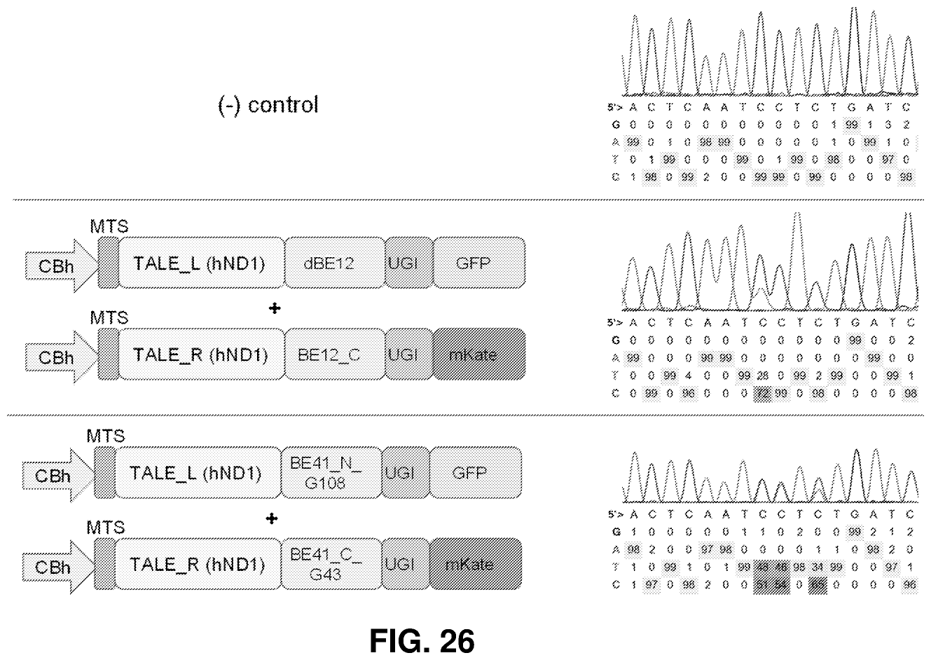

The window of activity for split BE12 vs. BE41 base editors was compared for editing hND1 target in the HEK293 mitochondria. The BE12 editor shows narrower window of activity, whereas BE41 editor result in more efficient editing and wider window of activity. The window of activity for both base editors is consistent with the editing window observed in in vitro experiments. Given the narrower window of activity of BE12 editor, this editor is more suited when minimizing bystander off-target edits is desired (FIG. 26).

### Example 9: Using Alternative DNA Binding Domains (TALEs, BATs, ZFs)

Several alternative DNA binding factors, including zinc finger (ZF), TALE and TALE-like (BAT) proteins were assessed for use in base editing using Mt-CBE.

### Zinc Fingers

Zinc Fingers (ZFs) were assessed as DNA binding factors. Each ZF repeat recognizes 3 nucleotides (triplet) as opposed to one nucleotide per repeat in case of TALE and TALE-like proteins (less repeats, likely to be more stable in cells). ZFs are smaller (two ZF-BEs can fit into a vector) than TALEs and TALE like which makes them better candidates for gene delivery by AAV, however, ZFs cannot be designed for any given target (there are 64 possible triplet nucleotides, but only ˜50 of them can be targeted by the existing ZFs).

### TALE and TALE Like Proteins

TALE and TALE like proteins were also assessed. These are repetitive DNA/RNA binding domains (many of which remains uncharacterized) with the same di-nucleotide binding code as TALEs:
TALEs (T0 rule. TALEs with natural N-ter domain require a T at the beginning of their binding sites for efficient binding. Mutant versions of TALE N-ter has been evolved to have relaxed specificity toward other nucleotides);
RipTALs (G0 rule. The first base in the binding site must be a G);
BATs (relaxed binding. The binding site can start with any nucleotide);
MorTLs (identified metagenome sequences);
Many other uncharacterized TALE-like proteins exist in the genomics databases;
Repeats are usually interchangeable (you can replace one or a few TALE repeat with a TALE-like repeat and they still bind to the same target).

### BATs

BATs are functional in mitochondria and can be used as alternative DNA binding domain for design of base editors. As discussed, using BATs would allow to tune the window of activity of base editors and minimize bystander off-targets. Additionally, BATs binding specificity is more relaxed than TALEs and ZFs. BATs, unlike TALEs that strictly require a T at the beginning of their binding sites (T0 rule), have more relaxed N-terminus binding specificity and do not follow T0 rule.

The binding site for BATs can start with any nucleotide not just T. Zinc Fingers can only target a subset of sequences (not every triplet nucleotides can be target with a ZF repeat). With their relaxed specificity and simple synonymous code as TALEs, BATs offer an interesting alternative DNA binding domain for design of base editors.

### Example 10: Expanding the Scope of Sequences that can be Targeted by dsDNA Base Editing: Engineering TALE N-Terminus, BATs, and ZFs

When designing base editors, the requirement for proximity of the DNA binding sites to the target base(s) to fall within the window of activity (e.g., ˜10 bps away from Left-side TALE binding site, ˜6 bps away from the Right-side TALE binding site, in a 16 bp target region) of the base editor imposes additional restriction on the position of DNA binding sites. For example, to achieve maximal base editing with BE12 base editors, the distance between the Left-side binding domain should be 9-11 bps. Furthermore, programmable DNA binding domains such as Zinc fingers and TALEs have some inherent limitations that could make targeting certain bases challenging. In the case of ZFs, a subset of sequences cannot be targeted since for ˜15/64 triplet nucleotides there's no ZF repeats that can recognize them. If any of these 15 nucleotide repeats occur in the vicinity of the potential binding site no ZF can be designed. On the other hand, T0 rule and a few other factors including the nature of the first few bps at the binding site are important for efficient binding by TALEs, requirements that may not be satisfied for every given target.

These limitations posed challenges for designing base editors to install m6589C>T mutation. Given the sequence context surrounding this target base, ZFs or TALEs couldn't be designed that provide a high binding score. Nevertheless, a series of base editors using the low score ZFs and TALEs were designed and tested experimentally, but did not observe high editing efficiency of the target base, likely because of low binding affinity of the DNA binding domains. Base editing on targets that lack a suitable context (e.g. presence of T0 at optimal distance from the target base in the case of natural TALE domains) was achieved by two parallel approaches:
1) using TALE with relaxing mutations in their N-terminus; and
2) Using BATs.

For the first approach, using TALE with relaxing mutations in their N-terminus, mutations in the N-terminus of TALE that relaxes T0 specificity and allow targeting binding sites that start with nucleotides other than T were previously identified (see Table 4, below). Incorporating these relaxing mutations into the TALE protein allowed to design TALEs with higher binding score (arrows show the position of binding sites), which were used for editing of the target nucleotide (FIGS. 23A-23B).

TABLE 4
D Optimal Variants
Mutations in TALE N-terminus that relax the T0
requirement (Lamb, et al., 2013, the contents of
which are incorporated herein by reference).

| | |
|---|---|
| TALE NT-T | Asp225-IVGVKQWSGARAL-Glu239 |
| TALE NT-G | Asp225-IVGVKSRSGARAL-Glu239 |
| TALE NT-αN | Asp225-IVGVERGAGARAL-Glu239 |
| TALE NT-βN | Asp225-IVGVKY-HGARAL-Glu239 |

For the second approach, using BATs instead of TALEs, preliminary studies had shown that, unlike TALEs, BATs have no apparent restriction on the starting nucleotide in their target sites. This relaxed specificity greatly expands the scope of DNA sequences that they can target. As the second approach, BATs with relatively high binding score were designed and were able to install C6589T mutation (FIGS. 27A-27B).

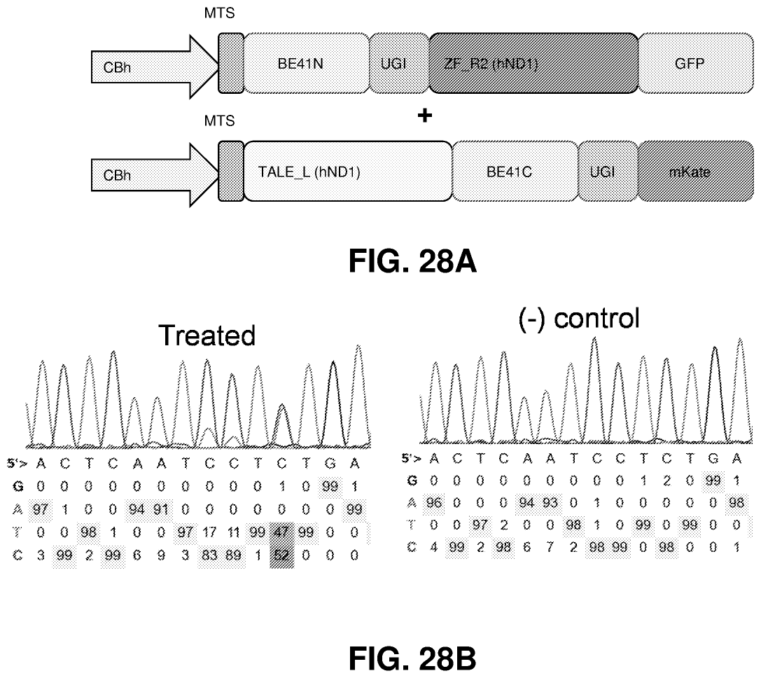

Furthermore, we demonstrated that ZFs can be used as DNA binding domains instead of TALEs (FIG. 28). Changing the type of DNA binding domain leads to the changes in the base editor window of activity, further suggesting that the DNA binding domain and its C-terminus could restrict deaminase domain. This finding could be used to tune window of activity of these deaminases and reduce bystander off-targets. Due to their smaller size, ZF-based editors are attractive for AAV delivery.

### Example 11: Single AAV Base Editor Design Using ZF Binding Domains

TALEs and BATs are relatively large proteins and only one of the two halves of the split base editors can fit within a single Adeno-associated virus (AAV) vector when these domains are used as DNA binding domains. On the other hand, ZFs are relatively smaller DNA binding domains, and it is possible to fit both halves of the base editor into a single AAV (which can accommodate ˜4.5 kb cargo between its LTR repeats).

Two different approaches to accommodate two halves of split ZF-deaminase into a single AAV were tested:
1) P2A peptide (that undergo translational skipping and allow polycistronic expression of multiple proteins from the same transcript in eukaryotes); and
2) Internal Ribosome Entry Site (IRES), that serves as internal initiation site and allow bicistronic expression of transcripts in mammalian cells.

Despite multiple attempts, it was not possible to clone the P2A constructs in E. coli (all obtained colonies contained deactivating mutations (frameshift or stop codons) that rendered the protein non-functional), suggesting that even basal/cryptic expression of the in-frame spilt deaminase is toxic to the cells.

Since in this design the N-ter and C-ter of the deaminase are translated into a single polypeptide, if expressed, they can spontaneously reconstitute the functional dsDNA-specific deaminase which is toxic to the cells.

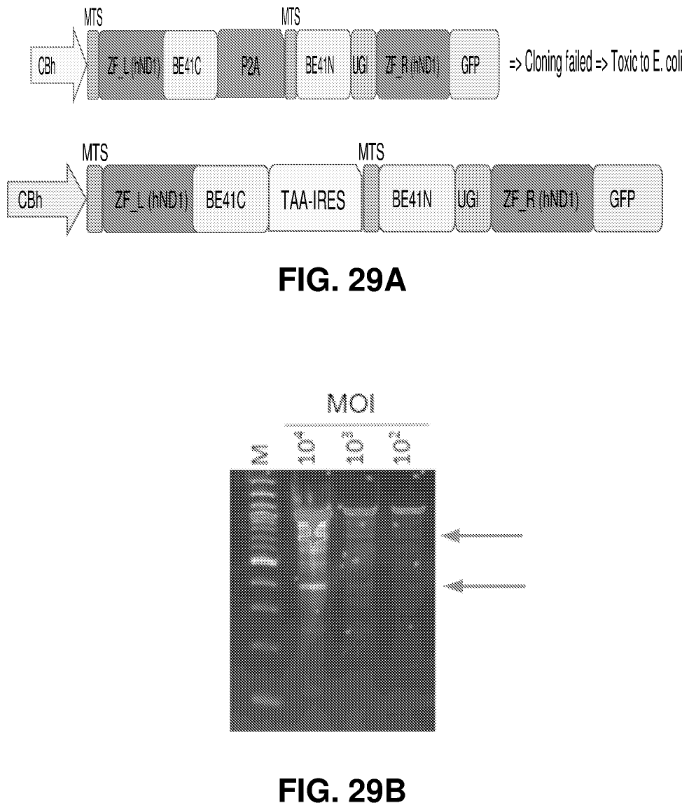

On the other hand, in the IRES design, the two split halves are expressed as two separate polypeptide chains, and can only colocalize and reconstitute the functional deaminase at the vicinity of the target region defined by the DNA binding domains they are attached to (there is a stop codon (TAA) before the IRES to ensure translation termination). It was possible to clone and sequence verify this construct, and confirm its activity in the mitochondria in mammalian cells. The IRES vector was packaged into AAV2 Capsid using HEK293 AAVpro cell line (Teknova) and the viral particles were used to transduce HEK293 cells at the indicated MOIs. Cells were harvested after two weeks and the editing of hND1 locus was assessed by T7 endonuclease assay. (FIG. 29A-29B)

### Example 12: Editing Mitochondrial Genome in the Mouse NIH3T3 and ES Cell Line

Base editing in the mouse NIH3T3 cell line was carried out by editing mND1 loci in NIH3T3 cells. Vectors encoding split BE41 base editor halves were delivered to NIH3T3 with either transfection or transduction (AAV2 capsid) with no selection. T7 endonuclease assay was used to detect outcome of editing. Editing was detected 5 days post transfection in transfected cells by T7 endonuclease assay. In the case of AAV transduction, editing was detected 2 weeks post transduction by T7 endonuclease assay. The observed delivery efficiency to NIH3T3 cell line was <20%, which to a large extent accounts for the relatively low apparent editing efficiency in compare to HEK cells.

Upon successful demonstration of base editing in the mouse NIH3T3 cell line, introducing these edits into mouse ES cells was further demonstrated. (FIG. 30)

### Installing Pathogenic NDI E24K Mutation (m.2820G>A) in Mouse ES Cells

### Experimental Design:

Split deaminase constructs (TALE-BE-left and TALE-BE-right targeting mouse ND1 gene) with a puromycin selection marker were delivered to C57BL/6J Embryonic Stem (ES) cells by electroporation.

Transfectants were selected at the presence of puromycin for a week, after which clonal populations were picked and transferred to individual wells of 96-well plate and their total DNA was extracted.

The target region was amplified using gene-specific primers and Illumina adapters were added to the amplicons by a second round of PCR. The amplicons were sequenced by Illumina MiSeq (2×100 bp Paired End). Reads were demultiplex, paired reads were merged, and analysed by Variation/SNP analysis module of the Geneious Prime.

No variant was detected above the limit of detection by NGS (0.1%) in the negative (GFP-treated) control

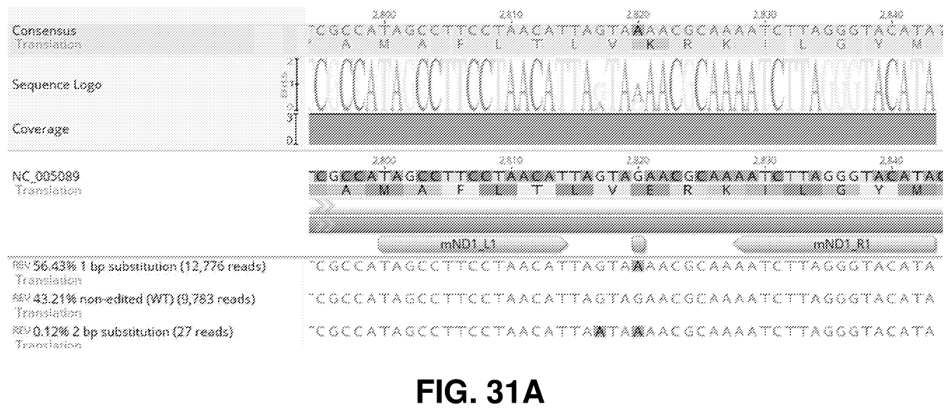

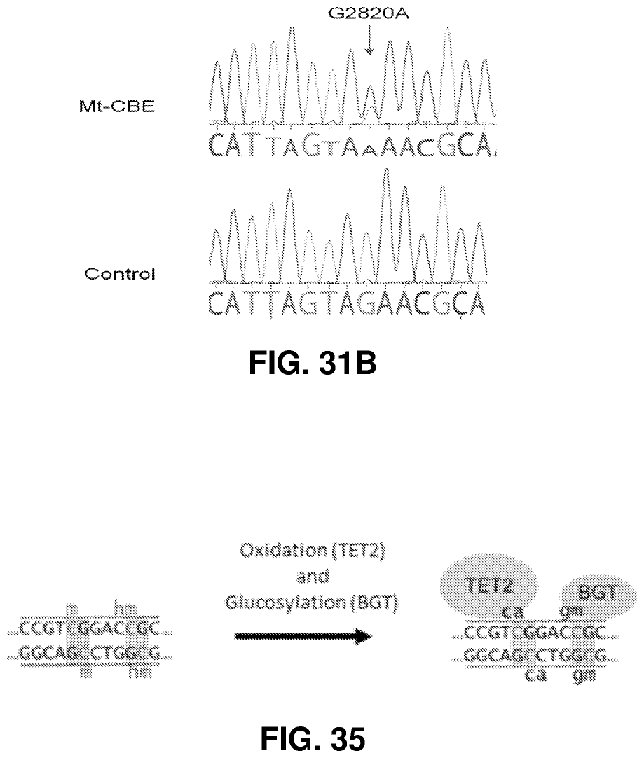

In cells treated with the base editor constructs, the allele harboring the on-target edit (m.2820G>A) comprised the main variant (56.43%). Very low level (0.12%) of a bystander mutation (m.2817G>A) was also detected. No indel (insertion/deletion) was detected above limit of detection (FIG. 31A-31B).

### Summary: Base Editors for Genome Engineering Applications

The data establish a robust system for genomic engineering, that enables context —specific editing, with few bystander edits, that can be used to edit both mitochondrial and nuclear genomes.

Mitochondrial genome editing has many implications, in cancer, aging, and other genetic diseases. In the absence of genetic tools that allow manipulation of mitochondrial genomes and performing forward genetics studies, the described systems for genomic editing enable enhanced understanding of genetic diseases that have thus far been limited to correlative studies. The disclosed Base editors facilitate studies of the effects of mitochondrial mutations with forward genetics, to gain clear insight into effect of mitochondria in these diseases, and develop appropriate therapies.

Analogous approaches can be used to develop double stranded DNA-specific Adenosine deaminase (dsADA), either by mining natural diversity or evolving an adenosine deaminase (ADA) that is active on dsDNA. Such dsADA couldenable A to G (and T-to-C) base editing analogous to what is demonstrated in the data with the C-to-T (and A-to-G) mutations with dsCDAs. Base editing viadsADAs have potential to address an additional 40 pathogenic mutations in mitochondria increasing the number of addressable mutations from 38/93 to 78/93.

The base editors utility is not limited to mitochondria or nuclear genomes, it can be used to edit other dsDNA moieties both inside and outside of the cells and within membranous organelles (e.g. chloroplasts and plastids).

Use of RNA-guided nuclease as DNA binding domain (instead of TALEs or ZFs): For nuclear genome engineering applications, RNA guided proteins (e.g. CRISPR-Cas9) can be used as DNA binding protein instead TALEs and ZFs. The context-specificity of dsCDAs could limit bystander mutations which could be advantageous over the use of ssDNA specific CDAs (e.g. APOBEC) as the deaminase domain (which is being used in the existing CRISPR-based base editing technologies.

Making animal models: Making animal models of mitochondrial genetic diseases: Given the absence of any reliable technology to introduce precise edits to mitochondrial genome, making animal models for mitochondrial genetic diseases has been extremely difficult if not impossible. The base editors not only could facilitate fixing genetic diseases, they can also be used to make animal models. This would enable forward genetics studies of these genetic diseases as well as mitochondrial physiology, and genetic heteroplasmy, which has been impossible to date due to lack of mitochondrial genetic engineering technologies.

Engineering mitochondria and chloroplasts in plants (and other organelles that encode their own genomes): Use of CRISPR for engineering other membranous organelles with their own genome (e.g. chloroplast and other plastids) faces same challenges as editing mitochondria. The protein-only editors (programmable DNA binding domains fused to dsDNA-specific deaminases could be used to edit these organelles genomes (e.g. to improve crops, or make them immune to certain genetic diseases like male sterility)

Functional genetic screening for the study of metabolic disorders, cancer, and aging or biotechnological applications (e.g., engineering ethanol tolerance or improving aerobic fermentation in yeast or improving crops): Due to the absence of methods to selectively mutagenize mitochondrial genome, it has not been possible to apply functional genetic screening strategies to mitochondrial genome. The identified deaminases can be expressed transiently in mitochondria of cells of interest (e.g., mammalian cells, yeast cells, etc.) to introduce genetic diversity into those mitochondria of those cells. These cells can then be subjected to a selective pressure or functional screening schemes (e.g., selecting for faster proliferation or presence of cancer markers, or aging markers, or tolerance to ethanol) to identify genetic variants that are involved in those diseases or processes.

### Example 13: Enzymatic Epigenetic Sequencing

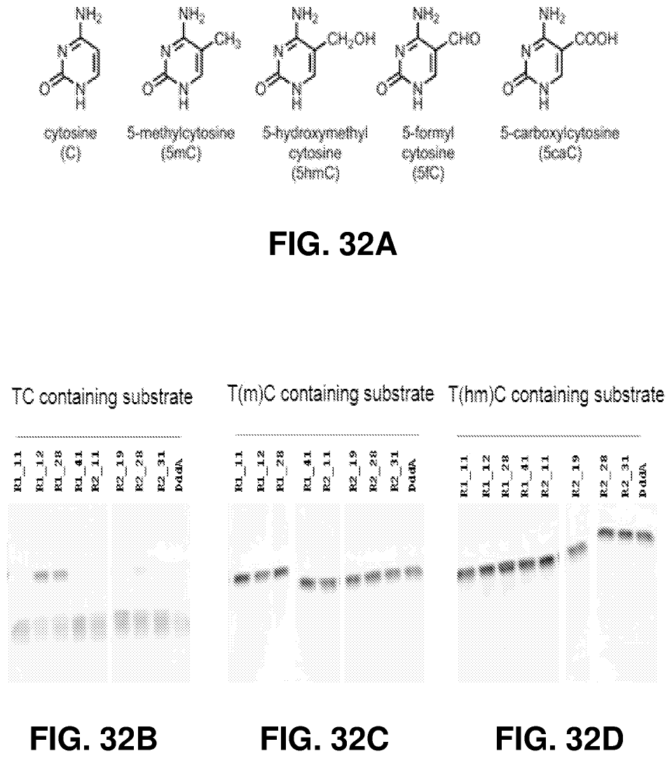

It has been established that different dsDNA-specific deaminases (dsCDAs) show different activities on cytidine and its various modifications, including epigenetic markers, such as 5mC, 5hmC, 5fC, 5caC (FIG. 32A). This feature can be leveraged to differentially mark various epigenetic cytidine modifications, which can then be read by sequencing methods.

### Methods

This Method Offers an Enzymatic Alternative to Bisulfite Sequencing, and Address shortcoming and technical limitations associated with bisulfite treatment of DNA, thus minimizing generating better quality results.

### Deamination Assay

The activity of dsDNA-specific deaminases was tested on non-methylated and methylated cytidine (5mC and 5hmC) by deamination assay. [A15]TC[A15] (SEQ ID NO: 272), [A15]T(5mC)[A15], and [A15]T(5hmC)[A15] annealed to the complementary sequences were used as the substrates.

Assay to Assess dsCDA Activity on Modified Nucleotides

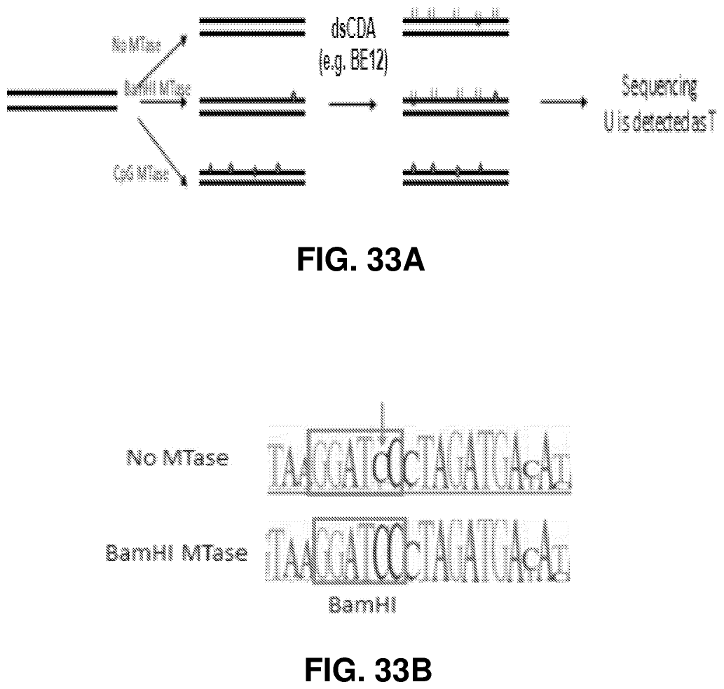

To assess the activity of the dsCDAs on methyl cytidine (5mC), a ˜1 kb PCR fragment was methylated using BamHI Methyltransferase (site-specific MTase) and CpG Methyltransferase (that methylate DNA at CpG sequences) and used as substrates. Full length, isolated dsDNA-specific deaminase domains (dsCDAs) were expressed in the IVT system for two hours. The expressed dsCDAs were incubated with the substrate and for one hour, after which the substrate in the reactions were PCR amplified and the editing frequency was assessed by Sanger as well as NGS sequencing (FIG. 33A).

Assay to Assess Different dsCDA Activity on Modified Nucleotides

The deaminase assay was carried out using each of two DNA substrates, including GTACACCATCCGTCCC (SEQ ID NO:274) and GTGTTCTCTATTTCAC (SEQ ID NO:274), each modified to include either 5caC, 5fC, 5hmC or 5mC, respectively, with each of three dsDNA deaminases, including BE_R1_11, BE_R1_28, and BE_R1_41, over a period of 24 hours. Samples were sequenced following 15 mins, 45 mins, 2 hrs and 24 hrs of incubation.

### Enzymic Oxidation and Glucosylation

The DNA substrates containing GTACACCATCCGTCCC (SEQ ID NO:274) and GTGTTCTCTATTTCAC (SEQ ID NO:275) were oxidated by treatment with TET2 enzyme and glucosylated by treatment with BGT enzyme, then incubated with BE_R1_12 or BE_R1_41 deaminase for either one or two hours, to assess the efficacy of deamination.

### Results

The deamination assay demonstrated that deaminases are more active on non-methylated cytidines [(m)C] (FIG. 32B), but not on methylated cytidines (5mC and 5hmC) (FIGS. 32C-D).

The assay to identify DNA modifications demonstrated that editing efficiency (C-to-T conversion) was higher on non-methylated dC residues, suggesting that dsCDAs act differentially on non-methylated and methylated DNA, as demonstrated in the frequency sequence logo for NGS results for samples in which substrates treated with BamH1 methyltransferase followed by BE_R1_12 (FIG. 33B).

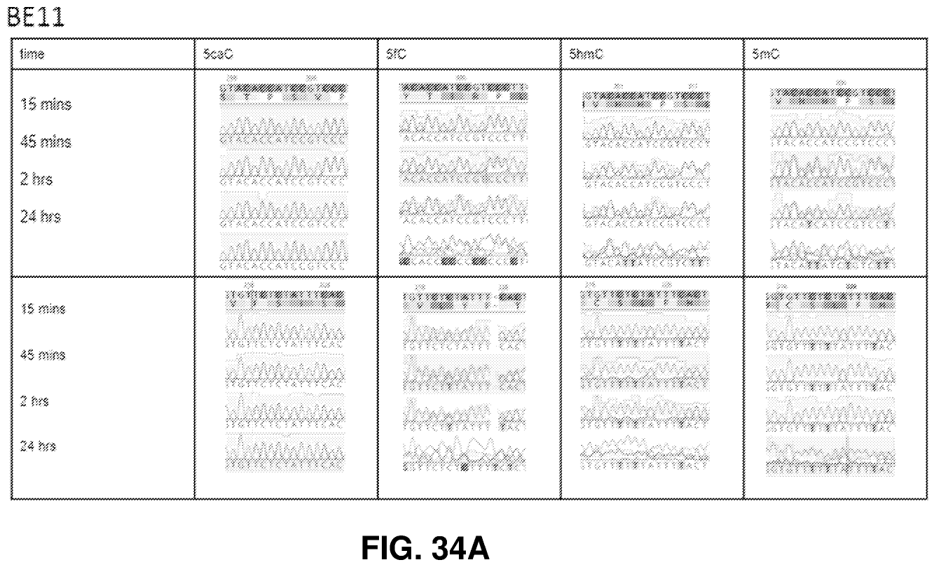

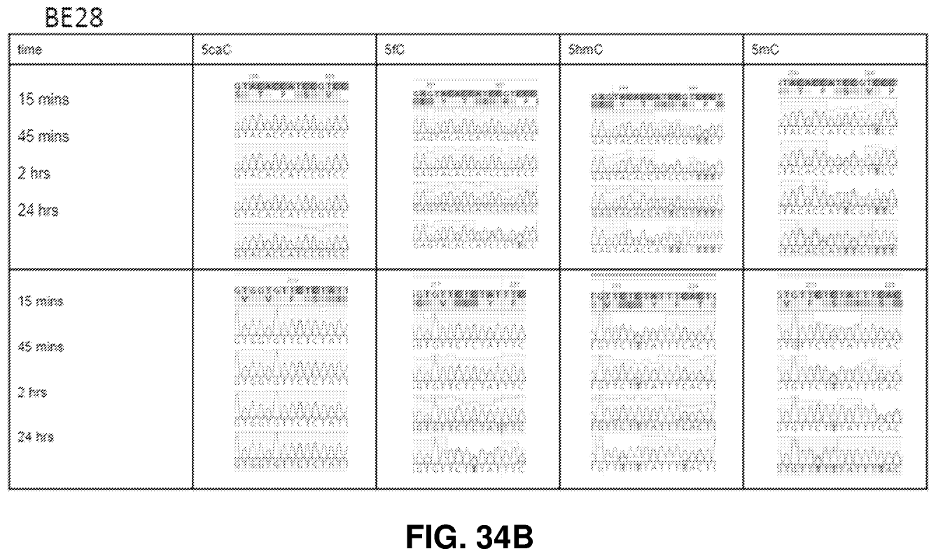

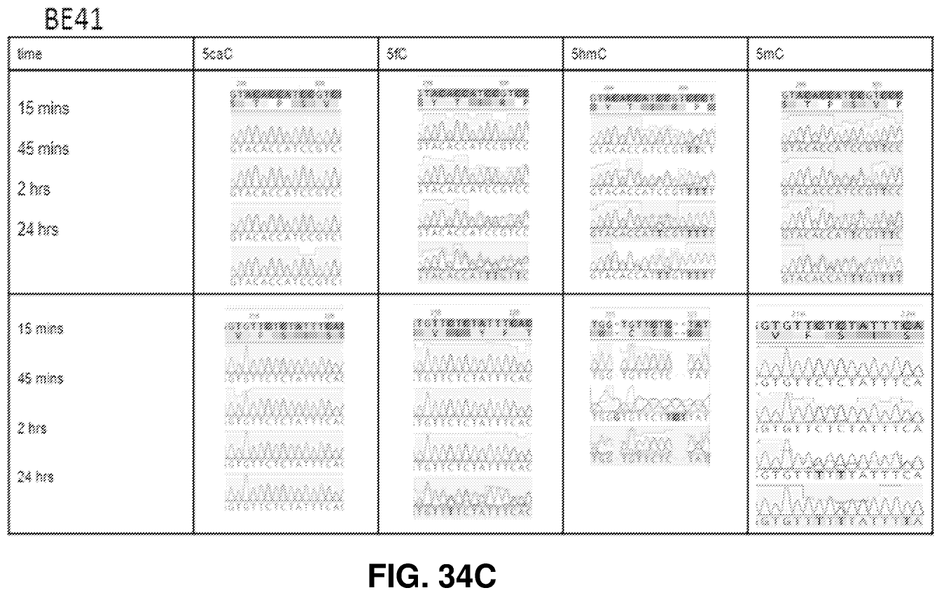

The results of the deaminase assay using each of the DNA substrates (SEQ ID NOs:274 and 275) are shown for BE_R1_11 (FIG. 34A), BE_R1_28 (FIG. 34B) and BE_R1_41 (FIG. 34C), respectively.

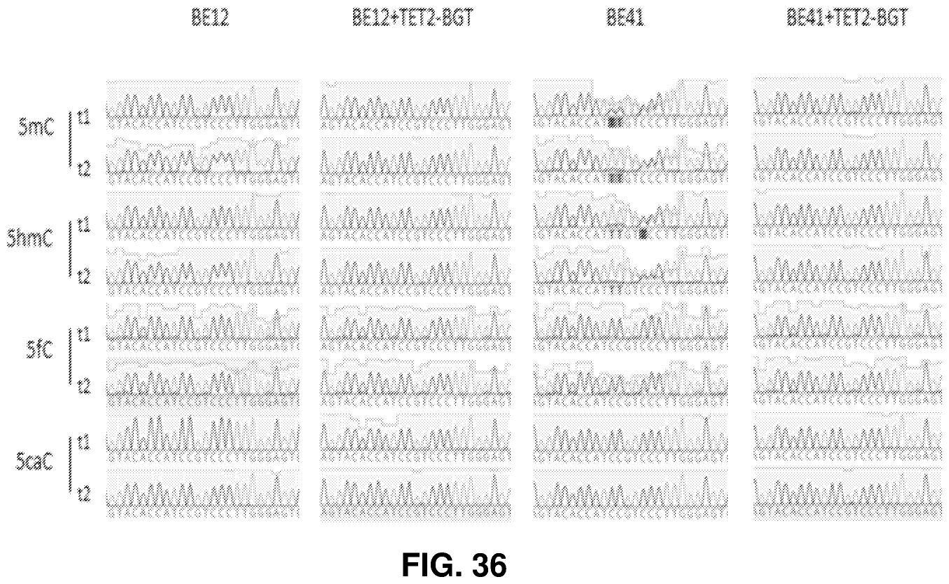

Oxidation and glucosylation enhanced deaminase protection, as indicated by the deamination of 5mC to T by BE_R1_41 in GTACACCATCCGTCCC (SEQ ID NO:274), yielding GTACACCATTTGTCCC (SEQ ID NO:276) and 5hmC to T by BE_R1_41 yielding GTACACCATTTGTCCC (SEQ ID NO:276) and GTACACCATTTGTTCC (SEQ ID NO:277) in the absence of Oxidation and glucosylation by TET2 and BGT (see FIG. 36).

Bisulfite damages and fragments the DNA. ssDNA deaminases require DNA denaturation and expose it to damage. Therefore, dsDNA deaminases provide a better solution, as modified cytosines are not deaminated and show up as a cytosine during sequencing. Unmodified cytosines are deaminated and show up as Uracil during sequencing.

DNA can be modified by treatment with Bisulfite or with dsCDA, then were PCR amplified and sequenced.

### Example 14: Diversity Generation in DNA

Methods for introducing diversity in DNA have been established.

### Methods

To generate diversity in a dsDNA of interest (e.g., a gene encoding a protein of interest), dsDNA was treated with the dsDNA-specific deaminase to create a library of variants of the gene of interest. The library is then subjected to various directed evolution strategies (e.g., ribosome display) or other selection/screening-based methods. Diversity generation can be performed in vitro (e.g., by putting in contact the deaminase protein with DNA substrate of interest) or in vivo, by putting the deaminase domain, either as an isolated domain, or in fusion with an addressing domain (e.g., DNA binding domain, RNA polymerase domain, transcription factor, or other DNA interacting domains).

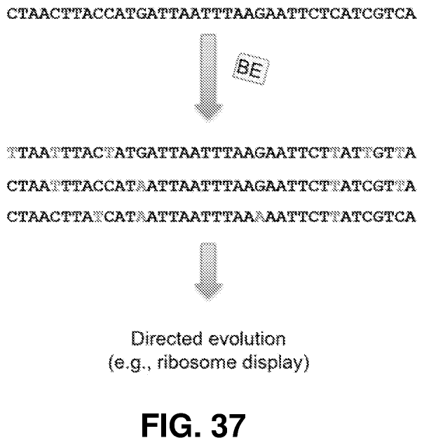

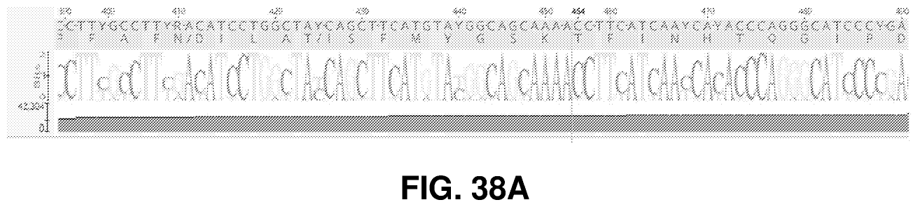

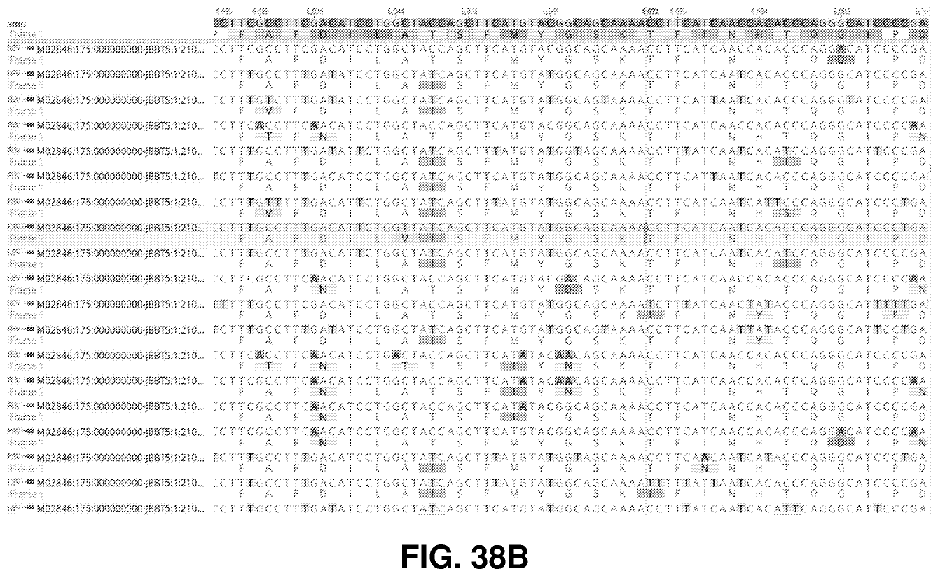

In a representative example, the activity of one or more deaminases on a substrate DNA CTAACTTACCATGATTAATTTAAGAATTCTCATCGTCA (SEQ ID NO:280), leads to three different deamination products TTAATTTACTATGATTAATTTAAGAATTCTTATTGTTA (SEQ ID NO:281), CTAATTTACCATAATTAATTTAAGAATTCTTATCGTTA (SEQ ID NO:282), and CTAACTTATCATAATTAATTTAAAAATTCTTATCGTCA (SEQ ID NO:283), respectively (FIG. 37A-B).

### Results

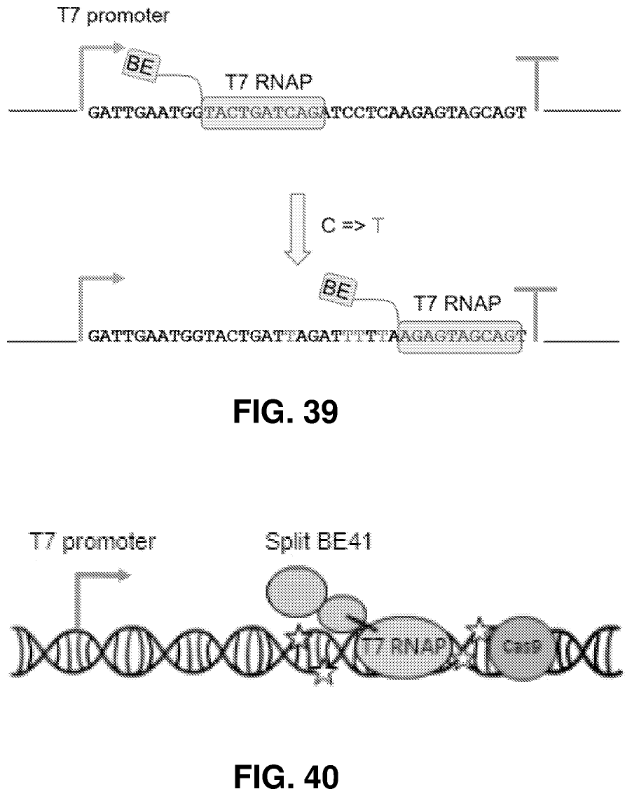

In vitro diversity generation: The frequency sequence logo and NGS reads for PCR fragments resulting from deaminase activity of BE_R1_12 deaminase on DNA substrate are shown in FIGS. 39A-39B, which demonstrate the varied deamination of C to T and G to A at different positions within a library of different sequences generated as a result of deaminase activity double-stranded DNA substrate. In brief, isolated BE_R1_12 was expressed in the IVT system for two hours at 37 C, and then the expressed deaminase was incubated for an hour with the dsDNA substrate. The edited/diversified substrate was assessed by NGS. This approach could serve as an alternative to error-prone PCR for making variant libraries of DNA of interest.

In vivo diversity generation assay: a full-length deaminase can be used for in vitro diversity generation; however, it may cause toxicity for in vivo applications. To circumvent this limitation, a split approach was used. One split half of the BE41 (BE41_G108_C) was fused to T7 RNA polymerase (which served as a targeting domain). The second half (BE41_G108_N) was expressed as a free-floating enzyme. A T7 promoter was appended to the upstream of the target sequence, which was then incubated with the BE41_G108_C-T7 fusion and BE41_G108_N proteins (FIG. 40). CRISPRi (i.e., gRNA/dCas9) was used to block the progress of T7 RNA polymerase on the target and delineate boundary of diversity generation downstream of the T7 promoter and, at the same time, to increase the residence time of the deaminase on the target region. This approach can be used for efficient diversity generation in defined region within living cells for continuous in vivo evolution of traits of interests and cellular barcoding. The activity of the disclosed deaminases on dsDNA would be advantageous for these applications in compare to the previously described applications based on ssDNA-specific deaminases, as the ssDNA substrates for the latter class of deaminases are generated transiently (within the transcriptional bubble) and remain largely with polymerase protein and thus inaccessible to the deaminase.

Other DNA interacting domains can be used as DNA targeting domains in analogous ways. In some form, a similar approach can be used to identify the genome-wide target sites of DNA interacting proteins of interest (e.g., transcription factors) as a high-throughput alternative to the traditional ChIP-Seq. To this end, a dsDNA-specific deaminase domain (either full length, or in split form) is fused to the DNA binding domain of interest and the fusion proteins is expressed in cells of interest (usually the native cell type of the DNA interacting protein of interest). The footprint (i.e. binding sites) of the DNA interacting domain can then be identified by sequencing the whole genome of the cells and looking for segments of the genome with elevated (C-to-T) mutations.

In the in vivo assay, gRNA/dCas9 was used to block progress of T7 polymerase on the target and increase the residence time of the deaminase on the target region (defined by T7 promoter and the gRNA binding site), giving rise to diversity in the substrate sequence.

It is understood that the disclosed methods and compositions are not limited to the particular methodology, protocols, and reagents described as these can vary. It is also to be understood that the terminology used herein is for the purpose of describing particular embodiments only and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a step is disclosed and discussed and a number of modifications that can be made to a number of components including the step are discussed, each and every combination and permutation of step and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D.

Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in algorithms or methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a,” “an,” and “the” include plural reference unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives.

Every composition disclosed herein is intended to be and should be considered to be specifically disclosed herein. Further, every subgroup that can be identified within this disclosure is intended to be and should be considered to be specifically disclosed herein. As a result, it is specifically contemplated that any composition, or subgroup of compositions can be either specifically included for or excluded from use or included in or excluded from a list of compositions. For example, any group or set of deaminases or deaminase domains can have specifically excluded the deaminase domain of DddA from Burkholderia cenocepacia, the deaminase domain of Uniprot ID NO.: AOAOK1EKV1_CHOCO from Chondromyces crocatus, Uniprot ID NO.: C5ALM7_BURGB from Burkholderia glumae (strain BGR1), or any combination of these.

Those skilled in the art will recognize, or be able to ascertain using no more than routine experimentation, many equivalents to the specific embodiments of the method and compositions described herein. Such equivalents are intended to be encompassed by the following claims.

We claim:

1. An isolated deaminase domain, wherein the deaminase domain can deaminate double-stranded DNA, wherein the deaminase domain has greater deaminase activity on double-stranded DNA comprising a target nucleotide sequence as compared to the deaminase activity of the deaminase domain on double-stranded DNA that does not comprise the target nucleotide sequence,
wherein the target nucleotides are each individually fully or partially defined and are in a fixed sequential relationship to each other, and
wherein the deaminase domain is not the deaminase domain of DddA from Burkholderia cenocepacia.

2. The deaminase domain of claim 1, wherein the target nucleotide sequence comprises two or more target nucleotides,
wherein the target nucleotides are each individually fully or partially defined and are in a fixed sequential relationship to each other.

3. The deaminase domain of claim 1 or 2, wherein the target nucleotides are GC, AC, or CC.

4. The deaminase domain of any one of claims 1-3, wherein the deaminase domain comprises two portions,
wherein the deaminase domain is only capable of deaminating when the two portions are combined together.

5. The deaminase domain of any one of claims 1-4, wherein the deaminase domain can deaminate cytosine nucleotides.

6. The deaminase domain of one of claims 1-5, wherein the target nucleotide sequence is AC.

7. The deaminase domain of one of claims 1-5, wherein the target nucleotide sequence is CC.

8. The deaminase domain of one of claims 1-5, wherein the target nucleotide sequence is GC.

9. The deaminase domain of claim 1 or 4, wherein the target nucleotide sequence is TC.

10. The deaminase domain of any one of claims 1-9, wherein deaminase domain comprises an amino acid sequence of any one of SEQ ID NOs:1-4, 9, 11, 14-16, or 40-67, or a fragment or variant thereof.

11. The deaminase domain of claim 10, wherein the deaminase domain comprises BE_R1_41, having an amino acid sequence of SEQ ID NO:4, or an amino acid having at least 70%, 75%, 80%, 85%, 90%, 95%, or 99% sequence identity to SEQ ID NO:4, or fragment thereof.

12. The deaminase domain of claim 11, wherein the deaminase domain comprises BE_R1_11, having an amino acid sequence of SEQ ID NO:1, or an amino acid having at least 70%, 75%, 80%, 85%, 90%, 95%, or 99% sequence identity to SEQ ID NO:1, or fragment thereof.

13. The deaminase domain of claim 11, wherein the deaminase domain comprises BE_R1_12, having an amino acid sequence of SEQ ID NO:2, or an amino acid having at least 70%, 75%, 80%, 85%, 90%, 95%, or 99% sequence identity to SEQ ID NO:2, or fragment thereof.

14. The deaminase domain of claim 11, wherein the deaminase domain comprises BE_R1_28, having an amino acid sequence of SEQ ID NO:3, or an amino acid having at least 70%, 75%, 80%, 85%, 90%, 95%, or 99% sequence identity to SEQ ID NO:3, or fragment thereof.

15. A targeted base editor comprising the deaminase domain of any one of claims 1-14 and a targeting domain, wherein the targeting domain specifically binds to a base editor target sequence.

16. The targeted base editor of claim 15, wherein the targeting domain comprises a TALE, BAT, CRISPR-Cas9, Cfp1, or Zinc finger.

17. The targeted base editor of claim 15 or 16, wherein the base editor target sequence is selected to be present in a target nucleic acid within 20 nucleotides of an instance of the target nucleotide sequence of the deaminase domain,
wherein the instance of the target nucleotide sequence is selected to be base edited by the targeted base editor.

18. The targeted base editor of claim 17, wherein the base editor target sequence within 20 nucleotides of the instance of the target nucleotide sequence selected to be base edited by the targeted base editor is the only base editor target sequence in the target nucleic acid that is within 20 nucleotides of any instance of target nucleotide sequence.

19. The targeted base editor of claim 17 or 18, wherein the instance of the target nucleotide sequence in the target nucleic acid is the only instance of the target nucleotide sequence of the deaminase domain within 20 nucleotides of the base editor target sequence in the target nucleic acid within 20 nucleotides of the instance of the target nucleotide sequence.

20. The targeted base editor of any one of claims 15-19, wherein the base editor target sequence is present in a mitochondrial DNA, or a chloroplast DNA, or plastid DNA.

21. The targeted base editor of any one of claims 15-20, wherein the base editor comprises two portions,
wherein the first portion includes a first split deaminase domain, and wherein the second portion comprises a second split deaminase domain.

22. The targeted base editor of claim 21, wherein the first portion comprises a split deaminase domain comprising an amino acid sequence of any one of SEQ ID NOs:122-181, and
wherein the second portion comprises a split deaminase domain comprising an amino acid sequence of any one of SEQ ID Nos:127-181, and
wherein the first and second split deaminase domains are inactive alone but are capable of deamination when brought into proximity together.

23. The targeted base editor of any one of claims 21-22, wherein the first split deaminase domain comprises an amino acid sequence of any one of SEQ ID Nos:122-126.

24. The targeted base editor of any one of claims 21-22, wherein both the first and second split deaminase domains comprises a wild-type deaminase domain active site.

25. The targeted base editor of any one of claims 21-24, wherein the first and second split deaminase domains each comprise a fragment or variant of BE_R1_11.

26. The targeted base editor of claim 25, wherein the first split deaminase domain comprises any one of SEQ ID NOs:122, or 127-135, or 150, and
wherein the second split deaminase domain comprises any one of SEQ ID NOs:127-135 or 150.

27. The targeted base editor of claim 25, wherein the first split deaminase domain comprises SEQ ID NO:122, and
wherein the second split deaminase domain comprises any one of SEQ ID NOs:127-134 or 150.

28. The targeted base editor of claim 25, wherein the first split deaminase domain comprises SEQ ID NO:129, and
wherein the second split deaminase domain comprises SEQ ID NO:150.

29. The targeted base editor of any one of claims 21 to 24, wherein the first and second split deaminase domains each comprise a fragment or variant of BE_R1_12.

30. The targeted base editor of claim 29, wherein the first split deaminase domain comprises any one of SEQ ID NOs:124, or 136-140, or 156-167, and
wherein the second split deaminase domain comprises any one of SEQ ID NOs:136-140, or 156-167.

31. The targeted base editor of claim 29 or 30, wherein the first split deaminase domain comprises SEQ ID NO:124, and wherein the second split deaminase domain comprises any one of SEQ ID NOs:156-166

32. The targeted base editor of claim 29 or 30, wherein the first split deaminase domain comprises SEQ ID NO:137, and
wherein the second split deaminase domain comprises SEQ ID NO:142.

33. The targeted base editor of claim 29 or 30, wherein the first split deaminase domain comprises SEQ ID NO:139, and
wherein the second split deaminase domain comprises SEQ ID NO:144.

34. The targeted base editor of claim 22, wherein the first and second split deaminase domains each comprise a fragment or variant of BE_R1_41.

35. The targeted base editor of claim 34, wherein the first split deaminase domain comprises any one of SEQ ID NOs:168-171, and
wherein the second split deaminase domain comprises any one of SEQ ID Nos: 172-175.

36. The targeted base editor of any one of claims 34-35, wherein the first split deaminase domain comprises SEQ ID NO:168, and
wherein the second split deaminase domain comprises SEQ ID NO:173

37. The targeted base editor of claim 34-35, wherein the first split deaminase domain comprises SEQ ID NO:171, and
wherein the second split deaminase domain comprises SEQ ID NO:175.

38. The targeted base editor of claim 34, wherein the first split deaminase domain comprises SEQ ID NO:171, and
wherein the second split deaminase domain comprises SEQ ID NO:173.

39. The targeted base editor of any one of claims 21 to 24, wherein the first and second split deaminase domains each comprise a fragment or variant of BE_R1_28.

40. The targeted base editor of claim 39, wherein the first split deaminase domain comprises any one of SEQ ID NOs:123, or 146-149, or 151-155, and
wherein the second split deaminase domain comprises any one of SEQ ID NOs:146-149, or 151-155.

41. The targeted base editor of claim 39 or 40, wherein the first split deaminase domain comprises SEQ ID NO:123, and
wherein the second split deaminase domain comprises any one of SEQ ID NOs:149, or 151-153.

42. The targeted base editor of any one of claims 21 to 24, wherein the first and second split deaminase domains each comprise a fragment or variant of BE_R4_21.

43. The targeted base editor of claim 42, wherein the first split deaminase domain comprises any one of SEQ ID NOs:125, or 176-177, and
wherein the second split deaminase domain comprises any one of SEQ ID NOs:176-177.

44. The targeted base editor of claim 42, wherein the first split deaminase domain comprises SEQ ID NO:125, and
wherein the second split deaminase domain comprises SEQ ID NO:177.

45. The targeted base editor of claim 42, wherein the first split deaminase domain comprises SEQ ID NO:176, and
wherein the second split deaminase domain comprises SEQ ID NO:177.

46. The targeted base editor of any one of claims 21 to 24, wherein the first and second split deaminase domains each comprise a fragment or variant of BE_R2_11.

47. The targeted base editor of claim 46, wherein the first split deaminase domain comprises any one of SEQ ID NOs:126, or 180-181, and
wherein the second split deaminase domain comprises any one of SEQ ID NOs:180-181.

48. The targeted base editor of claim 42, wherein the first split deaminase domain comprises SEQ ID NO:125, and
wherein the second split deaminase domain comprises any one of SEQ ID NOs:180-181.

49. The targeted base editor of claim 42, wherein the first split deaminase domain comprises SEQ ID NO:180, and
wherein the second split deaminase domain comprises SEQ ID NO:181.

50. The targeted base editor of any one of claims 22 to 49, wherein the first, or the second portion, or both the first and second portions comprises a programmable DNA binding domain selected from the group consisting of a TALE, BAT, CRISPR-Cas9, Cfp1, or Zinc finger.

51. The targeted base editor of claim 50, wherein one programmable DNA binding domain is a TALE selected from the group consisting of a Left hand side TALE and a Right hand side TALE.

52. The targeted base editor of claim 50 or 51, wherein one programmable DNA binding domain is a Left hand side TALE comprising an amino acid sequence of any one of SEQ ID NOs:90, 92, 95, 97-106.

53. The targeted base editor of any one of claims 50-52, wherein one programmable DNA binding domain is a Right hand side TALE comprising an amino acid sequence of any one of SEQ ID NOs:91, 93-94, 96, 108-113.

54. The targeted base editor of any one of claims 50-53, wherein one or more programmable DNA binding domain is TALE that binds to mitochondrial mND1 DNA, having an amino acid sequence comprising any one of SEQ ID NOS:95-96.

55. The targeted base editor of any one of claims 50-54, wherein one programmable DNA binding domain is a Right hand side TALE that binds to mitochondrial mND1 DNA, having an amino acid sequence comprising SEQ ID NO:96.

56. The targeted base editor of any one of claims 54 or 55, wherein one programmable DNA binding domain is a Left hand side TALE that binds to mitochondrial hND1 DNA, having an amino acid sequence comprising SEQ ID NO:95.

57. The targeted base editor of claim 51, wherein one or more programmable DNA binding domain is TALE that binds to mitochondrial mCOX1 DNA, having an amino acid sequence comprising any one of SEQ ID NOs:99-106, or 108-113.

58. The targeted base editor of claim 57, wherein one programmable DNA binding domain is a Right hand side TALE that binds to mitochondrial mCOX1 DNA, having an amino acid sequence comprising any one of SEQ ID NOs:108-113.

59. The targeted base editor of any one of claims 57 or 58, wherein one programmable DNA binding domain is a Left hand side TALE that binds to mitochondrial mCOX1 DNA, having an amino acid sequence comprising any one of SEQ ID NOs:90-106.

60. The targeted base editor of claim 50, wherein one or more programmable DNA binding domain is TALE that binds to h12 DNA, having an amino acid sequence comprising SEQ ID NO:98

61. The targeted base editor of claim 50, wherein one programmable DNA binding domain is a TALE with NT(G)N-terminal domain, having an amino acid sequence comprising SEQ ID NO:114.

62. The targeted base editor of any one of claim 50, wherein one programmable DNA binding domain is a TALE with NT(bn)N-terminal domain, having an amino acid sequence comprising SEQ ID NO:115.

63. The targeted base editor of claim 51, wherein one or more programmable DNA binding domain is TALE that binds to the mitochondrial ND6 DNA, having an amino acid sequence comprising any one of SEQ ID NOs:92-94.

64. The targeted base editor of claim 63, wherein one programmable DNA binding domain is a Right hand side TALE that binds to the mitochondrial ND6 DNA, having an amino acid sequence comprising any one of SEQ ID NOs:93-94.

65. The targeted base editor of any one of claims 63 or 64, wherein one programmable DNA binding domain is a Left hand side TALE that binds to mitochondrial mND6 DNA, having an amino acid sequence comprising SEQ ID NO:92.

66. The targeted base editor of claim 51, wherein one or more programmable DNA binding domain is TALE that binds to mitochondrial hND DNA, having an amino acid sequence comprising any one of SEQ ID NOs:90-91.

67. The targeted base editor of claim 66, wherein one programmable DNA binding domain is a Right hand side TALE that binds to mitochondrial hND DNA, having an amino acid sequence comprising SEQ ID NO:90.

68. The targeted base editor of any one of claims 66 or 67, wherein one programmable DNA binding domain is a Left hand side TALE that binds to mitochondrial hND DNA, having an amino acid sequence comprising SEQ ID NO:91.

69. The targeted base editor of claim 50, wherein one programmable DNA binding domain is a TALE that binds to h11 DNA, having an amino acid sequence comprising SEQ ID NO:97.

70. The targeted base editor of any one of claims 50-69, wherein one or both of the first and second portions independently comprise a zinc finger programmable DNA binding domain.

71. The targeted base editor of any one of claims 50-70, wherein one programmable DNA binding domain is a zinc finger selected from the group consisting of a Left hand side zinc finger and a Right hand side zinc finger.

72. The targeted base editor of any one of claims 50 or 57 or 70-71, wherein one programmable DNA binding domain is a zinc finger that binds to mCOX1 DNA, having an amino acid sequence comprising any one of SEQ ID NOs:82-89.

73. The targeted base editor of any one of claims 50, or 70-72, wherein one programmable DNA binding domain is a Right hand side zinc finger that binds to mCOX1 DNA, having an amino acid sequence of any one of SEQ ID NOS:82-86, or 87-89.

74. The targeted base editor of any one of claims 50 or 70-73, wherein one programmable DNA binding domain is a Left hand side zinc finger that binds to mCOX1 DNA, having an amino acid sequence comprising any one of SEQ ID NOs:82-86.

75. The targeted base editor of claims 50, or 66, or 70-71, wherein one programmable DNA binding domain is a zinc finger that binds to hND DNA, having an amino acid sequence comprising any one of SEQ ID NOs:74-81.

76. The targeted base editor of any one of claims 50 or 70 or 74-75, wherein one programmable DNA binding domain is a Right hand side zinc finger that binds to hND DNA, having an amino acid sequence of any one of SEQ ID NOs:78-81.

77. The targeted base editor of any one of claims 50 or 70, or 74-76, wherein one programmable DNA binding domain is a Left hand side zinc finger that binds to hND DNA, having an amino acid sequence comprising any one of SEQ ID NOs:74-77.

78. The targeted base editor of any one of claims 50-77, wherein one or both of the first and second portions independently comprise a BAT programmable DNA binding domain.

79. The targeted base editor of claim 50-78, wherein one programmable DNA binding domain is a BAT selected from the group consisting of a Left hand side BAT and a Right hand side BAT.

80. The targeted base editor of any one of claims 50 or 57 or 72, wherein one programmable DNA binding domain is a BAT that binds to mCOX1 DNA, having an amino acid sequence comprising any one of SEQ ID NOs:118-119.

81. The targeted base editor of any one of claims 50, or 57, or 70, or 72, or 80, wherein one programmable DNA binding domain is a Right hand side BAT that binds to mCOX1 DNA, having an amino acid sequence of any one of SEQ ID NO:119.

82. The targeted base editor of any one of claims 50, or 57, or 70, or 72, or 80-81 wherein one programmable DNA binding domain is a Left hand side BAT that binds to mCOX1 DNA, having an amino acid sequence comprising any one of SEQ ID NO:118.

83. The targeted base editor of claims 50, or 70, or 63, or, 78-79 wherein one programmable DNA binding domain is a BAT that binds to ND6 DNA, having an amino acid sequence comprising any one of SEQ ID NOs:120-121.

84. The targeted base editor of any one of claims 50, or 70, or 63, or, 78-79, or 83, wherein one programmable DNA binding domain is a Right hand side BAT that binds to hND DNA, having an amino acid sequence of any one of SEQ ID NO:121.

85. The targeted base editor of any one of claims 50, or 70, or 63, or, 78-79, or 83-84, wherein one programmable DNA binding domain is a Left hand side BAT that binds to hND DNA, having an amino acid sequence comprising any one of SEQ ID NO:120.

86. The targeted base editor of any one of claims 21-22, wherein the first portion comprises
(a) a first split deaminase domain comprising an amino acid sequence of SEQ ID NO:120, and
(b) a Left hand TALE programmable DNA binding domain; and
wherein the second portion comprises
(c) a second split deaminase domain comprising an amino acid sequence of any one of SEQ ID NOs:156, 158, 160 or 164, and
(d) a Right hand TALE programmable DNA binding domain.

87. The targeted base editor of any one of claims 21-22, wherein the first portion comprises
(a) a first split deaminase domain comprising an amino acid sequence of SEQ ID NO:169, and
(b) a Left hand TALE programmable DNA binding domain; and
wherein the second portion comprises
(c) a second split deaminase domain comprising an amino acid sequence of any one of SEQ ID NOs:173, or 175, and
(d) a Right hand TALE programmable DNA binding domain.

88. The targeted base editor of any one of claims 21-22, wherein the first portion comprises
(a) a first split deaminase domain comprising an amino acid sequence of SEQ ID NO:171, and
(b) a Left hand TALE programmable DNA binding domain; and
wherein the second portion comprises
(c) a second split deaminase domain comprising an amino acid sequence of any one of SEQ ID NO:175, and
(d) a Right hand TALE programmable DNA binding domain.

89. The targeted base editor of any one of claims 21-22, wherein the first portion comprises
(a) a first split deaminase domain comprising an amino acid sequence of SEQ ID NO:169, and
(b) a Left hand BAT programmable DNA binding domain; and
wherein the second portion comprises
(c) a second split deaminase domain comprising an amino acid sequence of any one of SEQ ID NOs:173, or 175, and
(d) a Right hand TALE programmable DNA binding domain.

90. The targeted base editor of any one of claims 21-22, wherein the first portion comprises
(a) a first split deaminase domain comprising an amino acid sequence of SEQ ID NO:169, and
(b) a first coiled coil domain, and
(c) optionally a Left hand TALE programmable DNA binding domain; and
wherein the second portion comprises
(d) a second split deaminase domain comprising an amino acid sequence of any one of SEQ ID NOs:173, or 175, and
(e) a second coiled coil domain, and
(f) optionally a Right hand TALE programmable DNA binding domain;
wherein the first and second coiled coil domains interact together upon combination of the first and second portions.

91. The targeted base editor of any one of claims 22-91, wherein one or both of the first and second portions comprises at least one linker.

92. The targeted base editor of any one of claims 50-90, wherein one or both of the first and second portions comprises at least one linker, and
wherein the linker is positioned between the programmable DNA binding domain and the split deaminase domain.

93. The targeted base editor of any one of claim 92, wherein both of the first and second portions comprise a linker between the programmable DNA binding domain and the split deaminase domain.

94. The targeted base editor of any one of any one of claims 91-93, wherein the linker is between 2 and 200 amino acids in length.

95. The targeted base editor of claim 94, wherein the linker is between 2 and 16 amino acids in length.

96. The targeted base editor of any one of claim 91-95, wherein the linker comprises an amino acid sequence of any of GS, GSG, GSS, or SEQ ID NOS:23-27 or 30.

97. The targeted base editor of any one of claims 50-96, wherein the base editor is configured such that the target nucleic acid is between 9 and 11 base pairs from a programmable binding domain binding site on a target DNA strand.

98. The targeted base editor of any one of claims 50-97, wherein the distance between two binding sites of two programmable binding domains on a target DNA strand is between 12 and 22 base pairs.

99. The targeted base editor of claim 98, wherein the distance between two binding sites of two programmable binding domains on a target DNA strand is between 14 and 19 base pairs.

100. The targeted base editor of any one of claims 22-99, wherein at least one of the first and second portions comprises a cellular targeting moiety.

101. The targeted base editor of claim 100, wherein both of the first and second portions comprises a cellular targeting moiety.

102. The targeted base editor of claim 101, wherein both of the first and second portions comprise the same cellular targeting moiety.

103. The targeted base editor of any one of claims 100-102, wherein cellular targeting moiety is selected from the group consisting of a mitochondrial targeting sequence (MTS), and a nuclear localization sequence (NLS).

104. The targeted base editor of claim 103, wherein the NLS comprises an amino acid sequence of any one of SEQ ID NOs:34-39.

105. The targeted base editor of claim 104, wherein the MTS comprises an amino acid sequence of any one of SEQ ID NOs:22, 69, 71, 182 or 183.

106. The targeted base editor of any one of claims 22-105, wherein at least one of the first and second portions comprises a base excision repair inhibitor.

107. The targeted base editor of claim 106, wherein the base excision repair inhibitor is a mammalian DNA glycosylase inhibitor.

108. The targeted base editor of claim 106 or 107, wherein the base excision repair inhibitor is a uracil glycosylase inhibitor.

109. The targeted base editor of any one of claims 106-108, wherein the base excision repair inhibitor has an amino acid sequence comprising any one of SEQ ID NO:21 or 70.

110. A method comprising
bringing into contact a target nucleic acid and a targeted base editor of any one of claims 17-109, wherein the target nucleic acid is double-stranded DNA, whereby the instance of the target nucleotide sequence is deaminated by the targeted base editor.

111. The method of claim 110, wherein the deaminated nucleotide in the target nucleotide sequence is converted to a thymine or a guanine nucleotide, wherein the conversion completes a base edit of the target nucleotide sequence.

112. The method of claim 110 or 111, wherein the target nucleic acid is mitochondrial DNA.

113. The method of any one of claims 110-112, wherein the target nucleotide sequence is AC.

114. The method of any one of claims 110-112, wherein the target nucleotide sequence is CC.

115. The method of any one of claims 110-112, wherein the target nucleotide sequence is GC.

116. The method of any one of claims 110-112, wherein the target nucleotide sequence is TC.

117. The method of any one of claims 110-116, wherein the last C in the target nucleotide sequence is deaminated by the targeted base editor.

118. The method of any one of claims 110-117, wherein the instance of the target nucleotide sequence in the target DNA is within 20 nucleotides of the base editor target sequence.

119. The method of any one of claims 110-118, wherein the target nucleic acid is in a cell, wherein bringing into contact the target nucleic acid and the targeted base editor is accomplished by facilitating entry of the targeted base editor into the cell.

120. The method of claim 119, wherein the cell is in an animal, wherein bringing into contact the target nucleic acid and the targeted base editor is accomplished by administering the targeted base editor to the animal.

121. A method comprising:
bringing into contact a target nucleic acid and one or more deaminase domain, wherein the target nucleic acid is double-stranded cytosine-methylated DNA, wherein the deaminase domain can deaminate double-stranded DNA, wherein the deaminase domain deaminates substantially only non-methylated cytosine nucleotides in the target nucleic acid,
wherein substantially all of the non-methylated cytosine nucleotides in the target nucleic acid are deaminated by the deaminase domain; and
sequencing the deaminated target nucleic acid, whereby methylated cytosine nucleotides in the target nucleic acid are identified.

122. The method of claim 121, wherein the deaminase domain deaminates 90% or more of the non-methylated cytosine nucleotides in the target nucleic acid.

123. A method comprising:
bringing into contact a deaminase domain and a plurality of copies of a target nucleic acid for a time and under conditions that results in deamination of an average of 0.1 to 5.0 nucleotides per copy of the target nucleic acid,
wherein the target nucleic acid is double-stranded DNA, wherein the deaminase domain can deaminate double-stranded DNA.

124. The method of claim 123, wherein the copies of the target nucleic acid are in vitro.

125. The method of claim 124, wherein the deaminated nucleotides in the copies of the target nucleic acid are converted to a thymine or a guanine nucleotide via an in vitro reaction.

126. The method any one of claims 121-125 further comprising subjecting the deaminated copies of the target nucleic acid to a selection procedure.

127. The method of claim 126, wherein the selection procedure comprises mRNA display, ribosome display, or SELEX, or cell-based selection assays.

128. The method of any one of claims 125-127, wherein the deaminated nucleotides in the copies of the target nucleic acid are converted to a thymine or a guanine nucleotide, wherein the conversion completes one or more base edits of some or all of the copies of target nucleic acid.

129. The method of claim 123, wherein the deaminated nucleotides in the copies of the target nucleic acid are converted to a thymine or a guanine nucleotide by incubating the copies of the target nucleic acid in cells followed by a DNA replication/amplification step.

130. The method of claim 123, wherein the copies of the target nucleic acid are in cells, wherein bringing into contact the deaminase domain and the copies of a target nucleic acid is accomplished by facilitating entry of the deaminase domain into the cells.

131. The method of claim 130, wherein the cells are in an animal, wherein bringing into contact the deaminase domain and the copies of a target nucleic acid is accomplished by administering the deaminase domain to the animal.

132. The method of claim 130, wherein the copies of the target nucleic acid are in cells, wherein the deaminase domain is encoded by a transgenic expression construct in the cells, wherein bringing into contact the deaminase domain and the copies of a target nucleic acid is accomplished by transiently expressing the deaminase domain in the cells.

133. A method of treating or preventing a mitochondrial genetic disease in a subject by editing one or more nucleic acids in mitochondrial DNA in a cell of the subject, comprising
introducing to the cell the targeted cytosine deaminase base editor of any one of claims 1-110,
wherein a target nucleic acid within mitochondrial DNA is deaminated by the targeted base editor.

134. The method of claim 133, wherein the deaminated nucleotide in the target nucleotide sequence is converted to a thymine or a guanine nucleotide.

135. The method of any one of claims 133-134, wherein one or more nucleic acids in the mitochondrial DNA is edited to a non-pathogenic form.

136. The method of any one of claims 133-135, wherein the deaminated nucleotide is at a position selected from m.583G>A, m.616T>C, m.1606G>A, m.1644G>A, m.3258T>C, m.3271T>C, m.3460G>A, m.4298G>A, m.5728T>C, m.5650G>A, m.3243A>G, m.8344A>G, m.14459G>A, m.11778G>A, m.14484T>C, m.8993T>C, m.14484T>C, m.3460G>A, and m.1555A>G.

137. The method of any one of claims 133-136, wherein the cell is selected from the group consisting of a fibroblast, lymphocyte, pancreatic cell, muscle cell, neuronal cell, and a stem cell.

138. A vector comprising or expressing the targeted base editor of any one of claims 22-110.

139. The vector of claim 138, wherein the vector is an altered adenovirus (AAV) vector, a Lentivirus vector, or a virus-like particle (VLP).

140. The vector of claim 138 or 139, wherein the targeted base editor is encapsulated within the vector.

141. The method of any one of claims 120, or 129-137, wherein the deaminase domain comprises a targeted base editor within a vector.

142. The targeted base editor of any one of claims 22 to 49, wherein the first and second portions each comprise a programmable DNA binding domain independently selected from the group consisting of a TALE, BAT, CRISPR-Cas9, Cfp1, and Zinc finger.

143. The targeted base editor of claim 50/142, wherein the first portion is a TALE and the second portion is a TALE, wherein the first portion is a TALE and the second portion is a BAT, wherein the first portion is a TALE and the second portion is a Zinc finger, wherein the first portion is a TALE and the second portion is a CRISPR-Cas9, wherein the first portion is a TALE and the second portion is a Cfp1, wherein the first portion is a BAT and the second portion is a TALE, wherein the first portion is a BAT and the second portion is a BAT, wherein the first portion is a BAT and the second portion is a Zinc finger, wherein the first portion is a BAT and the second portion is a CRISPR-Cas9, wherein the first portion is a BAT and the second portion is a Cfp1, wherein the first portion is a Zinc finger and the second portion is a TALE, wherein the first portion is a Zinc finger and the second portion is a BAT, wherein the first portion is a Zinc finger and the second portion is a Zinc finger, wherein the first portion is a Zinc finger and the second portion is a CRISPR-Cas9, wherein the first portion is a Zinc finger and the second portion is a Cfp1, wherein the first portion is a CRISPR-Cas9 and the second portion is a TALE, wherein the first portion is a CRISPR-Cas9 and the second portion is a BAT, wherein the first portion is a CRISPR-Cas9 and the second portion is a Zinc finger, wherein the first portion is a CRISPR-Cas9 and the second portion is a CRISPR-Cas9, wherein the first portion is a CRISPR-Cas9 and the second portion is a Cfp1, wherein the first portion is a Cfp1 and the second portion is a TALE, wherein the first portion is a Cfp1 and the second portion is a BAT, wherein the first portion is a Cfp1 and the second portion is a Zinc finger, wherein the first portion is a Cfp1 and the second portion is a CRISPR-Cas9, or wherein the first portion is a Cfp1 and the second portion is a Cfp1.

144. A method of editing one or more nucleic acids in mitochondrial DNA in a mitochondrion or chloroplast DNA in a chloroplast, comprising introducing to the mitochondrion or the chloroplast the targeted cytosine deaminase base editor of any one of claims 1-110,
wherein a target nucleic acid within mitochondrial or chloroplast DNA is deaminated by the targeted base editor.

145. The method of claim 144, wherein the mitochondrion or the chloroplast is in vitro.

146. The deaminase domain of claim 1 or 2, wherein the target nucleotides each exhibit a context specificity defined by the deaminase probability sequence logo at a defined editing threshold.


SEQUENCE LISTING (297 sequences)

SEQ ID NO: 1 - protein, 138 aa, Artificial Sequence
  Synthetic Peptide
TKSANSGGAAKDLAKYRERQGMPRAGSADDAHTAARLDVGGRSFYGHNAHGRNIDIKVNAQTKTHAEADVFQQAKNAKVSADRATLHVDRDLCDACGIKGGVGSLMRGVGISRLTVNSPSGRFEITASRPSVPRRING

SEQ ID NO: 2 - protein, 160 aa, Artificial Sequence
  Synthetic Peptide
FSKAESGYIEIQRFRRILNMPRYSLTNGRTGTVARVEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 3 - protein, 136 aa, Artificial Sequence
  Synthetic Peptide
GVGGAITATVGSTAGAAGRAAARAPSLPAYAGGKTSGVLRTTAGDTALLSGYKGPSASMPRGTPGMNGRIKSHVEAHAAAVMREQGMKEGTLYINRVPCSGATGCDAMLPRMLPPDAHLRVVGPNGYDQVFVGLPD

SEQ ID NO: 4 - protein, 166 aa, Artificial Sequence
  Synthetic Peptide
DPIGLMGGLNLYQYAPNSIAWTDWWGLAGSYTLGSYQISAPQLPAYNGQTVGTFYYVNGAGGLESRTFSSGGPTPYPNYANAGHVEGQSALFMRDNGISDGLVFHNNPEGTCGFCVNMTETLLPENSKLTVVPPEGAIPVKRGATGETRTFTGNSKSPKSPVKGEC

SEQ ID NO: 5-7 - skipped (3 entries)

SEQ ID NO: 8 - protein, 118 aa, Artificial Sequence
  Synthetic Peptide
MPPAGSETDKSTIAKLEISGQNFFGINSGSNPNPRQITFNVNPITKTHAEADAFQQAADVGIRGGKARLIVDRDLCAACGIRGGVNSMAWQLGIEELEIITPSVSKTIAVKPPNRRRQ

SEQ ID NO: 9 - protein, 162 aa, Artificial Sequence
  Synthetic Peptide
SQFDNVRKDMGLPARIGDDDPYTTSVLRIDGHEYWGKNGKWVTKGKTSNYTDKAHYDKVRKELGTSAEVPGHAEGVAFNKAYQVRKNTGTKGGNAVLYVDKIPCVMCKPGIATLMRSAKVDHLDLHYLQDGKMHHVQYVRNPDTDAVYNPFSGKWTKPSKKK

SEQ ID NO: 10 - protein, 206 aa, Artificial Sequence
  Synthetic Peptide
GRLKKDERVYRNAHQPFRLQNQYYDEETGLHYNLMRYYEPEAGRFVNQDPIGLLGGDNLYWFAPNAAMWLDPWGLAVVDAIFEMQGHTFTGTNPLDRNPRISSPIQGLSAVNNDKFKMHAEIDAMTQAHDKGLRGGKGVLKIKGKNACSYCKGDIKKMALKLDLDELEVHNHDGTVHKFSKGDLKPVKKGGKGWKKPKKSKKPGAC

SEQ ID NO: 11 - protein, 159 aa, Artificial Sequence
  Synthetic Peptide
RAPEAIQTLRDSYGTDLLGRPLLGDSDTVAHGIVDGETFMGVNSGAIVEYSQRDLNDAKRALIPLVRKRPDIMSTHNIGQRPNDALFHAESTVLLRAARANDGTLSGKVIDITVDRPICSSCKKVLPLIGQELGNPIVRFTEPSGRVRTMHNGEWKDQD

SEQ ID NO: 12-13 - skipped (2 entries)

SEQ ID NO: 14 - protein, 212 aa, Artificial Sequence
  Synthetic Peptide
GALDNLAQTVTVADNATPSSADIFAEIAKSGDNASQSTVDTFTDLAKSLDEAPPLDQSNAPNRTPWDTIDHFRSHKQGMAELGDAIPVKGDKLGTVAFVEIEGSKVFGVNSTALVDDADKALGRMWRDRLGFNSGQAQALFHGEAHSLMRAYEKFSGKLPKDLTLYVDRLTCGPCQGALPDLMKAMGIERLKIVTKSGRVGEISGGVFRWLE

SEQ ID NO: 15 - protein, 183 aa, Artificial Sequence
  Synthetic Peptide
GGGTVTVSSTASAQVYATAQTEVEVTKKTKELAAEQQQAQAYQCPVTGKACTGDPFNDLAAFRKRQGMPEAGTDADKDTAARLDVGGQIFYGRNGKGKVTDIPVNAYTRDHAEGDVFQQAKNAKITADRAVMYVDRPLCDGCGAYGGVGSLLRGTGIKEVVVVAPNGRFLITAARPSTPQPLD

SEQ ID NO: 16 - protein, 171 aa, Artificial Sequence
  Synthetic Peptide
GAASVGRGASHFSKAESGYIEIQRFRRILNMPRYSLTNGRTGTVARVEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 17-20 - skipped (4 entries)

SEQ ID NO: 21 - protein, 84 aa, Artificial Sequence
  Synthetic Peptide
MTNLSDIIEKETGKQLVIQESILMLPEEVEEVIGNKPESDILVHTAYDESTDENVMLLTSDAPEYKPWALVIQDSNGENKIKML

SEQ ID NO: 22 - protein, 27 aa, Artificial Sequence
  Synthetic Peptide
PEDEIWLPEPESVDVPAKPISTSSMMM

SEQ ID NO: 23 - protein, 4 aa, Artificial Sequence
  Synthetic Peptide
GGGS

SEQ ID NO: 24 - protein, 4 aa, Artificial Sequence
  Synthetic Peptide
SGGS

SEQ ID NO: 25 - protein, 5 aa, Artificial Sequence
  Synthetic Peptide
GGGGS

SEQ ID NO: 26 - protein, 5 aa, Artificial Sequence
  Synthetic Peptide
EAAAK

SEQ ID NO: 27 - protein, 16 aa, Artificial Sequence
  Synthetic Peptide
SGSETPGTSESATPES

SEQ ID NO: 28 - protein, 14 aa, Artificial Sequence
  Synthetic Peptide
  N (2)..(3): Any amino acid
  N (5)..(8): Any amino acid
  N (10)..(13): Any amino acid
CNNCNNNNHNNNNC

SEQ ID NO: 29 - protein, 25 aa, Artificial Sequence
  Synthetic Peptide
  N (2)..(6): Any Amino acid
  N (8)..(19): Any Amino acid
  N (21)..(24): Any Amino acid
CNNNNNCNNNNNNNNNNNNHNNNNC

SEQ ID NO: 30 - protein, 20 aa, Artificial Sequence
  Synthetic Peptide
PKKKRKVEASSPKKRKVEAS

SEQ ID NO: 31 - protein, 4 aa, Artificial Sequence
  Synthetic Peptide
  N (2)..(2): Lysine or Arginine
  X (3)..(3): Lysine or Arginine
  N (4)..(4): Lysine or Arginine
KNXN

SEQ ID NO: 32 - protein, 7 aa, Artificial Sequence
  Synthetic Peptide
PKKKRKV

SEQ ID NO: 33 - protein, 12 aa, Artificial Sequence
  Synthetic Peptide
PKKKRKVEDPYC

SEQ ID NO: 34 - protein, 33 aa, Artificial Sequence
  Synthetic Peptide
PKKKRKVGPKKKRKVGPKKKRKVGPKKKRKVGC

SEQ ID NO: 35 - protein, 12 aa, Artificial Sequence
  Synthetic Peptide
CYGRKKRRQRRR

SEQ ID NO: 36 - protein, 17 aa, Artificial Sequence
  Synthetic Peptide
CSIPPEVKFNKPFVYLI

SEQ ID NO: 37 - protein, 18 aa, Artificial Sequence
  Synthetic Peptide
DRQIKIWFQNRRMKVVKK

SEQ ID NO: 38 - protein, 12 aa, Artificial Sequence
  Synthetic Peptide
PKKKRKVEDPYC

SEQ ID NO: 39 - protein, 9 aa, Artificial Sequence
  Synthetic Peptide
PAAKRVKLD

SEQ ID NO: 40 - protein, 166 aa, Artificial Sequence
  Synthetic Peptide
MEMGTRSLPQETEYMREALKEAEKAYALGETPIGCVIVWRGEIIGRGYNRRAIDKSVLAHAEITAIAEAERYLADWRLEEATLYVTLEPCPMCAGAIVQARVGRVVYATANLKAGSAGTVIDMMHVAGFNHQVEVVGGILEKECTDLLKRFFRELRAEKDKPYPPK

SEQ ID NO: 41 - protein, 145 aa, Artificial Sequence
  Synthetic Peptide
EVQARLNGLAAEARQGLPPNKGNVAVAEINIPELADQPFITKAFSGYQTDKDGFVGKPSGNVDTWALQPQKSSPEFIGGPGAYFRDVDTEFKILENLAQKLGPNTNATGTVNLISEKVVCPSCTTVIMQFRERYPNIQLNIFTRD

SEQ ID NO: 42 - protein, 128 aa, Artificial Sequence
  Synthetic Peptide
INYAKENGITGGRNVAVFEYIDLNGKIQTIIKASERGKGHAERLIAMELQNKGIPNSNVTRIYSELEPCSAPGGYCSNMIKYGSPNGLGPYSNAKVTYSFSYGGNPHNAEAARQGVDALRKAREQQKR

SEQ ID NO: 43 - protein, 277 aa, Artificial Sequence
  Synthetic Peptide
GGTPSCSTTLDGLVPTDALEEFATRAYTQEEGACSGYYVVGSANSARVEGVLTACDATTTSVGNEWREEAGTTRACQLFGWPGAIPESVEIDRARCRLAEQDWARLQQRREDCGLPPRTLVPNDGHTVAILTTPGEDEITGLNGRTGGAQPYRARAVEEGTCPPPLTRTYGEDATRYRGAGPTHCHAEGDALEQLSVLRMREPGTPGAGDPRQGATGGRTTGSAELIVDRDPCAMSCAPRGVDRMRSIAGLEELIVRSPQGTRRYADGLPETGVPLD

SEQ ID NO: 44 - protein, 262 aa, Artificial Sequence
  Synthetic Peptide
GRLGSEVGEGVLAARPADGHTIKVTESGRIIRCSRCDDILDLLDEYRAVFADNPGYVERLGRIEDLADAARKARKAKNPNASQLADQAADDAAALLRDVRTSAQARGNLAREGQPLSGAGRLPAEVVQPISPARIQEGLNSLAAQRVQRGLPPAGSATDVSTVCRLDIGGESFYGVNAHHTTMDLHVNAQTATHAEGQAFQLGARSLPASRETRAVLYVDRELCRACGDFGGVESMAKQLGLLQLDVYTPNGLALTLDFAGR

SEQ ID NO: 45 - protein, 169 aa, Artificial Sequence
  Synthetic Peptide
GSYASPDPLGLEAAPNNHAYVANPATAADPTGLIPCDVADDLAAYRQRQGMPVAGSAEDAHTAARLDVDGQSFYGRNGHGMDIDIRANAQTKTHAEAQAFQEAKNAGVSGKTGTLYVDRDFCRACGPNGGVGSLMRGLGLERLEVHTPSGRYTIDATKRPSIPVPWSEG

SEQ ID NO: 46 - protein, 129 aa, Artificial Sequence
  Synthetic Peptide
MPVAGSVDDKHTAAKLIFGDNEYYGHNGHGMQDEVKGAFSVNAQTATHAEGLAFYNAKTSGVEGTSATLITDRPACASCGYYGGIRSMAKDMGINDLTVVSPNNAPITFNPQVKPIPNPFPKPVPKTIR

SEQ ID NO: 47 - protein, 175 aa, Artificial Sequence
  Synthetic Peptide
GLAGGEKPYAYVGNPAQAVDPLGLAGCEDPWKIVDRFRRSKNKMEPLGDRIPGAIDKDGLHTVAFFEMNGRRVFGVNSGTLYKKDKALGKQWNEKIDYLTKEEKGTSAFHAEGHALMRAHKKFGGVMPKEITMYVDRVTCNHCERFLPALMKEMGIEKLKLFSKNGTSSVLHAAR

SEQ ID NO: 48 - protein, 144 aa, Artificial Sequence
  Synthetic Peptide
GSNGAIYSDVAAAQKAATTASRIGFNDLATFRVQLGLPPAGTAADKSTLAVIEINGQKIYGVNAHGQPVSGVNAISSTHAEIDALNQIKQQGIDVSGQNLTLYVDRTPCAACGTNGGIRSMVEQLGLKQLTVVGPDGPMIVTPR

SEQ ID NO: 49 - protein, 141 aa, Artificial Sequence
  Synthetic Peptide
DKVADDVVEDAAKAIKGGSSSINLPEYDGKTTHGVLVLDDGTQVPFSSGNANPNYKNYIPASHVEGKSAIYMRENGINNGTVFHNNTDGTCPYCDKMLPTLLEEGSTLTVVPPANANAPKPSWVDTVKTYIGNDKIPKKPK

SEQ ID NO: 50 - protein, 125 aa, Artificial Sequence
  Synthetic Peptide
MSLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 51 - protein, 143 aa, Artificial Sequence
  Synthetic Peptide
MSITDRLAKQKEKQDNTNIIDNRPKLPDYDGKTTHGILVTPNSEHIPFSSGNPNPNYKNYIPASHVEGKSAIYMRENGITSGTIYYNNTDGTCPYCDKMLSTLLEEGSVLEVIPPINAKAPKPSWVDKPKTYIGNNKVPKPNK

SEQ ID NO: 52 - protein, 117 aa, Artificial Sequence
  Synthetic Peptide
ELPPYDGKTTYGVLILDDGKQYSFNSGKPAPIYRNYIPASHVEGKAAIYMRENKIQSGTVYHNNTDGTCPYCDKMLPTLLEKDSTLKVVPPQNATSSKKGWITNEKIYIGNDKIPKT

SEQ ID NO: 53 - protein, 146 aa, Artificial Sequence
  Synthetic Peptide
TDEFKLAYEQLKDIEQAYEYANIDKDKIDIPDFDGKITWGILVLEDGTCITFSSGNANPMFNHYIPASHAEGKAAIYMRQKGIKHGVIFHNNTDGTCPYCNTMLPTLLEENSTLIVVPPINAVAKKRGWIDKIKIYTGNNKIPKTN

SEQ ID NO: 54 - protein, 156 aa, Artificial Sequence
  Synthetic Peptide
GASGAAGHGLSTTGKNVLGHFEPTPTTPQGTSSDTIAEMLNSASQPGRTAGVLDIDGELTPLTSGRPSLPNYIASGHVEGQAAMIMRQQQVQSATVYHDNPNGTCGYCYSQLPTLLPEGAALDVVPPAGTVPPSNRWHNGGPSFIGNSSEPKPWPR

SEQ ID NO: 55 - protein, 159 aa, Artificial Sequence
  Synthetic Peptide
SHYAEEYKQLLKDIDTKREAEEAALLREAYPSMEGATLPPFDGKTTIGLMFYTDASGQYQVKKLFSGEKVLSNYDATGHVEGKAALIMRNEKITEAVVMHNHPSGTCNYCDKQVETLLPKNATLRVIPPENAKAPTSYWNDQPTTYRGDGKDPKAPSKK

SEQ ID NO: 56 - protein, 146 aa, Artificial Sequence
  Synthetic Peptide
ASASPSTNSAGSSGKNVRLPRDYASELPEYDGKTTYGVLVTNEGKVIQLRSGGKEVPYSGYKAVSASHVEGKAAIWIRENASSGGTVYHNNTTGTCGYCNSQVKALLPEGVELKIVPPANAVARNSQAKAIPTINVGNATQPGRKP

SEQ ID NO: 57 - protein, 149 aa, Artificial Sequence
  Synthetic Peptide
KPEALKDAREPKTKPPHNRVHQDPNTSWNPNNYPDTPSGQLPAYDGKNTLGRIEIDGEIYHVKNGKGQPGETLKTDPTVKAGAVSPSHAEGHAVAIMKETGTKEAVLDINHPTGPCGFCDKVLENMLPEGSKLTVNWPNGSQVFTGNSK

SEQ ID NO: 58 - protein, 157 aa, Artificial Sequence
  Synthetic Peptide
SHYAKEYKQLLADIDALAEAREDALLREQFPSMDAVTLPPFDGKTTIGYMFYTDANGQYHVRKLYSGGKVLSNYDSSGHVEGMAALIMRKGRITEAVVMHNHPSGTCHYCNGQVETLLPKNAKLKVIPPANAKAPTKYWYDQPVDYLGNSNDPKPPS

SEQ ID NO: 59 - protein, 153 aa, Artificial Sequence
  Synthetic Peptide
GGSAVVGGGIAATGAKALTTGKKLTESPGTLNAAQRLLASIGEEGKTAGVLEVDGALFPLVSGKSVLPNYAASGHVEGQAALLMQGMGATNGRLLIDNPNGICGYCTSQVPTLLPENAVLEVGTPLGTVTPSARWSASKPFIGNDREPKPWPR

SEQ ID NO: 60 - protein, 135 aa, Artificial Sequence
  Synthetic Peptide
IGKVGKLRFAPKVESAESMLRSLSQEGKTAGVLDINGELIPLVSGTSSLKNYAASGHVEGQAALIMRERGVASARLIIDNPSGICGYCRSQVPTLLPAGATLEVTTPRGTVPPTARWSNGKTFVGNENDPKPWPR

SEQ ID NO: 61 - protein, 171 aa, Artificial Sequence
  Synthetic Peptide
LEDKIDYDDLVRKREKAREDLLEAEKRLREEEIRAKYPTPEEAQLPPYDGDTTYALMYYTDEHGKSHVVELSSGGADDEHSNYAAAGHTEGQAAVIMRQRKITSAVVVHNNTDGTCPFCVAHLPTLLPSGAELRVVPPRSAKAKKPGWIDVSKTFEGNARKPLDNKNKKST

SEQ ID NO: 62 - protein, 153 aa, Artificial Sequence
  Synthetic Peptide
GGSAVVGAGVVATGAKAVTTGKSLSESQATLSVAQRLLATIGEEGKTAGVLELDGELIPLVSGKSSLPNYAASGHVEGQAALIMRDRGATSGRLLIDNPSGICGYCKSQVATLLPENATLQVGTPLGTVTPSSRWSASRTFTGNDRDPKPWPR

SEQ ID NO: 63 - protein, 152 aa, Artificial Sequence
  Synthetic Peptide
DSAVDRLEQELEKLDVRNFFEDESETESGSSSINLPEYDGKTTHGVLVLDDGTQVPFSSGNANPNYKNYIPASHVEGKSAIYMRENGINNGTVFHNNTDGTCPYCDKMLPTLLDEGSTLTVVPPTNASAPKPSWVDTVKTYIGNDKIPKKPK

SEQ ID NO: 64 - protein, 149 aa, Artificial Sequence
  Synthetic Peptide
SGYDSQYPCKEEMSAGAGESGRKTISLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 65 - protein, 147 aa, Artificial Sequence
  Synthetic Peptide
ASPAVGTNAAGSSGKNVRMPRDYASELPEYDGKTTHGVLVTNEGKVIQLRSGGKEEPYTGYKAVSASHVEGKAAIWIRENGSSGGTVYHNNTTGTCGYCNSQVKALLPEGVELKIVPPTNAVAKNAQARAVPTINVGNGTQPGRKQK

SEQ ID NO: 66 - protein, 136 aa, Artificial Sequence
  Synthetic Peptide
YVGENGVWVHNASSEYGEVPELPEFNGKKTEGVFRTADGKEIKFESGGSTEYKNPSASHAEGKAAIYMRENGIKEGTVFHNNPNGTCNYCDKGLATLLPEGARLTVVPPIGAVAPNKYWVDVPKTYTGNGNLPSMK

SEQ ID NO: 67 - protein, 140 aa, Artificial Sequence
  Synthetic Peptide
HVGKCRLLVHNANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSNSVANNVRAIPVPKTYIGNSTVPKIK

SEQ ID NO: 68 - protein, 676 aa, Artificial Sequence
  Synthetic Peptide
MALSRAVCGTSRQLAPVLGYLGSRQKHSLPDYPYDVPDYAGYPYDVPDYAGYPYDVPDYAMDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 69 - protein, 29 aa, Artificial Sequence
  Synthetic Peptide
MSVLTPLLLRGLTGSARRLPVPRAKIHSL

SEQ ID NO: 70 - protein, 83 aa, Artificial Sequence
  Synthetic Peptide
TNLSDIIEKETGKQLVIQESILMLPEEVEEVIGNKPESDILVHTAYDESTDENVMLLTSDAPEYKPWALVIQDSNGENKIKML

SEQ ID NO: 71 - protein, 30 aa, Artificial Sequence
  Synthetic Peptide
MLSRAVCGTSRQLAPVLGYLGSRQKHSLPD

SEQ ID NO: 72 - protein, 202 aa, Artificial Sequence
  Synthetic Peptide
MPDFTIIQPDRKFDAAAVAGIFVRSSTSSSFPSASSYIAAKKRKNVDNTSTRKPYSYKDRKRKNTEEIRNIKKKLFMDLGIVRTNCGIDNEKQDREKAMKRKVTETIVTTYCELCEQNFSSSKMLLLHRGKVHNTPYIECHLCMKLFSQTIQFNRHMKTHYGPNAKIYVQCELCDRQFKDKQSLRTHWDVSHGSGDNQAVLA

SEQ ID NO: 73 - DNA, 264 bp, Artificial Sequence
  Synthetic Peptide
taataattatattattattttaaattaattatttaaccgtggtgcgcggggtcgcccagcaatagtataggttgtcgagtatgaagggtctaaaagattttaagacaccttacggacgaagagtttctctcttagtcccctgatctgcagaacccaggatatcaagcacatttcacttcacgtgttttgatgaaactatacatcacccgcgccacaggcgctgtgcggtttataatatattataatttatatttatattaaatt

SEQ ID NO: 74 - protein, 120 aa, Artificial Sequence
  Synthetic Peptide
MEPGEKPYKCPECGKSFSTSGSLVRHQRTHTGEKPYKCPECGKSFSDCRDLARHQRTHTGEKPYKCPECGKSFSQNSTLTEHQRTHTGEKPYKCPECGKSFSERSHLREHQRTHTGKKTS

SEQ ID NO: 75 - protein, 148 aa, Artificial Sequence
  Synthetic Peptide
MEPGEKPYKCPECGKSFSRNDTLTEHQRTHTGEKPYKCPECGKSFSREDNLHTHQRTHTGEKPYKCPECGKSFSDCRDLARHQRTHTGEKPYKCPECGKSFSQNSTLTEHQRTHTGEKPYKCPECGKSFSTKNSLTEHQRTHTGKKTS

SEQ ID NO: 76 - protein, 120 aa, Artificial Sequence
  Synthetic Peptide
MEPGEKPYKCPECGKSFSDPGHLVRHQRTHTGEKPYKCPECGKSFSQNSTLTEHQRTHTGEKPYKCPECGKSFSRSDKLTEHQRTHTGEKPYKCPECGKSFSQRANLRAHQRTHTGKKTS

SEQ ID NO: 77 - protein, 148 aa, Artificial Sequence
  Synthetic Peptide
MEPGEKPYKCPECGKSFSQLAHLRAHQRTHTGEKPYKCPECGKSFSTSGELVRHQRTHTGEKPYKCPECGKSFSREDNLHTHQRTHTGEKPYKCPECGKSFSDPGHLVRHQRTHTGEKPYKCPECGKSFSDSGNLRVHQRTHTGKKTS

SEQ ID NO: 78 - protein, 120 aa, Artificial Sequence
  Synthetic Peptide
MEPGEKPYKCPECGKSFSTKNSLTEHQRTHTGEKPYKCPECGKSFSSKKALTEHQRTHTGEKPYKCPECGKSFSTSGELVRHQRTHTGEKPYKCPECGKSFSTSGNLVRHQRTHTGKKTS

SEQ ID NO: 79 - protein, 148 aa, Artificial Sequence
  Synthetic Peptide
MEPGEKPYKCPECGKSFSTSGNLVRHQRTHTGEKPYKCPECGKSFSTKNSLTEHQRTHTGEKPYKCPECGKSFSSKKALTEHQRTHTGEKPYKCPECGKSFSTSGELVRHQRTHTGEKPYKCPECGKSFSTSGNLVRHQRTHTGKKTS

SEQ ID NO: 80 - protein, 120 aa, Artificial Sequence
  Synthetic Peptide
MEPGEKPYKCPECGKSFSTSGNLTEHQRTHTGEKPYKCPECGKSFSRSDNLVRHQRTHTGEKPYKCPECGKSFSTSGHLVRHQRTHTGEKPYKCPECGKSFSRADNLTEHQRTHTGKKTS

SEQ ID NO: 81 - protein, 148 aa, Artificial Sequence
  Synthetic Peptide
MEPGEKPYKCPECGKSFSTSGNLTEHQRTHTGEKPYKCPECGKSFSRSDNLVRHQRTHTGEKPYKCPECGKSFSTSGHLVRHQRTHTGEKPYKCPECGKSFSRADNLTEHQRTHTGEKPYKCPECGKSFSTSGNLVRHQRTHTGKKTS

SEQ ID NO: 82 - protein, 148 aa, Artificial Sequence
  Synthetic Peptide
MEPGEKPYKCPECGKSFSHKNALQNHQRTHTGEKPYKCPECGKSFSTSGNLTEHQRTHTGEKPYKCPECGKSFSTSGNLTEHQRTHTGEKPYKCPECGKSFSHTGHLLEHQRTHTGEKPYKCPECGKSFSTTGALTEHQRTHTGKKTS

SEQ ID NO: 83 - protein, 148 aa, Artificial Sequence
  Synthetic Peptide
MEPGEKPYKCPECGKSFSSRRTCRAHQRTHTGEKPYKCPECGKSFSHKNALQNHQRTHTGEKPYKCPECGKSFSTSGNLTEHQRTHTGEKPYKCPECGKSFSTSGNLTEHQRTHTGEKPYKCPECGKSFSHTGHLLEHQRTHTGKKTS

SEQ ID NO: 84 - protein, 148 aa, Artificial Sequence
  Synthetic Peptide
MEPGEKPYKCPECGKSFSRSDHLTNHQRTHTGEKPYKCPECGKSFSSRRTCRAHQRTHTGEKPYKCPECGKSFSHKNALQNHQRTHTGEKPYKCPECGKSFSTSGNLTEHQRTHTGEKPYKCPECGKSFSTSGNLTEHQRTHTGKKTS

SEQ ID NO: 85 - protein, 148 aa, Artificial Sequence
  Synthetic Peptide
MEPGEKPYKCPECGKSFSERSHLREHQRTHTGEKPYKCPECGKSFSRSDHLTNHQRTHTGEKPYKCPECGKSFSSRRTCRAHQRTHTGEKPYKCPECGKSFSHKNALQNHQRTHTGEKPYKCPECGKSFSTSGNLTEHQRTHTGKKTS

SEQ ID NO: 86 - protein, 148 aa, Artificial Sequence
  Synthetic Peptide
MEPGEKPYKCPECGKSFSRRDELNVHQRTHTGEKPYKCPECGKSFSRRDELNVHQRTHTGEKPYKCPECGKSFSTTGNLTVHQRTHTGEKPYKCPECGKSFSRTDTLRDHQRTHTGEKPYKCPECGKSFSTKNSLTEHQRTHTGKKTS

SEQ ID NO: 87 - protein, 148 aa, Artificial Sequence
  Synthetic Peptide
MEPGEKPYKCPECGKSFSQLAHLRAHQRTHTGEKPYKCPECGKSFSQRAHLERHQRTHTGEKPYKCPECGKSFSRSDNLVRHQRTHTGEKPYKCPECGKSFSTSGSLVRHQRTHTGEKPYKCPECGKSFSTTGNLTVHQRTHTGKKTS

SEQ ID NO: 88 - protein, 148 aa, Artificial Sequence
  Synthetic Peptide
MEPGEKPYKCPECGKSFSRRDELNVHQRTHTGEKPYKCPECGKSFSQLAHLRAHQRTHTGEKPYKCPECGKSFSQRAHLERHQRTHTGEKPYKCPECGKSFSRSDNLVRHQRTHTGEKPYKCPECGKSFSTSGSLVRHQRTHTGKKTS

SEQ ID NO: 89 - protein, 148 aa, Artificial Sequence
  Synthetic Peptide
MEPGEKPYKCPECGKSFSRRDELNVHQRTHTGEKPYKCPECGKSFSTSGSLVRHQRTHTGEKPYKCPECGKSFSTTGNLTVHQRTHTGEKPYKCPECGKSFSRKDNLKNHQRTHTGEKPYKCPECGKSFSRSDKLVRHQRTHTGKKTS

SEQ ID NO: 90 - protein, 675 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNGGGKQALETVQRLLPVLCQAHGLTPQQVVAIASNIGGKQALETVQRLLPVLCQAHGLTPEQVVAIASNNGGKQALETVQALLPVLCQAHGLTPEQVVAIASHDGGKQALETVQALLPVLCQAHGLTPEQVVAIASHDGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNNGGKQALETVQRLLPVLCQAHGLTPEQVVAIASHDGGKQALETVQALLPVLCQAHGLTPEQVVAIASHDGGKQALETVQALLPVLCQAHGLTPEQVVAIASNNGGKQALETVQALLPVLCQAHGLTPEQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLG

SEQ ID NO: 91 - protein, 777 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQALLPVLCQAHGLTPQQVVAIASNIGGKQALETVQRLLPVLCQAHGLTPQQVVAIASNNGGKQALETVQRLLPVLCQAHGLTPEQVVAIASNGGGKQALETVQALLPVLCQAHGLTPEQVVAIASNGGGKQALETVQALLPVLCQAHGLTPEQVVAIASNGGGKQALETVQALLPVLCQAHGLTPEQVVAIASNNGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNNGGKQALETVQALLPVLCQAHGLTPEQVVAIASHDGGKQALETVQALLPVLCQAHGLTPEQVVAIASNGGGKQALETVQALLPVLCQAHGLTPEQVVAIASHDGGKQALETVQRLLPVLCQAHGLTPQQVVAIASNIGGKQALETVQRLLPVLCQAHGLTPEQVVAIASHDGGKQALETVQALLPVLCQAHGLTPQQVVAIASHDGGKQALETVQRLLPVLCQAHGLTPQQVVAIASHDGGKQALETVQRLLPVLCQAHGLTPQQVVAIASNGGGRPALESIVAQLSRPDPALAALTNDHLVALACLGGRPALDAVKKGLG

SEQ ID NO: 92 - protein, 675 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNGGGKQALETVQALLPVLCQAHGLTPEQVVAIASNGGGKQALETVQALLPVLCQAHGLTPEQVVAIASHDGGKQALETVQRLLPVLCQAHGLTPEQVVAIASHDGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQALLPVLCHAHGLTPEQVVAIASNIGGKQALETVQRLLPVLCQAHGLTPEQVVAIASNGGGKQALETVQALLPVLCQAHGLTPQQVVAIASNGGGRPALESIVAQLSRPDPALAALTNDHLVALACLGGRPALDAVKKGLG

SEQ ID NO: 93 - protein, 675 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASNIGGKQALETVQRLLPVLCQAHGLTPEQVVAIASNNGGKQALETVQALLPVLCQAHGLTPEQVVAIASHDGGKQALETVQRLLPVLCQAHGLTPEQVVAIASHDGGKQALETVQALLPVLCQAHGLTPEQVVAIASNGGGKQALETVQALLPVLCQAHGLTPEQVVAIASNGGGKQALETVQALLPVLCHAHGLTPEQVVAIASHDGGKQALETVQRLLPVLCQAHGLTPEQVVAIASHDGGKQALETVQALLPVLCQAHGLTPQQVVAIASNGGGRPALESIVAQLSRPDPALAALTNDHLVALACLGGRPALDAVKKGLG

SEQ ID NO: 94 - protein, 673 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNIGGKQALETVQRLLPVLCQAHGLTPQQVVAIASNIGGKQALETVQRLLPVLCQAHGLTPEQVVAIASHDGGKQALETVQALLPVLCQAHGLTPEQVVAIASHDGGKQALETVQALLPVLCQAHGLTPEQVVAIASNGGGKQALETVQALLPVLCQAHGLTPEQVVAIASHDGGKQALETVQRLLPVLCQAHGLTPEQVVAIASHDGGKQALETVQALLPVLCQAHGLTPEQVVAIASNIGGKQALETVQRLLPVLCQAHGLTPEQVVAIASNGGGKQALETVQALLPVLCQAHGLTPEQVVAIASNIGGKQALETVQALLPVLCQAHGLTPEQVVAIASNIGGKQALETVQALLPVLCHAHGLTPEQVVAIASNIGGKQALETVQRLLPVLCQAHGLTPQQVVAIASNGGGRPALESIVAQLSRPDPALAALTNDHLVALACLGGRPALDAVKKGLGGSAIPVKRGATGETKVFTGNSNSPKSPTKGGC

SEQ ID NO: 95 - protein, 675 aa, Artificial Sequence; identical to SEQ ID NO: 92
  Synthetic Peptide

SEQ ID NO: 96 - protein, 675 aa, Artificial Sequence; identical to SEQ ID NO: 93
  Synthetic Peptide

SEQ ID NO: 97 - protein, 641 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNIGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNGGGKQALETVQALLPVLCQAHGLTPEQVVAIASNGGGKQALETVQALLPVLCQAHGLTPEQVVAIASNNGGKQALETVQRLLPVLCQAHGLTPEQVVAIASHDGGKQALETVQALLPVLCQAHGLTPEQVVAIASNGGGKQALETVQRLLPVLCQAHGLTPEQVVAIASNGGGKQALETVQALLPVLCQAHGLTPEQVVAIASHDGGKQALETVQALLPVLCQAHGLTPEQVVAIASHDGGKQALETVQALLPVLCHAHGLTPEQVVAIASNNGGKQALETVQRLLPVLCQAHGLTPQQVVAIASNGGGRPALESIVAQLSRPDPALAALTNDHLVALACLGGRPALDAVKKGLG

SEQ ID NO: 98 - protein, 641 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNIGGKQALETVQRLLPVLCQAHGLTPQQVVAIASNIGGKQALETVQRLLPVLCQAHGLTPEQVVAIASNIGGKQALETVQALLPVLCQAHGLTPEQVVAIASNNGGKQALETVQALLPVLCQAHGLTPEQVVAIASNGGGKQALETVQALLPVLCQAHGLTPEQVVAIASNIGGKQALETVQRLLPVLCQAHGLTPEQVVAIASNGGGKQALETVQALLPVLCQAHGLTPEQVVAIASNGGGKQALETVQRLLPVLCQAHGLTPEQVVAIASNGGGKQALETVQALLPVLCQAHGLTPEQVVAIASNIGGKQALETVQALLPVLCQAHGLTPEQVVAIASNNGGKQALETVQALLPVLCHAHGLTPEQVVAIASHDGGKQALETVQRLLPVLCQAHGLTPQQVVAIASNGGGRPALESIVAQLSRPDPALAALTNDHLVALACLGGRPALDAVKKGLG

SEQ ID NO: 99 - protein, 844 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNGGGKQALETVQALLPVLCQAHGLTPQQVVAIASNGGGKQALETVQRLLPVLCQAHGLTPQQVVAIASHDGGKQALETVQRLLPVLCQAHGLTPEQVVAIASNNGGKQALETVQALLPVLCQAHGLTPEQVVAIASNGGGKQALETVQALLPVLCQAHGLTPEQVVAIASNIGGKQALETVQALLPVLCQAHGLTPEQVVAIASNNGGKQALETVQRLLPVLCQAHGLTPEQVVAIASNNGGKQALETVQALLPVLCQAHGLTPEQVVAIASNIGGKQALETVQRLLPVLCQAHGLTPQQVVAIASNNGGRPALESIVAQLSRPDPALAALTNDHLVALACLGGRPALDAVKKGL

SEQ ID NO: 100 - protein, 811 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNGGGKQALETVQALLPVLCQAHGLTPQQVVAIASNGGGKQALETVQRLLPVLCQAHGLTPQQVVAIASHDGGKQALETVQRLLPVLCQAHGLTPEQVVAIASNNGGKQALETVQALLPVLCQAHGLTPEQVVAIASNGGGKQALETVQALLPVLCQAHGLTPEQVVAIASNIGGKQALETVQALLPVLCQAHGLTPEQVVAIASNNGGKQALETVQRLLPVLCQAHGLTPEQVVAIASNNGGKQALETVQALLPVLCQAHGLTPQQVVAIASNIGGRPALESIVAQLSRPDPALAALTNDHLVALACLGGRPALDAVKKGLG

SEQ ID NO: 101 - protein, 777 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNGGGKQALETVQALLPVLCQAHGLTPQQVVAIASNGGGKQALETVQRLLPVLCQAHGLTPQQVVAIASHDGGKQALETVQRLLPVLCQAHGLTPEQVVAIASNNGGKQALETVQALLPVLCQAHGLTPEQVVAIASNGGGKQALETVQALLPVLCQAHGLTPEQVVAIASNIGGKQALETVQALLPVLCQAHGLTPEQVVAIASNNGGKQALETVQRLLPVLCQAHGLTPQQVVAIASNNGGRPALESIVAQLSRPDPALAALTNDHLVALACLGGRPALDAVKKGLG

SEQ ID NO: 102 - protein, 675 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNGGGKQALETVQALLPVLCQAHGLTPQQVVAIASNGGGKQALETVQRLLPVLCQAHGLTPQQVVAIASHDGGKQALETVQRLLPVLCQAHGLTPEQVVAIASNNGGKQALETVQALLPVLCQAHGLTPQQVVAIASNGGGRPALESIVAQLSRPDPALAALTNDHLVALACLGGRPALDAVKKGLG

SEQ ID NO: 103 - protein, 539 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLG

SEQ ID NO: 104 - protein, 641 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQALLPVLCQAHGLTPQQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQALLPVLCQAHGLTPEQVVAIASNNGGKQALETVQALLPVLCQAHGLTPEQVVAIASNIGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPEQVVAIASNGGGKQALETVQALLPVLCQAHGLTPEQVVAIASNNGGKQALETVQALLPVLCHAHGLTPEQVVAIASNIGGKQALETVQRLLPVLCQAHGLTPQQVVAIASNGGGRPALESIVAQLSRPDPALAALTNDHLVALACLGGRPALDAVKKGLG

SEQ ID NO: 105 - protein, 777 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKSRSGARALEALLTVAGELRGPPLQLDTGQLLKIAKRGGVTAVEAVHAWRNALTGAPLNLTPEQVVAIASHDGGKQALETVQALLPVLCQAHGLTPEQVVAIASHDGGKQALETVQALLPVLCQAHGLTPEQVVAIASHDGGKQALETVQALLPVLCHAHGLTPEQVVAIASNIGGKQALETVQRLLPVLCQAHGLTPEQVVAIASHDGGKQALETVQALLPVLCQAHGLTPEQVVAIASNGGGKQALETVQALLPVLCQAHGLTPQQVVAIASNGGGKQALETVQRLLPVLCQAHGLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLG

SEQ ID NO: 106 - protein, 776 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASHDGGKQALETVQALLPVLCQAHGLTPEQVVAIASHDGGKQALETVQALLPVLCQAHGLTPEQVVAIASHDGGKQALETVQALLPVLCHAHGLTPEQVVAIASNIGGKQALETVQRLLPVLCQAHGLTPEQVVAIASHDGGKQALETVQALLPVLCQAHGLTPEQVVAIASNGGGKQALETVQALLPVLCQAHGLTPQQVVAIASNGGGKQALETVQRLLPVLCQAHGLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLG

SEQ ID NO: 107 - skipped

SEQ ID NO: 108 - protein, 777 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQALLPVLCQAHGLTPQQVVAIASNIGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNNGGKQALETVQALLPVLCQAHGLTPEQVVAIASNIGGKQALETVQALLPVLCQAHGLTPEQVVAIASNIGGKQALETVQRLLPVLCQAHGLTPEQVVAIASNNGGKQALETVQALLPVLCQAHGLTPEQVVAIASNIGGKQALETVQRLLPVLCQAHGLTPEQVVAIASNIGGKQALETVQALLPVLCQAHGLTPEQVVAIASNGGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNGGGKQALETVQALLPVLCQAHGLTPEQVVAIASNIGGKQALETVQALLPVLCQAHGLTPEQVVAIASNGGGKQALETVQALLPVLCQAHGLTPEQVVAIASNNGGKQALETVQRLLPVLCQAHGLTPQQVVAIASNGGGRPALESIVAQLSRPDPALAALTNDHLVALACLGGRPALDAVKKGLG

SEQ ID NO: 109 - protein, 675 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQALLPVLCQAHGLTPQQVVAIASNIGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNNGGKQALETVQALLPVLCQAHGLTPEQVVAIASNIGGKQALETVQALLPVLCQAHGLTPEQVVAIASNIGGKQALETVQRLLPVLCQAHGLTPEQVVAIASNNGGKQALETVQALLPVLCQAHGLTPEQVVAIASNIGGKQALETVQRLLPVLCQAHGLTPEQVVAIASNIGGKQALETVQALLPVLCQAHGLTPEQVVAIASNGGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNGGGKQALETVQALLPVLCQAHGLTPQQVVAIASNIGGRPALESIVAQLSRPDPALAALTNDHLVALACLGGRPALDAVKKGLG

SEQ ID NO: 110 - protein, 641 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQALLPVLCQAHGLTPQQVVAIASNIGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNNGGKQALETVQALLPVLCQAHGLTPEQVVAIASNIGGKQALETVQALLPVLCQAHGLTPEQVVAIASNIGGKQALETVQRLLPVLCQAHGLTPEQVVAIASNNGGKQALETVQALLPVLCQAHGLTPEQVVAIASNIGGKQALETVQRLLPVLCQAHGLTPEQVVAIASNIGGKQALETVQALLPVLCQAHGLTPEQVVAIASNGGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLG

SEQ ID NO: 111 - protein, 607 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQALLPVLCQAHGLTPQQVVAIASNIGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNNGGKQALETVQALLPVLCQAHGLTPEQVVAIASNIGGKQALETVQALLPVLCQAHGLTPEQVVAIASNIGGKQALETVQRLLPVLCQAHGLTPEQVVAIASNNGGKQALETVQALLPVLCQAHGLTPEQVVAIASNIGGKQALETVQRLLPVLCQAHGLTPEQVVAIASNIGGKQALETVQALLPVLCQAHGLTPEQVVAIASNGGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLG

SEQ ID NO: 112 - protein, 539 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQALLPVLCQAHGLTPQQVVAIASNIGGKQALETVQRLLPVLCQAHGLTPQQVVAIASNNGGKQALETVQRLLPVLCQAHGLTPEQVVAIASNNGGKQALETVQALLPVLCQAHGLTPEQVVAIASNNGGKQALETVQALLPVLCQAHGLTPEQVVAIASNIGGKQALETVQALLPVLCQAHGLTPEQVVAIASNIGGKQALETVQRLLPVLCQAHGLTPEQVVAIASNNGGKQALETVQALLPVLCQAHGLTPEQVVAIASNIGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLG

SEQ ID NO: 113 - protein, 607 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNIGGKQALETVQRLLPVLCQAHGLTPQQVVAIASHDGGKQALETVQRLLPVLCQAHGLTPEQVVAIASNIGGKQALETVQALLPVLCQAHGLTPEQVVAIASNGGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNGGGKQALETVQALLPVLCQAHGLTPEQVVAIASNGGGKQALETVQRLLPVLCQAHGLTPEQVVAIASHDGGKQALETVQALLPVLCQAHGLTPEQVVAIASHDGGKQALETVQALLPVLCQAHGLTPEQVVAIASHDGGKQALETVQALLPVLCHAHGLTPQQVVAIASNGGGRPALESIVAQLSRPDPALAALTNDHLVALACLGGRPALDAVKKGLG

SEQ ID NO: 114 - protein, 138 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKSRSGARALEALLTVAGELRGPPLQLDTGQLLKIAKRGGVTAVEAVHAWRNALTGAPLN

SEQ ID NO: 115 - protein, 137 aa, Artificial Sequence
  Synthetic Peptide
DIADLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLN

SEQ ID NO: 116 - protein, 639 aa, Artificial Sequence
  Synthetic Peptide
STAFVDQDKQMANRLNLSPLERSKIEKQYGGATTLAFISNKQNELAQILSRADILKIASYDCAAHALQAVLDCGPMLGKRGFSQSDIVKIAGNGGGAQALQAVLDLESMLGKRGFSRDDIAKMAGHDGGAQTLQAVLDLESAFRERGFSQADIVKIAGNGGGAQALYSVLDVEPTLGKRGFSRADIVKIAGNIGGAQALHTVLDLEPALGKRGFSRIDIVKIAANNGGAQALHAVLDLGPTLRECGFSQATIAKIAGHDGGAQALQMVLDLGPALGKRGFSQATIAKIAGHDGGAQALQTVLDLEPALCERGFGQATIAKMAGNGGGAQALQTVLDLEPALRKRDFRQADIIKIAGNIGGAQALQAVIEHGPTLRQHGFNLADIVKMAGNNGGAQALQAVLDLKPVLDEHGFSQADIVKIAGHDGGTQALHAVLDLERMLGERGFSRADIVNVAGHDGGAQALKAVLEHEATLNERGFSRADIVKIAGNNGGAQALKAVLEHEATLDERGFSRADIVNVAGNGGGAQALKAVLEHEATLNERGFNLTDIVEMAANGGGAQALKAVLEHGPTLRQRGLSLIDIVEIAGNGGGAQALKAVLKYGPVLMQAGRSNEEIVHVAARRGGAGRIRKMVAPLLERQ

SEQ ID NO: 117 - protein, 639 aa, Artificial Sequence
  Synthetic Peptide
STAFVDQDKQMANRLNLSPLERSKIEKQYGGATTLAFISNKQNELAQILSRADILKIASYDCAAHALQAVLDCGPMLGKRGFSQSDIVKIAGNNGGAQALQAVLDLESMLGKRGFSRDDIAKMAGNGGGAQTLQAVLDLESAFRERGFSQADIVKIAGNGGGAQALYSVLDVEPTLGKRGFSRADIVKIAGNGGGAQALHTVLDLEPALGKRGFSRIDIVKIAANNGGAQALHAVLDLGPTLRECGFSQATIAKIAGNIGGAQALQMVLDLGPALGKRGFSQATIAKIAGNGGGAQALQTVLDLEPALCERGFGQATIAKMAGNNGGAQALQTVLDLEPALRKRDFRQADIIKIAGHDGGAQALQAVIEHGPTLRQHGFNLADIVKMAGNGGGAQALQAVLDLKPVLDEHGFSQADIVKIAGHDGGTQALHAVLDLERMLGERGFSRADIVNVAGNIGGAQALKAVLEHEATLNERGFSRADIVKIAGHDGGAQALKAVLEHEATLDERGFSRADIVNVAGHDGGAQALKAVLEHEATLNERGFNLTDIVEMAAHDGGAQALKAVLEHGPTLRQRGLSLIDIVEIAGNGGGAQALKAVLKYGPVLMQAGRSNEEIVHVAARRGGAGRIRKMVAPLLERQ

SEQ ID NO: 118 - protein, 639 aa, Artificial Sequence
  Synthetic Peptide
STAFVDQDKQMANRLNLSPLERSKIEKQYGGATTLAFISNKQNELAQILSRADILKIASYDCAAHALQAVLDCGPMLGKRGFSQSDIVKIAGHDGGAQALQAVLDLESMLGKRGFSRDDIAKMAGNIGGAQTLQAVLDLESAFRERGFSQADIVKIAGHDGGAQALYSVLDVEPTLGKRGFSRADIVKIAGNGGGAQALHTVLDLEPALGKRGFSRIDIVKIAANGGGAQALHAVLDLGPTLRECGFSQATIAKIAGHDGGAQALQMVLDLGPALGKRGFSQATIAKIAGNNGGAQALQTVLDLEPALCERGFGQATIAKMAGHDGGAQALQTVLDLEPALRKRDFRQADIIKIAGHDGGAQALQAVIEHGPTLRQHGFNLADIVKMAGNIGGAQALQAVLDLKPVLDEHGFSQADIVKIAGNGGGTQALHAVLDLERMLGERGFSRADIVNVAGHDGGAQALKAVLEHEATLNERGFSRADIVKIAGNIGGAQALKAVLEHEATLDERGFSRADIVNVAGNGGGAQALKAVLEHEATLNERGFNLTDIVEMAANIGGAQALKAVLEHGPTLRQRGLSLIDIVEIAGNGGGAQALKAVLKYGPVLMQAGRSNEEIVHVAARRGGAGRIRKMVAPLLERQ

SEQ ID NO: 119 - protein, 639 aa, Artificial Sequence
  Synthetic Peptide
STAFVDQDKQMANRLNLSPLERSKIEKQYGGATTLAFISNKQNELAQILSRADILKIASYDCAAHALQAVLDCGPMLGKRGFSQSDIVKIAGNGGGAQALQAVLDLESMLGKRGFSRDDIAKMAGNGGGAQTLQAVLDLESAFRERGFSQADIVKIAGNNGGAQALYSVLDVEPTLGKRGFSRADIVKIAGNIGGAQALHTVLDLEPALGKRGFSRIDIVKIAANNGGAQALHAVLDLGPTLRECGFSQATIAKIAGNNGGAQALQMVLDLGPALGKRGFSQATIAKIAGNNGGAQALQTVLDLEPALCERGFGQATIAKMAGNIGGAQALQTVLDLEPALRKRDFRQADIIKIAGNIGGAQALQAVIEHGPTLRQHGFNLADIVKMAGNNGGAQALQAVLDLKPVLDEHGFSQADIVKIAGNIGGTQALHAVLDLERMLGERGFSRADIVNVAGNIGGAQALKAVLEHEATLNERGFSRADIVKIAGNGGGAQALKAVLEHEATLDERGFSRADIVNVAGNNGGAQALKAVLEHEATLNERGFNLTDIVEMAANGGGAQALKAVLEHGPTLRQRGLSLIDIVEIAGNGGGAQALKAVLKYGPVLMQAGRSNEEIVHVAARRGGAGRIRKMVAPLLERQ

SEQ ID NO: 120 - protein, 639 aa, Artificial Sequence
  Synthetic Peptide
STAFVDQDKQMANRLNLSPLERSKIEKQYGGATTLAFISNKQNELAQILSRADILKIASYDCAAHALQAVLDCGPMLGKRGFSQSDIVKIAGNGGGAQALQAVLDLESMLGKRGFSRDDIAKMAGHDGGAQTLQAVLDLESAFRERGFSQADIVKIAGNGGGAQALYSVLDVEPTLGKRGFSRADIVKIAGNGGGAQALHTVLDLEPALGKRGFSRIDIVKIAANNGGAQALHAVLDLGPTLRECGFSQATIAKIAGNNGGAQALQMVLDLGPALGKRGFSQATIAKIAGNNGGAQALQTVLDLEPALCERGFGQATIAKMAGNGGGAQALQTVLDLEPALRKRDFRQADIIKIAGNGGGAQALQAVIEHGPTLRQHGFNLADIVKMAGNIGGAQALQAVLDLKPVLDEHGFSQADIVKIAGNNGGTQALHAVLDLERMLGERGFSRADIVNVAGHDGGAQALKAVLEHEATLNERGFSRADIVKIAGNIGGAQALKAVLEHEATLDERGFSRADIVNVAGNGGGAQALKAVLEHEATLNERGFNLTDIVEMAANGGGAQALKAVLEHGPTLRQRGLSLIDIVEIAGNIGGAQALKAVLKYGPVLMQAGRSNEEIVHVAARRGGAGRIRKMVAPLLERQ

SEQ ID NO: 121 - protein, 639 aa, Artificial Sequence
  Synthetic Peptide
STAFVDQDKQMANRLNLSPLERSKIEKQYGGATTLAFISNKQNELAQILSRADILKIASYDCAAHALQAVLDCGPMLGKRGFSQSDIVKIAGNGGGAQALQAVLDLESMLGKRGFSRDDIAKMAGNIGGAQTLQAVLDLESAFRERGFSQADIVKIAGNIGGAQALYSVLDVEPTLGKRGFSRADIVKIAGNIGGAQALHTVLDLEPALGKRGFSRIDIVKIAAHDGGAQALHAVLDLGPTLRECGFSQATIAKIAGHDGGAQALQMVLDLGPALGKRGFSQATIAKIAGNGGGAQALQTVLDLEPALCERGFGQATIAKMAGNIGGAQALQTVLDLEPALRKRDFRQADIIKIAGNIGGAQALQAVIEHGPTLRQHGFNLADIVKMAGNIGGAQALQAVLDLKPVLDEHGFSQADIVKIAGHDGGTQALHAVLDLERMLGERGFSRADIVNVAGHDGGAQALKAVLEHEATLNERGFSRADIVKIAGNGGGAQALKAVLEHEATLDERGFSRADIVNVAGHDGGAQALKAVLEHEATLNERGFNLTDIVEMAAHDGGAQALKAVLEHGPTLRQRGLSLIDIVEIAGNIGGAQALKAVLKYGPVLMQAGRSNEEIVHVAARRGGAGRIRKMVAPLLERQ

SEQ ID NO: 122 - protein, 138 aa, Artificial Sequence
  Synthetic Peptide
TKSANSGGAAKDLAKYRERQGMPRAGSADDAHTAARLDVGGRSFYGHNAHGRNIDIKVNAQTKTHAAADVFQQAKNAKVSADRATLHVDRDLCDACGIKGGVGSLMRGVGISRLTVNSPSGRFEITASRPSVPRRING

SEQ ID NO: 123 - protein, 136 aa, Artificial Sequence
  Synthetic Peptide
GVGGAITATVGSTAGAAGRAAARAPSLPAYAGGKTSGVLRTTAGDTALLSGYKGPSASMPRGTPGMNGRIKSHVAAHAAAVMREQGMKEGTLYINRVPCSGATGCDAMLPRMLPPDAHLRVVGPNGYDQVFVGLPD

SEQ ID NO: 124 - protein, 150 aa, Artificial Sequence
  Synthetic Peptide
IQRFRRILNMPRYSLTNGRTGTVARVEVNGRRIFGVNTSLIKNSKYAPRDMDLRRRWLREVNWVPPKKNKPNHLGHAQSLSHAASHALIRAYERMERLGGQLPKKLTMVVDRPTCNICRGEMPALLKRLGIEELTIYSGGRDAIIIKAIK

SEQ ID NO: 125 - protein, 153 aa, Artificial Sequence
  Synthetic Peptide
GGSAVVGAGVVATGAKAVTTGKSLSESQATLSVAQRLLATIGEEGKTAGVLELDGELIPLVSGKSSLPNYAASGHVAGQAALIMRDRGATSGRLLIDNPSGICGYCKSQVATLLPENATLQVGTPLGTVTPSSRWSASRTFTGNDRDPKPWPR

SEQ ID NO: 126 - protein, 162 aa, Artificial Sequence
  Synthetic Peptide
SQFDNVRKDMGLPARIGDDDPYTTSVLRIDGHEYWGKNGKWVTKGKTSNYTDKAHYDKVRKELGTSAEVPGHAAGVAFNKAYQVRKNTGTKGGNAVLYVDKIPCVMCKPGIATLMRSAKVDHLDLHYLQDGKMHHVQYVRNPDTDAVYNPFSGKWTKPSKKK

SEQ ID NO: 127 - protein, 40 aa, Artificial Sequence
  Synthetic Peptide
TKSANSGGAAKDLAKYRERQGMPRAGSADDAHTAARLDVG

SEQ ID NO: 128 - protein, 51 aa, Artificial Sequence
  Synthetic Peptide
TKSANSGGAAKDLAKYRERQGMPRAGSADDAHTAARLDVGGRSFYGHNAHG

SEQ ID NO: 129 - protein, 80 aa, Artificial Sequence
  Synthetic Peptide
TKSANSGGAAKDLAKYRERQGMPRAGSADDAHTAARLDVGGRSFYGHNAHGRNIDIKVNAQTKTHAEADVFQQAKNAKVS

SEQ ID NO: 130 - protein, 99 aa, Artificial Sequence
  Synthetic Peptide
TKSANSGGAAKDLAKYRERQGMPRAGSADDAHTAARLDVGGRSFYGHNAHGRNIDIKVNAQTKTHAEADVFQQAKNAKVSADRATLHVDRDLCDACGIK

SEQ ID NO: 131 - protein, 110 aa, Artificial Sequence
  Synthetic Peptide
TKSANSGGAAKDLAKYRERQGMPRAGSADDAHTAARLDVGGRSFYGHNAHGRNIDIKVNAQTKTHAEADVFQQAKNAKVSADRATLHVDRDLCDACGIKGGVGSLMRGVG

SEQ ID NO: 132 - protein, 98 aa, Artificial Sequence
  Synthetic Peptide
GRSFYGHNAHGRNIDIKVNAQTKTHAEADVFQQAKNAKVSADRATLHVDRDLCDACGIKGGVGSLMRGVGISRLTVNSPSGRFEITASRPSVPRRING

SEQ ID NO: 133 - protein, 87 aa, Artificial Sequence
  Synthetic Peptide
RNIDIKVNAQTKTHAEADVFQQAKNAKVSADRATLHVDRDLCDACGIKGGVGSLMRGVGISRLTVNSPSGRFEITASRPSVPRRING

SEQ ID NO: 134 - protein, 39 aa, Artificial Sequence
  Synthetic Peptide
GGVGSLMRGVGISRLTVNSPSGRFEITASRPSVPRRING

SEQ ID NO: 135 - protein, 28 aa, Artificial Sequence
  Synthetic Peptide
ISRLTVNSPSGRFEITASRPSVPRRING

SEQ ID NO: 136 - protein, 31 aa, Artificial Sequence
  Synthetic Peptide
FSKAESGYIEIQRFRRILNMPRYSLTNGRTG

SEQ ID NO: 137 - protein, 40 aa, Artificial Sequence
  Synthetic Peptide
FSKAESGYIEIQRFRRILNMPRYSLTNGRTGTVARVEVNG

SEQ ID NO: 138 - protein, 86 aa, Artificial Sequence
  Synthetic Peptide
FSKAESGYIEIIQRFRRILNMPRYSLTNGRTGTVARVEVNGRRIFGVNTSLIKNSKYAPRDMDLRRRWLREVNWVPPKKNKPNHLG

SEQ ID NO: 139 - protein, 111 aa, Artificial Sequence
  Synthetic Peptide
FSKAESGYIEIIQRFRRILNMPRYSLTNGRTGTVARVEVNGRRIFGVNTSLIKNSKYAPRDMDLRRRWLREVNWVPPKKNKPNHLGHAQSLSHAESHALIRAYERMERLGG

SEQ ID NO: 140 - protein, 141 aa, Artificial Sequence
  Synthetic Peptide
FSKAESGYIEIIQRFRRILNMPRYSLTNGRTGTVARVEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLG

SEQ ID NO: 141 - protein, 129 aa, Artificial Sequence
  Synthetic Peptide
TVARVEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 142 - protein, 120 aa, Artificial Sequence
  Synthetic Peptide
RRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 143 - protein, 75 aa, Artificial Sequence
  Synthetic Peptide
HAQSLSHAESHALIRAYERMERLGGQLPKKLTMVVDRPTCNICRGEMPALLKRLGIEELTIYSGGRDAIIIKAIK

SEQ ID NO: 144 - protein, 50 aa, Artificial Sequence
  Synthetic Peptide
QLPKKLTMVVDRPTCNICRGEMPALLKRLGIEELTIYSGGRDAIIIKAIK

SEQ ID NO: 145 - protein, 20 aa, Artificial Sequence
  Synthetic Peptide
IEELTIYSGGRDAIIIKAIK

SEQ ID NO: 146 - protein, 33 aa, Artificial Sequence
  Synthetic Peptide
GVGGAITATVGSTAGAAGRAAARAPSLPAYAGG

SEQ ID NO: 147 - protein, 51 aa, Artificial Sequence
  Synthetic Peptide
GVGGAITATVGSTAGAAGRAAARAPSLPAYAGGKTSGVLRTTAGDTALLSG

SEQ ID NO: 148 - protein, 71 aa, Artificial Sequence
  Synthetic Peptide
GVGGAITATVGSTAGAAGRAAARAPSLPAYAGGKTSGVLRTTAGDTALLSGYKGPSASMPRGTPGMNGRIK

SEQ ID NO: 149 - protein, 101 aa, Artificial Sequence
  Synthetic Peptide
GVGGAITATVGSTAGAAGRAAARAPSLPAYAGGKTSGVLRTTAGDTALLSGYKGPSASMPRGTPGMNGRIKSHVEAHAAAVMREQGMKEGTLYINRVPCSG

SEQ ID NO: 150 - protein, 58 aa, Artificial Sequence
  Synthetic Peptide
ADRATLHVDRDLCDACGIKGGVGSLMRGVGISRLTVNSPSGRFEITASRPSVPRRING

SEQ ID NO: 151 - protein, 101 aa, Artificial Sequence
  Synthetic Peptide
KTSGVLRTTAGDTALLSGYKGPSASMPRGTPGMNGRIKSHVEAHAAAVMREQGMKEGTLYINRVPCSGATGCDAMLPRMLPPDAHLRVVGPNGYDQVFVGL

SEQ ID NO: 152 - protein, 83 aa, Artificial Sequence
  Synthetic Peptide
YKGPSASMPRGTPGMNGRIKSHVEAHAAAVMREQGMKEGTLYINRVPCSGATGCDAMLPRMLPPDAHLRVVGPNGYDQVFVGL

SEQ ID NO: 153 - protein, 63 aa, Artificial Sequence
  Synthetic Peptide
SHVEAHAAAVMREQGMKEGTLYINRVPCSGATGCDAMLPRMLPPDAHLRVVGPNGYDQVFVGL

SEQ ID NO: 154 - protein, 33 aa, Artificial Sequence
  Synthetic Peptide
ATGCDAMLPRMLPPDAHLRVVGPNGYDQVFVGL

SEQ ID NO: 155 - protein, 8 aa, Artificial Sequence
  Synthetic Peptide
YDQVFVGL

SEQ ID NO: 156 - protein, 141 aa, Artificial Sequence
  Synthetic Peptide
MPRYSLTNGRTGTVARVEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 157 - protein, 135 aa, Artificial Sequence
  Synthetic Peptide
TNGRTGTVARVEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 158 - protein, 130 aa, Artificial Sequence
  Synthetic Peptide
GTVARVEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 159 - protein, 125 aa, Artificial Sequence
  Synthetic Peptide
VEVNGRRIFGVNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 160 - protein, 120 aa, Artificial Sequence; identical to SEQ ID NO: 142
  Synthetic Peptide

SEQ ID NO: 161 - protein, 115 aa, Artificial Sequence
  Synthetic Peptide
VNTSLIKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 162 - protein, 110 aa, Artificial Sequence
  Synthetic Peptide
IKNSKYAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 163 - protein, 105 aa, Artificial Sequence
  Synthetic Peptide
YAPRDMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 164 - protein, 100 aa, Artificial Sequence
  Synthetic Peptide
MDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 165 - protein, 90 aa, Artificial Sequence
  Synthetic Peptide
VNWVPPKKNKPNHLGHAQSLSHAESHALIRAYERMERLGGQLPKKLTMVVDRPTCNICRGEMPALLKRLGIEELTIYSGGRDAIIIKAIK

SEQ ID NO: 166 - protein, 85 aa, Artificial Sequence
  Synthetic Peptide
PKKNKPNHLGHAQSLSHAESHALIRAYERMERLGGQLPKKLTMVVDRPTCNICRGEMPALLKRLGIEELTIYSGGRDAIIIKAIK

SEQ ID NO: 167 - protein, 65 aa, Artificial Sequence
  Synthetic Peptide
HALIRAYERMERLGGQLPKKLTMVVDRPTCNICRGEMPALLKRLGIEELTIYSGGRDAIIIKAIK

SEQ ID NO: 168 - protein, 33 aa, Artificial Sequence
  Synthetic Peptide
GSYTLGSYQISAPQLPAYNGQTVGTFYYVNGAG

SEQ ID NO: 169 - protein, 43 aa, Artificial Sequence
  Synthetic Peptide
GSYTLGSYQISAPQLPAYNGQTVGTFYYVNGAGGLESRTFSSG

SEQ ID NO: 170 - protein, 69 aa, Artificial Sequence
  Synthetic Peptide
GSYTLGSYQISAPQLPAYNGQTVGTFYYVNGAGGLESRTFSSGGPTPYPNYANAGHVEGQSALFMRDNG

SEQ ID NO: 171 - protein, 108 aa, Artificial Sequence
  Synthetic Peptide
GSYTLGSYQISAPQLPAYNGQTVGTFYYVNGAGGLESRTFSSGGPTPYPNYANAGHVEGQSALFMRDNGISDGLVFHNNPEGTCGFCVNMTETLLPENSKLTVVPPEG

SEQ ID NO: 172 - protein, 105 aa, Artificial Sequence
  Synthetic Peptide
GLESRTFSSGGPTPYPNYANAGHVEGQSALFMRDNGISDGLVFHNNPEGTCGFCVNMTETLLPENSKLTVVPPEGAIPVKRGATGETRTFTGNSKSPKSPVKGEC

SEQ ID NO: 173 - protein, 95 aa, Artificial Sequence
  Synthetic Peptide
GPTPYPNYANAGHVEGQSALFMRDNGISDGLVFHNNPEGTCGFCVNMTETLLPENSKLTVVPPEGAIPVKRGATGETRTFTGNSKSPKSPVKGEC

SEQ ID NO: 174 - protein, 72 aa, Artificial Sequence
  Synthetic Peptide
DNGISDGLVFHNNPEGTCGFCVNMTETLLPENSKLTVVPPEGAIPVKRGATGETRTFTGNSKSPKSPVKGEC

SEQ ID NO: 175 - protein, 30 aa, Artificial Sequence
  Synthetic Peptide
AIPVKRGATGETRTFTGNSKSPKSPVKGEC

SEQ ID NO: 176 - protein, 62 aa, Artificial Sequence
  Synthetic Peptide
GGSAVVGAGVVATGAKAVTTGKSLSESQATLSVAQRLLATIGEEGKTAGVLELDGELIPLVS

SEQ ID NO: 177 - protein, 127 aa, Artificial Sequence
  Synthetic Peptide
GGSAVVGAGVVATGAKAVTTGKSLSESQATLSVAQRLLATIGEEGKTAGVLELDGELIPLVSGKSSLPNYAASGHVEGQAALIMRDRGATSGRLLIDNPSGICGYCKSQVATLLPENATLQVGTPLG

SEQ ID NO: 178 - protein, 91 aa, Artificial Sequence
  Synthetic Peptide
GKSSLPNYAASGHVEGQAALIMRDRGATSGRLLIDNPSGICGYCKSQVATLLPENATLQVGTPLGTVTPSSRWSASRTFTGNDRDPKPWPR

SEQ ID NO: 179 - protein, 26 aa, Artificial Sequence
  Synthetic Peptide
TVTPSSRWSASRTFTGNDRDPKPWPR

SEQ ID NO: 180 - protein, 108 aa, Artificial Sequence
  Synthetic Peptide
HYDKVRKELGTSAEVPGHAEGVAFNKAYQVRKNTGTKGGNAVLYVDKIPCVMCKPGIATLMRSAKVDHLDLHYLQDGKMHHVQYVRNPDTDAVYNPFSGKWTKPSKKK

SEQ ID NO: 181 - protein, 123 aa, Artificial Sequence
  Synthetic Peptide
KWVTKGKTSNYTDKAHYDKVRKELGTSAEVPGHAEGVAFNKAYQVRKNTGTKGGNAVLYVDKIPCVMCKPGIATLMRSAKVDHLDLHYLQDGKMHHVQYVRNPDTDAVYNPFSGKWTKPSKKK

SEQ ID NO: 182 - protein, 29 aa, Artificial Sequence
  Synthetic Peptide
LCRAACSTGRRLGPVAGAAGSRHKHSLPD

SEQ ID NO: 183 - protein, 28 aa, Artificial Sequence
  Synthetic Peptide
SVLTPLLLRSLTGSARRLMVPRAQVHSK

SEQ ID NO: 184 - protein, 38 aa, Artificial Sequence
  Synthetic Peptide
GGGSGGSGEIAALEAKNAALKAEIAALEAKIAALKAGY

SEQ ID NO: 185 - protein, 37 aa, Artificial Sequence
  Synthetic Peptide
GGSGGSYKIAALKAENAALEAKIAALKAEIAALEAGC

SEQ ID NO: 186 - protein, 182 aa, Artificial Sequence
  Synthetic Peptide
KSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHS

SEQ ID NO: 187 - protein, 19 aa, Artificial Sequence
  Synthetic Peptide
ATNFSLLKQAGDVEENPGP

SEQ ID NO: 188 - DNA, 462 bp, Artificial Sequence
  Synthetic Peptide
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataa

SEQ ID NO: 189 - DNA, 816 bp, Artificial Sequence
  Synthetic Peptide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 190 - DNA, 225 bp, Artificial Sequence
  Synthetic Peptide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 191 - DNA, 16 bp, Artificial Sequence
  Synthetic Peptide
acacacacacacacac

SEQ ID NO: 192 - DNA, 16 bp, Artificial Sequence
  Synthetic Peptide
acgtgtacacgtacgt

SEQ ID NO: 193 - DNA, 17 bp, Artificial Sequence
  Synthetic Peptide
gcgcgcgcgcgcgcgcg

SEQ ID NO: 194 - DNA, 16 bp, Artificial Sequence
  Synthetic Peptide
ccggccggccggccgg

SEQ ID NO: 195 - DNA, 16 bp, Artificial Sequence
  Synthetic Peptide
tcgagatctcgatcga

SEQ ID NO: 196 - DNA, 16 bp, Artificial Sequence
  Synthetic Peptide
tctctctctctctctc

SEQ ID NO: 197 - DNA, 16 bp, Artificial Sequence
  Synthetic Peptide
cccccccccccccccc

SEQ ID NO: 198 - DNA, 16 bp, Artificial Sequence
  Synthetic Peptide
cccccccttttttccc

SEQ ID NO: 199 - DNA, 16 bp, Artificial Sequence
  Synthetic Peptide
cccccctttttttccc

SEQ ID NO: 200 - DNA, 16 bp, Artificial Sequence
  Synthetic Peptide
acgtacgtacgtacgt

SEQ ID NO: 201 - DNA, 16 bp, Artificial Sequence
  Synthetic Peptide
ccggccggccggccgg

SEQ ID NO: 202 - DNA, 16 bp, Artificial Sequence
  Synthetic Peptide
gcgcgcgcgcgcgcgc

SEQ ID NO: 203 - DNA, 16 bp, Artificial Sequence
  Synthetic Peptide
ctctctctctctctct

SEQ ID NO: 204 - DNA, 16 bp, Artificial Sequence
  Synthetic Peptide
tcgatcgatcgatcga

SEQ ID NO: 205 - DNA, 16 bp, Artificial Sequence
  Synthetic Peptide
cccccccttttttccc

SEQ ID NO: 206 - DNA, 16 bp, Artificial Sequence
  Synthetic Peptide
acgtgtatatgtacgt

SEQ ID NO: 207 - DNA, 16 bp, Artificial Sequence
  Synthetic Peptide
gcgcgcgcgtgcgcgc

SEQ ID NO: 208 - DNA, 16 bp, Artificial Sequence
  Synthetic Peptide
tctttttttttttctc

SEQ ID NO: 209 - DNA, 16 bp, Artificial Sequence
  Synthetic Peptide
tcgagatcttgatcga

SEQ ID NO: 210 - DNA, 16 bp, Artificial Sequence
  Synthetic Peptide
ccccccttttttcccc

SEQ ID NO: 211 - DNA, 16 bp, Artificial Sequence
  Synthetic Peptide
cccccccttttttttc

SEQ ID NO: 212 - DNA, 16 bp, Artificial Sequence
  Synthetic Peptide
gagagagagagagaga

SEQ ID NO: 213 - DNA, 16 bp, Artificial Sequence
  Synthetic Peptide
ccccccctttttcccc

SEQ ID NO: 214 - DNA, 15 bp, Artificial Sequence
  Synthetic Peptide
cccccccccttttcc

SEQ ID NO: 215 - DNA, 16 bp, Artificial Sequence
  Synthetic Peptide
acacacacatacacac

SEQ ID NO: 216 - DNA, 16 bp, Artificial Sequence
  Synthetic Peptide
ccggccggttggccgg

SEQ ID NO: 217 - DNA, 16 bp, Artificial Sequence
  Synthetic Peptide
tctttttttttttctc

SEQ ID NO: 218 - DNA, 16 bp, Artificial Sequence
  Synthetic Peptide
tctctctctttctctc

SEQ ID NO: 219 - DNA, 16 bp, Artificial Sequence
  Synthetic Peptide
gagaaaaaaaaagaga

SEQ ID NO: 220 - DNA, 16 bp, Artificial Sequence
  Synthetic Peptide
tcgagattttgatcga

SEQ ID NO: 221 - DNA, 16 bp, Artificial Sequence
  Synthetic Peptide
gggggggggggggggg

SEQ ID NO: 222 - DNA, 16 bp, Artificial Sequence
  Synthetic Peptide
ccccccccccccctcc

SEQ ID NO: 223 - DNA, 16 bp, Artificial Sequence
  Synthetic Peptide
ggaggggggggggggg

SEQ ID NO: 224 - DNA, 51 bp, Artificial Sequence
  Synthetic Peptide
  n (17)..(32): Any nucleotide
  misc_feature (17)..(32): n is a, c, g, or t
tctagcctagccgtttnnnnnnnnnnnnnnnnagggtgagcatcaaactca

SEQ ID NO: 225-229 - skipped (5 entries)

SEQ ID NO: 230 - DNA, 10 bp, Artificial Sequence
  Synthetic Peptide
cccccccccc

SEQ ID NO: 231 - DNA, 11 bp, Artificial Sequence
  Synthetic Peptide
ccccccccccc

SEQ ID NO: 232 - DNA, 12 bp, Artificial Sequence
  Synthetic Peptide
cccccccccccc

SEQ ID NO: 233 - DNA, 13 bp, Artificial Sequence
  Synthetic Peptide
ccccccccccccc

SEQ ID NO: 234 - DNA, 14 bp, Artificial Sequence
  Synthetic Peptide
cccccccccccccc

SEQ ID NO: 235 - DNA, 15 bp, Artificial Sequence
  Synthetic Peptide
ccccccccccccccc

SEQ ID NO: 236 - DNA, 16 bp, Artificial Sequence
  Synthetic Peptide
cccccccccccccccc

SEQ ID NO: 237 - DNA, 17 bp, Artificial Sequence
  Synthetic Peptide
ccccccccccccccccc

SEQ ID NO: 238 - DNA, 18 bp, Artificial Sequence
  Synthetic Peptide
cccccccccccccccccc

SEQ ID NO: 239 - DNA, 19 bp, Artificial Sequence
  Synthetic Peptide
ccccccccccccccccccc

SEQ ID NO: 240 - DNA, 20 bp, Artificial Sequence
  Synthetic Peptide
cccccccccccccccccccc

SEQ ID NO: 241 - DNA, 14 bp, Artificial Sequence
  Synthetic Peptide
cccccctttttccc

SEQ ID NO: 242 - DNA, 16 bp, Artificial Sequence
  Synthetic Peptide
ccccccctttttcccc

SEQ ID NO: 243 - DNA, 17 bp, Artificial Sequence
  Synthetic Peptide
cccccccctttttcccc

SEQ ID NO: 244 - DNA, 19 bp, Artificial Sequence
  Synthetic Peptide
cccccccctttttttcccc

SEQ ID NO: 245 - DNA, 20 bp, Artificial Sequence
  Synthetic Peptide
ccccccccccctttcccccc

SEQ ID NO: 246 - DNA, 15 bp, Artificial Sequence
  Synthetic Peptide
cccccctttttcccc

SEQ ID NO: 247 - DNA, 15 bp, Artificial Sequence
  Synthetic Peptide
ccccccccctttccc

SEQ ID NO: 248 - DNA, 16 bp, Artificial Sequence
  Synthetic Peptide
ccccccccctttcccc

SEQ ID NO: 249 - DNA, 18 bp, Artificial Sequence
  Synthetic Peptide
ccccccccctttttcccc

SEQ ID NO: 250 - DNA, 18 bp, Artificial Sequence
  Synthetic Peptide
cccccccccttttccccc

SEQ ID NO: 251 - DNA, 16 bp, Artificial Sequence
  Synthetic Peptide
ccccccctttcccccc

SEQ ID NO: 252 - DNA, 16 bp, Artificial Sequence
  Synthetic Peptide
cccccctttttttccc

SEQ ID NO: 253 - DNA, 16 bp, Artificial Sequence
  Synthetic Peptide
cccccccttttttttc

SEQ ID NO: 254 - DNA, 16 bp, Artificial Sequence
  Synthetic Peptide
ttttttttttttcccc

SEQ ID NO: 255 - DNA, 15 bp, Artificial Sequence
  Synthetic Peptide
gtaggagcaacataa

SEQ ID NO: 256 - DNA, 15 bp, Artificial Sequence
  Synthetic Peptide
cgtaggagcaacata

SEQ ID NO: 257 - DNA, 15 bp, Artificial Sequence
  Synthetic Peptide
tcgtaggagcaacat

SEQ ID NO: 258 - DNA, 15 bp, Artificial Sequence
  Synthetic Peptide
ttcgtaggagcaaca

SEQ ID NO: 259 - DNA, 15 bp, Artificial Sequence
  Synthetic Peptide
attcgtaggagcaac

SEQ ID NO: 260 - DNA, 16 bp, Artificial Sequence
  Synthetic Peptide
tcgtaggagtaaacat

SEQ ID NO: 261 - DNA, 16 bp, Artificial Sequence
  Synthetic Peptide
actcaatcctctgatc

SEQ ID NO: 262 - DNA, 15 bp, Artificial Sequence
  Synthetic Peptide
cattagtagaacgca

SEQ ID NO: 263 - DNA, 15 bp, Artificial Sequence
  Synthetic Peptide
cattagtaaaacgca

SEQ ID NO: 264 - DNA, 4262 bp, Artificial Sequence
  Synthetic Peptide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagcagagctggtttagtggatatcttaagccaccatggcctctgtcctgacgccactgctgctgaggagcctgaccggctcggcccggcggctcatggtgccgcgggctcaggtccactcgaagtctagagatatcgccgacctcagaaccctgggttacagtcagcagcaacaggagaagataaaacctaaggtgcgctccactgttgctcaacatcatgaggcattggtgggccacggatttacacacgcccatatagtagccttgtcccaacaccccgctgctcttggtactgttgctgtaaaatatcaagacatgatagcagcattgcctgaagccactcacgaggctatcgttggagtaggaaagtatcatggggctcgcgcacttgaggctttgctcaccgttgcaggtgaacttcgaggcccacctcttcagctcgacaccggacaattgctcaagattgccaagcgagggggggtcaccgccgtagaagccgtccatgcttggcgcaacgcactcactggggcccccctgaacttaacgcccgagcaggtggttgctatagcgtcgcacgatggcggtaagcaagcccttgaaacagttcaggccttgttacctgtcttatgccaggcacatggactgactcctgaacaggtagttgcgattgcctcacatgacggaggtaaacaagctttagaaacagtgcaggctttgctcccggttctttgtcaggcgcatggcttgactccggaacaggttgtcgctattgcttcacacgatgggggtaaacaagccctcgaaacagtgcaagcccttttaccggtcctatgccacgcacacggtttgacaccagaacaggtagtagctatagcctcgaatattggtggtaagcaagccttagagaccgtgcagcggttactgcctgtactgtgtcaagctcacgggcttacacctgagcaagtagttgcaatagcaagtcacgacggcggtaaacaagccttggagaccgttcaagctctccttccagtattgtgtcaagcacatggcctaactcccgagcaggtagtggctatcgctagtaacggtggtgggaaacaggcactagagacagttcaagctctacttccagtgttgtgccaggctcacgggctcacaccccaacaagttgtcgccatcgccagtaatggaggtggaaagcaggccctcgaaaccgtgcaacggctccttccagtgctctgccaagcgcatggacttacgccagagcaggtggtggcaatagcctcgcatgacggcggcaagcaggcgttggagaccgtccaagcattgctgccagttttatgtcaggcacatggtttaacaccacaacaggtagtcgcaatagctagcaacaatggcggaaaacaggctctggaaactgtccaacgattgctacccgttctgtgtcaggcccatggattgacgccgcaacaagtggtcgcgattgcgagtcacgacggaggtaaacaggccctggaaacggtgcagagactactccccgtcctctgccaagcccacggtctcacgcctgagcaggtagtagcgatagcatctcacgacggtggtaagcaagcgttagagacagtacaagcgttactaccagttctctgtcaagctcatgggctaacgccggaacaggttgtcgctattgcaagcaacatcggcgggaaacaggcattagagacggtccaagcgctgttgcccgtactgtgtcaggcgcatggtctgacaccggagcaagttgtggccatcgcgtccaacggtggtggtaaacaggcattggaaaccgtacaggcgcttttgcctgtgctttgtcaagcgcacggacttactccggaacaggtagtggcgatcgcaagccatgatggaggaaaacaagcacttgagactgttcaaagattattgccagtgctatgtcaagcacacggtcttaccccagaacaggtcgtagccatagcttctaatattggaggcaaacaagccttagaaacagtccaagctttattacccgtgttatgtcaggctcacggcctcactcccgaacaagtcgttgccattgcatcgaacggcggtggaaagcaagctctggagacggtacaacgtttgcttccggtactttgccaggcacacggattaacgcccgagcaggtggttgctatagcgtcgaacattggcggtaagcaagcccttgaaacagttcaggccttgttacctgtcttatgccaggcacatggactgacgcctcagcaagtagtggctattgcttccaacggcggcggacgcccagcactcgagagtatcgtagcacagctcagtcgcccagatcccgccttggctgccctcaccaatgatcaccttgtggcactcgcttgccttgggggtcgccctgctctggatgcagttaagaaaggcctaggcggcagcttcagcaaagcggaatctgggtatattgagatacaacgcttcaggagaattctcaacatgccccgctattcacttacgaatggccgtactggtacggtggcgcgtgtggaggtaaacgggcgtcgcattttcggggttaatacttcgttgattaagaactctaagtatgctccgcgcgacatggacttacgccgccgttggctgcgcgaggttaactgggtgcccccaaaaaaaaacaaaccaaaccacttaggacacgcgcagagcctgtcgcacgccgcatcccacgctttgatccgcgcatacgaacgtatggagcgtcttgggggtcagttaccaaagaaacttactatggtagtcgatcgccccacctgcaatatctgtcgcggggagatgcccgcgctactaaagcgcctggggattgaagaacttaccatctattcaggtggccgcgatgcaatcatcattaaggcgattaagtccggagggtcgactaatctgagcgacattatagaaaaagaaacaggtaagcagttggtcatccaagagagtattttgatgctgccagaggaagtcgaggaggtaattggtaacaaaccagagagtgacattcttgtgcataccgcttatgacgagtcaactgacgagaatgttatgctcttgacctctgatgcacccgaatacaaaccttgggcactcgttatccaggacagtaatggagaaaataaaataaaaatgttgtaatgagctcggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 265 - protein, 1057 aa, Artificial Sequence
  Synthetic Peptide
MASVLTPLLLRSLTGSARRLMVPRAQVHSKSRDIADLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASHDGGKQALETVQALLPVLCQAHGLTPEQVVAIASHDGGKQALETVQALLPVLCQAHGLTPEQVVAIASHDGGKQALETVQALLPVLCHAHGLTPEQVVAIASNIGGKQALETVQRLLPVLCQAHGLTPEQVVAIASHDGGKQALETVQALLPVLCQAHGLTPEQVVAIASNGGGKQALETVQALLPVLCQAHGLTPQQVVAIASNGGGKQALETVQRLLPVLCQAHGLTPEQVVAIASHDGGKQALETVQALLPVLCQAHGLTPQQVVAIASNNGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNIGGKQALETVQALLPVLCQAHGLTPEQVVAIASNGGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGGSFSKAESGYIEIQRFRRILNMPRYSLTNGRTGTVARVEVNGRRIFGVNTSLIKNSKYAPRDMDLRRRWLREVNWVPPKKNKPNHLGHAQSLSHAASHALIRAYERMERLGGQLPKKLTMVVDRPTCNICRGEMPALLKRLGIEELTIYSGGRDAIIIKAIKSGGSTNLSDIIEKETGKQLVIQESILMLPEEVEEVIGNKPESDILVHTAYDESTDENVMLLTSDAPEYKPWALVIQDSNGENKIKML

SEQ ID NO: 266 - DNA, 3680 bp, Artificial Sequence
  Synthetic Peptide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagcagagctggtttagtggatatcttaagccaccatggcctctgtcctgacgccactgctgctgaggagcctgaccggctcggcccggcggctcatggtgccgcgggctcaggtccactcgaagtctagatccactgctttcgttgatcaggacaaacagatggccaaccgtctgaacctgtctccgctggaacgctccaaaatcgagaaacagtacggcggtgccactaccctggccttcatttctaacaagcaaaatgaactggcgcagatcctgagccgcgcggatatcctgaagatcgcgtcttatgattgcgcggcacacgcgttgcaggctgttctggattgcggcccgatgctgggcaagcgtggcttttcccaatctgacatcgtcaagattgcgggcaatggtggcggtgcccaggctctgcaggcagttctggatctggaaagcatgctgggtaaacgcggtttcagccgtgatgacatagcgaaaatggcaggtaacggcggcggtgcacaaactctgcaagccgtactggatctggagtccgcgtttagagagcgtggcttttctcaagcagacattgtaaagatagcgggcaacaatgggggtgctcaagcactatatagcgtcctggacgtagagccgaccctgggtaaacgtggtttctcacgtgctgacatcgtgaagatcgccggcaacatcggtggcgcccaggccctgcacactgtgcttgatctggagcctgcactaggaaaacgaggattttcccgtattgacatcgttaaaatcgcggccaacaatggtggcgcgcaagcattgcacgctgttttagacctgggtccgacgctgcgtgagtgtggtttcagtcaggcgaccatcgcgaagattgctggtaataatggaggagcacaagcactgcaaatggtacttgacctgggacccgcattaggcaaaaggggcttctcccaggcaactattgctaaaattgctggtaacaatggaggggctcaagcactgcagaccgttcttgacctggaaccggctctgtgcgagcgtggttttggccaagcaacaattgccaaaatggctggaaatatcgggggtgcgcaggcattacaaacagtattggatttagaaccagcgctgcgaaaacgagacttcagacaggccgatattataaaaattgcgggaaatattggtggagctcaggctctacaggcggttattgaacacggaccgactttgagacaacatggctttaacctggcggacatcgtgaaaatggctgggaacaatggcggggcccaagcgcttcaggccgtcttagatttaaaacccgtcttggatgagcacggcttcagccaggctgacatcgtcaaaatcgcaggcaatatcggtgggacccaagcgctgcatgcggtgctggatttggagcgtatgctgggggagcgcggtttcagcagagcagacatcgtgaatgtggcgggaaacattggtggtgcacaggctctaaaggcggtattagagcatgaagctactcttaatgaaagaggattctcccgcgccgacatcgttaaaatcgctggcaacggtggcggtgcccaagctcttaaagcagttcttgagcacgaggcaacactggatgaacgcggtttctcgcgcgcggatattgtaaatgttgccgggaacaacggaggcgcacaggcgctgaaagcagtgttggaacacgaggcgacgttaaacgaacgtgggtttaatctgacagacatcgtggagatggctgctaacggcggtggcgcacaggcattaaaggctgtccttgagcatggtccgacccttcgccagcgcggcttgagcttgattgacattgtcgaaattgccgggaatggcggaggagcacaagcgttgaaagcagtcttaaagtatggaccggtccttatgcaggccggccgtagtaatgaagaaatcgtccacgtagcggcgcgacgtggtggagcaggtcgtattcgtaaaatggtagctccgctgctcgagcgtcagggcctaggcggcagcatggacttgaggagacgctggctgcgggaggtgaattgggtgcctccgaagaaaaataagccaaaccacctgggccacgctcagtccctttctcacgctgaatctcacgccctgattagagcttatgaacgcatggagcgcctcgggggccaactgcctaagaaactgacaatggtggttgaccgccctacttgtaacatttgcaggggcgagatgcctgccctcctgaaacgcttgggcattgaagagctgaccatctactccggcgggcgcgacgccatcattatcaaggccatcaaatccggagggtcgactaatctgagcgacattatagaaaaagaaacaggtaagcagttggtcatccaagagagtattttgatgctgccagaggaagtcgaggaggtaattggtaacaaaccagagagtgacattcttgtgcataccgcttatgacgagtcaactgacgagaatgttatgctcttgacctctgatgcacccgaatacaaaccttgggcactcgttatccaggacagtaatggagaaaataaaataaaaatgttgtaatgagctcggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 267 - protein, 863 aa, Artificial Sequence
  Synthetic Peptide
MASVLTPLLLRSLTGSARRLMVPRAQVHSKSRSTAFVDQDKQMANRLNLSPLERSKIEKQYGGATTLAFISNKQNELAQILSRADILKIASYDCAAHALQAVLDCGPMLGKRGFSQSDIVKIAGNGGGAQALQAVLDLESMLGKRGFSRDDIAKMAGNGGGAQTLQAVLDLESAFRERGFSQADIVKIAGNNGGAQALYSVLDVEPTLGKRGFSRADIVKIAGNIGGAQALHTVLDLEPALGKRGFSRIDIVKIAANNGGAQALHAVLDLGPTLRECGFSQATIAKIAGNNGGAQALQMVLDLGPALGKRGFSQATIAKIAGNNGGAQALQTVLDLEPALCERGFGQATIAKMAGNIGGAQALQTVLDLEPALRKRDFRQADIIKIAGNIGGAQALQAVIEHGPTLRQHGFNLADIVKMAGNNGGAQALQAVLDLKPVLDEHGFSQADIVKIAGNIGGTQALHAVLDLERMLGERGFSRADIVNVAGNIGGAQALKAVLEHEATLNERGFSRADIVKIAGNGGGAQALKAVLEHEATLDERGFSRADIVNVAGNNGGAQALKAVLEHEATLNERGFNLTDIVEMAANGGGAQALKAVLEHGPTLRQRGLSLIDIVEIAGNGGGAQALKAVLKYGPVLMQAGRSNEEIVHVAARRGGAGRIRKMVAPLLERQGLGGSMDLRRRWLREVNWVPPKKNKPNHLGHAQSLSHAESHALIRAYERMERLGGQLPKKLTMVVDRPTCNICRGEMPALLKRLGIEELTIYSGGRDAIIIKAIKSGGSTNLSDIIEKETGKQLVIQESILMLPEEVEEVIGNKPESDILVHTAYDESTDENVMLLTSDAPEYKPWALVIQDSNGENKIKML

SEQ ID NO: 268 - DNA, 36 bp, Artificial Sequence
  Synthetic Peptide
aaaaaaaaaaaaaaatgcgccaaaaaaaaaaaaaaa

SEQ ID NO: 269 - DNA, 32 bp, Artificial Sequence
  Synthetic Peptide
aaaaaaaaaaaaaaaacaaaaaaaaaaaaaaa

SEQ ID NO: 270 - DNA, 32 bp, Artificial Sequence
  Synthetic Peptide
aaaaaaaaaaaaaaaccaaaaaaaaaaaaaaa

SEQ ID NO: 271 - DNA, 32 bp, Artificial Sequence
  Synthetic Peptide
aaaaaaaaaaaaaaagcaaaaaaaaaaaaaaa

SEQ ID NO: 272 - DNA, 32 bp, Artificial Sequence
  Synthetic Peptide
aaaaaaaaaaaaaaatcaaaaaaaaaaaaaaa

SEQ ID NO: 273 - DNA, 37 bp, Artificial Sequence
  Synthetic Peptide
aaaaaaaaaaaaaaaacccctcaaaaaaaaaaaaaaa

SEQ ID NO: 274 - DNA, 16 bp, Artificial Sequence
  Synthetic Peptide
gtacaccatccgtccc

SEQ ID NO: 275 - DNA, 16 bp, Artificial Sequence
  Synthetic Peptide
gtgttctctatttcac

SEQ ID NO: 276 - DNA, 16 bp, Artificial Sequence
  Synthetic Peptide
gtacaccatttgtccc

SEQ ID NO: 277 - DNA, 16 bp, Artificial Sequence
  Synthetic Peptide
gtacaccatttgttcc

SEQ ID NO: 278 - DNA, 12 bp, Artificial Sequence
  Synthetic Peptide
ccgtcggaccgc

SEQ ID NO: 279 - DNA, 12 bp, Artificial Sequence
  Synthetic Peptide
ccgtcggaccgc

SEQ ID NO: 280 - DNA, 38 bp, Artificial Sequence
  Synthetic Peptide
ctaacttaccatgattaatttaagaattctcatcgtca

SEQ ID NO: 281 - DNA, 38 bp, Artificial Sequence
  Synthetic Peptide
ttaatttactatgattaatttaagaattcttattgtta

SEQ ID NO: 282 - DNA, 38 bp, Artificial Sequence
  Synthetic Peptide
ctaatttaccataattaatttaagaattcttatcgtta

SEQ ID NO: 283 - DNA, 38 bp, Artificial Sequence
  Synthetic Peptide
ctaacttatcataattaatttaaaaattcttatcgtca

SEQ ID NO: 284 - DNA, 38 bp, Artificial Sequence
  Synthetic Peptide
gattgaatggtactgatcagatcctcaagagtagcagt

SEQ ID NO: 285 - DNA, 38 bp, Artificial Sequence
  Synthetic Peptide
gattgaatggtactgattagatttttaagagtagcagt

SEQ ID NO: 286 - DNA, 32 bp, Artificial Sequence; identical to SEQ ID NO: 272
  Synthetic peptide
  c (17)..(17): 5-methylcytidine

SEQ ID NO: 287 - DNA, 14 bp, Artificial Sequence
  Synthetic Peptide
atccgatcagagct

SEQ ID NO: 288 - DNA, 32 bp, Artificial Sequence; identical to SEQ ID NO: 272
  synthetic peptide
  c (17)..(17): 5-hydroxymethylcytosine

SEQ ID NO: 289 - DNA, 14 bp, Artificial Sequence
  Synthetic Peptide
atttgattagagtt

SEQ ID NO: 290 - DNA, 14 bp, Artificial Sequence
  Synthetic Peptide
taggctagttttga

SEQ ID NO: 291 - DNA, 57 bp, Artificial Sequence
  Synthetic Peptide
atgaatcggccaacgcgcggggagaggcggtttgcgtattgggcgccagggtggttt

SEQ ID NO: 292 - DNA, 57 bp, Artificial Sequence
  Synthetic Peptide
atgaatcggtcaatgcgtggggagaggtggtttgtgtattgggtgccagggtggttt

SEQ ID NO: 293 - protein, 15 aa, Artificial Sequence
  Synthetic peptide
DIVGVKQWSGARALE

SEQ ID NO: 294 - protein, 15 aa, Artificial Sequence
  Synthetic peptide
DIVGVKSRSGARALE

SEQ ID NO: 295 - protein, 15 aa, Artificial Sequence
  Synthetic peptide
DIVGVKRGAGARALE

SEQ ID NO: 296 - protein, 14 aa, Artificial Sequence
  Synthetic peptide
DIVGVKYHGARALE

SEQ ID NO: 297 - DNA, 16 bp, Artificial Sequence
  Synthetic peptide
cccccccctttttttc